SEQUENCE LISTING (130 sequences)

SEQ ID NO: 1 - protein, 121 aa, synthetic construct
  source 1..121: note = NJO60988.1 hypothetical protein [Richelia sp. RM2_1_2]
RSPNSVHANSIINTTLGQIAVEDLFHSAPIKWQDGEKEYAVDERVQVATFDPDDNIDKFEQINYIYRHRVNKEAWRITDEDGNEIIITEDHSVMIERNGEIIAVKPTEILEDDLLIGVNDA

SEQ ID NO: 2 - protein, 47 aa, synthetic construct
  source 1..47: note = NJO60986.1 hypothetical protein [Richelia sp. RM2_1_2]
RSPCMIKKTKIKKVEKLPNFQNEYVYDIGMRGPNPYFFANNILVHNS

SEQ ID NO: 3 - protein, 115 aa, synthetic construct
  source 1..115: note = Candidatus MBC cysteine-less split-intein system
INGDTIHKTNWGELTVEELFNRGTRYWSEDNSKEYSANDELKVLTFDPVKDEAYYGNINYIYRHKVSKEQWEIEDEAGNTIRVTGDHSIMIERDGQLMDVKPRDMLDTDVLIVVD

SEQ ID NO: 4 - protein, 44 aa, synthetic construct
  source 1..44: note = Candidatus MBC cysteine-less split-intein system
ICVKRSKIKSIKQLDDFNDEYVYDIGIKGDTPYFFGNNILVHNS

SEQ ID NO: 5 - protein, 116 aa, synthetic construct
  source 1..116: note = Pseudomonas QCG cysteine-less split-intein system
INSVDGSTILNTSLGKITIEELFNVSDKHVVHAEKEFASNEDVMVMSWDNSAKQPYMGHINYVYRHEVEKELFEIEDNSGNKVIVTEDHSIMVIRNAELLEVKPTDLTDSDIILSI

SEQ ID NO: 6 - protein, 42 aa, synthetic construct
  source 1..42: note = Pseudomonas QCG cysteine-less split-intein system
ICLGKVSKVTNLGKKKQYVYDIGMKNPDNPYFFGNNILVHNS

SEQ ID NO: 7 - protein, 118 aa, synthetic construct
  source 1..118: note = Synthetic Chimera N-terminal
SETGDTIHKTNWGELTVEELFNRGTRYWSEDNSKEYSANDELKVLTFDPVKDEAYYGNINYIYRHKVSKEQWEIEDEAGNTIRVTGDHSIMIERDGQLMDVKPRDMLDTDVLIVVDHN

SEQ ID NO: 8 - protein, 45 aa, synthetic construct
  source 1..45: note = Synthetic Chimera C-terminal
MKVKRSKIKSIKQLDDFNDEYVYDIGMKNPDNPYFFGNNILVHNS

SEQ ID NO: 9 - protein, 14 aa, synthetic construct
  source 1..14: note = Proline that increases activity
NPYFFANNILVHNS

SEQ ID NO: 10 - protein, 14 aa, synthetic construct
  VARIANT 1: note = X is selected independently from any amino acid or X is selected from N or T
  VARIANT 6: note = X is selected independently from any amino acid or X is A or G
XPYFFXNNILVHNS

SEQ ID NO: 11 - protein, 21 aa, synthetic construct
  source 1..21: note = Gly-Ser linker
GSGSGSGSGSGSGSGSGSGSG

SEQ ID NO: 12 - protein, 18 aa, synthetic construct
  source 1..18: note = Asparagine-Serine linker
ASASASASASASASASAS

SEQ ID NO: 13 - protein, 59 aa, synthetic construct
  source 1..59: note = N-terminus Rsp
SVHANSIINTTLGQIAVEDLFHSAPIKWQDGEKEYAVDERVQVATFDPDDNIDKFEQIN

SEQ ID NO: 14 - protein, 59 aa, synthetic construct
  source 1..59: note = N-terminus Pae
SVDGSTILNTSLGKITIEELFNVSDKHVVHAEKEFASNEDVMVMSWDNSAKQPYMGHIN

SEQ ID NO: 15 - protein, 60 aa, synthetic construct
  source 1..60: note = N-terminus Cand
SETGDTIHKTNWGELTVEELFNRGTRYWSEDNSKEYSANDELKVLTFDPVKDEAYYGNIN

SEQ ID NO: 16 - protein, 58 aa, synthetic construct
  source 1..58: note = N-terminus Rsp
YIYRHRVNKEAWRITDEDGNEIIITEDHSVMIERNGEIIAVKPTEILEDDLLIGVNDA

SEQ ID NO: 17 - protein, 58 aa, synthetic construct
  source 1..58: note = N-terminus Pae
YVYRHEVEKELFEIEDNSGNKVIVTEDHSIMVIRNAELLEVKPTDLTDSDIILSIVYE

SEQ ID NO: 18 - protein, 58 aa, synthetic construct
  source 1..58: note = N-terminus Cand
YIYRHKVSKEQWEIEDEAGNTIRVTGDHSIMIERDGQLMDVKPRDMLDTDVLIVVDHN

SEQ ID NO: 19 - protein, 43 aa, synthetic construct
  source 1..43: note = C-terminus Rsp
MIKKTKIKKVEKLPNFQNEYVYDIGMRGPNPYFFANNILVHNS

SEQ ID NO: 20 - protein, 47 aa, synthetic construct
  source 1..47: note = C-terminus Pae
MNKFNVSLGKVSKVTNLGKKKQYVYDIGMKNPDNPYFFGNNILVHNS

SEQ ID NO: 21 - protein, 44 aa, synthetic construct
  source 1..44: note = Cand
MKVKRSKIKSIKQLDDFNDEYVYDIGIKGDTPYFFGNNILVHNS

SEQ ID NO: 22 - protein, 43 aa, synthetic construct; identical to SEQ ID NO: 19
  source 1..43: note = C-terminal intein half of Rsp

SEQ ID NO: 23 - protein, 47 aa, synthetic construct; identical to SEQ ID NO: 20
  source 1..47: note = C-terminal intein half of Pae

SEQ ID NO: 24 - protein, 44 aa, synthetic construct; identical to SEQ ID NO: 21
  source 1..44: note = C-terminal intein half of Cand

SEQ ID NO: 25 - protein, 40 aa, synthetic construct
  source 1..40: note = C-terminal intein half of Aes
MIEFIEFEIEDLGVMEIDVYDIEVDGNHNFFGNDILVHNS

SEQ ID NO: 26 - protein, 38 aa, synthetic construct
  source 1..38: note = C-terminal intein half of GP41-1
MMLKKILKIEELDERELIDIEVSGNHLFYANDILTHNS

SEQ ID NO: 27 - protein, 44 aa, synthetic construct; identical to SEQ ID NO: 21

SEQ ID NO: 28 - protein, 47 aa, synthetic construct; identical to SEQ ID NO: 20

SEQ ID NO: 29 - protein, 45 aa, synthetic construct
MKVKRSKISKIKQLDDFNDEYVYDIGMKNPDNPYFFGNNILVHNS

SEQ ID NO: 30 - protein, 4 aa, synthetic construct
  source 1..4: note = Gly-Ser Linker
GSGS

SEQ ID NO: 31 - protein, 6 aa, synthetic construct
  source 1..6: note = Gly-Ser linker
GSGSGS

SEQ ID NO: 32 - protein, 8 aa, synthetic construct
  source 1..8: note = Gly-Ser linker
GSGSGSGS

SEQ ID NO: 33 - protein, 10 aa, synthetic construct
  source 1..10: note = Gly-Ser linker
GSGSGSGSGS

SEQ ID NO: 34 - protein, 12 aa, synthetic construct
  source 1..12: note = Gly-Ser linker
GSGSGSGSGSGS

SEQ ID NO: 35 - protein, 14 aa, synthetic construct
  source 1..14: note = Gly-Ser linker
GSGSGSGSGSGSGS

SEQ ID NO: 36 - protein, 16 aa, synthetic construct
  source 1..16: note = Gly-Ser linker
GSGSGSGSGSGSGSGS

SEQ ID NO: 37 - protein, 18 aa, synthetic construct
  source 1..18: note = Gly-Ser linker
GSGSGSGSGSGSGSGSGS

SEQ ID NO: 38 - protein, 20 aa, synthetic construct
  source 1..20: note = Gly-Ser linker
GSGSGSGSGSGSGSGSGSGS

SEQ ID NO: 39 - protein, 33 aa, synthetic construct
  source 1..33: note = linker
GAAPAAAPAKQEAAAPAPAAKAEAPAAAPAAKA

SEQ ID NO: 40 - protein, 15 aa, synthetic construct
  source 1..15: note = Gly-Ser linker
GGGGSGGGGSGGGGS

SEQ ID NO: 41 - protein, 8 aa, synthetic construct
  source 1..8: note = Gly linker
GGGGGGGG

SEQ ID NO: 42 - protein, 6 aa, synthetic construct
  source 1..6: note = Gly linker
GGGGGG

SEQ ID NO: 43 - protein, 15 aa, synthetic construct
  source 1..15: note = linker
EAAAKEAAAKEAAAK

SEQ ID NO: 44 - protein, 5 aa, synthetic construct
  source 1..5: note = linker
EAAAK

SEQ ID NO: 45 - protein, 10 aa, synthetic construct
  source 1..10: note = linker
EAAAKEAAAK

SEQ ID NO: 46 - protein, 46 aa, synthetic construct
  source 1..46: note = linker
AEAAAKEAAAKEAAAKEAAAKALEAEAAAKEAAAKEAAAKEAAAKA

SEQ ID NO: 47 - protein, 5 aa, synthetic construct
  source 1..5: note = Gly-Ser linker
GGGGS

SEQ ID NO: 48 - protein, 5 aa, synthetic construct
  source 1..5: note = PAPAP
PAPAP

SEQ ID NO: 49 - protein, 12 aa, synthetic construct
  source 1..12: note = linker
AEAAAKEAAAKA

SEQ ID NO: 50 - protein, 10 aa, synthetic construct
  source 1..10: note = Gly-Ser linker
GGGGSGGGGS

SEQ ID NO: 51 - protein, 20 aa, synthetic construct
  source 1..20: note = Gly-Ser linker
GGGGSGGGGSGGGGSGGGGS

SEQ ID NO: 52 - protein, 25 aa, synthetic construct
  source 1..25: note = Gly-Ser linker
GGGGSGGGGSGGGGSGGGGSGGGGS

SEQ ID NO: 53 - protein, 30 aa, synthetic construct
  source 1..30: note = Gly-Ser linker
GGGGSGGGGSGGGGSGGGGSGGGGSGGGGS

SEQ ID NO: 54 - protein, 35 aa, synthetic construct
  source 1..35: note = Gly-Ser linker
GGGGSGGGGSGGGGSGGGGSGGGGSGGGGSGGGGS

SEQ ID NO: 55 - protein, 40 aa, synthetic construct
  source 1..40: note = Gly-Ser linker
GGGGSGGGGSGGGGSGGGGSGGGGSGGGGSGGGGSGGGGS

SEQ ID NO: 56 - protein, 45 aa, synthetic construct
  source 1..45: note = Gly-Ser linker
GGGGSGGGGSGGGGSGGGGSGGGGSGGGGSGGGGSGGGGSGGGGS

SEQ ID NO: 57 - protein, 50 aa, synthetic construct
  source 1..50: note = Gly-Ser linker
GGGGSGGGGSGGGGSGGGGSGGGGSGGGGSGGGGSGGGGSGGGGSGGGGS

SEQ ID NO: 58 - protein, 20 aa, synthetic construct
  source 1..20: note = Ala-Pro linker
APAPAPAPAPAPAPAPAPAP

SEQ ID NO: 59 - protein, 22 aa, synthetic construct
  source 1..22: note = Ala-Pro linker
APAPAPAPAPAPAPAPAPAPAP

SEQ ID NO: 60 - protein, 24 aa, synthetic construct
  source 1..24: note = Ala-Pro linker
APAPAPAPAPAPAPAPAPAPAPAP

SEQ ID NO: 61 - protein, 26 aa, synthetic construct
  source 1..26: note = Ala-Pro linker
APAPAPAPAPAPAPAPAPAPAPAPAP

SEQ ID NO: 62 - protein, 28 aa, synthetic construct
  source 1..28: note = Ala-Pro linker
APAPAPAPAPAPAPAPAPAPAPAPAPAP

SEQ ID NO: 63 - protein, 30 aa, synthetic construct
  source 1..30: note = Ala-Pro linker
APAPAPAPAPAPAPAPAPAPAPAPAPAPAP

SEQ ID NO: 64 - protein, 32 aa, synthetic construct
  source 1..32: note = Ala-Pro linker
APAPAPAPAPAPAPAPAPAPAPAPAPAPAPAP

SEQ ID NO: 65 - protein, 34 aa, synthetic construct
  source 1..34: note = Ala-Pro linker
APAPAPAPAPAPAPAPAPAPAPAPAPAPAPAPAP

SEQ ID NO: 66 - protein, 36 aa, synthetic construct
  source 1..36: note = Ala-Pro linker
APAPAPAPAPAPAPAPAPAPAPAPAPAPAPAPAPAP

SEQ ID NO: 67 - protein, 38 aa, synthetic construct
  source 1..38: note = Ala-Pro linker
APAPAPAPAPAPAPAPAPAPAPAPAPAPAPAPAPAPAP

SEQ ID NO: 68 - protein, 40 aa, synthetic construct
  source 1..40: note = Ala-Pro linker
APAPAPAPAPAPAPAPAPAPAPAPAPAPAPAPAPAPAPAP

SEQ ID NO: 69 - protein, 42 aa, synthetic construct
  source 1..42: note = Ala-Pro linker
APAPAPAPAPAPAPAPAPAPAPAPAPAPAPAPAPAPAPAPAP

SEQ ID NO: 70 - protein, 44 aa, synthetic construct
  source 1..44: note = Ala-Pro linker
APAPAPAPAPAPAPAPAPAPAPAPAPAPAPAPAPAPAPAPAPAP

SEQ ID NO: 71 - protein, 46 aa, synthetic construct
  source 1..46: note = Ala-Pro linker
APAPAPAPAPAPAPAPAPAPAPAPAPAPAPAPAPAPAPAPAPAPAP

SEQ ID NO: 72 - protein, 48 aa, synthetic construct
  source 1..48: note = Ala-Pro linker
APAPAPAPAPAPAPAPAPAPAPAPAPAPAPAPAPAPAPAPAPAPAPAP

SEQ ID NO: 73 - protein, 50 aa, synthetic construct
  source 1..50: note = Ala-Pro linker
APAPAPAPAPAPAPAPAPAPAPAPAPAPAPAPAPAPAPAPAPAPAPAPAP

SEQ ID NO: 74 - protein, 52 aa, synthetic construct
  source 1..52: note = Ala-Pro linker
APAPAPAPAPAPAPAPAPAPAPAPAPAPAPAPAPAPAPAPAPAPAPAPAPAP

SEQ ID NO: 75 - protein, 54 aa, synthetic construct
  source 1..54: note = Ala-Pro linker
APAPAPAPAPAPAPAPAPAPAPAPAPAPAPAPAPAPAPAPAPAPAPAPAPAPAP

SEQ ID NO: 76 - protein, 56 aa, synthetic construct
  source 1..56: note = Ala-Pro linker
APAPAPAPAPAPAPAPAPAPAPAPAPAPAPAPAPAPAPAPAPAPAPAPAPAPAPAP

SEQ ID NO: 77 - protein, 58 aa, synthetic construct
  source 1..58: note = Ala-Pro linker
APAPAPAPAPAPAPAPAPAPAPAPAPAPAPAPAPAPAPAPAPAPAPAPAPAPAPAPAP

SEQ ID NO: 78 - protein, 60 aa, synthetic construct
  source 1..60: note = Ala-Pro linker
APAPAPAPAPAPAPAPAPAPAPAPAPAPAPAPAPAPAPAPAPAPAPAPAPAPAPAPAPAP

SEQ ID NO: 79 - protein, 62 aa, synthetic construct
  source 1..62: note = Ala-Pro linker
APAPAPAPAPAPAPAPAPAPAPAPAPAPAPAPAPAPAPAPAPAPAPAPAPAPAPAPAPAPAP

SEQ ID NO: 80 - protein, 64 aa, synthetic construct
  source 1..64: note = Ala-Pro linker
APAPAPAPAPAPAPAPAPAPAPAPAPAPAPAPAPAPAPAPAPAPAPAPAPAPAPAPAPAPAPAP

SEQ ID NO: 81 - protein, 20 aa, synthetic construct
  source 1..20: note = linker
LEAGCKNFFPRSFTSCGSLE

SEQ ID NO: 82 - protein, 12 aa, synthetic construct
  source 1..12: note = linker
CRRRRRREAEAC

SEQ ID NO: 83 - protein, 6 aa, synthetic construct
  source 1..6: note = linker
GGGLLK

SEQ ID NO: 84 - protein, 25 aa, synthetic construct
  source 1..25: note = linker
GGGGGSGGSGGSGGSGGSGGSGGSK

SEQ ID NO: 85 - protein, 7 aa, synthetic construct
  source 1..7: note = linker
LPSTGGK

SEQ ID NO: 86 - protein, 17 aa, synthetic construct
  source 1..17: note = linker
  SITE 9..10: note = Protease-sensitive cleavage site
VSQTSKLTRAETVFPDV

SEQ ID NO: 87 - protein, 6 aa, synthetic construct
  source 1..6: note = linker
  SITE 3..4: note = Protease-sensitive cleavage site
PLGLWA

SEQ ID NO: 88 - protein, 6 aa, synthetic construct
  source 1..6: note = linker
  SITE 3..4: note = Protease-sensitive cleavage site
RVLAEA

SEQ ID NO: 89 - protein, 10 aa, synthetic construct
  source 1..10: note = linker
  SITE 6..7: note = Protease-sensitive cleavage site
EDVVCCSMSY

SEQ ID NO: 90 - protein, 8 aa, synthetic construct
  source 1..8: note = linker
  SITE 6..7: note = Protease-sensitive cleavage site
GGIEGRGS

SEQ ID NO: 91 - protein, 10 aa, synthetic construct
  source 1..10: note = linker
  SITE 7..8: note = Protease-sensitive cleavage site
TRHRQPRGWE

SEQ ID NO: 92 - protein, 10 aa, synthetic construct
  source 1..10: note = linker
  SITE 7..8: note = Protease-sensitive cleavage site
AGNRVRRSVG

SEQ ID NO: 93 - protein, 9 aa, synthetic construct
  source 1..9: note = linker
  SITE 7..8: note = Protease-sensitive cleavage site
  SITE 8..9: note = Protease-sensitive cleavage site
RRRRRRRRR

SEQ ID NO: 94 - protein, 4 aa, synthetic construct
  source 1..4: note = linker
  SITE 4: note = Protease-sensitive cleavage site
GFLG

SEQ ID NO: 95 - protein, 7 aa, Simian virus 40
PKKKRKV

SEQ ID NO: 96 - protein, 10 aa, Simian virus 40
PKKKRKVEAS

SEQ ID NO: 97 - protein, 16 aa, synthetic construct
  source 1..16: note = Nucleoplasmin bipartite NLS
KRPAATKKAGQAKKKK

SEQ ID NO: 98 - protein, 9 aa, synthetic construct
  source 1..9: note = c-myc NLS
PAAKRVKLD

SEQ ID NO: 99 - protein, 11 aa, synthetic construct
  source 1..11: note = c-myc NLS
RQRRNELKRSP

SEQ ID NO: 100 - protein, 38 aa, synthetic construct
  source 1..38: note = hRNPA1 M9 NLS
NQSSNFGPMKGGNFGGRSSGPYGGGGQYFAKPRNQGGY

SEQ ID NO: 101 - protein, 42 aa, synthetic construct
  source 1..42: note = IBB domain from importin-alpha
RMRIZFKNKGKDTAELRRRRVEVSVELRKAKKDEQILKRRNV

SEQ ID NO: 102 - protein, 8 aa, Homo sapiens
  source 1..8: note = Myoma T
VSRKRPRP

SEQ ID NO: 103 - protein, 8 aa, Homo sapiens
PQPKKKPL

SEQ ID NO: 104 - protein, 12 aa, Mus sp.
SALIKKKKKMAP

SEQ ID NO: 105 - protein, 5 aa, Influenza virus
DRLRR

SEQ ID NO: 106 - protein, 7 aa, Influenza virus
PKQKKRK

SEQ ID NO: 107 - protein, 10 aa, Hepatitis D virus
RKLKKKIKKL

SEQ ID NO: 108 - protein, 10 aa, Mus sp.
REKKKFLKRR

SEQ ID NO: 109 - protein, 20 aa, Homo sapiens
KRKGDEVDGVDEVAKKKSKK

SEQ ID NO: 110 - protein, 17 aa, Homo sapiens
RKCLQAGMNLEARKTKK

SEQ ID NO: 111 - protein, 10 aa, synthetic construct
  VARIANT 2..4: note = X is any amino acid
  VARIANT 6..7: note = X is any amino acid
  VARIANT 9: note = X is any amino acid
LXXXLXXLXL

SEQ ID NO: 112 - protein, 15 aa, synthetic construct
  source 1..15: note = Gly linker
GGGGGGGGGGGGGGG

SEQ ID NO: 113 - protein, 8 aa, synthetic construct
GPLGIAGQ

SEQ ID NO: 114 - DNA, 39 bp, synthetic construct
  source 1..39: mol_type = other DNA
ctttaagaaggagatataccatggtttccaaaggggagg

SEQ ID NO: 115 - DNA, 18 bp, synthetic construct
  source 1..18: mol_type = other DNA
tcccgcgtcgttcacacc

SEQ ID NO: 116 - DNA, 37 bp, synthetic construct
  source 1..37: mol_type = other DNA
gtgaacgacgcgggacaccaccaccaccaccactgag

SEQ ID NO: 117 - DNA, 16 bp, synthetic construct
  source 1..16: mol_type = other DNA
catgtatatctccttc

SEQ ID NO: 118 - DNA, 29 bp, synthetic construct
  source 1..29: mol_type = other DNA
gaaggagatataccatgattaaaaagacg

SEQ ID NO: 119 - DNA, 33 bp, synthetic construct
  source 1..33: mol_type = other DNA
gttagcagccggatctcagtgatggtggtgatg

SEQ ID NO: 120 - DNA, 24 bp, synthetic construct
  source 1..24: mol_type = other DNA
tgagatccggctgctaacaaagcc

SEQ ID NO: 121 - DNA, 29 bp, synthetic construct
  source 1..29: mol_type = other DNA
cgtctttttaatcatggtatatctccttc

SEQ ID NO: 122 - DNA, 30 bp, synthetic construct
  source 1..30: mol_type = other DNA
gaaggagatataccatgatgggcgcgagtg

SEQ ID NO: 123 - DNA, 42 bp, synthetic construct
  source 1..42: mol_type = other DNA
ggctttgttagcagccggatctcaatggtgatgatgatggtg

SEQ ID NO: 124 - DNA, 33 bp, synthetic construct
  source 1..33: mol_type = other DNA
acacctgacggttcccaccaccaccaccaccac

SEQ ID NO: 125 - DNA, 16 bp, synthetic construct
  source 1..16: mol_type = other DNA
catgtatatctccttc

SEQ ID NO: 126 - DNA, 43 bp, synthetic construct
  source 1..43: mol_type = other DNA
gaaggagatataccatggggcaccatcatcatcaccattctgg

SEQ ID NO: 127 - DNA, 44 bp, synthetic construct
  source 1..44: mol_type = other DNA
ggctttgttagcagccggatctcacttgtataattcgtccatgc

SEQ ID NO: 128 - DNA, 41 bp, synthetic construct
  source 1..41: mol_type = other DNA
cacggtaaacgaagtctaccccatggtgatgatgatggtgc

SEQ ID NO: 129 - DNA, 24 bp, synthetic construct
  source 1..24: mol_type = other DNA
tgagatccggctgctaacaaagcc

SEQ ID NO: 130 - protein, 8 aa, Homo sapiens
PPKKARED

(19) United States
(12) Patent Application Publication
(10) Pub. No.: US 2025/0353884 A1
(43) Pub. Date: Nov. 20, 2025

(54) INTEIN SYSTEMS AND USES THEREOF

(71) Applicant: CORNELL UNIVERSITY, Ithaca, NY (US)

(72) Inventors: Jeongmin Song, Ithaca, NY (US); Tri Nguyen, Archdale, NC (US)

(21) Appl. No.: 18/706,877

(22) PCT Filed: Nov. 2, 2022
(86) PCT No.: PCT/US2022/079164

§ 371 Date: May 2, 2024

Related U.S. Application Data
(60) Provisional application No. 63/274,799, filed on Nov. 2, 2021.

(51) Int. Cl. C07K 14/195

(52) U.S. Cl. CPC C07K 14/195

(57) ABSTRACT

Described in several example embodiments herein are engineered split intein polypeptides and systems thereof. Also described in several example embodiments, herein are methods of using the engineered split intein polypeptides and systems thereof, such as to catalyze a bioconjugation reaction.

### CROSS-REFERENCE TO RELATED APPLICATIONS

This application claims the benefit of and priority to U.S. Provisional Patent Application No. 63/274,799, filed on Nov. 2, 2021, the contents of which is incorporated by reference herein in its entirety.

### STATEMENT REGARDING FEDERALLY SPONSORED RESEARCH

This invention was made with government support under Grant No. AI137345, AI139625, and AI141514 awarded by National Institutes of Health. The government has certain rights in the invention.

### SEQUENCE LISTING

This application contains a sequence listing filed in electronic form as an .xml file entitled CORNL-0645WP_ST26.xml, created on Nov. 2, 2022 and having a size of 139,495 bytes. The content of the sequence listing is incorporated herein in its entirety.

### TECHNICAL FIELD

The subject matter disclosed herein is generally directed to engineered split-intein systems and uses thereof.

### BACKGROUND

Inteins are protein splicing sequences that are post-translationally excised out in an auto-catalytic manner to produce mature host proteins whose genetic information is split into two parts at the DNA level. In many cases, the resulting mature host proteins called mature exteins are the enzymes involved in DNA processing, such as DNA polymerases, helicases, and endonucleases. Inteins are considered to be of very ancient origin and often have been described as selfish genetic materials due to the lack of apparent cellular functions. Consistently, although inteins are found in all three domains of life, higher organisms, including humans and other animals, do not encode the intein systems in their genome. While inteins can be found in many different forms, split-inteins are encoded in two separate genetic locations translating to two respective polypeptides. The N-terminal and C-terminal split extein-intein halves recognize each other and catalyze protein trans-splicing reactions leading to mature proteins consisting of the N-terminal and C-terminal extein halves without intein polypeptides.

Nearly all native split-intein polypeptides contain multiple cysteine residues. This feature limits the flexibility of extein choices in taking advantage of these otherwise highly applicable bioconjugation systems. This limitation is associated with the vital role of cysteine residues in protein structure and function. The addition of reducing agents is often required for the cysteine-containing split-intein-mediated trans-splicing reactions, which can, unfortunately, render mature proteins non-functional. Consistently, there have been searches for the cysteine-less (CL) split-intein systems primarily by two approaches: (1) point-mutations of cysteine residues found in the native cysteine-containing split-inteins and (2) hunts for new CL split-inteins. Despite such efforts, CL split-intein systems are rare. Thus, there exists a need for additional split-intein systems, particularly CL split-intein systems.

Citation or identification of any document in this application is not an admission that such a document is available as prior art to the present invention.

### SUMMARY

Described in certain example embodiments herein are engineered intein system comprising a recombinant first amino acid sequence comprising an N-terminal intein sequence; and a recombinant second amino acid sequence comprising a C-terminal intein sequence, wherein the N-terminal intein sequence, the C-terminal intein sequence, or both are derived from a split intein of Richelia sp., Pseudomonas aeruginosa 18, Candidatus Brocadiales, or any combination thereof.

In certain example embodiments, the split intein is a cysteine-less split intein.

In certain example embodiments, the N-terminal intein sequence comprises an amino acid sequence having at least 80%, 85%, 90%, 95%, 98%, or 99% sequence identity to any one of SEQ ID NO: 1, 3, 5, or 7.

In certain example embodiments, the C-terminal intein sequence comprises an amino acid sequence having at least 80%, 85%, 90%, 95%, 98%, or 99% sequence identity to any one of SEQ ID NO: 2, 4, 6, or 8.

In certain example embodiments, the C-terminal intein sequence comprises X₁PYFFX₂NNILVHNS (SEQ ID NO: 10), wherein X₁ and X₂ are each independently selected from any amino acid. In certain example embodiments, (a) wherein X₁ is selected from N or T, (b) wherein X₂ is selected from A or G, or (c) both (a) and (b). In certain example embodiments, wherein the C-terminal sequence comprises SEQ ID NO: 9.

In certain example embodiments, the wherein the N-terminal intein sequence is attached to a C-terminus of the first amino acid sequence with a peptide bond.

In certain example embodiments, the C-terminal intein sequence is attached to a N-terminus of the first amino acid sequence with a peptide bond.

In certain example embodiments, the system further comprises a linker between the first amino acid sequence and the N-terminal intein sequence, optionally wherein the linker is a peptide linker. In some embodiments, the linker is a flexible linker. In some embodiments, the linker is a rigid linker.

In certain example embodiments, the system further comprises a linker between the first amino acid sequence and the C-terminal intein sequence, optionally wherein the linker is a peptide linker. In some embodiments, the linker is a flexible linker. In some embodiments, the linker is a rigid linker.

In certain example embodiments, the linker is not more than 1, 2, 3, 4, 5, 6, 7, 8, 9, 10, 11, 12, 13, 14, 15, or 20 amino acids in length.

In certain example embodiments, linker is a Gly-Ser linker, optionally wherein the linker comprises an amino acid sequence of at least 80%, 85%, 90%, 95%, 98% sequence identity to GSGSGSGSGSGSGSGSGSGSG (SEQ ID NO: 11).

In certain example embodiments, the linker is an Asparagine-Serine linker, optionally wherein the linker comprises an amino acid sequence of at least 80%, 85%, 90%, 95%, 98% sequence identity to ASASASASASASASASAS (SEQ ID NO: 12).

In certain example embodiments, the engineered intein system further comprises a targeting moiety localization tag, affinity tag, reporter tag, or any combination thereof, wherein the localization tag, affinity tag, reporter tag, or any combination thereof is operatively coupled to the first amino acid sequence, the second amino acid sequence, or both.

In certain example embodiments, the system is capable of catalyzing a bioconjugation reaction at a pH ranging from about 6 to about 8.

In certain example embodiments, the system is capable of catalyzing a bioconjugation reaction at a temperature ranging from about 20° C. to about 50° C.

In certain example embodiments, the system is capable of catalyzing a bioconjugation reaction, wherein the system is capable of catalyzing a bioconjugation reaction in the presence of a reducing agent, optionally wherein the reducing agent is dithiothreitol (DTT), beta mercaptoethanol (BME), tris(2-carboxyethyl)phosphine (TCEP), or cysteine.

In certain example embodiments, the system is capable of catalyzing a bioconjugation reaction in the presence of about 0.05 M NaCl to about 2 M NaCl.

Described in certain example embodiments herein are engineered polynucleotide encoding the engineered intein system of the present disclosure or a component thereof.

Described in certain example embodiments herein are vectors or vector systems comprising one or more engineered polynucleotides of the present disclosure, optionally wherein at least one of the one or more engineered polynucleotides is operatively coupled to a regulatory element.

Described in certain example embodiments herein are cells or populations thereof comprising: a. engineered intein system of any one of the present disclosure; b. one or more engineered polynucleotides of the present disclosure; c. one or more vector or vector systems of the present disclosure; or d. any combination of (a)-(c).

Described herein are non-human organisms comprising: a. engineered intein system of any one of the present disclosure; b. one or more engineered polynucleotides of the present disclosure; c. one or more vector or vector systems of the present disclosure; or d. cell or population thereof of the present disclosure; or e. any combination of (a)-(d).

Described herein are formulations comprising: a. engineered intein system of any one of the present disclosure; b. one or more engineered polynucleotides of the present disclosure; c. one or more vector or vector systems of the present disclosure; d. cell or population thereof of the present disclosure; or e. any combination of (a)-(d); and a carrier. In certain example embodiments, the carrier is a pharmaceutically acceptable carrier.

Described in certain example embodiments herein are kits comprising: a. engineered intein system of the present disclosure; b. one or more engineered polynucleotides of the present disclosure; c. one or more vector or vector systems of the present disclosure; d. cell or population thereof of the disclosure; e. a formulation of the present disclosure; or f. any combination of (a)-(e).

Described in certain example embodiments herein are method of bioconjugation comprising: mixing a recombinant first amino acid sequence comprising an N-terminal intein sequence with a recombinant second amino acid sequence comprising a C-terminal intein sequence under conditions sufficient to allow bioconjugation of the first recombinant amino acid sequence and the second recombinant amino acid sequence, wherein the N-terminal intein sequence, the C-terminal intein sequence, or both are derived from a split intein of Richelia sp., Pseudomonas aeruginosa 18, Candidatus Brocadiales, or any combination thereof.

In certain example embodiments, the split intein is a cysteine-less split intein.

In certain example embodiments, the N-terminal intein sequence comprises an amino acid sequence having at least 80%, 85%, 90%, 95%, 98% sequence identity to any one of SEQ ID NO: 1, 3, 5, or 7.

In certain example embodiments, the C-terminal intein sequence comprises an amino acid sequence having at least 80%, 85%, 90%, 95%, 98% sequence identity to any one of SEQ ID NO: 2, 4, 6, or 8.

In certain example embodiments, the N-terminal intein sequence is attached to a C-terminus of the first amino acid sequence with a peptide bond.

In certain example embodiments, the C-terminal intein sequence is attached to a N-terminus of the first amino acid sequence with a peptide bond.

In certain example embodiments, a linker is operatively coupled between the first amino acid sequence and the N-terminal intein sequence, optionally wherein the linker is a peptide linker.

In certain example embodiments, a linker is operatively coupled between the first amino acid sequence and the C-terminal intein sequence, optionally wherein the linker is a peptide linker.

In certain example embodiments, the linker is not more than 1, 2, 3, 4, 5, 6, 7, 8, 9, 10, 11, 12, 13, 14, 15, or 20 amino acids in length.

In certain example embodiments, linker is a Gly-Ser linker, optionally wherein the linker comprises an amino acid sequence of at least 80%, 85%, 90%, 95%, 98% sequence identity to GSGSGSGSGSGSGSGSGSGSG (SEQ ID NO: 11).

In certain example embodiments, the linker is an Asparagine-Serine linker, optionally wherein the linker comprises an amino acid sequence of at least 80%, 85%, 90%, 95%, 98% sequence identity to ASASASASASASASASAS (SEQ ID NO: 12).

In certain example embodiments, a localization tag, affinity tag, reporter tag, or any combination thereof, is operatively coupled to the first amino acid sequence, the second amino acid sequence, or both.

In certain example embodiments, the C-terminal intein sequence comprises X₁PYFFX₂NNILVHNS (SEQ ID NO: 10), wherein X₁ and X₂ are each independently selected from any amino acid.

In certain example embodiments, (a) wherein X₁ is selected from N or T, (b) wherein X₂ is selected from A or G, or (c) both (a) and (b).

In certain example embodiments, the C-terminal sequence comprises SEQ ID NO: 9.

In certain example embodiments, the conditions sufficient to allow bioconjugation comprise a pH ranging from about 6 to about 8.

In certain example embodiments, the conditions sufficient to allow bioconjugation comprise a temperature ranging from about 20° C. to about 50° C.

In certain example embodiments, the conditions sufficient to allow bioconjugation comprise a reducing agent, optionally wherein the reducing agent is dithiothreitol (DTT), beta mercaptoethanol (BME), tris(2-carboxyethyl)phosphine (TCEP), or cysteine.

In certain example embodiments, the conditions sufficient to allow bioconjugation comprise NaCl at a concentration ranging from about 0.05 M NaCl to about 2 M NaCl.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1A-1C—Cyanobacteria Richelia sp. RM2_1_2 encodes an unusual CL split-intein pair, the Rsp CL system. FIG. 1A. Genomic locations (top panel) and relevant information (bottom panel) of nine intein halves found in Richelia sp. RM2_1_2. NJO60988.1 and NJO60986.1 are the N- and C-terminal split-intein halves, respectively. FIG. 1B. Phylogenetic tree comparing the NJO60988.1 N-terminal split-intein amino acid sequence and the eight hits identified via Protein BLAST using NJO60986.1 as the query. FIG. 1C. Phylogenetic tree comparing the NJO60986.1 C-terminal split-intein amino acid sequence and the eight hits identified via Protein BLAST using NJO60986.1 as the query. The distance scale is shown on the top of the figure panels. See also Tables 1 and 2.

FIG. 2A-2D—The Rsp CL intein system is equipped with extein-tolerance and catalyzes the reaction to completion. FIG. 2A. A chematic diagram of a dual fluorescent report assay involving two different fluorescent proteins (mTurquoise2 and mCherry2) serving as the N-terminal extein and C-terminal extein respectively. This assay explains the protein trans-splicing reaction (PTS) of the Rsp CL biosystem. Rsp CL split-intein N-terminal half (I^{N}) was fused to mTurquoise2 (mTQ), and Rsp CL split-intein C-terminal half (IC) was linked to mCherry2 (mCH). When two protein preparations were mixed in a single tube, the indicated PTS occurs, resulting in PTS products—two exteins fused in a precise manner. FIG. 2B. PTS of the Rsp CL split-intein system at different temperatures and ratios (FIGS. 6A-6B and 7A-7B). mTQ::I^{N} and I^{C}::mCH were mixed at a ratio of 1:1 (as well as 1:4 and 4:1 in FIG. 7A-7B), incubated at indicated temperatures for 6 hrs in a pH 7.5 buffer, separated in SDS-PAGE, and visualized by a fluorescent scanner. PTS, mTQ::mCH fused PTS product. FIG. 2C-2D. PTS reaction kinetics of the Rsp CL split-intein system at different temperatures. mTQ::I^{N} and I^{C}::mCH were mixed at a ratio of 1:3, incubated at indicated temperatures (37° C. for the left panel and 4° C. for the right panel) for indicated durations in a pH 7.5 buffer. FIG. 2C, Representative results of the summary graph in FIG. 2D. FIG. 2D. Summary of three independent experiments. Each data point represents average±standard deviation (SD). See also FIGS. 6A-6B, 7A-7B, and 8A-8B and Table 2.

FIG. 3A-3B—The Rsp CL intein system completes its protein trans-splicing reaction within minutes to hours. PTS reaction kinetics of the Rsp CL split-intein system at different pH. mTQ::I^{N} and I^{C}::mCH were mixed at a ratio of 1:3, incubated at 37° C. for indicated durations in a buffer of indicated pH. FIG. 3A. Representative results of the summary graph in FIG. 3B. FIG. 3B. Summary of three independent experiments. Each data point represents average±SD. See also FIGS. 9A-9B and 10A-10C.

FIG. 4A-4H—The Rsp CL intein system completes its protein trans-splicing reaction to completion in a wide range of temperatures, reducing agents, salts, and denaturing agents. mTQ::I^{N} and I^{C}::mCH were mixed at a ratio of 1:3 and incubated for 1 hr using a pH 6.0 buffer. Reaction samples were separated in SDS-PAGE gels and visualized by a fluorescent or digital scanner for fluorescent proteins or Coomassie-stained proteins, respectively. FIG. 4A-4B. PTS of the Rsp CL split-intein system at various temperatures. *, PTS products. Shown are representative results (FIG. 4A) and summary graph obtained from three independent experiments (FIG. 4B). FIG. 4C-4D. PTS of the Rsp CL split-intein system in the presence of various reducing agents. Representative results (FIG. 4C) and summary graph obtained from three independent experiments (FIG. 4D) are shown. FIG. 4E-4F. PTS of the Rsp CL split-intein system in the presence of various salt concentrations. Shown are representative results (FIG. 4E) and summary graph obtained from three independent experiments (FIG. 4F). FIG. 4G-4H. PTS of the Rsp CL split-intein system in the presence of various denaturing agents. Shown are representative results (FIG. 4G) and summary graph obtained from three independent experiments (FIG. 411). *, PTS products. Bars represent average±SD. See also FIGS. 11, 12A-12B, 13A-13D, and 14.

FIG. 5A-5B—The Rsp CL intein system completes its protein trans-splicing reaction in a precise manner. All reactions were carried out for 1 hr using a pH 6.0 buffer. FIG. 5A-5B. PTS reactions of the Rsp CL split-intein system in the presence of various host proteins and/or other split-intein halves. Rsp^{N} (mTQ::I^{N}) and Rsp^{C} (I^{C}::mCH) were mixed at a ratio of 1:3, incubated at 37° C., separated in SDS-PAGE gels, and visualized by a fluorescent (FIG. 5A) or digital scanner for Coomassie (FIG. 5B). * in orange, Rsp PTS products. * in green, Cfa PTS products. Shown are representative results from three independent experiments (FIG. 16). Cell lysate, 50 μg/ml total human intestinal epithelial Henle-407 cell lysate. Cfa^{N}, Aes^{C}::Cfa^{N}. Cfa^{C}, Cfa^{C}::Gamillus. See also FIGS. 15A-15B and 16 and Table 2.

FIG. 6A-6B—Protein trans-splicing reaction (PTS) of the Rsp CL split-intein system. Rsp CL N-terminal intein was fused to mTurquoise (mTQ::IN), and Rsp CL C-terminal intein was linked to mCherry (IC::mCH). These two proteins were mixed at a ratio of 1:3, incubated for 6 hrs in a pH 7.5 buffer, separated in SDS-PAGE gels, and visualized by a fluorescent (FIG. 6A) or digital scanner for Coomassie (FIG. 6B). When indicated, the samples were boiled before applying to SDS-PAGE gels. See also FIG. 2A-2D.

FIG. 7A-7B—PTS reaction of the Rsp CL split-intein system at different temperatures and ratios. mTQ::IN and IC::mCH were mixed at a ratio of 1:1, 1:4, and 4:1, incubated at indicated temperatures for 6 hrs in a pH 7.5 buffer, separated in SDS-PAGE gels, and visualized by a fluorescent (FIG. 7A) or digital scanner for Coomassie (FIG. 7B). See also FIG. 2A-2D.

FIG. 8A-8B—PTS reaction kinetics of the Rsp CL split-intein system at different temperatures. mTQ::IN and IC::mCH were mixed at a ratio of 1:3, incubated at 37° C. (FIG. 8A) and 4° C. (FIG. 8B) for indicated durations in a pH 7.5 buffer, separated in SDS-PAGE gels, and visualized by a digital scanner for Coomassie. Three independent experimental results are shown. See also FIG. 2A-2D.

FIG. 9A-9B—The Rsp CL split-intein system catalyzes the PTS reaction in a wide range of pH conditions. mTQ::IN and IC::mCH were mixed at a ratio of 1:3, incubated at 37° C. for 1 hr (FIG. 9A) or 6 hr (FIG. 9B) in a buffer of indicated pH, separated in SDS-PAGE gels, boiled, and visualized by a digital scanner for Coomassie. See also FIG. 3A-3B.

FIG. 10A-10C—PTS reaction kinetics of the Rsp CL split-intein system at different pH. mTQ::IN and IC::mCH were mixed at a ratio of 1:3, incubated at 37° C. for indicated durations in a buffer of indicated pH (pH 6.0 for FIG. 10A, pH 6.5 for FIG. 10B, and pH 7.0 for FIG. 10C), separated in SDS-PAGE gels, and visualized by a fluorescent (top panel) or digital scanner for Coomassie (bottom panel). Three independent experimental results are shown. See also FIG. 3A-3B.

FIG. 11—The Rsp CL split-intein system catalyzes the PTS reaction at various temperatures. mTQ::IN and IC::mCH were mixed at a ratio of 1:3, incubated at indicated temperatures for 1 hr in a pH 6.0 buffer, separated in SDS-PAGE gels, and visualized by a digital scanner for Coomassie. Three independent experimental results are shown. See also FIG. 4A-4H.

FIG. 12A-12B—The Rsp CL split-intein system catalyzes the PTS reaction in the presence of reducing agents. mTQ::IN and IC::mCH were mixed at a ratio of 1:3, incubated at 37° C. for 1 hr in a pH 6.0 buffer containing indicated reducing agents, separated in SDS-PAGE gels, and visualized by a fluorescent (top panel) or digital scanner for Coomassie (bottom panel). Three independent experimental results are shown. See also FIG. 4A-4H.

FIG. 13A-13D—The Rsp CL split-intein biosystem catalyzes the PTS reaction in the presence of various salt concentrations. mTQ::IN and IC::mCH were mixed at a ratio of 1:3, incubated at 37° C. for 1 hr in a pH 6.0 buffer containing indicated salt concentrations, separated in SDS-PAGE gels, and visualized by a fluorescent scanner (FIGS. 13A and 13C) and a digital scanner for Coomassie (FIGS. 13B and 13D). Three independent experimental results are shown. See also FIG. 4A-4H.

FIG. 14—The Rsp CL split-intein biosystem tolerates some denaturing conditions. mTQ::IN and IC::mCH were mixed at a ratio of 1:3, incubated at 37° C. for 6 hr in a pH 6.0 buffer containing indicated denaturing agents, separated in SDS-PAGE gels, and visualized by a digital scanner for Coomassie. Three independent experimental results are shown. See also FIG. 4A-4H.

FIG. 15A-15B—Protein trans-splicing reaction of the Cfa split-intein system. Aes^{C}::Cfa^{N} and Cfa^{C}::Gamillus were mixed, incubated at 37° C. for 1 hr in a pH 6.0 buffer, separated in SDS-PAGE gels, and visualized by a fluorescent scanner (FIG. 15A) and a digital scanner for Coomassie (FIG. 15B). * indicates AesC::CfaN, CfaC::Gamillus, and PTS product in respective lanes. See also FIG. 5A-5B.

FIG. 16—The Rsp CL intein system completes its protein trans-splicing reaction in a precise manner. Indicated components were mixed, incubated at 37° C. for 1 hr in a pH 6.0 buffer, separated in SDS-PAGE gels, and visualized by a fluorescent (top panel) or digital scanner for Coomassie (bottom panel). * in orange, Rsp PTS products. * in green, Cfa PTS products. Cell lysate, 50 μg/ml total human intestinal epithelial Henle-407 cell lysate. CfaN, AesC::CfaN. CfaC, CfaC::Gamillus. Three independent experimental results are shown. See also FIG. 5A-5B.

FIG. 17—Split-intein is one of the common bioconjugation tool for conjugation of proteins as well as other macromolecules. The intein reaction involving the two intein halves: N-intein and C-intein coming together. This is followed by the trans-splicing reaction of the N-Extein and C-Exteins leading to the release of the intein complex and formation of a peptide bond between exteins. Most inteins required the presence of reducing agents for activity since they contain cysteine residues in the catalytic sites. In extremely rare cases, cysteine-less intein are reported that capable of carry out the reaction without reducing agents, thus preserving essential disulfide bonds on the exteins.

FIG. 18—An intein system found in phage that infect Aeromonas salmonicida. This intein system in its native form could not complete the reaction beyond 30%. They resolve to engineer a new artificial split site within the intein complex and demonstrated that the reaction went to near completion within 3 hours. They were able to demonstrate the use of this system in various purpose from antibodies engineering as well as in vitro and in vivo conjugative labeling. This engineered system is however reported to be highly unstable and slow.

FIG. 19—Three additional Cysteine-less intein template Rsp, Pae, and C and Intein identified by Applicant.

FIG. 20A-20C—The Rsp CL intein system is equipped with extein tolerance and catalyzes the reaction to completion. FIG. 20A. Benchmark reaction of previously reported engineered Aes CL intein with intein reaction being characterized by quantification of coumassie stained SDS-PAGE after samples were heated for 5 min at 95 degrees C. following addition of SDS-loading dye (heated) versus intein reaction being characterized by quantification of fluorescent signal obtained from SDS-PAGE after sample was not heated following addition of SDS-loading dye (non-heated). Splicing kinetic from triplicate experiment is shown for each quantification method. FIG. 20B. PTS reaction kinetics of the Rsp CL split intein-system at different temperature mTQ::RspN and RspC::mCH were mixed at a ratio of 1:3, incubated at indicated temperature (37 degrees C. for the left panel and 4 degrees C. for the right panel) for indicated durations in a pH 7.5 buffer. FIG. 20C. Representative results of the summary graph.

FIG. 21A-21C—Two other cysteine-less intein system, identified through sequence similarity search based upon Rsp CL intein sequences, demonstrate robust reactivity. FIG. 21A (SEQ ID NO: 13-21). Clustal alignment of sequence of Rsp CL intein found in Richelia sp. RM2_1_2 and Pae CL intein found in the jumbo phage vB_PaeM_MIJ3 infecting Pseudomonas aeruginosa and Cand CL intein found in the unassigned Candidatus Brocadiales bacterium. FIG. 21B. PTS reaction kinetics of Pae CL split intein-system at different pH. Summary graphs obtained from three independent experiment are shown. Each data point represents average±standard deviation. FIG. 21C. PTS reaction kinetics of Cand CL split intein-system at different pH. Summary graphs obtained from three independent experiment are shown. Each data point represents average±standard deviation.

FIG. 22A-22C—mTQ::IN and IC::mCH were mixed at a ratio of 1:3 and incubated for 1 hr using a pH 6.0 buffer for Rsp CL and Pae CL inteins and pH 7.0 buffer for Cand CL intein containing the noted denaturant concentration. Reaction was quenched by addition of SDS-loading dye and heated for 5 minutes at 95 degrees C. Reaction samples were separated in SDS-PAGE gels and visualized and splicing kinetic was quantified. Bars represent average±SD. See also FIGS. 8A-8B, 10A-10C, and 11. mTQ::IN and IC::mCH were mixed at a ratio of 1:3 and incubated for 1 hr at various temperatures using a pH 6.0 buffer for Rsp CL and Pae CL inteins and pH 7.0 buffer for Cand CL intein. Reaction was quenched by addition of SDS-loading dye and heated for 5 minutes at 95 degrees C. Reaction samples were separated in SDS-PAGE gels and visualized with Coomassie staining and splicing kinetic was quantified. Bars represent average±SD. See also FIGS. 8A-8B, 10A-10C, and 11. Mixture of Rsp CL, Pae CL, and Cand CL intein-halves were incubated 1:3 (IN to IC to test for cross reactivity for 1 hour at 37 degrees C. using pH 7.0 buffer. Reaction was quenched by addition of SDS-loading dye and heated for 5 minutes at 95 degrees C. Reaction samples were separated in SDS-PAGE gels and visualized with Coomassie staining.

FIG. 23A-23G—Crystal structures of Rsp CL intein and Pae intein reveal the conserved intein/hedgehog fold. FIG. 23A. Crystal structure of Rsp intein obtained from purified PTS by-product of Rsp CL intein reactions. RspN is colored green and RspC is colored pink. Ser1 is colored cyan. FIG. 23B. Summary of interaction between RspC flexible loop (Met1-Glu19) with RspN. Hydrophobic interaction is colored in yellow and charge interaction is colored in blue. FIG. 23C. Crystal structure of Pae intein obtained from recombinantly purified S1A mutant of Pae CL intein with its N- and C-termini linked with a glycine flanked by its native intein linker. PaeN is colored light green, PaeC is colored salmon, exteins are colored purple. S1A residue is colored cyan, S+1 residue is colored yellow. FIG. 23D. Summary of interaction between PaeC flexible loop (M1-E19) with PaeN. Hydrophobic interaction is colored in yellow and charge interaction is colored in blue. FIG. 23E. Amino acids involve in coordination of S1 in Rsp intein. FIG. 23F. Amino acids involve in the catalytic active sites between S1 and S+1. FIG. 23G. Mutagenesis study of Pae CL intein with actives site residues derived from Rsp crystal structure: PaeN S1A and H87A, PaeC D26A and N46A.

FIG. 24A-24E—Active site of the new cysteine-less intein systems contain unique conserved proline residue. A conserved proline residue is observed in the interaction with the penultimate histidine and catalytic asparagine residues of the C-terminal intein half. FIG. 24A. Structure of Rsp Intein with the unique proline is highlighted in red, the C-terminal intein half is colored pink, the N-terminal intein half is colored green, the Ser residue is colored cyan. FIG. 24B. Structure of Pae intein with the unique proline is highlighted in red, the C-terminal intein half is colored salmon, the N-terminal intein half is colored light green, the Ser+1 residue is colored yellow, the Ser1Ala residue is colored cyan and the exteins are colored purple. FIG. 24C. Structure of GP41-1 intein with the Histidine residue of interest highlighted in red, The C-terminal intein half is colored purple, the N-terminal intein half is colored yellow and the Cys1Ala residue is colored cyan. FIG. 24D (SEQ ID NO: 22-26). Amino acid sequence comparison of C-terminal intein half of Rsp, Pae, Cand, Aes and GP41-1 intein. FIG. 24E. Comparison in splicing kinetic between Pae^{N} and Pae^{C} wild-type versus Pae^{C} P35H mutant. Summary of three independent experiments. Each data point represents average±standard deviation (SD).

FIG. 25A-25F—F-block sequences of new cysteine-less intein systems contribute to splicing reactivity. FIG. 25A. AlphaFold prediction of F-block fold in relation to S+1 residue and C-terminus extein. The F-block of Rsp intein is colored cyan, of Pae intein is colored salmon, and of Cand intein is colored green. FIG. 25B (SEQ ID NO: 27-29). Sequence comparison of Cand^{C} and PaeC and the design of Chim^{C} which is a hybrid of CandC and PaeC. FIG. 25C. Splicing kinetic of Chim CL intein system at different pH. Summary of three independent experiments. Each data point represents average±standard deviation (SD). FIG. 25D. mTQ::Chim^{N} and Chim^{C}::mCH were mixed at a ratio of 1:3 and incubated for 1 hr using a pH 6.0 buffer containing the noted denaturant concentration. Reaction was quenched by addition of SDS-loading dye and heated for 5 minutes at 95 degrees C. Reaction samples were separated in SDS-PAGE gels and visualized and splicing kinetic was quantified. Bars represent average±SD. See also FIG. 13A-13D. FIG. 25D. mTQ::Chim^{N} and Chim^{C}::mCH were mixed at a ratio of 1:3 and incubated for 1 hr at various temperatures using a pH 6.0 buffer. Reaction was quenched by addition of SDS-loading dye and heated for 5 minutes at 95 degrees C. Reaction samples were separated in SDS-PAGE gels and visualized with Coomassie staining and splicing kinetic was quantified. Bars represent average±SD. See also FIG. 13A-13D. FIG. 25F. Mixture of Rsp CL, Pae CL, and Chim CL intein-halves were incubated 1:3 (IN to IC) to test for cross reactivity for 1 hour at 37 degrees C. using pH 7.0 buffer. Reaction was quenched by addition of SDS-loading dye and heated for 5 minutes at 95 degrees C. Reaction samples were separated in SDS-PAGE gels and visualized with Coomassie staining.

The figures herein are for illustrative purposes only and are not necessarily drawn to scale.

### DETAILED DESCRIPTION OF THE EXAMPLE EMBODIMENTS

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlett, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

Definitions of common terms and techniques in chemistry and organic chemistry can be found in Smith. Organic Synthesis, published by Academic Press. 2016; Tinoco et al. Physical Chemistry, 5^{th} edition (2013) published by Pearson; Brown et al., Chemistry, The Central Science 14^{th} ed. (2017), published by Pearson, Clayden et al., Organic Chemistry, 2^{nd} ed. 2012, published by Oxford University Press; Carey and Sunberg, Advanced Organic Chemistry, Part A: Structure and Mechanisms, 5^{th} ed. 2008, published by Springer; Carey and Sunberg, Advanced Organic Chemistry, Part B: Reactions and Synthesis, 5^{th} ed. 2010, published by Springer, and Vollhardt and Schore, Organic Chemistry, Structure and Function; 8^{th} ed. (2018) published by W. H. Freeman.

Definitions of common terms, analysis, and techniques in genetics can be found in e.g., Hartl and Clark. Principles of Population Genetics. 4^{th} Ed. 2006, published by Oxford University Press. Published by Booker. Genetics: Analysis and Principles, 7^{th} Ed. 2021, published by McGraw Hill; Isik et la., Genetic Data Analysis for Plant and Animal Breeding. First ed. 2017. published by Springer International Publishing AG; Green, E. L. Genetics and Probability in Animal Breeding Experiments. 2014, published by Palgrave; Bourdon, R. M. Understanding Animal Breeding. 2000 2^{nd} Ed. published by Prentice Hall; Pal and Chakravarty. Genetics and Breeding for Disease Resistance of Livestock. First Ed. 2019, published by Academic Press; Fasso, D. Classification of Genetic Variance in Animals. First Ed. 2015, published by Callisto Reference; Megahed, M. Handbook of Animal Breeding and Genetics, 2013, published by Omniscriptum Gmbh & Co. Kg., LAP Lambert Academic Publishing; Reece. Analysis of Genes and Genomes. 2004, published by John Wiley & Sons. Inc; Deonier et al., Computational Genome Analysis. 5^{th} Ed. 2005, published by Springer-Verlag, New York; Meneely, P. Genetic Analysis: Genes, Genomes, and Networks in Eukaryotes. 3^{rd} Ed. 2020, published by Oxford University Press.

As used herein, the singular forms “a” “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g., given data set, art accepted standard, and/or with e.g. a given confidence interval (e.g., 90%, 95%, or more confidence interval from the mean), such as variations of +/−10% or less, +/−5% or less, +/−1% or less, and +/−0.1% or less of and from the specified value, insofar such variations are appropriate to perform in the disclosed invention. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

As used herein, a “biological sample” refers to a sample obtained from, made by, secreted by, excreted by, or otherwise containing part of or from a biologic entity. A biologic sample can contain whole cells and/or live cells and/or cell debris, and/or cell products, and/or virus particles. The biological sample can contain (or be derived from) a “bodily fluid”. The biological sample can be obtained from an environment (e.g., water source, soil, air, and the like). Such samples are also referred to herein as environmental samples. As used herein “bodily fluid” refers to any non-solid excretion, secretion, or other fluid present in an organism and includes, without limitation unless otherwise specified or is apparent from the description herein, amniotic fluid, aqueous humor, vitreous humor, bile, blood or component thereof (e.g. plasma, serum, etc.), breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from an organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some, but not other features included in other embodiments, combinations of features of different embodiments are meant to be within the scope of the invention. For example, in the appended claims, any of the claimed embodiments can be used in any combination.

As used herein, “attached” refers to covalent or non-covalent interaction between two or more molecules. Non-covalent interactions can include ionic bonds, electrostatic interactions, van der Walls forces, dipole-dipole interactions, dipole-induced-dipole interactions, London dispersion forces, hydrogen bonding, halogen bonding, electromagnetic interactions, π-π interactions, cation-π interactions, anion-π interactions, polar π-interactions, and hydrophobic effects. As used herein “attached” as applied to capture molecules of an array or other device refers to a covalent interaction or bond between a molecule on the surface of the support and the capture molecule so as to immobilize the capture molecule on the surface of the support.

As used herein “essentially discrete” as applied to features of an array refers to the situation where 90% or more of the features of an array are not in direct contact with other features of the same array.

As used herein, “expression” refers to the process by which polynucleotides are transcribed into RNA transcripts. In the context of mRNA and other translated RNA species, “expression” also refers to the process or processes by which the transcribed RNA is subsequently translated into peptides, polypeptides, or proteins. In some instances, “expression” can also be a reflection of the stability of a given RNA. For example, when one measures RNA, depending on the method of detection and/or quantification of the RNA as well as other techniques used in conjunction with RNA detection and/or quantification, it can be that increased/decreased RNA transcript levels are the result of increased/decreased transcription and/or increased/decreased stability and/or degradation of the RNA transcript. One of ordinary skill in the art will appreciate these techniques and the relation “expression” in these various contexts to the underlying biological mechanisms.

As used herein, “fragment” as used throughout this specification with reference to a peptide, polypeptide, or protein generally denotes a portion of the peptide, polypeptide, or protein, such as typically an N- and/or C-terminally truncated form of the peptide, polypeptide, or protein. Preferably, a fragment may comprise at least about 30%, e.g., at least about 50% or at least about 70%, preferably at least about 80%, e.g., at least about 85%, more preferably at least about 90%, and yet more preferably at least about 95% or even about 99% of the amino acid sequence length of said peptide, polypeptide, or protein. For example, insofar not exceeding the length of the full-length peptide, polypeptide, or protein, a fragment may include a sequence of ≥5 consecutive amino acids, or ≥10 consecutive amino acids, or ≥20 consecutive amino acids, or ≥30 consecutive amino acids, e.g., ≥40 consecutive amino acids, such as for example ≥50 consecutive amino acids, e.g., ≥60, ≥70, ≥80, ≥90, ≥100, ≥200, ≥300, ≥400, ≥500 or ≥600 consecutive amino acids of the corresponding full-length peptide, polypeptide, or protein. The term “fragment” with reference to a nucleic acid (polynucleotide) generally denotes a 5′- and/or 3′-truncated form of a nucleic acid. Preferably, a fragment may comprise at least about 30%, e.g., at least about 50% or at least about 70%, preferably at least about 80%, e.g., at least about 85%, more preferably at least about 90%, and yet more preferably at least about 95% or even about 99% of the nucleic acid sequence length of said nucleic acid. For example, insofar not exceeding the length of the full-length nucleic acid, a fragment may include a sequence of ≥5 consecutive nucleotides, or ≥10 consecutive nucleotides, or ≥20 consecutive nucleotides, or ≥30 consecutive nucleotides, e.g., ≥40 consecutive nucleotides, such as for example ≥50 consecutive nucleotides, e.g., ≥60, ≥70, ≥80, ≥90, ≥100, ≥200, ≥300, ≥400, ≥500 or ≥600 consecutive nucleotides of the corresponding full-length nucleic acid. The terms encompass fragments arising by any mechanism, in vivo and/or in vitro, such as, without limitation, by alternative transcription or translation, exo- and/or endo-proteolysis, exo- and/or endo-nucleolysis, or degradation of the peptide, polypeptide, protein, or nucleic acid, such as, for example, by physical, chemical and/or enzymatic proteolysis or nucleolysis.

As used herein, “gene” refers to a hereditary unit corresponding to a sequence of DNA that occupies a specific location on a chromosome and that contains the genetic instruction for a characteristic(s) or trait(s) in an organism. The term gene can refer to translated and/or untranslated regions of a genome. “Gene” can refer to the specific sequence of DNA that is transcribed into an RNA transcript that can be translated into a polypeptide or be a catalytic RNA molecule, including but not limited to, tRNA, siRNA, piRNA, miRNA, long-non-coding RNA, shRNA, and/or the like.

As used herein, “identity,” refers to a relationship between two or more nucleotide or polypeptide sequences, as determined by comparing the sequences. In the art, “identity” also refers to the degree of sequence relatedness between polynucleotide or polypeptide sequences as determined by the match between strings of such sequences. “Identity” can be readily calculated by known methods, including, but not limited to, those described in (Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J. Applied Math. 1988, 48: 1073. Preferred methods to determine identity are designed to give the largest match between the sequences tested. Methods to determine identity are codified in publicly available computer programs. The percent identity between two sequences can be determined by using analysis software (e.g., Sequence Analysis Software Package of the Genetics Computer Group, Madison Wis.) that incorporates the Needelman and Wunsch, (J. Mol. Biol., 1970, 48: 443-453,) algorithm (e.g., NBLAST, and XBLAST). The default parameters are used to determine the identity for the polypeptides or polynucleotides of the present disclosure, unless stated otherwise.

An “individual discrete volume” is a discrete volume or discrete space, such as a container, receptacle, or other defined volume or space that can be defined by properties that prevent and/or inhibit migration of nucleic acids and reagents necessary to carry out the methods disclosed herein, for example a volume or space defined by physical properties such as walls, for example the walls of a well, tube, or a surface of a droplet, which may be impermeable or semipermeable, or as defined by other means such as chemical, diffusion rate limited, electro-magnetic, or light illumination, or any combination thereof. By “diffusion rate limited” (for example diffusion defined volumes) is meant spaces that are only accessible to certain molecules or reactions because diffusion constraints effectively defining a space or volume as would be the case for two parallel laminar streams where diffusion will limit the migration of a target molecule from one stream to the other. By “chemical” defined volume or space is meant spaces where only certain target molecules can exist because of their chemical or molecular properties, such as size, where for example gel beads may exclude certain species from entering the beads but not others, such as by surface charge, matrix size or other physical property of the bead that can allow selection of species that may enter the interior of the bead. By “electro-magnetically” defined volume or space is meant spaces where the electro-magnetic properties of the target molecules or their supports such as charge or magnetic properties can be used to define certain regions in a space such as capturing magnetic particles within a magnetic field or directly on magnets. By “optically” defined volume is meant any region of space that may be defined by illuminating it with visible, ultraviolet, infrared, or other wavelengths of light such that only target molecules within the defined space or volume may be labeled. One advantage to the used of non-walled, or semipermeable is that some reagents, such as buffers, chemical activators, or other agents maybe passed in through the discrete volume, while other material, such as target molecules, maybe maintained in the discrete volume or space. Typically, a discrete volume will include a fluid medium, (for example, an aqueous solution, an oil, a buffer, and/or a media capable of supporting cell growth) suitable for labeling of the target molecule with the indexable nucleic acid identifier under conditions that permit labeling. Exemplary discrete volumes or spaces useful in the disclosed methods include droplets (for example, microfluidic droplets and/or emulsion droplets), hydrogel beads or other polymer structures (for example poly-ethylene glycol di-acrylate beads or agarose beads), tissue slides (for example, fixed formalin paraffin embedded tissue slides with particular regions, volumes, or spaces defined by chemical, optical, or physical means), microscope slides with regions defined by depositing reagents in ordered arrays or random patterns, tubes (such as, centrifuge tubes, microcentrifuge tubes, test tubes, cuvettes, conical tubes, and the like), bottles (such as glass bottles, plastic bottles, ceramic bottles, Erlenmeyer flasks, scintillation vials and the like), wells (such as wells in a plate), plates, pipettes, or pipette tips among others. In certain example embodiments, the individual discrete volumes are the wells of a microplate. In certain example embodiments, the microplate is a 96 well, a 384 well, or a 1536 well microplate.

The terms “nanoparticle” or “microparticle” as used herein includes a nanoscale or microscale, respectively, deposit of a homogenous or heterogeneous material. Nanoparticles and microparticles may be regular or irregular in shape and may be formed from a plurality of co-deposited particles that form a composite nanoscale or microscale particle. Nanoparticles and microparticles may be generally spherical in shape or have a composite shape formed from a plurality of co-deposited generally spherical particles. Exemplary shapes for the nanoparticles and microparticles include, but are not limited to, spherical, rod, elliptical, cylindrical, disc, and the like. In some embodiments, the nanoparticles or microparticles have a substantially spherical shape.

As used herein, “nucleic acid,” “nucleotide sequence,” and “polynucleotide” can be used interchangeably herein and can generally refer to a string of at least two base-sugar-phosphate combinations and refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, polynucleotide as used herein can refer to triple-stranded regions comprising RNA or DNA or both RNA and DNA. The strands in such regions can be from the same molecule or from different molecules. The regions may include all of one or more of the molecules, but more typically involve only a region of some of the molecules. One of the molecules of a triple-helical region often is an oligonucleotide. “Polynucleotide” and “nucleic acids” also encompasses such chemically, enzymatically or metabolically modified forms of polynucleotides, as well as the chemical forms of DNA and RNA characteristic of viruses and cells, including simple and complex cells, inter alia. For instance, the term polynucleotide as used herein can include DNAs or RNAs as described herein that contain one or more modified bases. Thus, DNAs or RNAs including unusual bases, such as inosine, or modified bases, such as tritylated bases, to name just two examples, are polynucleotides as the term is used herein. “Polynucleotide”, “nucleotide sequences” and “nucleic acids” also includes PNAs (peptide nucleic acids), phosphorothioates, and other variants of the phosphate backbone of native nucleic acids. Natural nucleic acids have a phosphate backbone, artificial nucleic acids can contain other types of backbones, but contain the same bases. Thus, DNAs or RNAs with backbones modified for stability or for other reasons are “nucleic acids” or “polynucleotides” as that term is intended herein. As used herein, “nucleic acid sequence” and “oligonucleotide” also encompasses a nucleic acid and polynucleotide as defined elsewhere herein.

As used herein, a “population” of cells is any number of cells greater than 1, but is preferably at least 1×10³ cells, at least 1×10⁴ cells, at least at least 1×10⁵ cells, at least 1×10⁶ cells, at least 1×10⁷ cells, at least 1×10⁸ cells, at least 1×10⁹ cells, or at least 1×10¹⁰ cells.

As used herein, “polypeptides” or “proteins” refers to amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V). “Protein” and “Polypeptide” can refer to a molecule composed of one or more chains of amino acids in a specific order. The term protein is used interchangeable with “polypeptide.” The order is determined by the base sequence of nucleotides in the gene coding for the protein. Proteins can be required for the structure, function, and regulation of the body's cells, tissues, and organs.

As used herein, the term “recombinant” or “engineered” can generally refer to a non-naturally occurring nucleic acid, nucleic acid construct, or polypeptide. Such non-naturally occurring nucleic acids may include natural nucleic acids that have been modified, for example that have deletions, substitutions, inversions, insertions, etc., and/or combinations of nucleic acid sequences of different origin that are joined using molecular biology technologies (e.g., a nucleic acid sequences encoding a fusion protein (e.g., a protein or polypeptide formed from the combination of two different proteins or protein fragments), the combination of a nucleic acid encoding a polypeptide to a promoter sequence, where the coding sequence and promoter sequence are from different sources or otherwise do not typically occur together naturally (e.g., a nucleic acid and a constitutive promoter), etc. Recombinant or engineered can also refer to the polypeptide encoded by the recombinant nucleic acid. Non-naturally occurring nucleic acids or polypeptides include nucleic acids and polypeptides modified by man.

As used herein, the term “specific binding” refers to non-covalent physical association of a first and a second moiety wherein the association between the first and second moieties is at least 2 times as strong, at least 5 times as strong as, at least 10 times as strong as, at least 50 times as strong as, at least 100 times as strong as, or stronger than the association of either moiety with most or all other moieties present in the environment in which binding occurs. Binding of two or more entities may be considered specific if the equilibrium dissociation constant, Kd, is 10⁻³ M or less, 10⁻⁴ M or less, 10⁻⁵ M or less, 10⁻⁶ M or less, 10⁻⁷ M or less, 10⁻⁸ M or less, 10⁻⁹ M or less, 10⁻¹⁰ M or less, 10⁻¹¹ M or less, or 10⁻¹² M or less under the conditions employed, e.g., under physiological conditions such as those inside a cell or consistent with cell survival. In some embodiments, specific binding can be accomplished by a plurality of weaker interactions (e.g., a plurality of individual interactions, wherein each individual interaction is characterized by a Kd of greater than 10⁻³ M). In some embodiments, specific binding, which can be referred to as “molecular recognition,” is a saturable binding interaction between two entities that is dependent on complementary orientation of functional groups on each entity. Examples of specific binding interactions include primer-polynucleotide interaction, aptamer-aptamer target interactions, antibody-antigen interactions, avidin-biotin interactions, ligand-receptor interactions, metal-chelate interactions, hybridization between complementary nucleic acids, etc.

As used herein in the context of polynucleotides and polypeptides, “variant” can refer to a polynucleotide or polypeptide that differs from a reference polynucleotide or polypeptide, but retains essential and/or characteristic properties (structural and/or functional) of the reference polynucleotide or polypeptide. A typical variant of a polypeptide differs in amino acid sequence from another, reference polypeptide. The differences can be limited so that the sequences of the reference polypeptide and the variant are closely similar overall and, in many regions, identical. A variant and reference polypeptide may differ in nucleic or amino acid sequence by one or more modifications at the sequence level or post-transcriptional or post-translational modifications (e.g., substitutions, additions, deletions, methylation, glycosylations, etc.). A substituted nucleic acid may or may not be an unmodified nucleic acid of adenine, thiamine, guanine, cytosine, uracil, including any chemically, enzymatically or metabolically modified forms of these or other nucleotides. A substituted amino acid residue may or may not be one encoded by the genetic code. A variant of a polypeptide may be naturally occurring such as an allelic variant, or it may be a variant that is not known to occur naturally. “Variant” includes functional and structural variants.

As used herein, the terms “weight percent,” “wt %,” and “wt. %,” which can be used interchangeably, indicate the percent by weight of a given component based on the total weight of a composition of which it is a component, unless otherwise specified. That is, unless otherwise specified, all wt % values are based on the total weight of the composition. It should be understood that the sum of wt % values for all components in a disclosed composition or formulation are equal to 100. Alternatively, if the wt % value is based on the total weight of a subset of components in a composition, it should be understood that the sum of wt % values the specified components in the disclosed composition or formulation are equal to 100.

As used herein, the term “effective proximity” refers to the distance, region, or area surrounding a reference point, molecule, compound, or object in which a desired effect or activity occurs. The effective proximity can be determined by measuring the desired effect or activity in a representative number of species in the area surrounding the reference point or object. By way of non-limiting examples, an agent can be delivered to a specific point in a tissue of a subject and can be diffused through the surrounding tissue and cause effects in cells at a distance from the initial point of delivery. Cells that are affected by the agent can be determined and thus the region of effective proximity can be determined. Cells within that region are said to be within effective proximity to the initial delivery point. Similarly, if a cell is engineered to produce a product and secretes it into the surrounding environment, cells in the surrounding environment that are affected by the secreted product are said to be within effective proximity to the producing cell (or reference point). Likewise, if two (or more) molecules, compounds, compositions, objects, and/or the like are in effective proximity to one another, such a distance, region, or area can be defined and/or determined by measuring a change in one or more of the molecules, compounds, compositions, objects, and/or the like, a product produced from the molecules, compounds, compositions, objects, and/or the like (e.g., light, heat, or product compound, composition and/or the like). The molecules, compounds, compositions, objects, and/or the like are in “effective proximity” at the physical distance(s), position(s), etc. where a change, reaction, product, and/or the like is produced. In some embodiments, effective proximity ranges from 0 to 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1100, 1110, 1120, 1130, 1140, 1150, 1160, 1170, 1180, 1190, 1200, 1210, 1220, 1230, 1240, 1250, 1260, 1270, 1280, 1290, 1300, 1310, 1320, 1330, 1340, 1350, 1360, 1370, 1380, 1390, 1400, 1410, 1420, 1430, 1440, 1450, 1460, 1470, 1480, 1490, 1500, 1510, 1520, 1530, 1540, 1550, 1560, 1570, 1580, 1590, 1600, 1610, 1620, 1630, 1640, 1650, 1660, 1670, 1680, 1690, 1700, 1710, 1720, 1730, 1740, 1750, 1760, 1770, 1780, 1790, 1800, 1810, 1820, 1830, 1840, 1850, 1860, 1870, 1880, 1890, 1900, 1910, 1920, 1930, 1940, 1950, 1960, 1970, 1980, 1990, 2000 angstroms, pm, microns, or mm away from the reference point. In some embodiments, direct contact or bonding (i.e., effective proximity is 0).

All publications, published patent documents, and patent applications cited herein are hereby incorporated by reference to the same extent as though each individual publication, published patent document, or patent application was specifically and individually indicated as being incorporated by reference.

### Overview

Inteins are protein splicing sequences capable of self-excision and ligation of the fused extein proteins in a precise manner. Cysteine-less (CL) split inteins are of particular interest, although very rare, since there are almost no restrictions in using such systems for wide protein engineering and therapeutic applications. Described in several example embodiments herein are engineered split intein systems that include an RspCL split-intein encoded by cyanobacteria Richelia sp and uses thereof. Applicants describe and demonstrate embodiments of an Rsp CL split-intein system encoded by cyanobacteria Richelia sp. that equipped with an unusual protein trans-splicing activity. Investigation by a dual-fluorescent reporter system generated by Applicant demonstrated, inter alia, the protein trans-splicing activity of the Rsp CL split-intein in a wide range of conditions representing various pH, temperatures, reducing agents, salts, and denaturing agents, covering many relevant conditions used for protein engineering and therapeutic applications. In such situations, the Rsp CL intein catalyzes its protein bioconjugation (such as a trans-splicing reaction) to completion within minutes to hours in a highly specific manner.

Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### Engineered Split-Intein Systems

Described in certain example embodiments herein are engineered intein system comprising a recombinant first amino acid sequence comprising an N-terminal intein sequence; and a recombinant second amino acid sequence comprising a C-terminal intein sequence, wherein the N-terminal intein sequence, the C-terminal intein sequence, or both are derived from a split intein of Richelia sp., Pseudomonas aeruginosa 18, Candidatus Brocadiales, or any combination thereof. The term “amino acid sequence” and/or the like, such as when referred to a specific sequence (e.g., an N-terminal intein sequence), refers to a polypeptide having an amino acid sequence. Thus, a first amino acid sequence comprising an N-terminal intein sequence, as the phrase is used herein, refers to a first polypeptide having an amino acid sequence comprising an N-terminal intein polypeptide that has an N-terminal intein amino acid sequence.

Also described herein are recombinant first polypeptides that contain an N-terminal intein polypeptide, wherein the N-terminal intein polypeptide is derived from a split intein of Richelia sp., Pseudomonas aeruginosa 18, or Candidatus Brocadiales. Also described herein are recombinant second polypeptides that contain an N-terminal intein polypeptide, wherein the C-terminal intein polypeptide is derived from a split intein of Richelia sp., Pseudomonas aeruginosa 18, or Candidatus Brocadiales. The recombinant first and second polypeptides can each further comprise one or more additional polypeptides in addition to the N-terminal intein or the C-terminal intein polypeptides. In some embodiments, the one or more additional polypeptides are proteins of interest. The term “protein of interest” (used interchangeably with “polypeptide of interest”) refers to proteins that are identified as those in which it is desirable to bioconjugate to another polypeptide via the engineered intein system herein. In some embodiments, the engineered intein system contains one protein of interest. In some embodiments, the engineered intein system contains two proteins of interest. In some embodiments, one or each of the first or second amino acid sequences comprises a protein of interest.

Also described in several embodiments herein are engineered polynucleotides encoding the engineered intein systems and/or components thereof. Also described in several embodiments herein are vectors and vector systems containing the engineered polynucleotide(s) encoding the engineered intein system(s) and/or components thereof.

### Engineered Intein Systems and System Polypeptides

In some embodiments, the engineered intein system is composed of comprising a recombinant first amino acid sequence comprising an N-terminal intein sequence; and a recombinant second amino acid sequence comprising a C-terminal intein sequence, wherein the N-terminal intein sequence, the C-terminal intein sequence, or both are derived from a split intein of Richelia sp., Pseudomonas aeruginosa 18, Candidatus Brocadiales, or any combination thereof. In some embodiments, the first amino acid sequence comprises a protein amino acid sequence that is not an N-terminal intein sequence. In some embodiments, the first amino acid sequence comprises a protein amino acid sequence that is not an N-terminal intein sequence.

In certain example embodiments, the split intein is a cysteine-less split intein. In certain example embodiments, the N-terminal intein sequence comprises an amino acid sequence having about 80% to 100% sequence identity to any one of SEQ ID NO: 1, 3, 5, or 7. In certain example embodiments, the N-terminal intein sequence comprises an amino acid sequence having at least 80%, 85%, 90%, 95%, 98% 99% or 100% sequence identity to any one of SEQ ID NO: 1, 3, 5, or 7. In some embodiments, the N-terminal intein sequence comprises an amino acid sequence having about 80%, 81%, 82%, 83%, 84%, 85%, 86%, 87%, 88%, 89%, 90%, 91%, 92%, 93%, 94%, 95%, 96%, 97%, 98%, 99%, to/or 100% identity to any one of SEQ ID NO: 1, 3, 5, or 7.

In certain example embodiments, the C-terminal intein sequence comprises an amino acid sequence having about 80% to 100% sequence identity to any one of SEQ ID NO: 2, 4, 6, or 8. In certain example embodiments, the C-terminal intein sequence comprises an amino acid sequence having at least 80%, 85%, 90%, 95%, 98% 99% or 100% sequence identity to any one of SEQ ID NO: 2, 4, 6, or 8. In some embodiments, the C-terminal intein sequence comprises an amino acid sequence having about 80%, 81%, 82%, 83%, 84%, 85%, 86%, 87%, 88%, 89%, 90%, 91%, 92%, 93%, 94%, 95%, 96%, 97%, 98%, 99%, to/or 100% identity to any one of SEQ ID NO: SEQ ID NO: 2, 4, 6, or 8.

In certain example embodiments, the C-terminal intein sequence comprises X₁PYFFX₂NNILVHNS (SEQ ID NO: 10), wherein X₁ and X₂ are each independently selected from any amino acid.

In certain example embodiments, (a) wherein X₁ is selected from N or T, (b) wherein X₂ is selected from A or G, or (c) both (a) and (b).

In certain example embodiments, the C-terminal sequence comprises SEQ ID NO: 9.

In some embodiments, the N-terminal intein sequence is operatively coupled to a C-terminus of the first amino acid sequence. In some embodiments, the N-terminal sequence is operatively coupled to a C-terminus of the first amino acid sequence via a peptide bond. In some embodiments, the N-terminal intein sequence is operatively coupled to a C-terminus of a second polypeptide sequence of the first amino acid sequence. In some embodiments, the N-terminal intein sequence is fused to a C-terminus of a second polypeptide sequence of the first amino acid sequence. In some embodiments, the N-terminal sequence is operatively coupled to the C-terminus of the first amino acid sequence via a linker.

In some embodiments, the C-terminal intein sequence is operatively coupled to a N-terminus of the second amino acid sequence. In some embodiments, the C-terminal sequence is operatively coupled to a N-terminus of the second amino acid sequence via a peptide bond. In some embodiments, the C-terminal intein sequence is operatively coupled to a N-terminus of a second polypeptide sequence of the second amino acid sequence. In some embodiments, the C-terminal intein sequence is fused to a N-terminus of a second polypeptide sequence of the second amino acid sequence.

In some embodiments, the N-terminal sequence is operatively coupled to the C-terminus of the first amino acid sequence via a linker. In some embodiments, the C-terminal sequence is operatively coupled to the N-terminus of the second amino acid sequence via a linker. In some embodiments, the linker operatively coupling the N-terminal sequence to the C-terminus of the first amino acid sequence, the linker operatively coupling the C-terminal sequence to the N-terminus of the second amino acid sequence, or both, are peptide linkers. In some embodiments, the linker operatively coupling the N-terminal sequence to the C-terminus of the first amino acid sequence, the linker operatively coupling the C-terminal sequence to the N-terminus of the second amino acid sequence, or both, is/are not more than 1, 2, 3, 4, 5, 6, 7, 8, 9, 10, 11, 12, 13, 14, 15, or 20 amino acids in length. In some embodiments, the linker is a flexible linker. In some embodiments, the linker is a rigid linker.

In some embodiments, the linker operatively coupling the N-terminal sequence to the C-terminus of the first amino acid sequence, the linker operatively coupling the C-terminal sequence to the N-terminus of the second amino acid sequence, or both, is/are a Gly-Ser linker. In some embodiments, the linker comprises or is composed only of an amino acid sequence of at least 80%, 85%, 90%, 95%, 98%, 99% or 100% sequence identity to GSGSGSGSGSGSGSGSGSGSG (SEQ ID NO: 11). In some embodiments, the linker comprises or is composed only of an amino acid sequence of at least 80%, 81%, 82%, 83%, 84%, 85%, 86%, 87%, 88%, 89%, 90%, 91%, 92%, 93%, 94%, 95%, 96%, 97%, 98%, 99%, to/or 100% sequence identity to GSGSGSGSGSGSGSGSGSGSG (SEQ ID NO: 11).

In some embodiments, the linker operatively coupling the N-terminal sequence to the C-terminus of the first amino acid sequence, the linker operatively coupling the C-terminal sequence to the N-terminus of the second amino acid sequence, or both, is/are an Asparagine-Serine linker. In some embodiments, the linker comprises or is composed only of an amino acid sequence of at least 80%, 85%, 90%, 95%, 98%, 99% or 100% sequence identity to ASASASASASASASASAS (SEQ ID NO: 12). In some embodiments, the linker comprises or is composed only of an amino acid sequence of at least 80%, 81%, 82%, 83%, 84%, 85%, 86%, 87%, 88%, 89%, 90%, 91%, 92%, 93%, 94%, 95%, 96%, 97%, 98%, 99%, to/or 100% sequence identity to ASASASASASASASASAS (SEQ ID NO: 12).

Additional exemplary linkers include those set forth in Chen et al., Adv Drug Deliv Rev. 2013 Oct. 15; 65(10): 1357-1369; Rosmalen et al., Biochem. 2017, 56, 50, 6565-6574; a Proline 9 (P9) linker, (GS)ₙ (n=1-10) (SEQ ID NO: 30-38), GAAPAAAPAKQEAAAPAPAAKAEAPAAAPAAKA (SEQ ID NO: 39), (GGGGS)₃ (SEQ ID NO: 40), (G)₈ (SEQ ID NO: 41), (G)₆ (SEQ ID NO: 42), (EAAAK)₃ (SEQ ID NO: 43), (EAAAK)ₙ (n=1-3) (SEQ ID NO: 44-45, 43), A(EAAAK)₄ALEA(EAAAK)₄A (SEQ ID NO: 46), GGGGS (SEQ ID NO: 47), PAPAP (SEQ ID NO: 48), AEAAAKEAAAKA (SEQ ID NO: 49), (GGGGS)ₙ (n=1-10) (SEQ ID NO: 47, 50, 40, 51-57), (Ala-Pro)n (n=10-32) (SEQ ID NO: 58-80), disulfide, LE, LEAGCKNFFPR!SFTSCGSLE (SEQ ID NO: 81), or CRRRRRREAEAC (SEQ ID NO: 82). Other suitable linkers will be appreciated by those of ordinary skill in the art in view of the description herein.

The engineered intein systems described herein can be capable of catalyzing a bioconjugation reaction (such as a protein trans-splicing reaction) under a broad range of conditions. In certain example embodiments, the system is capable of catalyzing a bioconjugation reaction at a pH ranging from about 6 to about 8. In certain example embodiments, the system is capable of catalyzing a bioconjugation reaction at a pH of about 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, to/or 8.

In certain example embodiments, the system is capable of catalyzing a bioconjugation reaction at a temperature ranging from about 20° C. to about 50° C. In certain example embodiments, the system is capable of catalyzing a bioconjugation reaction at a temperature of about 20° C., 20.5° C., 21° C., 21.5° C., 22° C., 22.5° C., 23° C., 23.5° C., 24° C., 24.5° C., 25° C., 25.5° C., 26° C., 26.5° C., 27° C., 27.5° C., 28° C., 28.5° C., 29° C., 29.5° C., 30° C., 30.5° C., 31° C., 31.5° C., 32° C., 32.5° C., 33° C., 33.5° C., 34° C., 34.5° C., 35° C., 35.5° C., 36° C., 36.5° C., 37° C., 37.5° C., 38° C., 38.5° C., 39° C., 39.5° C., 40° C., 40.5° C., 41° C., 41.5° C., 42° C., 42.5° C., 43° C., 43.5° C., 44° C., 44.5° C., 45° C., 45.5° C., 46° C., 46.5° C., 47° C., 47.5° C., 48° C., 48.5° C., 49° C., 49.5° C., to/or 50° C. In some embodiments, the system is capable of catalyzing a bioconjugation reaction at a temperature of about 25° C., 25.5° C., 26° C., 26.5° C., 27° C., 27.5° C., 28° C., 28.5° C., 29° C., 29.5° C., 30° C., 30.5° C., 31° C., 31.5° C., 32° C., 32.5° C., 33° C., 33.5° C., 34° C., 34.5° C., 35° C., 35.5° C., 36° C., 36.5° C., to/or 37° C.

In certain example embodiments, the system is capable of catalyzing a bioconjugation reaction, wherein the system is capable of catalyzing a bioconjugation reaction in the presence of a reducing agent, optionally wherein the reducing agent is dithiothreitol (DTT), beta mercaptoethanol (BME), tris(2-carboxyethyl)phosphine (TCEP), or cysteine.

In certain example embodiments, the system is capable of catalyzing a bioconjugation reaction in the presence of about 0.05 M NaCl to about 2 M NaCl. In some embodiments, the system is capable of catalyzing a bioconjugation reaction in the presence of about 0.05 M, 0.06 M, 0.07 M, 0.08 M, 0.09 M, 0.1 M, 0.11 M, 0.12 M, 0.13 M, 0.14 M, 0.15 M, 0.16 M, 0.17 M, 0.18 M, 0.19 M, 0.2 M, 0.21 M, 0.22 M, 0.23 M, 0.24 M, 0.25 M, 0.26 M, 0.27 M, 0.28 M, 0.29 M, 0.3 M, 0.31 M, 0.32 M, 0.33 M, 0.34 M, 0.35 M, 0.36 M, 0.37 M, 0.38 M, 0.39 M, 0.4 M, 0.41 M, 0.42 M, 0.43 M, 0.44 M, 0.45 M, 0.46 M, 0.47 M, 0.48 M, 0.49 M, 0.5 M, 0.51 M, 0.52 M, 0.53 M, 0.54 M, 0.55 M, 0.56 M, 0.57 M, 0.58 M, 0.59 M, 0.6 M, 0.61 M, 0.62 M, 0.63 M, 0.64 M, 0.65 M, 0.66 M, 0.67 M, 0.68 M, 0.69 M, 0.7 M, 0.71 M, 0.72 M, 0.73 M, 0.74 M, 0.75 M, 0.76 M, 0.77 M, 0.78 M, 0.79 M, 0.8 M, 0.81 M, 0.82 M, 0.83 M, 0.84 M, 0.85 M, 0.86 M, 0.87 M, 0.88 M, 0.89 M, 0.9 M, 0.91 M, 0.92 M, 0.93 M, 0.94 M, 0.95 M, 0.96 M, 0.97 M, 0.98 M, 0.99 M, 1 M, 1.01 M, 1.02 M, 1.03 M, 1.04 M, 1.05 M, 1.06 M, 1.07 M, 1.08 M, 1.09 M, 1.1 M, 1.11 M, 1.12 M, 1.13 M, 1.14 M, 1.15 M, 1.16 M, 1.17 M, 1.18 M, 1.19 M, 1.2 M, 1.21 M, 1.22 M, 1.23 M, 1.24 M, 1.25 M, 1.26 M, 1.27 M, 1.28 M, 1.29 M, 1.3 M, 1.31 M, 1.32 M, 1.33 M, 1.34 M, 1.35 M, 1.36 M, 1.37 M, 1.38 M, 1.39 M, 1.4 M, 1.41 M, 1.42 M, 1.43 M, 1.44 M, 1.45 M, 1.46 M, 1.47 M, 1.48 M, 1.49 M, 1.5 M, 1.51 M, 1.52 M, 1.53 M, 1.54 M, 1.55 M, 1.56 M, 1.57 M, 1.58 M, 1.59 M, 1.6 M, 1.61 M, 1.62 M, 1.63 M, 1.64 M, 1.65 M, 1.66 M, 1.67 M, 1.68 M, 1.69 M, 1.7 M, 1.71 M, 1.72 M, 1.73 M, 1.74 M, 1.75 M, 1.76 M, 1.77 M, 1.78 M, 1.79 M, 1.8 M, 1.81 M, 1.82 M, 1.83 M, 1.84 M, 1.85 M, 1.86 M, 1.87 M, 1.88 M, 1.89 M, 1.9 M, 1.91 M, 1.92 M, 1.93 M, 1.94 M, 1.95 M, 1.96 M, 1.97 M, 1.98 M, 1.99 M, to/or 2.00 M.

Also described herein are polynucleotides encoding one or more of the engineered intein system polypeptides described herein. As used herein, the term “encode” refers to principle that DNA can be transcribed into RNA, which can then be translated into amino acid sequences that can form proteins. Encoding polynucleotides can be DNA or RNA. In some embodiments, the encoding polynucleotides are codon optimized. Codon optimization of polynucleotides are described in greater detail elsewhere herein.

### Tags

In certain example embodiments, the system further comprises a targeting moiety, localization tag, affinity tag, reporter tag, or any combination thereof, wherein the localization tag, affinity tag, reporter tag, or any combination thereof is operatively coupled to the first amino acid sequence, the second amino acid sequence, or both. In certain example embodiments, the system further comprises a localization tag, affinity tag, reporter tag, or any combination thereof, wherein the localization tag, affinity tag, reporter tag, or any combination thereof is operatively coupled to the first amino acid sequence, the second amino acid sequence, or both via a linker. In some embodiments, the linker is a flexible linker or a rigid linker. In some embodiments, the linker is a peptide linker. In some embodiments, the linker is a non-cleavable linker. In some embodiments, the linker is a cleavable linker. In some embodiments, the cleavable linker is cleaved by an enzyme, light, radiation, a chemical reaction, and/or the like.

In some embodiments, the peptide linker has a sequence of GGGLLK (SEQ ID NO: 83). In some embodiments, the peptide linker has a sequence of GGGLLK (SEQ ID NO: 83), wherein L4 and/or L5 are D-Leu. In some embodiments, the peptide linker has a sequence of GGG[GGS]₇K (SEQ ID NO: 84). In some embodiments, the peptide linker has a sequence of GGG[GGS]₇K (SEQ ID NO: 84), where S is L-Ser and/or K is L-Lys. In some embodiments, the peptide linker contains an N^{ε}-linked α-bromoacetyl group. In some embodiments, the peptide linker contains an N^{ε}-linked maleimide group. In some embodiments, the peptide linker is linker peptide 1, 2, or 3 of Lu et al., ACS Cent. Sci. 2021. 7:365-378. In some embodiments, the peptide linker comprises LPSTGGK (SEQ ID NO: 85). Additional exemplary linkers include those set forth in Chen et al., Adv Drug Deliv Rev. 2013 Oct. 15; 65(10): 1357-1369; Rosmalen et al., Biochem. 2017, 56, 50, 6565-6574; a Proline 9 (P9) linker, GAAPAAAPAKQEAAAPAPAAKAEAPAAAPAAKA (SEQ ID NO: 39), (GGGGS)₃ (SEQ ID NO: 40), (G)₈ (SEQ ID NO: 41), (G)₆ (SEQ ID NO: 42), (GS)ₙ (n=1-10) (SEQ ID NO: 30-38) (EAAAK)₃ (SEQ ID NO: 43), (EAAAK)ₙ (n=1-3) (SEQ ID NO: 44-45, 43), A(EAAAK)₄ALEA(EAAAK)₄A (SEQ ID NO: 46), GGGGS (SEQ ID NO: 47), PAPAP (SEQ ID NO: 48), AEAAAKEAAAKA (SEQ ID NO: 49), (GGGGS)ₙ (n=1-10) (SEQ ID NO: 47, 50, 40, 51-57), (Ala-Pro)n (n=10-32) (SEQ ID NO: 58-80), disulfide, VSQTSKLTR↓AETVFPDV (SEQ ID NO: 86), PLG↓LWA (SEQ ID NO: 87), RVL↓AEA (SEQ ID NO: 88); EDVVCC↓SMSY (SEQ ID NO: 89); GGIEGR↓GS (SEQ ID NO: 90), TRHRQPR↓GWE (SEQ ID NO: 91); AGNRVRR↓SVG (SEQ ID NO: 92); RRRRRRR↓R↓R (SEQ ID NO: 93), GFLG↓ (SEQ ID NO: 94), LE, LEAGCKNFFPR!SFTSCGSLE (SEQ ID NO: 81), CRRRRRREAEAC (SEQ ID NO: 82), (Protease-sensitive cleavage sites are indicated with “↓”) or any combination thereof. In some embodiments, the linker is or comprises LPSTGGK (SEQ ID NO: 85). Other suitable linkers will be appreciated by those of ordinary skill in the art in view of the description herein.

### Targeting Moieties

As used herein, “targeting moiety” refers to molecules, complexes, agents, and the like that is capable of specifically or selectively interacting with, binding with, acting on or with, or otherwise associating or recognizing a target molecule, agent, and/or complex that is associated with, part of, coupled to, another object, complex, surface, and the like, such as a cell or cell population, tissue, organ, subcellular locale, object surface, particle etc. Targeting moieties can be chemical, biological, metals, polymers, or other agents and molecules with targeting capabilities. Targeting moieties can be amino acids, peptides, polypeptides, nucleic acids, polynucleotides, lipids, sugars, metals, small molecule chemicals, combinations thereof, and the like. Targeting moieties can be antibodies or fragments thereof, aptamers, DNA, RNA such as guide RNA for a RNA guided nuclease or system, ligands, substrates, enzymes, combinations thereof, and the like. The specificity or selectivity of a targeting moiety can be determined by any suitable method or technique that will be appreciated by those of ordinary skill in the art. For example, in some embodiments, the methods described herein include determining the disassociation constant for the targeting moiety and target. In some embodiments, the targeting moiety has a specificity the equilibrium dissociation constant, Kd, is 10⁻³ M or less, 10⁻⁴ M or less, 10⁻⁵ M or less, 10⁻⁶ M or less, 10⁻⁷ M or less, 10⁻⁸ M or less, 10⁻⁹ M or less, 10⁻¹⁰ M or less, 10⁻¹¹ M or less, or 10⁻¹² M or less under the conditions employed, e.g., under physiological conditions such as those inside a cell or consistent with cell survival. In some embodiments, specific binding can be accomplished by a plurality of weaker interactions (e.g., a plurality of individual interactions, wherein each individual interaction is characterized by a Kd of greater than 10⁻³ M). In some embodiments, the targeting moiety has increased binding with, association with, interaction with, activity on as compared to non-targets, such as a 1 to 500 or more fold increase. Targets of targeting moieties can be amino acids, peptides, polypeptides, nucleic acids, polynucleotides, lipids, sugars, metals, small molecule chemicals, combinations thereof, and the like. Targets can be receptors, biomarkers, transporters, antigens, complexes, combinations thereof, and the like.

In some embodiments, the targeting moiety targets a specific cell or tissue type and/or cell state. As used herein, “cell state” is used to describe transient elements of a cell's identity. Cell state can be thought of as the transient characteristic profile or phenotype of a cell. Cell states arise transiently during time-dependent processes, either in a temporal progression that is unidirectional (e.g., during differentiation, or following an environmental stimulus) or in a state vacillation that is not necessarily unidirectional and in which the cell may return to the origin state. Vacillating processes can be oscillatory (e.g., cell-cycle or circadian rhythm) or can transition between states with no predefined order (e.g., due to stochastic, or environmentally controlled, molecular events). These time-dependent processes may occur transiently within a stable cell type (as in a transient environmental response), or may lead to a new, distinct type (as in differentiation). View Wagner et al., 2016. Nat Biotechnol. 34(11): 1145-1160.

### Reporter and Affinity Molecules and Tags

In certain example embodiments, the engineered intein system or polypeptides thereof further comprises one or more reporter molecule operatively coupled to the first amino acid sequence, the second amino acid sequence or both.

Exemplary reporter proteins and tags include, but are not limited to, affinity tags, such as chitin binding protein (CBP), maltose binding protein (MBP), glutathione-S-transferase (GST), poly(His) tag; solubilization tags such as thioredoxin (TRX) and poly(NANP), MBP, and GST; chromatography tags such as those consisting of polyanionic amino acids, such as FLAG-tag; epitope tags such as V5-tag, Myc-tag, HA-tag and NE-tag; protein tags that can allow specific enzymatic modification (such as biotinylation by biotin ligase) or chemical modification (such as reaction with FlAsH-EDT2 for fluorescence imaging), DNA and/or RNA segments that contain restriction enzyme or other enzyme cleavage sites; DNA segments that encode products that provide resistance against otherwise toxic compounds including antibiotics, such as spectinomycin, ampicillin, kanamycin, tetracycline, Basta, neomycin phosphotransferase II (NEO), hygromycin phosphotransferase (HPT) and the like; DNA and/or RNA segments that encode products that are otherwise lacking in the recipient cell (e.g., tRNA genes, auxotrophic markers); DNA and/or RNA segments that encode products which can be readily identified (e.g., phenotypic markers such as β-galactosidase, GUS); fluorescent proteins such as green fluorescent protein (GFP), cyan fluorescent protein (CFP), yellow fluorescent protein (YFP), red fluorescent protein (RFP), mCherry, or other optically active proteins e.g., luciferase, and cell surface proteins); optically active dyes (e.g., fluorescent, UV, IR, and NIR dyes), polynucleotides that can generate one or more new primer sites for PCR (e.g., the juxtaposition of two DNA sequences not previously juxtaposed), DNA sequences not acted upon or acted upon by a restriction endonuclease or other DNA modifying enzyme, chemical, etc.; epitope tags (e.g., GFP, FLAG- and His-tags), and, DNA sequences that make a molecular barcode or unique molecular identifier (UMI), DNA sequences required for a specific modification (e.g., methylation) that allows its identification. Other suitable markers will be appreciated by those of skill in the art.

### Localization Signals

In some embodiments, the engineered intein system or component thereof includes one or more nuclear localization signals at the C-terminus, the N-terminus, or both the N- and C-terminus of the first and/or the second amino acid sequence of an engineered intein system. Without being bound by theory the localization signal can provide localization of an engineered intein system or component thereof to a location within a cell, such as a nucleus, Golgi, endoplasmic reticulum, cytoskeleton, gap junctions etc.

In some embodiments, the engineered intein system or component thereof includes one or more nuclear localization signals (NLSs) at the C-terminus, the N-terminus, or both the N- and C-terminus of the first and/or the second amino acid sequence of an engineered intein system. Without being bound by theory, such sequences may increase the transport of the engineered intein system or component thereof to the nucleus of a cell.

In some embodiments, the NLSs used in the context of the present disclosure are heterologous to the proteins. Non-limiting examples of NLSs include an NLS sequence derived from: the NLS of the SV40 virus large T-antigen, having the amino acid sequence PKKKRKV (SEQ ID NO: 95) or PKKKRKVEAS (SEQ ID NO: 96); the NLS from nucleoplasmin (e.g., the nucleoplasmin bipartite NLS with the sequence KRPAATKKAGQAKKKK (SEQ ID NO: 97)); the c-myc NLS having the amino acid sequence PAAKRVKLD (SEQ ID NO: 98) or RQRRNELKRSP (SEQ ID NO: 99); the hRNPA1 M9 NLS having the sequence NQSSNFGPMKGGNFGGRSSGPYGGGGQYFAKPRNQGGY (SEQ ID NO: 100); the sequence RMRIZFKNKGKDTAELRRRRVEVSVELRKAKKDEQILKRRNV (SEQ ID NO: 101) of the IBB domain from importin-alpha; the sequences VSRKRPRP (SEQ ID NO: 102) and PPKKARED (SEQ ID NO:9) of the myoma T protein; the sequence PQPKKKPL (SEQ ID NO: 103) of human p53; the sequence SALIKKKKKMAP (SEQ ID NO: 104) of mouse c-abl IV; the sequences DRLRR (SEQ ID NO: 105) and PKQKKRK (SEQ ID NO: 106) of the influenza virus NS1; the sequence RKLKKKIKKL (SEQ ID NO: 107) of the Hepatitis virus delta antigen; the sequence REKKKFLKRR (SEQ ID NO: 108) of the mouse Mx1 protein; the sequence KRKGDEVDGVDEVAKKKSKK (SEQ ID NO: 109) of the human poly(ADP-ribose) polymerase; and the sequence RKCLQAGMNLEARKTKK (SEQ ID NO: 110) of the steroid hormone receptors (human) glucocorticoid, TAT, and R10, or any combination thereof. Additional NLSs that are suitable for use with the present invention as described herein are any of those in Srivaths et al. Bioinformation 2018, 14(3), 132; Physiol. Res. 67 (Suppl. 2): S267-S279, 2018; and Lange et al. J. Biol. Chem. 2007, 282(8), 5101-5105.

Exemplary localization tags for targeting the Golgi and endoplasmic reticulum includes, but are not limited to, the internal region spanning HT18 and -19 of mTOR (see e.g., Liu and Zheng. Mol Biol Cell. 2007 March; 18(3): 1073-1082), altORF peptide (see e.g., Navarro and Cheeseman. 2022 MCB. 33(12) https://doi.org/10.1091/mbc.E22-03-0091).

### Delivery

The present disclosure also provides delivery systems for introducing components of the engineered intein systems and compositions herein to cells, tissues, organs, or organisms. A delivery system may comprise one or more delivery vehicles and/or cargos. In some embodiments, the cargos can be an engineered intein system or component thereof, encoding polynucleotide(s), vector(s), and/or vector systems of the present invention. Exemplary delivery systems and methods include those described herein and in paragraphs [00117] to [00278] of Feng Zhang et al., (WO2016106236A1), which is incorporated by reference herein in their entireties.

In some embodiments, the delivery systems may be used to introduce the components of the engineered intein systems, encoding polynucleotides, and compositions to plant cells. For example, the components may be delivered to plant using electroporation, microinjection, aerosol beam injection of plant cell protoplasts, biolistic methods, DNA particle bombardment, and/or Agrobacterium-mediated transformation. Examples of methods and delivery systems for plants include those described in Fu et al., Transgenic Res. 2000 February; 9(1):11-9; Klein R M, et al., Biotechnology. 1992; 24:384-6; Casas A M et al., Proc Natl Acad Sci USA. 1993 Dec. 1; 90(23): 11212-11216; and U.S. Pat. No. 5,563,055, Davey M R et al., Plant Mol Biol. 1989 September; 13(3):273-85, which are incorporated by reference herein in their entireties.

### Cargos

The delivery systems may comprise one or more cargos. The one or more cargos can comprise or consist of one or more engineered intein systems or component(s) thereof, encoding polynucleotide(s), vector(s), and/or vector system(s) of the present invention.

### Physical Delivery

In some embodiments, the cargos may be introduced to cells by physical delivery methods. Examples of physical methods include microinjection, electroporation, and hydrodynamic delivery. Both nucleic acid and proteins may be delivered using such methods. For example, engineered intein system proteins may be prepared in vitro, isolated, (refolded, purified if needed), and introduced to cells.

### Microinjection

Microinjection of the cargo directly to cells can achieve high efficiency, e.g., above 90% or about 100%. In some embodiments, microinjection may be performed using a microscope and a needle (e.g., with 0.5-5.0 m in diameter) to pierce a cell membrane and deliver the cargo directly to a target site within the cell. Microinjection may be used for in vitro and ex vivo delivery.

Plasmids or other vectors comprising coding sequences for engineered intein system proteins may be microinjected. In some cases, microinjection may be used i) to deliver DNA directly to a cell nucleus, and/or ii) to deliver mRNA (e.g., in vitro transcribed) to a cell nucleus or cytoplasm. In certain examples, microinjection may be used to deliver directly to the nucleus or cytoplasm engineered intein system proteins. In certain examples, microinjection may be used to deliver engineered intein system protein-encoding mRNA directly to the cytoplasm.

Microinjection may be used to generate genetically modified animals. For example, gene editing cargos may be injected into zygotes to allow for efficient germline modification. Such approach can yield normal embryos and full-term mouse pups harboring the desired modification(s). Microinjection can also be used to provide transient delivery of the engineered intein system proteins.

### Electroporation

In some embodiments, the cargos and/or delivery vehicles may be delivered by electroporation. Electroporation may use pulsed high-voltage electrical currents to transiently open nanometer-sized pores within the cellular membrane of cells suspended in buffer, allowing for components with hydrodynamic diameters of tens of nanometers to flow into the cell. In some cases, electroporation may be used on various cell types and efficiently transfer cargo into cells. Electroporation may be used for in vitro and ex vivo delivery.

Electroporation may also be used to deliver the cargo to into the nuclei of mammalian cells by applying specific voltage and reagents, e.g., by nucleofection. Such approaches include those described in Wu Y, et al. (2015). Cell Res 25:67-79; Ye L, et al. (2014). Proc Natl Acad Sci USA 111:9591-6; Choi P S, Meyerson M. (2014). Nat Commun 5:3728; Wang J, Quake S R. (2014). Proc Natl Acad Sci 111:13157-62. Electroporation may also be used to deliver the cargo in vivo, e.g., with methods described in Zuckermann M, et al. (2015). Nat Commun 6:7391.

### Hydrodynamic Delivery

Hydrodynamic delivery may also be used for delivering the cargos, e.g., for in vivo delivery. In some examples, hydrodynamic delivery may be performed by rapidly pushing a large volume (8-10% body weight) solution containing the gene editing cargo into the bloodstream of a subject (e.g., an animal or human), e.g., for mice, via the tail vein. As blood is incompressible, the large bolus of liquid may result in an increase in hydrodynamic pressure that temporarily enhances permeability into endothelial and parenchymal cells, allowing for cargo not normally capable of crossing a cellular membrane to pass into cells. This approach may be used for delivering naked DNA plasmids and proteins. The delivered cargos may be enriched in liver, kidney, lung, muscle, and/or heart.

### Transfection

The cargos, e.g., nucleic acids and/or polypeptides of the present invention, may be introduced to cells by transfection methods for introducing nucleic acids into cells. Examples of transfection methods include calcium phosphate-mediated transfection, cationic transfection, liposome transfection, dendrimer transfection, heat shock transfection, magnetofection, lipofection, impalefection, optical transfection, proprietary agent-enhanced uptake of nucleic acid.

### Transduction

The cargos, e.g., nucleic acids and/or polypeptides of the present invention, can be introduced to cells by transduction by a viral or pseudoviral particle. Methods of packaging the cargos in viral particles can be accomplished using any suitable viral vector or vector systems. Such viral vector and vector systems are described in greater detail elsewhere herein. As used in this context herein “transduction” refers to the process by which foreign nucleic acids and/or proteins are introduced to a cell (prokaryote or eukaryote) by a viral or pseudo viral particle. After packaging in a viral particle or pseudo viral particle, the viral particles can be exposed to cells (e.g., in vitro, ex vivo, or in vivo) where the viral or pseudoviral particle infects the cell and delivers the cargo to the cell via transduction. Viral and pseudoviral particles can be optionally concentrated prior to exposure to target cells. In some embodiments, the virus titer of a composition containing viral and/or pseudoviral particles can be obtained and a specific titer be used to transduce cells.

### Biolistics

The cargos, e.g., nucleic acids and/or polypeptides of the present invention, can be introduced to cells using a biolistic method or technique. The term of art “biolistic”, as used herein refers to the delivery of nucleic acids to cells by high-speed particle bombardment. In some embodiments, the cargo(s) can be attached, associated with, or otherwise coupled to particles, which than can be delivered to the cell via a gene-gun (see e.g., Liang et al. 2018. Nat. Protocol. 13:413-430; Svitashev et al. 2016. Nat. Comm. 7:13274; Ortega-Escalante et al., 2019. Plant. J. 97:661-672). In some embodiments, the particles can be gold, tungsten, palladium, rhodium, platinum, or iridium particles.

### Implantable Devices

In some embodiments, the delivery system can include an implantable device that incorporates or is coated with an engineered intein system or component thereof described herein. Various implantable devices are described in the art, and include any device, graft, sensor, or other composition or device that can be implanted into a subject.

### Delivery Vehicles

The delivery systems may comprise one or more delivery vehicles. The delivery vehicles may deliver the cargo into cells, tissues, organs, or organisms (e.g., animals or plants). The cargos may be packaged, carried, or otherwise associated with the delivery vehicles. The delivery vehicles may be selected based on the types of cargo to be delivered, and/or the delivery is in vitro and/or in vivo. Examples of delivery vehicles include vectors, viruses (e.g., virus particles), virus-like particles, non-viral vehicles, and other delivery reagents described herein.

The delivery vehicles described herein can have a greatest dimension or greatest average dimension (e.g., diameter or greatest average diameter) of less than 100 microns (μm). In some embodiments, the delivery vehicles have a greatest dimension or greatest average dimension of less than 10 μm. In some embodiments, the delivery vehicles may have a greatest dimension or greatest average dimension of less than 2000 nanometers (nm). In some embodiments, the delivery vehicles may have a greatest dimension or greatest average dimension of less than 1000 nanometers (nm). In some embodiments, the delivery vehicles may have a greatest dimension or greatest average dimension (e.g., diameter or average diameter) of less than 900 nm, less than 800 nm, less than 700 nm, less than 600 nm, less than 500 nm, less than 400 nm, less than 300 nm, less than 200 nm, less than 150 nm, or less than 100 nm, less than 50 nm. In some embodiments, the delivery vehicles may have a greatest dimension or greatest average dimension ranging between 25 nm and 200 nm.

In some embodiments, the delivery vehicles may be or comprise particles. For example, the delivery vehicle may be or comprise nanoparticles (e.g., particles with a greatest dimension or greatest average dimension (e.g., diameter or greatest average diameter) no greater than 1000 nm. The particles may be provided in different forms, e.g., as solid particles (e.g., metal such as silver, gold, iron, titanium), non-metal, lipid-based solids, polymers), suspensions of particles, or combinations thereof. Metal, dielectric, and semiconductor particles may be prepared, as well as hybrid structures (e.g., core-shell particles).

Nanoparticles may also be used to deliver the compositions and systems to cells, as described in WO 2008042156, US 20130185823, and WO2015089419. In general, a “nanoparticle” refers to any particle having a diameter of less than 1000 nm. In certain embodiments, nanoparticles of the invention have a greatest dimension or greatest average dimension (e.g., diameter or average diameter) of 500 nm or less. In other embodiments, nanoparticles of the invention have a greatest dimension or greatest average dimension ranging between 25 nm and 200 nm. In other embodiments, nanoparticles of the invention have a greatest dimension or greatest average dimension of 100 nm or less. In other embodiments, nanoparticles of the invention have a greatest dimension or greatest average dimensions ranging between 35 nm and 60 nm. It will be appreciated that reference made herein to particles or nanoparticles can be interchangeable, where appropriate. Nanoparticles made of semiconducting material may also be labeled quantum dots if they are small enough (typically sub 10 nm) that quantization of electronic energy levels occurs. Such nanoscale particles are used in biomedical applications as drug carriers or imaging agents and may be adapted for similar purposes in the present invention. Semi-solid and soft nanoparticles have been manufactured and are within the scope of the present invention. Nanoparticles with one half hydrophilic and the other half hydrophobic are termed Janus particles and are particularly effective for stabilizing emulsions. They can self-assemble at water/oil interfaces and act as solid surfactants.

Particle characterization (including e.g., characterizing morphology, dimension, etc.) is done using a variety of different techniques. Common techniques are electron microscopy (TEM, SEM), atomic force microscopy (AFM), dynamic light scattering (DLS), X-ray photoelectron spectroscopy (XPS), powder X-ray diffraction (XRD), Fourier transform infrared spectroscopy (FTIR), matrix-assisted laser desorption/ionization time-of-flight mass spectrometry (MALDI-TOF), ultraviolet-visible spectroscopy, dual polarization interferometry and nuclear magnetic resonance (NMR). Characterization (dimension measurements) may be made as to native particles (i.e., preloading) or after loading of the cargo (herein cargo refers to e.g., one or more components of engineered intein system and may include additional carriers and/or excipients) to provide particles of an optimal size for delivery for any in vitro, ex vivo and/or in vivo application of the present invention. In certain preferred embodiments, particle dimension (e.g., diameter) characterization is based on measurements using dynamic laser scattering (DLS). Mention is made of U.S. Pat. Nos. 8,709,843; 6,007,845; 5,855,913; 5,985,309; 5,543,158; and the publication by James E. Dahlman and Carmen Barnes et al. Nature Nanotechnology (2014) published online 11 May 2014, doi:10.1038/nnano.2014.84, describing particles, methods of making and using them and measurements thereof.

### Vectors and Vector Systems

Also provided herein are vectors that can contain one or more of the engineered intein system encoding polynucleotides described herein. In certain embodiments, the vector can contain one or more polynucleotides encoding one or more elements of an engineered intein system described herein. The vectors can be useful in producing bacterial, fungal, yeast, plant cells, animal cells, and transgenic animals that can express one or more components of the engineered intein system described herein. Within the scope of this disclosure are vectors containing one or more of the polynucleotide sequences described herein. The vectors and/or vector systems can be used, for example, to express one or more of the polynucleotides in a cell, such as a producer cell, to produce engineered intein system containing virus or virus-like particles described elsewhere herein. Other uses for the vectors and vector systems described herein are also within the scope of this disclosure. In general, and throughout this specification, the term “vector” refers to a tool that allows or facilitates the transfer of an entity from one environment to another. In some contexts which will be appreciated by those of ordinary skill in the art, “vector” can be a term of art to refer to a nucleic acid molecule capable of transporting another nucleic acid to which it has been linked. A vector can be a replicon, such as a plasmid, phage, or cosmid, into which another DNA segment may be inserted so as to bring about the replication of the inserted segment. Generally, a vector is capable of replication when associated with the proper control elements.

Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can be composed of a nucleic acid (e.g., a polynucleotide) of the invention in a form suitable for expression of the nucleic acid in a host cell, which means that the recombinant expression vectors include one or more regulatory elements, which can be selected on the basis of the host cells to be used for expression, that is operatively-linked to the nucleic acid sequence to be expressed. Within a recombinant expression vector, “operably linked” and “operatively-linked” are used interchangeably herein and further defined elsewhere herein. In the context of a vector, the term “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell). Advantageous vectors include lentiviruses and adeno-associated viruses, and types of such vectors can also be selected for targeting particular types of cells. These and other embodiments of the vectors and vector systems are described elsewhere herein.

In some embodiments, the vector can be a bicistronic vector. In some embodiments, a bicistronic vector can be used for one or more elements of the engineered intein system described herein. In some embodiments, expression of elements of the engineered intein system described herein can be driven by a ubiquitous promoter. Where the engineered intein system component (s) polynucleotide is an RNA to be expressed, its expression can be driven by a Pol III promoter, such as a U6 promoter. In some embodiments, the two are combined.

In some embodiments, expression of the engineered intein system or component thereof is driven by a minimal promoter. Thus, in some embodiments, an engineered intein system encoding polynucleotide is operatively coupled to a minimal promoter.

In one embodiment, the invention provides a vector system comprising one or more vectors. In some embodiments, all components of the engineered intein system are encoded by polynucleotides on the same vector. In some embodiments, all components of the engineered intein system are encoded by polynucleotides on different vectors. In some embodiments, each of the components of the engineered intein system are encoded by polynucleotides that are each operatively coupled to different promoters (e.g., different promoter types) so as to reduce promoter competition. In some embodiments, the constructs for expression of each of the engineered intein system are positioned in reverse orientation relative to each other. These and others are further detailed and described elsewhere herein.

### Cell-Based Vector Amplification and Expression

Vectors may be introduced and propagated in a prokaryote or prokaryotic cell. In some embodiments, a prokaryote is used to amplify copies of a vector to be introduced into a eukaryotic cell or as an intermediate vector in the production of a vector to be introduced into a eukaryotic cell (e.g., amplifying a plasmid as part of a viral vector packaging system). The vectors can be viral-based or non-viral based. In some embodiments, a prokaryote is used to amplify copies of a vector and express one or more nucleic acids, such as to provide a source of one or more proteins for delivery to a host cell or host organism.

Vectors can be designed for expression of one or more elements of the engineered intein system described herein (e.g., nucleic acid transcripts, proteins, enzymes, and combinations thereof) in a suitable host cell. In some embodiments, the suitable host cell is a prokaryotic cell. Suitable host cells include, but are not limited to, bacterial cells, yeast cells, insect cells, and mammalian cells. In some embodiments, the suitable host cell is a eukaryotic cell.

In some embodiments, the suitable host cell is a suitable bacterial cell. Suitable bacterial cells include, but are not limited to, bacterial cells from the bacteria of the species Escherichia coli. Many suitable strains of E. coli are known in the art for expression of vectors. These include, but are not limited to Pirn, Stbl2, Stbl3, Stbl4, TOP10, XL1 Blue, and XL10 Gold. In some embodiments, the host cell is a suitable insect cell. Suitable insect cells include those from Spodoptera frugiperda. Suitable strains of S. frugiperda cells include, but are not limited, to Sf9 and Sf21. In some embodiments, the host cell is a suitable yeast cell. In some embodiments, the yeast cell can be from Saccharomyces cerevisiae. In some embodiments, the host cell is a suitable mammalian cell. Many types of mammalian cells have been developed to express vectors. Suitable mammalian cells include, but are not limited to, HEK293, Chinese Hamster Ovary Cells (CHOs), mouse myeloma cells, HeLa, U2OS, A549, HT1080, CAD, P19, NIH 3T3, L929, N2a, MCF-7, Y79, SO-Rb50, HepG G2, DIKX-X11, J558L, Baby hamster kidney cells (BHK), and chicken embryo fibroblasts (CEFs). Suitable host cells are discussed further in Goeddel, GENE EXPRESSION TECHNOLOGY: METHODS IN ENZYMOLOGY 185, Academic Press, San Diego, Calif (1990).

In some embodiments, the vector can be a yeast expression vector. Examples of vectors for expression in yeast Saccharomyces cerevisiae include pYepSec1 (Baldari, et al., 1987. EMBO J. 6: 229-234), pMFa (Kuijan and Herskowitz, 1982. Cell 30: 933-943), pJRY88 (Schultz et al., 1987. Gene 54: 113-123), pYES2 (Invitrogen Corporation, San Diego, Calif), and picZ (InVitrogen Corp, San Diego, Calif). As used herein, a “yeast expression vector” refers to a nucleic acid that contains one or more sequences encoding an RNA and/or polypeptide and may further contain any desired elements that control the expression of the nucleic acid(s), as well as any elements that enable the replication and maintenance of the expression vector inside the yeast cell. Many suitable yeast expression vectors and features thereof are known in the art; for example, various vectors and techniques are illustrated in in Yeast Protocols, 2nd edition, Xiao, W., ed. (Humana Press, New York, 2007) and Buckholz, R. G. and Gleeson, M. A. (1991) Biotechnology (NY) 9(11): 1067-72. Yeast vectors can contain, without limitation, a centromeric (CEN) sequence, an autonomous replication sequence (ARS), a promoter, such as an RNA Polymerase III promoter, operably linked to a sequence or gene of interest, a terminator such as an RNA polymerase III terminator, an origin of replication, and a marker gene (e.g., auxotrophic, antibiotic, or other selectable markers). Examples of expression vectors for use in yeast may include plasmids, yeast artificial chromosomes, 2μ plasmids, yeast integrative plasmids, yeast replicative plasmids, shuttle vectors, and episomal plasmids.

In some embodiments, the vector is a baculovirus vector or expression vector and can be suitable for expression of polynucleotides and/or proteins in insect cells. In some embodiments, the suitable host cell is an insect cell. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39). rAAV (recombinant Adeno-associated viral) vectors are preferably produced in insect cells, e.g., Spodoptera frugiperda Sf9 insect cells, grown in serum-free suspension culture. Serum-free insect cells can be purchased from commercial vendors, e.g., Sigma Aldrich (EX-CELL 405).

In some embodiments, the vector is a mammalian expression vector. In some embodiments, the mammalian expression vector is capable of expressing one or more polynucleotides and/or polypeptides in a mammalian cell. Examples of mammalian expression vectors include, but are not limited to, pCDM8 (Seed, 1987. Nature 329: 840) and pMT2PC (Kaufman, et al., 1987. EMBO J. 6: 187-195). The mammalian expression vector can include one or more suitable regulatory elements capable of controlling expression of the one or more polynucleotides and/or proteins in the mammalian cell. For example, commonly used promoters are derived from polyoma, adenovirus 2, cytomegalovirus, simian virus 40, and others disclosed herein and known in the art. More detail on suitable regulatory elements is described elsewhere herein.

For other suitable expression vectors and vector systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In some embodiments, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Baneiji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546). With regards to these prokaryotic and eukaryotic vectors, mention is made of U.S. Pat. No. 6,750,059, the contents of which are incorporated by reference herein in their entirety. Other embodiments can utilize viral vectors, with regards to which mention is made of U.S. patent application Ser. No. 13/092,085, the contents of which are incorporated by reference herein in their entirety. Tissue-specific regulatory elements are known in the art and in this regard, mention is made of U.S. Pat. No. 7,776,321, the contents of which are incorporated by reference herein in their entirety. In some embodiments, a regulatory element can be operably linked to one or more elements of an engineered intein system so as to drive expression of the one or more elements of the engineered intein system described herein.

In some embodiments, the vector can be a fusion vector or fusion expression vector. In some embodiments, fusion vectors add a number of amino acids to a protein encoded therein, such as to the amino terminus, carboxy terminus, or both of a recombinant protein. Such fusion vectors can serve one or more purposes, such as: (i) to increase expression of recombinant protein; (ii) to increase the solubility of the recombinant protein; and (iii) to aid in the purification of the recombinant protein by acting as a ligand in affinity purification. In some embodiments, expression of polynucleotides (such as non-coding polynucleotides) and proteins in prokaryotes can be carried out in Escherichia coli with vectors containing constitutive or inducible promoters directing the expression of either fusion or non-fusion polynucleotides and/or proteins. In some embodiments, the fusion expression vector can include a proteolytic cleavage site, which can be introduced at the junction of the fusion vector backbone or other fusion moiety and the recombinant polynucleotide or protein to enable separation of the recombinant polynucleotide or protein from the fusion vector backbone or other fusion moiety subsequent to purification of the fusion polynucleotide or protein. Such enzymes, and their cognate recognition sequences, include Factor Xa, thrombin and enterokinase. Example fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein. Examples of suitable inducible non-fusion E. coli expression vectors include pTrc (Amrann et al., (1988) Gene 69:301-315) and pET lid (Studier et al., GENE EXPRESSION TECHNOLOGY: METHODS IN ENZYMOLOGY 185, Academic Press, San Diego, Calif. (1990) 60-89).

In some embodiments, two or more of the elements expressed from the same or different regulatory element(s), can be combined in a single vector, with one or more additional vectors providing any components of the system not included in the first vector. engineered intein system polynucleotides that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In some embodiments, a single promoter drives expression of a transcript encoding one or more engineered intein system proteins, embedded within one or more intron sequences (e.g., each in a different intron, two or more in at least one intron, or all in a single intron). In some embodiments, the engineered intein system polynucleotide(s) can be operably linked to and expressed from the same promoter.

### Cell-Free Vector and Polynucleotide Expression

In some embodiments, the polynucleotide encoding one or more features of the engineered intein system can be expressed from a vector or suitable polynucleotide in a cell-free in vitro system. In other words, the polynucleotide can be transcribed and optionally translated in vitro. In vitro transcription/translation systems and appropriate vectors are generally known in the art and commercially available. Generally, in vitro transcription and in vitro translation systems replicate the processes of RNA and protein synthesis, respectively, outside of the cellular environment. Vectors and suitable polynucleotides for in vitro transcription can include T7, SP6, T3, promoter regulatory sequences that can be recognized and acted upon by an appropriate polymerase to transcribe the polynucleotide or vector.

In vitro translation can be stand-alone (e.g., translation of a purified polyribonucleotide) or linked/coupled to transcription. In some embodiments, the cell-free (or in vitro) translation system can include extracts from rabbit reticulocytes, wheat germ, and/or E. coli. The extracts can include various macromolecular components that are needed for translation of exogenous RNA (e.g., 70S or 80S ribosomes, tRNAs, aminoacyl-tRNA, synthetases, initiation, elongation factors, termination factors, etc.). Other components can be included or added during the translation reaction, including but not limited to, amino acids, energy sources (ATP, GTP), energy regenerating systems (creatine phosphate and creatine phosphokinase (eukaryotic systems)) (phosphoenol pyruvate and pyruvate kinase for bacterial systems), and other co-factors (Mg2+, K+, etc.). As previously mentioned, in vitro translation can be based on RNA or DNA starting material. Some translation systems can utilize an RNA template as starting material (e.g., reticulocyte lysates and wheat germ extracts). Some translation systems can utilize a DNA template as a starting material (e.g., E coli-based systems). In these systems transcription and translation are coupled and DNA is first transcribed into RNA, which is subsequently translated. Suitable standard and coupled cell-free translation systems are generally known in the art and are commercially available.

### Vector Features

The vectors can include additional features that can confer one or more functionalities to the vector, the polynucleotide to be delivered, a virus particle produced there from, or polypeptide expressed thereof. Such features include, but are not limited to, regulatory elements, selectable markers, molecular identifiers (e.g., molecular barcodes), stabilizing elements, and the like. It will be appreciated by those skilled in the art that the design of the expression vector and additional features included can depend on such factors as the choice of the host cell to be transformed, the level of expression desired, etc.

### Regulatory Elements

In certain embodiments, the polynucleotides and/or vectors thereof described herein (such as the engineered intein system polynucleotides of the present invention) can include one or more regulatory elements that can be operatively linked to the polynucleotide. The term “regulatory element” is intended to include promoters, enhancers, internal ribosomal entry sites (IRES), other expression control elements (e.g., transcription termination signals, such as polyadenylation signals and poly-U sequences) and cellular localization signals (e.g., nuclear localization signals). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter can direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) (see, e.g., Boshart et al, Cell, 41:521-530 (1985)), the SV40 promoter, the dihydrofolate reductase promoter, the R-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981).

In some embodiments, the regulatory sequence can be a regulatory sequence described in U.S. Pat. No. 7,776,321, U.S. Pat. Pub. No. 2011/0027239, and International Patent Publication No. WO 2011/028929, the contents of which are incorporated by reference herein in their entirety. In some embodiments, the vector can contain a minimal promoter. In some embodiments, the minimal promoter is the Mecp2 promoter, tRNA promoter, or U6. In a further embodiment, the minimal promoter is tissue specific. In some embodiments, the length of the vector polynucleotide the minimal promoters and polynucleotide sequences is less than 4.4 Kb.

To express a polynucleotide, the vector can include one or more transcriptional and/or translational initiation regulatory sequences, e.g., promoters, that direct the transcription of the gene and/or translation of the encoded protein in a cell. In some embodiments a constitutive promoter may be employed. Suitable constitutive promoters for mammalian cells are generally known in the art and include, but are not limited to SV40, CAG, CMV, EF-1α, β-actin, RSV, and PGK. Suitable constitutive promoters for bacterial cells, yeast cells, and fungal cells are generally known in the art, such as a T-7 promoter for bacterial expression and an alcohol dehydrogenase promoter for expression in yeast.

In some embodiments, the regulatory element can be a regulated promoter. “Regulated promoter” refers to promoters that direct gene expression not constitutively, but in a temporally- and/or spatially-regulated manner, and includes tissue-specific, tissue-preferred and inducible promoters. Regulated promoters include conditional promoters and inducible promoters. In some embodiments, conditional promoters can be employed to direct expression of a polynucleotide in a specific cell type, under certain environmental conditions, and/or during a specific state of development. Suitable tissue specific promoters can include, but are not limited to, liver specific promoters (e.g., APOA2, SERPIN A1 (hAAT), CYP3A4, and MIR122), pancreatic cell promoters (e.g., INS, IRS2, Pdx1, Alx3, Ppy), cardiac specific promoters (e.g. Myh6 (alpha MHC), MYL2 (MLC-2v), TNI3 (cTnl), NPPA (ANF), Slc8a1 (Ncx1)), central nervous system cell promoters (SYN1, GFAP, INA, NES, MOBP, MBP, TH, FOXA2 (HNF3 beta)), skin cell specific promoters (e.g. FLG, K14, TGM3), immune cell specific promoters, (e.g., ITGAM, CD43 promoter, CD14 promoter, CD45 promoter, CD68 promoter), urogenital cell specific promoters (e.g., Pbsn, Upk2, Sbp, Fer1l4), endothelial cell specific promoters (e.g., ENG), pluripotent and embryonic germ layer cell specific promoters (e.g., Oct4, NANOG, Synthetic Oct4, T brachyury, NES, SOX17, FOXA2, MIR122), and muscle cell specific promoter (e.g., Desmin). Other tissue and/or cell specific promoters are generally known in the art and are within the scope of this disclosure.

Inducible/conditional promoters can be positively inducible/conditional promoters (e.g., a promoter that activates transcription of the polynucleotide upon appropriate interaction with an activated activator, or an inducer (compound, environmental condition, or other stimulus) or a negative/conditional inducible promoter (e.g., a promoter that is repressed (e.g., bound by a repressor) until the repressor condition of the promotor is removed (e.g., inducer binds a repressor bound to the promoter stimulating release of the promoter by the repressor or removal of a chemical repressor from the promoter environment). The inducer can be a compound, environmental condition, or other stimulus. Thus, inducible/conditional promoters can be responsive to any suitable stimuli such as chemical, biological, or other molecular agents, temperature, light, and/or pH. Suitable inducible/conditional promoters include, but are not limited to, Tet-On, Tet-Off, Lac promoter, pBad, AlcA, LexA, Hsp70 promoter, Hsp90 promoter, pDawn, XVE/OlexA, GVG, and pOp/LhGR.

Where expression in a plant cell is desired, the components of the engineered intein system described herein are typically placed under control of a plant promoter, i.e., a promoter operable in plant cells. The use of different types of promoters is envisaged.

A constitutive plant promoter is a promoter that is able to express the open reading frame (ORF) that it controls in all or nearly all of the plant tissues during all or nearly all developmental stages of the plant (referred to as “constitutive expression”). One non-limiting example of a constitutive promoter is the cauliflower mosaic virus 35S promoter. Different promoters may direct the expression of a gene in different tissues or cell types, or at different stages of development, or in response to different environmental conditions. In particular embodiments, one or more of the engineered intein system components are expressed under the control of a constitutive promoter, such as the cauliflower mosaic virus 35S promoter issue-preferred promoters can be utilized to target enhanced expression in certain cell types within a particular plant tissue, for instance vascular cells in leaves or roots or in specific cells of the seed. Examples of particular promoters for use in the engineered intein system are found in Kawamata et al., (1997) Plant Cell Physiol 38:792-803; Yamamoto et al., (1997) Plant J 12:255-65; Hire et al, (1992) Plant Mol Biol 20:207-18, Kuster et al, (1995) Plant Mol Biol 29:759-72, and Capana et al., (1994) Plant Mol Biol 25:681-91.

Examples of promoters that are inducible and that can allow for spatiotemporal control of gene editing or gene expression may use a form of energy. The form of energy may include but is not limited to sound energy, electromagnetic radiation, chemical energy and/or thermal energy. Examples of inducible systems include tetracycline inducible promoters (Tet-On or Tet-Off), small molecule two-hybrid transcription activations systems (FKBP, ABA, etc.), or light inducible systems (Phytochrome, LOV domains, or cryptochrome), such as a Light Inducible Transcriptional Effector (LITE) that direct changes in transcriptional activity in a sequence-specific manner. The components of a light inducible system may include one or more elements of the engineered intein system described herein, a light-responsive cytochrome heterodimer (e.g., from Arabidopsis thaliana), and a transcriptional activation/repression domain. In some embodiments, the vector can include one or more of the inducible DNA binding proteins provided in International Patent Publication No. WO 2014/018423 and US Patent Publication Nos., 2015/0291966, 2017/0166903, 2019/0203212, which describe e.g., embodiments of inducible DNA binding proteins and methods of use and can be adapted for use with the present invention.

In some embodiments, transient or inducible expression can be achieved by including, for example, chemical-regulated promotors, i.e., whereby the application of an exogenous chemical induces gene expression. Modulation of gene expression can also be obtained by including a chemical-repressible promoter, where application of the chemical represses gene expression. Chemical-inducible promoters include, but are not limited to, the maize ln2-2 promoter, activated by benzene sulfonamide herbicide safeners (De Veylder et al., (1997) Plant Cell Physiol 38:568-77), the maize GST promoter (GST-ll-27, WO93/01294), activated by hydrophobic electrophilic compounds used as pre-emergent herbicides, and the tobacco PR-1 a promoter (Ono et al., (2004) Biosci Biotechnol Biochem 68:803-7) activated by salicylic acid. Promoters which are regulated by antibiotics, such as tetracycline-inducible and tetracycline-repressible promoters (Gatz et al., (1991) Mol Gen Genet 227:229-37; U.S. Pat. Nos. 5,814,618 and 5,789,156) can also be used herein.

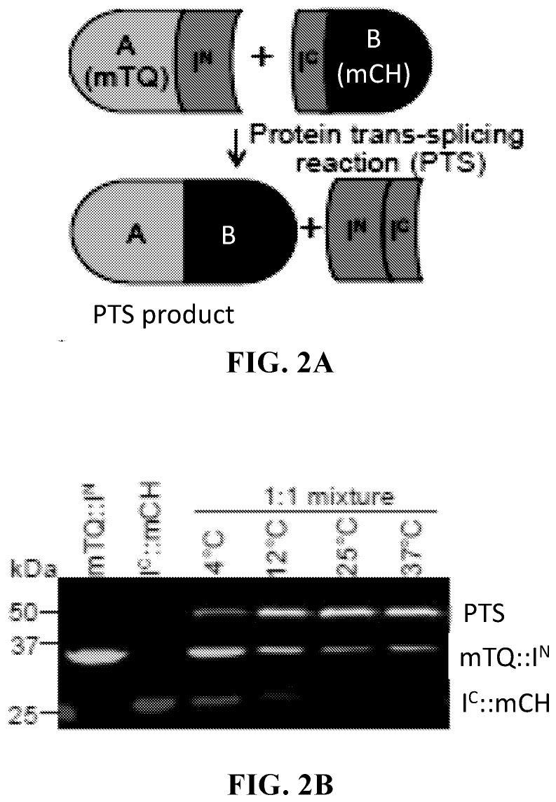

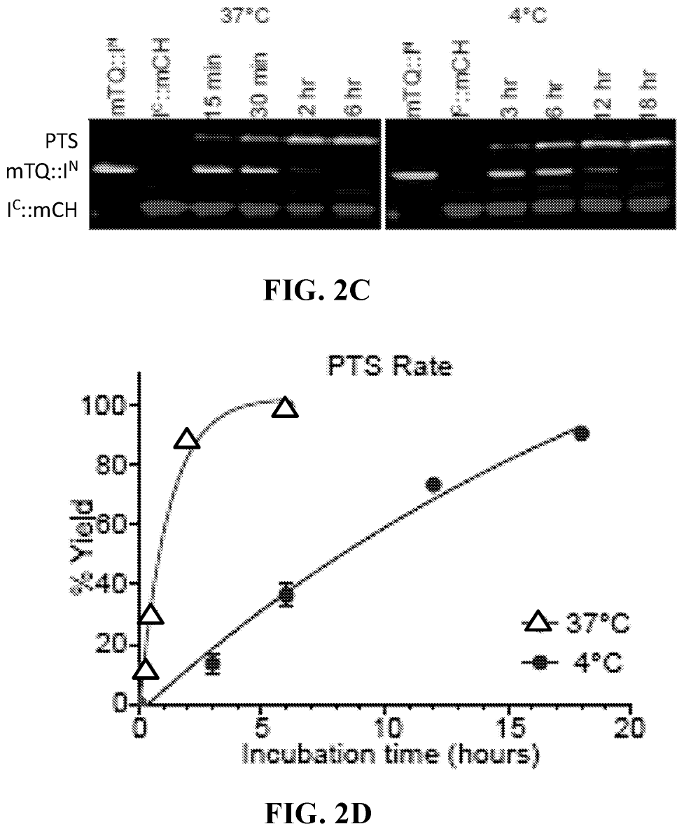

In some embodiments, the polynucleotide, vector or system thereof can include one or more elements capable of translocating and/or expressing an engineered intein system polynucleotide to/in a specific cell component or organelle. Such organelles can include, but are not limited to, nucleus, ribosome, endoplasmic reticulum, Golgi apparatus, chloroplast, mitochondria, vacuole, lysosome, cytoskeleton, plasma membrane, cell wall, peroxisome, centrioles, etc. Such regulatory elements can include, but are not limited to, nuclear localization signals (examples of which are described in greater detail elsewhere herein), any such as those that are annotated in the LocSigDB database (see e.g., http://genome.unmc.edu/LocSigDB/and Negi et al., 2015. Database. 2015: bav003; doi: 10.1093/database/bav003), nuclear export signals (e.g., LXXXLXXLXL (SEQ ID NO: 111) and others described elsewhere herein), endoplasmic reticulum localization/retention signals (e.g., KDEL, KDXX, KKXX, KXX, and others described elsewhere herein; and see e.g., Liu et al. 2007 Mol. Biol. Cell. 18(3):1073-1082 and Gorleku et al., 2011. J. Biol. Chem. 286:39573-39584), mitochondria (see e.g., Cell Reports. 22:2818-2826, particularly at FIG. 2; Doyle et al. 2013. PLoS ONE 8, e67938; Funes et al. 2002. J. Biol. Chem. 277:6051-6058; Matouschek et al. 1997. PNAS USA 85:2091-2095; Oca-Cossio et al., 2003. 165:707-720; Waltner et al., 1996. J. Biol. Chem. 271:21226-21230; Wilcox et al., 2005. PNAS USA 102:15435-15440; Galanis et al., 1991. FEBS Lett 282:425-430, peroxisome (e.g., (S/A/C)-(K/R/H)-(L/A), SLK, (R/K)-(L/V/I)-XXXXX-(H/Q)-(L/A/F). Suitable protein targeting motifs can also be designed or identified using any suitable database or prediction tool, including but not limited to Minimotif Miner (http:minimotifminer.org, http://mitominer.mrc-mbu.cam.ac.uk/release-4.0/embodiment.do?name=Protein%20MTS), LocDB (see above), PTSs predictor ( ), TargetP-2.0 (http://www.cbs.dtu.dk/services/TargetP/), ChloroP (http://www.cbs.dtu.dk/services/ChloroP/); NetNES (http://www.cbs.dtu.dk/services/NetNES/), Predotar (https://urgi.versailles.inra.fr/predotar/), and SignalP (http://www.cbs.dtu.dk/services/SignalP/).

### Selectable Markers and Tags

One or more of the engineered intein system polynucleotides can be operably linked, fused to, or otherwise modified to include a polynucleotide that encodes or is a selectable marker or tag, which can be a polynucleotide or polypeptide. In some embodiments, the polypeptide encoding a polypeptide selectable marker can be incorporated in the engineered intein system polynucleotide such that the selectable marker polypeptide, when translated, is inserted between two amino acids between the N- and C-terminus of an engineered intein system polypeptide or at the N- and/or C-terminus of an engineered intein system polypeptide. In some embodiments, the selectable marker or tag is a polynucleotide barcode or unique molecular identifier (UMI).

It will be appreciated that the polynucleotide encoding such selectable markers or tags can be incorporated into a polynucleotide encoding one or more components of the engineered intein system described herein in an appropriate manner to allow expression of the selectable marker or tag. Such techniques and methods are described elsewhere herein and will be instantly appreciated by one of ordinary skill in the art in view of this disclosure. Many such selectable markers and tags are generally known in the art and are intended to be within the scope of this disclosure.

Suitable selectable markers and tags include, but are not limited to, affinity tags, such as chitin binding protein (CBP), maltose binding protein (MBP), glutathione-S-transferase (GST), poly(His) tag; solubilization tags such as thioredoxin (TRX) and poly(NANP), MBP, and GST; chromatography tags such as those consisting of polyanionic amino acids, such as FLAG-tag; epitope tags such as V5-tag, Myc-tag, HA-tag and NE-tag; protein tags that can allow specific enzymatic modification (such as biotinylation by biotin ligase) or chemical modification (such as reaction with FlAsH-EDT2 for fluorescence imaging), DNA and/or RNA segments that contain restriction enzyme or other enzyme cleavage sites; DNA segments that encode products that provide resistance against otherwise toxic compounds including antibiotics, such as, spectinomycin, ampicillin, kanamycin, tetracycline, Basta, neomycin phosphotransferase II (NEO), hygromycin phosphotransferase (HPT)) and the like; DNA and/or RNA segments that encode products that are otherwise lacking in the recipient cell (e.g., tRNA genes, auxotrophic markers); DNA and/or RNA segments that encode products which can be readily identified (e.g., phenotypic markers such as β-galactosidase, GUS; fluorescent proteins such as green fluorescent protein (GFP), cyan (CFP), yellow (YFP), red (RFP), luciferase, and cell surface proteins); polynucleotides that can generate one or more new primer sites for PCR (e.g., the juxtaposition of two DNA sequences not previously juxtaposed), DNA sequences not acted upon or acted upon by a restriction endonuclease or other DNA modifying enzyme, chemical, etc.; epitope tags (e.g., GFP, FLAG- and His-tags), and, DNA sequences that make a molecular barcode or unique molecular identifier (UMI), DNA sequences required for a specific modification (e.g., methylation) that allows its identification. Other suitable markers will be appreciated by those of skill in the art.

Selectable markers and tags can be operably linked to one or more components of the engineered intein system of the present invention described herein via suitable linkers, such as a glycine or glycine serine linkers as short as GS or GG up to (GGGGG)₃ (SEQ ID NO: 112) or (GGGGS)₃ (SEQ ID NO: 40). Other suitable linkers are described elsewhere herein.

The vector or vector system can include one or more polynucleotides encoding one or more targeting moieties. In some embodiments, the targeting moiety encoding polynucleotides can be included in the vector or vector system, such as a viral vector system, such that they are expressed within and/or on the virus particle(s) produced such that the virus particles can be targeted to specific cells, tissues, organs, etc. In some embodiments, the targeting moiety encoding polynucleotides can be included in the vector or vector system such that the engineered intein system polynucleotide(s) and/or products expressed therefrom include the targeting moiety and can be targeted to specific cells, tissues, organs, etc. In some embodiments, such as non-viral carriers, the targeting moiety can be attached to the carrier (e.g., polymer, lipid, inorganic molecule etc.) and can be capable of targeting the carrier and any attached or associated engineered intein system polynucleotide(s) to specific cells, tissues, organs, etc.

### Codon Optimization of Vector Polynucleotides

As described elsewhere herein, the polynucleotide encoding one or more embodiments of the engineered intein system described herein can be codon optimized. In some embodiments, one or more polynucleotides contained in a vector (“vector polynucleotides”) described herein that are in addition to an optionally codon optimized polynucleotide encoding embodiments of the engineered intein system or components thereof described herein can be codon optimized. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, PA), are also available. In some embodiments, one or more codons (e.g., 1, 2, 3, 4, 5, 10, 15, 20, 25, 50, or more, or all codons) in a sequence encoding a DNA/RNA-targeting Cas protein corresponds to the most frequently used codon for a particular amino acid. As to codon usage in yeast, reference is made to the online Yeast Genome database available at http://www.yeastgenome.org/community/codon_usage.shtml, or Codon selection in yeast, Bennetzen and Hall, J Biol Chem. 1982 Mar. 25; 257(6):3026-31. As to codon usage in plants including algae, reference is made to Codon usage in higher plants, green algae, and cyanobacteria, Campbell and Gowri, Plant Physiol. 1990 January; 92(1): 1-11.; as well as Codon usage in plant genes, Murray et al, Nucleic Acids Res. 1989 Jan. 25; 17(2):477-98; or Selection on the codon bias of chloroplast and cyanelle genes in different plant and algal lineages, Morton B R, J Mol Evol. 1998 April; 46(4):449-59.

The vector polynucleotide can be codon optimized for expression in a specific cell-type, tissue type, organ type, and/or subject type. In some embodiments, a codon optimized sequence is a sequence optimized for expression in a eukaryote, e.g., humans (i.e., being optimized for expression in a human or human cell), or for another eukaryote, such as another animal (e.g., a mammal or avian) as is described elsewhere herein. Such codon optimized sequences are within the ambit of the ordinary skilled artisan in view of the description herein. In some embodiments, the polynucleotide is codon optimized for a specific cell type. Such cell types can include, but are not limited to, epithelial cells (including skin cells, cells lining the gastrointestinal tract, cells lining other hollow organs), nerve cells (nerves, brain cells, spinal column cells, nerve support cells (e.g., astrocytes, glial cells, Schwann cells etc.), muscle cells (e.g., cardiac muscle, smooth muscle cells, and skeletal muscle cells), connective tissue cells (fat and other soft tissue padding cells, bone cells, tendon cells, cartilage cells), blood cells, stem cells and other progenitor cells, immune system cells, germ cells, and combinations thereof. Such codon optimized sequences are within the ambit of the ordinary skilled artisan in view of the description herein. In some embodiments, the polynucleotide is codon optimized for a specific tissue type. Such tissue types can include, but are not limited to, muscle tissue, connective tissue, connective tissue, nervous tissue, and epithelial tissue. Such codon optimized sequences are within the ambit of the ordinary skilled artisan in view of the description herein. In some embodiments, the polynucleotide is codon optimized for a specific organ. Such organs include, but are not limited to, muscles, skin, intestines, liver, spleen, brain, lungs, stomach, heart, kidneys, gallbladder, pancreas, bladder, thyroid, bone, blood vessels, blood, and combinations thereof. Such codon optimized sequences are within the ambit of the ordinary skilled artisan in view of the description herein.

In some embodiments, a vector polynucleotide is codon optimized for expression in particular cells, such as prokaryotic or eukaryotic cells. The eukaryotic cells may be those of or derived from a particular organism, such as a plant or a mammal, including but not limited to human, or non-human eukaryote or animal or mammal as discussed herein, e.g., mouse, rat, rabbit, dog, livestock, or non-human mammal or primate.

### Vector Construction

The vectors described herein can be constructed using any suitable process or technique. In some embodiments, one or more suitable recombination and/or cloning methods or techniques can be used to the vector(s) described herein. Suitable recombination and/or cloning techniques and/or methods can include, but not limited to, those described in U.S. Patent Publication No. US 2004/0171156 A1. Other suitable methods and techniques are described elsewhere herein.

Construction of recombinant AAV vectors is described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989). Any of the techniques and/or methods can be used and/or adapted for constructing an AAV or other vectors described herein. Null AAV (nAAV) vectors are discussed elsewhere herein.

In some embodiments, a vector comprises one or more insertion sites, such as a restriction endonuclease recognition sequence (also referred to as a “cloning site”). In some embodiments, one or more insertion sites (e.g., about or more than about 1, 2, 3, 4, 5, 6, 7, 8, 9, 10, or more insertion sites) are located upstream and/or downstream of one or more sequence elements of one or more vectors. When multiple different guide polynucleotides are used, a single expression construct may be used to target nucleic acid-targeting activity to multiple different, corresponding target sequences within a cell. For example, a single vector may comprise about or more than about 1, 2, 3, 4, 5, 6, 7, 8, 9, 10, 15, 20, or more guide polynucleotides. In some embodiments, about or more than about 1, 2, 3, 4, 5, 6, 7, 8, 9, 10, or more such guide-polynucleotide-containing vectors may be provided, and optionally delivered to a cell.

Delivery vehicles, vectors, particles, nanoparticles, formulations and components thereof for expression of one or more elements of an engineered intein system described herein are as used in the foregoing documents, such as International Patent Publication No. WO 2014/093622 (PCT/US2013/074667) and are discussed in greater detail herein.

### Viral Vectors

In some embodiments, the vector is a viral vector. The term of art “viral vector” and as used herein in this context refers to polynucleotide based vectors that contain one or more elements from or based upon one or more elements of a virus that can be capable of expressing and packaging a polynucleotide, such as a engineered intein system polynucleotide of the present invention, into a virus particle and producing said virus particle when used alone or with one or more other viral vectors (such as in a viral vector system). Viral vectors and systems thereof can be used for producing viral particles for delivery of and/or expression of one or more components of the engineered intein system described herein. The viral vector can be part of a viral vector system involving multiple vectors. In some embodiments, systems incorporating multiple viral vectors can increase the safety of these systems. Suitable viral vectors can include retroviral-based vectors, lentiviral-based vectors, adenoviral-based vectors, adeno associated vectors, helper-dependent adenoviral (HdAd) vectors, hybrid adenoviral vectors, herpes simplex virus-based vectors, poxvirus-based vectors, and Epstein-Barr virus-based vectors. Other embodiments of viral vectors and viral particles produce therefrom are described elsewhere herein. In some embodiments, the viral vectors are configured to produce replication incompetent viral particles for improved safety of these systems.

In certain embodiments, the virus structural component, which can be encoded by one or more polynucleotides in a viral vector or vector system, comprises one or more capsid proteins including an entire capsid. In certain embodiments, such as wherein a viral capsid comprises multiple copies of different proteins, the delivery system can provide one or more of the same protein or a mixture of such proteins. For example, AAV comprises 3 capsid proteins, VP1, VP2, and VP3, thus delivery systems of the invention can comprise one or more of VP1, and/or one or more of VP2, and/or one or more of VP3. Accordingly, the present invention is applicable to a virus within the family Adenoviridae, such as Atadenovirus, e.g., Ovine atadenovirus D, Aviadenovirus, e.g., Fowl aviadenovirus A, Ichtadenovirus, e.g., Sturgeon ichtadenovirus A, Mastadenovirus (which includes adenoviruses such as all human adenoviruses), e.g., Human mastadenovirus C, and Siadenovirus, e.g., Frog siadenovirus A. Thus, a virus of within the family Adenoviridae is contemplated as within the invention with discussion herein as to adenovirus applicable to other family members. Target-specific AAV capsid variants can be used or selected. Non-limiting examples include capsid variants selected to bind to chronic myelogenous leukemia cells, human CD34 PBPC cells, breast cancer cells, cells of lung, heart, dermal fibroblasts, melanoma cells, stem cell, glioblastoma cells, coronary artery endothelial cells and keratinocytes. See, e.g., Buning et al, 2015, Current Opinion in Pharmacology 24, 94-104. From teachings herein and knowledge in the art as to modifications of adenovirus (see, e.g., U.S. Pat. Nos. 9,410,129, 7,344,872, 7,256,036, 6,911,199, 6,740,525; Matthews, “Capsid-Incorporation of Antigens into Adenovirus Capsid Proteins for a Vaccine Approach,” Mol Pharm, 8(1): 3-11 (2011)), as well as regarding modifications of AAV, the skilled person can readily obtain a modified adenovirus that has a large payload protein or a engineered intein system protein, despite that heretofore it was not expected that such a large protein could be provided on an adenovirus. And as to the viruses related to adenovirus mentioned herein, as well as to the viruses related to AAV mentioned elsewhere herein, the teachings herein as to modifying adenovirus and AAV, respectively, can be applied to those viruses without undue experimentation from this disclosure and the knowledge in the art.

In some embodiments, the viral vector is configured such that when the cargo is packaged the cargo(s) (e.g., one or more components of the engineered intein system), is external to the capsid or virus particle. In the sense that it is not inside the capsid (enveloped or encompassed with the capsid) but is externally exposed so that it can contact the target genomic DNA. In some embodiments, the viral vector is configured such that all the cargo(s) are contained within the capsid after packaging.

### Split Viral Vector Systems

When the engineered intein system viral vector or vector system (be it a retroviral (e.g., AAV) or lentiviral vector) is designed so as to position the cargo(s) at the internal surface of the capsid once formed, the cargo(s) will fill most or all of internal volume of the capsid. In other embodiments, the engineered intein system may be modified or divided so as to occupy a less of the capsid internal volume. Accordingly, in certain embodiments, the engineered intein system can be divided in two portions (e.g., the N-terminal intein containing first amino acid sequence and the C-terminal intein containing second amino acid sequence), one portion is contained in one viral particle or capsid and the second portion contained in a second viral particle or capsid. In certain embodiments, by splitting the engineered intein system in two portions, space is made available to link one or more heterologous domains (e.g., reporter proteins or other tags, or other functional domains) to one or both engineered intein system component portions. Such systems can be referred to as “split vector systems”. This approach can reduce the payload of any one vector. This approach can facilitate delivery of systems where the total system size is close to or exceeds the packaging capacity of the vector.

### Retroviral and Lentiviral Vectors

In some embodiments, the vector is a retroviral vector. Retroviral vectors can be composed of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Suitable retroviral vectors for the engineered intein systems can include those based upon murine leukemia virus (MuLV), gibbon ape leukemia virus (GaLV), Simian immunodeficiency virus (SIV), human immunodeficiency virus (HIV), and combinations thereof (see, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700). Selection of a retroviral gene transfer system may therefore depend on the target tissue.

The tropism of a retrovirus can be altered by incorporating foreign envelope proteins, expanding the potential target population of target cells. Lentiviral vectors are retroviral vectors that are able to transduce or infect non-dividing cells and are described in greater detail elsewhere herein. A retrovirus can also be engineered to allow for conditional expression of the inserted transgene, such that only certain cell types are infected by the lentivirus.

In some embodiments, the retroviral vector is a lentiviral vector. Lentiviruses are complex retroviruses that have the ability to infect and express their genes in both mitotic and post-mitotic cells. Advantages of using a lentiviral approach can include the ability to transduce or infect non-dividing cells and their ability to typically produce high viral titers, which can increase efficiency or efficacy of production and delivery. Suitable lentiviral vectors include, but are not limited to, human immunodeficiency virus (HIV)-based lentiviral vectors, feline immunodeficiency virus (FIV)-based lentiviral vectors, simian immunodeficiency virus (SIV)-based lentiviral vectors, Moloney Murine Leukaemia Virus (Mo-MLV), Visna.maedi virus (VMV)-based lentiviral vector, carpine arthritis-encephalitis virus (CAEV)-based lentiviral vector, bovine immune deficiency virus (BIV)-based lentiviral vector, and Equine infectious anemia (EIAV)-based lentiviral vector. In some embodiments, an HIV-based lentiviral vector system can be used. In some embodiments, a FIV-based lentiviral vector system can be used.

In some embodiments, the lentiviral vector is an EIAV-based lentiviral vector or vector system. EIAV vectors have been used to mediate expression, packaging, and/or delivery in other contexts, such as for ocular gene therapy (see, e.g., Balagaan, J Gene Med 2006; 8: 275-285). In another embodiment, RetinoStat®, (see, e.g., Binley et al., HUMAN GENE THERAPY 23:980-991 (September 2012)), which describes RetinoStat®, an equine infectious anemia virus-based lentiviral gene therapy vector that expresses angiostatic proteins endostatin and angiostatin that is delivered via a subretinal injection for the treatment of the wet form of age-related macular degeneration. Any of these vectors described in these publications can be modified for the elements of the engineered intein system described herein.

In some embodiments, the lentiviral vector or vector system thereof can be a first-generation lentiviral vector or vector system thereof. First-generation lentiviral vectors can contain a large portion of the lentivirus genome, including the gag and pol genes, other additional viral proteins (e.g., VSV-G) and other accessory genes (e.g., vif, vprm vpu, nef, and combinations thereof), regulatory genes (e.g., tat and/or rev) as well as the gene of interest between the LTRs. First generation lentiviral vectors can result in the production of virus particles that can be capable of replication in vivo, which may not be appropriate for some instances or applications.

In some embodiments, the lentiviral vector or vector system thereof can be a second-generation lentiviral vector or vector system thereof. Second-generation lentiviral vectors do not contain one or more accessory virulence factors and do not contain all components necessary for virus particle production on the same lentiviral vector. This can result in the production of a replication-incompetent virus particle and thus increase the safety of these systems over first-generation lentiviral vectors. In some embodiments, the second-generation vector lacks one or more accessory virulence factors (e.g., vif, vprm, vpu, nef, and combinations thereof). Unlike the first-generation lentiviral vectors, no single second generation lentiviral vector includes all features necessary to express and package a polynucleotide into a virus particle. In some embodiments, the envelope and packaging components are split between two different vectors with the gag, pol, rev, and tat genes being contained on one vector and the envelope protein (e.g., VSV-G) are contained on a second vector. The gene of interest, its promoter, and LTRs can be included on a third vector that can be used in conjunction with the other two vectors (packaging and envelope vectors) to generate a replication-incompetent virus particle.

In some embodiments, the lentiviral vector or vector system thereof can be a third-generation lentiviral vector or vector system thereof. Third-generation lentiviral vectors and vector systems thereof have increased safety over first- and second-generation lentiviral vectors and systems thereof because, for example, the various components of the viral genome are split between two or more different vectors but used together in vitro to make virus particles, they can lack the tat gene (when a constitutively active promoter is included up-stream of the LTRs), and they can include one or more deletions in the 3′ LTR to create self-inactivating (SIN) vectors having disrupted promoter/enhancer activity of the LTR. In some embodiments, a third-generation lentiviral vector system can include (i) a vector plasmid that contains the polynucleotide of interest and upstream promoter that are flanked by the 5′ and 3′ LTRs, which can optionally include one or more deletions present in one or both of the LTRs to render the vector self-inactivating; (ii) a “packaging vector(s)” that can contain one or more genes involved in packaging a polynucleotide into a virus particle that is produced by the system (e.g., gag, pol, and rev) and upstream regulatory sequences (e.g., promoter(s)) to drive expression of the features present on the packaging vector, and (iii) an “envelope vector” that contains one or more envelope protein genes and upstream promoters. In certain embodiments, the third-generation lentiviral vector system can include at least two packaging vectors, with the gag-pol being present on a different vector than the rev gene.

In some embodiments, self-inactivating lentiviral vectors with an siRNA targeting a common exon shared by HIV tat/rev, a nucleolar-localizing TAR decoy, and an anti-CCR5-specific hammerhead ribozyme (see, e.g., DiGiusto et al. (2010) Sci Transl Med 2:36ra43) can be used/and or adapted to the engineered intein system of the present invention.

In some embodiments, the pseudotype and infectivity or tropism of a lentivirus particle can be tuned by altering the type of envelope protein(s) included in the lentiviral vector or system thereof. As used herein, an “envelope protein” or “outer protein” means a protein exposed at the surface of a viral particle that is not a capsid protein. For example, envelope or outer proteins typically comprise proteins embedded in the envelope of the virus. In some embodiments, a lentiviral vector or vector system thereof can include a VSV-G envelope protein. VSV-G mediates viral attachment to an LDL receptor (LDLR) or an LDLR family member present on a host cell, which triggers endocytosis of the viral particle by the host cell. Because LDLR is expressed by a wide variety of cells, viral particles expressing the VSV-G envelope protein can infect or transduce a wide variety of cell types. Other suitable envelope proteins can be incorporated based on the host cell that a user desires to be infected by a virus particle produced from a lentiviral vector or system thereof described herein and can include, but are not limited to, feline endogenous virus envelope protein (RD 114) (see e.g., Hanawa et al. Molec. Ther. 2002 5(3) 242-251), modified Sindbis virus envelope proteins (see e.g., Morizono et al. 2010. J. Virol. 84(14) 6923-6934; Morizono et al. 2001. J. Virol. 75:8016-8020; Morizono et al. 2009. J. Gene Med. 11:549-558; Morizono et al. 2006 Virology 355:71-81; Morizono et al J. Gene Med. 11:655-663, Morizono et al. 2005 Nat. Med. 11:346-352), baboon retroviral envelope protein (see e.g., Girard-Gagnepain et al. 2014. Blood. 124: 1221-1231); Tupaia paramyxovirus glycoproteins (see e.g., Enkirch T. et al., 2013. Gene Ther. 20:16-23); measles virus glycoproteins (see e.g., Funke et al. 2008. Molec. Ther. 16(8): 1427-1436), rabies virus envelope proteins, MLV envelope proteins, Ebola envelope proteins, baculovirus envelope proteins, filovirus envelope proteins, hepatitis E1 and E2 envelope proteins, gp41 and gp120 of HIV, hemagglutinin, neuraminidase, M2 proteins of influenza virus, and combinations thereof.

In some embodiments, the tropism of the resulting lentiviral particle can be tuned by incorporating cell targeting peptides into a lentiviral vector such that the cell targeting peptides are expressed on the surface of the resulting lentiviral particle. In some embodiments, a lentiviral vector can contain an envelope protein that is fused to a cell targeting protein (see e.g. Buchholz et al. 2015. Trends Biotechnol. 33:777-790; Bender et al. 2016. PLoS Pathog. 12(e1005461); and Friedrich et al. 2013. Mol. Ther. 2013. 21: 849-859.

In some embodiments, a covalent-bond-forming protein-peptide pair can be incorporated into one or more of the lentiviral vectors described herein to conjugate a cell targeting peptide to the virus particle (see e.g., Kasaraneni et al. 2018. Sci. Reports (8) No. 10990). In some embodiments, a lentiviral vector can include an N-terminal PDZ domain of InaD protein (PDZ1) and its pentapeptide ligand (TEFCA) from NorpA, which can conjugate the cell targeting peptide to the virus particle via a covalent bond (e.g., a disulfide bond). In some embodiments, the PDZ1 protein can be fused to an envelope protein, which can optionally be binding deficient and/or fusion competent virus envelope protein and included in a lentiviral vector. In some embodiments, the TEFCA can be fused to a cell targeting peptide and the TEFCA-CPT fusion construct can be incorporated into the same or a different lentiviral vector as the PDZ1-envelope protein construct. During virus production, specific interaction between the PDZ1 and TEFCA facilitates producing virus particles covalently functionalized with the cell targeting peptide and thus capable of targeting a specific cell-type based upon a specific interaction between the cell targeting peptide and cells expressing its binding partner. This approach can be advantageous for use where surface-incompatibilities can restrict the use of, e.g., cell targeting peptides.

Lentiviral vectors have been disclosed as in the treatment for Parkinson's Disease, see, e.g., US Patent Publication No. 20120295960 and U.S. Pat. Nos. 7,303,910 and 7,351,585. Lentiviral vectors have also been disclosed for the treatment of ocular diseases, see e.g., US Patent Publication Nos. 20060281180, 20090007284, US20110117189; US20090017543; US20070054961, US20100317109. Lentiviral vectors have also been disclosed for delivery to the brain, see, e.g., US Patent Publication Nos. US20110293571; US20110293571, US20040013648, US20070025970, US20090111106 and U.S. Pat. No. 7,259,015. Any of these systems or a variant thereof can be used to deliver an engineered intein system polynucleotide described herein to a cell.

In some embodiments, a lentiviral vector system can include one or more transfer plasmids. Transfer plasmids can be generated from various other vector backbones and can include one or more features that can work with other retroviral and/or lentiviral vectors in the system that can, for example, improve safety of the vector and/or vector system, increase virial titers, and/or increase or otherwise enhance expression of the desired insert to be expressed and/or packaged into the viral particle. Suitable features that can be included in a transfer plasmid can include, but are not limited to, 5′LTR, 3′LTR, SIN/LTR, origin of replication (Ori), selectable marker genes (e.g., antibiotic resistance genes), Psi (ψ), RRE (rev response element), cPPT (central polypurine tract), promoters, WPRE (woodchuck hepatitis post-transcriptional regulatory element), SV40 polyadenylation signal, pUC origin, SV40 origin, F1 origin, and combinations thereof.

In another embodiment, Cocal vesiculovirus envelope pseudotyped retroviral or lentiviral vector particles are contemplated (see, e.g., US Patent Publication No. 20120164118 assigned to the Fred Hutchinson Cancer Research Center). Cocal virus is in the Vesiculovirus genus and is a causative agent of vesicular stomatitis in mammals. Cocal virus was originally isolated from mites in Trinidad (Jonkers et al., Am. J. Vet. Res. 25:236-242 (1964)), and infections have been identified in Trinidad, Brazil, and Argentina from insects, cattle, and horses. Many of the vesiculoviruses that infect mammals have been isolated from naturally infected arthropods, suggesting that they are vector-borne. Antibodies to vesiculoviruses are common among people living in rural areas where the viruses are endemic and laboratory-acquired; infections in humans usually result in influenza-like symptoms. The Cocal virus envelope glycoprotein shares 71.5% identity at the amino acid level with VSV-G Indiana, and phylogenetic comparison of the envelope gene of vesiculoviruses shows that Cocal virus is serologically distinct from, but most closely related to, VSV-G Indiana strains among the vesiculoviruses. Jonkers et al., Am. J. Vet. Res. 25:236-242 (1964) and Travassos da Rosa et al., Am. J. Tropical Med. & Hygiene 33:999-1006 (1984). The Cocal vesiculovirus envelope pseudotyped retroviral vector particles may include for example, lentiviral, alpharetroviral, betaretroviral, gammaretroviral, deltaretroviral, and epsilonretroviral vector particles that may comprise retroviral Gag, Pol, and/or one or more accessory protein(s) and a Cocal vesiculovirus envelope protein. In certain embodiments of these embodiments, the Gag, Pol, and accessory proteins are lentiviral and/or gammaretroviral. In some embodiments, a retroviral vector can contain encoding polypeptides for one or more Cocal vesiculovirus envelope proteins such that the resulting viral or pseudoviral particles are Cocal vesiculovirus envelope pseudotyped.

### Adenoviral Vectors, Helper-Dependent Adenoviral Vectors, and Hybrid Adenoviral Vectors

In some embodiments, the vector can be an adenoviral vector. In some embodiments, the adenoviral vector can include elements such that the virus particle produced using the vector or system thereof can be serotype 2 or serotype 5. In some embodiments, the polynucleotide to be delivered via the adenoviral particle can be up to about 8 kb. Thus, in some embodiments, an adenoviral vector can include a DNA polynucleotide to be delivered that can range in size from about 0.001 kb to about 8 kb. Adenoviral vectors have been used successfully in several contexts (see e.g., Teramato et al. 2000. Lancet. 355:1911-1912; Lai et al. 2002. DNA Cell. Biol. 21:895-913; Flotte et al., 1996. Hum. Gene. Ther. 7:1145-1159; and Kay et al. 2000. Nat. Genet. 24:257-261.

In some embodiments, the vector can be a helper-dependent adenoviral vector or system thereof. These are also referred to in the art as “gutless” or “gutted” vectors and are a modified generation of adenoviral vectors (see e.g., Thrasher et al. 2006. Nature. 443:E5-7). In certain embodiments of the helper-dependent adenoviral vector system one vector (the helper) can contain all the viral genes required for replication but contains a conditional gene defect in the packaging domain. The second vector of the system can contain only the ends of the viral genome, one or more engineered intein system polynucleotides, and the native packaging recognition signal, which can allow selective packaged release from the cells (see e.g., Cideciyan et al. 2009. N Engl J Med. 361:725-727). Helper-dependent adenoviral vector systems have been successful for gene delivery in several contexts (see e.g., Simonelli et al. 2010. J Am Soc Gene Ther. 18:643-650; Cideciyan et al. 2009. N Engl J Med. 361:725-727; Crane et al. 2012. Gene Ther. 19(4):443-452; Alba et al. 2005. Gene Ther. 12:18-S27; Croyle et al. 2005. Gene Ther. 12:579-587; Amalfitano et al. 1998. J. Virol. 72:926-933; and Morral et al. 1999. PNAS. 96:12816-12821). The techniques and vectors described in these publications can be adapted for inclusion and delivery of the engineered intein system polynucleotides described herein. In some embodiments, the polynucleotide to be delivered via the viral particle produced from a helper-dependent adenoviral vector or system thereof can be up to about 37 kb. Thus, in some embodiments, an adenoviral vector can include a DNA polynucleotide to be delivered that can range in size from about 0.001 kb to about 37 kb (see e.g. Rosewell et al. 2011. J. Genet. Syndr. Gene Ther. Suppl. 5:001).

In some embodiments, the vector is a hybrid-adenoviral vector or system thereof. Hybrid adenoviral vectors are composed of the high transduction efficiency of a gene-deleted adenoviral vector and the long-term genome-integrating potential of adeno-associated, retroviruses, lentivirus, and transposon based-gene transfer. In some embodiments, such hybrid vector systems can result in stable transduction and limited integration site. See e.g., Balague et al. 2000. Blood. 95:820-828; Morral et al. 1998. Hum. Gene Ther. 9:2709-2716; Kubo and Mitani. 2003. J. Virol. 77(5): 2964-2971; Zhang et al. 2013. PloS One. 8(10) e76771; and Cooney et al. 2015. Mol. Ther. 23(4):667-674), whose techniques and vectors described therein can be modified and adapted for use with the engineered intein system of the present invention. In some embodiments, a hybrid-adenoviral vector can include one or more features of a retrovirus and/or an adeno-associated virus. In some embodiments, the hybrid-adenoviral vector can include one or more features of a spuma retrovirus or foamy virus (FV). See e.g., Ehrhardt et al. 2007. Mol. Ther. 15:146-156 and Liu et al. 2007. Mol. Ther. 15:1834-1841, whose techniques and vectors described therein can be modified and adapted for use with the engineered intein system of the present invention. Advantages of using one or more features from the FVs in the hybrid-adenoviral vector or system thereof can include the ability of the viral particles produced therefrom to infect a broad range of cells, a large packaging capacity as compared to other retroviruses, and the ability to persist in quiescent (non-dividing) cells. See also e.g., Ehrhardt et al. 2007. Mol. Ther. 156:146-156 and Shuji et al. 2011. Mol. Ther. 19:76-82, whose techniques and vectors described therein can be modified and adapted for use in the engineered intein system of the present invention.

### Adeno Associated Viral (AAV) Vectors

In an embodiment, the vector can be an adeno-associated virus (AAV) vector. See, e.g., West et al., Virology 160:38-47 (1987); U.S. Pat. No. 4,797,368; WO 93/24641; Kotin, Human Gene Therapy 5:793-801 (1994); and Muzyczka, J. Clin. Invest. 94:1351 (1994). Although similar to adenoviral vectors in some of their features, AAVs have some deficiency in their replication and/or pathogenicity and thus can be safer that adenoviral vectors. In some embodiments the AAV can integrate into a specific site on chromosome 19 of a human cell with no observable side effects. In some embodiments, the capacity of the AAV vector, system thereof, and/or AAV particles can be up to about 4.7 kb. The AAV vector or system thereof can include one or more regulatory molecules. In some embodiments the regulatory molecules can be promoters, enhancers, repressors and the like, which are described in greater detail elsewhere herein. In some embodiments, the AAV vector or system thereof can include one or more polynucleotides that can encode one or more regulatory proteins. In some embodiments, the one or more regulatory proteins can be selected from Rep78, Rep68, Rep52, Rep40, variants thereof, and combinations thereof.

The AAV vector or system thereof can include one or more polynucleotides that can encode one or more capsid proteins. The capsid proteins can be selected from VP1, VP2, VP3, and combinations thereof. The capsid proteins can be capable of assembling into a protein shell of the AAV virus particle. In some embodiments, the AAV capsid can contain 60 capsid proteins. In some embodiments, the ratio of VP1:VP2:VP3 in a capsid can be about 1:1:10.

In some embodiments, the AAV vector or system thereof can include one or more adenovirus helper factors or polynucleotides that can encode one or more adenovirus helper factors. Such adenovirus helper factors can include, but are not limited, E1A, E1B, E2A, E40RF6, and VA RNAs. In some embodiments, a producing host cell line expresses one or more of the adenovirus helper factors.

The AAV vector or system thereof can be configured to produce AAV particles having a specific serotype. According to the present disclosure, the AAV particles may utilize or be based on a serotype selected from any of the following serotypes, and variants thereof including but not limited to AAV1, AAV10, AAV106.1/hu.37, AAV11, AAV114.3/hu.40, AAV12, AAV127.2/hu.41, AAV127.5/hu.42, AAV128.1/hu.43, AAV128.3/hu.44, AAV130.4/hu.48, AAV145.1/hu.53, AAV145.5/hu.54, AAV145.6/hu.55, AAV16.12/hu.11, AAV16.3, AAV16.8/hu.10, AAV161.10/hu.60, AAV161.6/hu.61, AAV1-7/rh.48, AAV1-8/rh.49, AAV2, AAV2.5T, AAV2-15/rh.62, AAV223.1, AAV223.2, AAV223.4, AAV223.5, AAV223.6, AAV223.7, AAV2-3/rh.61, AAV24.1, AAV2-4/rh.50, AAV2-5/rh.51, AAV27.3, AAV29.3/bb.1, AAV29.5/bb.2, AAV2G9, AAV-2-pre-miRNA-101, AAV3, AAV3.1/hu.6, AAV3.1/hu.9, AAV3-11/rh.53, AAV3-3, AAV33.12/hu.17, AAV33.4/hu.15, AAV33.8/hu.16, AAV3-9/rh.52, AAV3a, AAV3b, AAV4, AAV4-19/rh.55, AAV42.12, AAV42-10, AAV42-11, AAV42-12, AAV42-13, AAV42-15, AAV42-1b, AAV42-2, AAV42-3a, AAV42-3b, AAV42-4, AAV42-5a, AAV42-5b, AAV42-6b, AAV42-8, AAV42-aa, AAV43-1, AAV43-12, AAV43-20, AAV43-21, AAV43-23, AAV43-25, AAV43-5, AAV4-4, AAV44.1, AAV44.2, AAV44.5, AAV46.2/hu.28, AAV46.6/hu.29, AAV4-8/r11.64, AAV4-8/rh.64, AAV4-9/rh.54, AAV5, AAV52.1/hu.20, AAV52/hu.19, AAV5-22/rh.58, AAV5-3/rh.57, AAV54.1/hu.21, AAV54.2/hu.22, AAV54.4R/hu.27, AAV54.5/hu.23, AAV54.7/hu.24, AAV58.2/hu.25, AAV6, AAV6.1, AAV6.1.2, AAV6.2, AAV7, AAV7.2, AAV7.3/hu.7, AAV8, AAV-8b, AAV-8h, AAV9, AAV9.11, AAV9.13, AAV9.16, AAV9.24, AAV9.45, AAV9.47, AAV9.61, AAV9.68, AAV9.84, AAV9.9, AAVA3.3, AAVA3.4, AAVA3.5, AAVA3.7, AAV-b, AAVC1, AAVC2, AAVC5, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAV-DJ, AAV-DJ8, AAVF3, AAVF5, AAV-h, AAVH-1/hu.1, AAVH2, AAVH-5/hu.3, AAVH6, AAVhE1.1, AAVhER1.14, AAVhEr1.16, AAVhEr1.18, AAVhER1.23, AAVhEr1.35, AAVhEr1.36, AAVhEr1.5, AAVhEr1.7, AAVhEr1.8, AAVhEr2.16, AAVhEr2.29, AAVhEr2.30, AAVhEr2.31, AAVhEr2.36, AAVhEr2.4, AAVhEr3.1, AAVhu.1, AAVhu.10, AAVhu.11, AAVhu.11, AAVhu.12, AAVhu.13, AAVhu.14/9, AAVhu.15, AAVhu.16, AAVhu.17, AAVhu.18, AAVhu.19, AAVhu.2, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.3, AAVhu.31, AAVhu.32, AAVhu.34, AAVhu.35, AAVhu.37, AAVhu.39, AAVhu.4, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.5, AAVhu.51, AAVhu.52, AAVhu.53, AAVhu.54, AAVhu.55, AAVhu.56, AAVhu.57, AAVhu.58, AAVhu.6, AAVhu.60, AAVhu.61, AAVhu.63, AAVhu.64, AAVhu.66, AAVhu.67, AAVhu.7, AAVhu.8, AAVhu.9, AAVhu.t 19, AAVLG-10/rh.40, AAVLG-4/rh.38, AAVLG-9/hu.39, AAVLG-9/hu.39, AAV-LK01, AAV-LK02, AAVLK03, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK17, AAV-LK18, AAV-LK19, AAVN721-8/rh.43, AAV-PAEC, AAV-PAEC11, AAV-PAEC12, AAV-PAEC2, AAV-PAEC4, AAV-PAEC6, AAV-PAEC7, AAV-PAEC8, AAVpi.1, AAVpi.2, AAVpi.3, AAVrh.10, AAVrh.12, AAVrh.13, AAVrh.13R, AAVrh.14, AAVrh.17, AAVrh.18, AAVrh.19, AAVrh.2, AAVrh.20, AAVrh.21, AAVrh.22, AAVrh.23, AAVrh.24, AAVrh.25, AAVrh.2R, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.43, AAVrh.44, AAVrh.45, AAVrh.46, AAVrh.47, AAVrh.48, AAVrh.48, AAVrh.48.1, AAVrh.48.1.2, AAVrh.48.2, AAVrh.49, AAVrh.50, AAVrh.51, AAVrh.52, AAVrh.53, AAVrh.54, AAVrh.55, AAVrh.56, AAVrh.57, AAVrh.58, AAVrh.59, AAVrh.60, AAVrh.61, AAVrh.62, AAVrh.64, AAVrh.64R1, AAVrh.64R2, AAVrh.65, AAVrh.67, AAVrh.68, AAVrh.69, AAVrh.70, AAVrh.72, AAVrh.73, AAVrh.74, AAVrh.8, AAVrh.8R, AAVrh8R, AAVrh8R A586R mutant, AAVrh8R R533A mutant, BAAV, BNP61 AAV, BNP62 AAV, BNP63 AAV, bovine AAV, caprine AAV, Japanese AAV 10, true type AAV (ttAAV), UPENN AAV 10, AAV-LK16, AAAV, AAV Shuffle 100-1, AAV Shuffle 100-2, AAV Shuffle 100-3, AAV Shuffle 100-7, AAV Shuffle 10-2, AAV Shuffle 10-6, AAV Shuffle 10-8, AAV SM 100-10, AAV SM 100-3, AAV SM 10-1, AAV SM 10-2, AAV SM 10-8, or any combination thereof (such as in a hybrid AAV vector).

A tabulation of certain AAV serotypes as to these cells can be found in Grimm, D. et al, J. Virol. 82: 5887-5911 (2008) at Table 3.

In some embodiments, the AAV vector or system thereof is configured as a “gutless” vector, similar to that described in connection with a retroviral vector. In some embodiments, the “gutless” AAV vector or system thereof can have the cis-acting viral DNA elements involved in genome amplification and packaging in linkage with the heterologous sequences of interest (e.g., the engineered intein system polynucleotide(s)).

In some embodiments, the AAV vectors are produced in in insect cells, e.g., Spodoptera frugiperda Sf9 insect cells, grown in serum-free suspension culture. Serum-free insect cells can be purchased from commercial vendors, e.g., Sigma Aldrich (EX-CELL 405).

In another embodiment, the invention provides a non-naturally occurring or engineered intein system protein associated with Adeno Associated Virus (AAV), e.g., an AAV comprising an engineered intein system protein as a fusion, with or without a linker, to or with an AAV capsid protein such as VP1, VP2, and/or VP3. More in particular, modifying the knowledge in the art, e.g., Rybniker et al., “Incorporation of Antigens into Viral Capsids Augments Immunogenicity of Adeno-Associated Virus Vector-Based Vaccines,” J Virol. Dec 2012; 86(24): 13800-13804, Lux K, et al. 2005. Green fluorescent protein-tagged adeno-associated virus particles allow the study of cytosolic and nuclear trafficking. J. Virol. 79:11776-11787, Munch R C, et al. 2012. “Displaying high-affinity ligands on adeno-associated viral vectors enables tumor cell-specific and safe gene transfer.” Mol. Ther. [Epub ahead of print.] doi:10.1038/mt.2012.186 and Warrington K H, Jr, et al. 2004. Adeno-associated virus type 2 VP2 capsid protein is nonessential and can tolerate large peptide insertions at its N terminus. J. Virol. 78:6595-6609, each incorporated herein by reference, one can obtain a modified AAV capsid of the invention. It will be understood by those skilled in the art that the modifications described herein if inserted into the AAV cap gene may result in modifications in the VP1, VP2 and/or VP3 capsid subunits. Alternatively, the capsid subunits can be expressed independently to achieve modification in only one or two of the capsid subunits (VP1, VP2, VP3, VP1+VP2, VP1+VP3, or VP2+VP3). One can modify the cap gene to have expressed at a desired location a non-capsid protein advantageously a large payload protein, such as an engineered intein system protein. Likewise, these can be fusions, with the protein, e.g., large payload protein fused in a manner analogous to prior art fusions. See, e.g., US Patent Publication 20090215879; Nance et al., “Perspective on Adeno-Associated Virus Capsid Modification for Duchenne Muscular Dystrophy Gene Therapy,” Hum Gene Ther. 26(12):786-800 (2015) and documents cited therein, incorporated herein by reference. The skilled person, from this disclosure and the knowledge in the art can make and use modified AAV or AAV capsid as in the herein invention, and through this disclosure one knows now that large payload proteins can be fused to the AAV capsid. The instant invention is also applicable to a virus in the genus Dependoparvovirus or in the family Parvoviridae, for instance, AAV, or a virus of Amdoparvovirus, e.g., Carnivore amdoparvovirus 1, a virus of Aveparvovirus, e.g., Galliform aveparvovirus 1, a virus of Bocaparvovirus, e.g., Ungulate bocaparvovirus 1, a virus of Copiparvovirus, e.g., Ungulate copiparvovirus 1, a virus of Dependoparvovirus, e.g., Adeno-associated dependoparvovirus A, a virus of Erythroparvovirus, e.g., Primate erythroparvovirus 1, a virus of Protoparvovirus, e.g., Rodent protoparvovirus 1, a virus of Tetraparvovirus, e.g., Primate tetraparvovirus 1. Thus, a virus of within the family Parvoviridae or the genus Dependoparvovirus or any of the other foregoing genera within Parvoviridae is contemplated as within the invention as the discussion herein as to AAV is applicable to such other viruses.

In some embodiments, the engineered intein system protein(s) is/are external to the capsid or virus particle in the sense that it is not inside the capsid (enveloped or encompassed with the capsid), but is externally exposed so that it can contact a target or other engineered intein system protein. In some embodiments, the engineered intein system protein(s) is/are associated with the AAV VP1, VP2, or VP3 domain by way of a fusion protein. In some embodiments, the association may be considered to be a modification of the VP1, VP2, or VP3 domain. Where reference is made herein to a modified VP1, VP2, or VP3 domain, then this will be understood to include any association discussed herein of the VP1, VP2, or VP3 domain and the engineered intein system protein(s) is/are. In some embodiments, the AAV VP1, VP2, or VP3 domain may be associated (or tethered) to the engineered intein system protein(s) via a connector protein, for example using a system such as the streptavidin-biotin system. In an embodiment, the present invention provides a polynucleotide encoding the engineered intein system protein(s) and an associated AAV VP1, VP2, or VP3 domain. In one embodiment, the invention provides a non-naturally occurring modified AAV having a VP1, VP2, or VP3-engineered intein system capsid protein, wherein the engineered intein system protein(s) is part of or tethered to the VP1, VP2, or VP3 domain.

In certain embodiments, the positioning of the engineered intein system protein(s) is/are such that the engineered intein system protein(s) is/are at the internal surface of the viral capsid once formed. In one embodiment, the invention provides a non-naturally occurring or engineered composition comprising an engineered intein system protein(s) associated with an internal surface of an AAV capsid domain. Here again, associated may mean in some embodiments fused, or in some embodiments bound to, or in some embodiments tethered to. The engineered intein system protein(s) may, in some embodiments, be tethered to the VP1, VP2, or VP3 domain such that it locates to the internal surface of the viral capsid once formed. This may be via a connector protein or tethering system such as the biotin-streptavidin system as described above and/or elsewhere herein.

### Herpes Simplex Viral Vectors

In some embodiments, the vector is a Herpes Simplex Viral (HSV)-based vector or system thereof. HSV systems can include the disabled infections single copy (DISC) viruses, which are composed of a glycoprotein H defective mutant HSV genome. When the defective HSV is propagated in complementing cells, virus particles can be generated that are capable of infecting subsequent cells permanently replicating their own genome but are not capable of producing more infectious particles. See e.g., 2009. Trobridge. Exp. Opin. Biol. Ther. 9:1427-1436, whose techniques and vectors described therein can be modified and adapted for use in the engineered intein system of the present invention. In some embodiments where an HSV vector or system thereof is utilized, the host cell can be a complementing cell. In some embodiments, HSV vector or system thereof can be capable of producing virus particles capable of delivering a polynucleotide cargo of up to 150 kb. Thus, in some embodiment the engineered intein system polynucleotide(s) included in the HSV-based viral vector or system thereof can sum from about 0.001 to about 150 kb. HSV-based vectors and systems thereof have been successfully used in several contexts including various models of neurologic disorders. See e.g. Cockrell et al. 2007. Mol. Biotechnol. 36:184-204; Kafri T. 2004. Mol. Biol. 246:367-390; Balaggan and Ali. 2012. Gene Ther. 19:145-153; Wong et al. 2006. Hum. Gen. Ther. 2002. 17:1-9; Azzouz et al. J. Neruosci. 22L10302-10312; and Betchen and Kaplitt. 2003. Curr. Opin. Neurol. 16:487-493, whose techniques and vectors described therein can be modified and adapted for use in the engineered intein system of the present invention.

### Poxvirus Vectors

In some embodiments, the vector can be a poxvirus vector or system thereof. In some embodiments, the poxvirus vector can result in cytoplasmic expression of one or more engineered intein system polynucleotides of the present invention. In some embodiments the capacity of a poxvirus vector or system thereof can be about 25 kb or more. In some embodiments, a poxvirus vector or system thereof can include one or more engineered intein system polynucleotides described herein.

### Viral Vectors for Delivery to Plants

The systems and compositions of the present invention may be delivered to plant cells using viral vehicles. In particular embodiments, the compositions and systems may be introduced in the plant cells using a plant viral vector (e.g., as described in Scholthof et al. 1996, Annu Rev Phytopathol. 1996; 34:299-323). Such viral vector may be a vector from a DNA virus, e.g., geminivirus (e.g., cabbage leaf curl virus, bean yellow dwarf virus, wheat dwarf virus, tomato leaf curl virus, maize streak virus, tobacco leaf curl virus, or tomato golden mosaic virus) or nanovirus (e.g., Faba bean necrotic yellow virus). The viral vector may be a vector from an RNA virus, e.g., tobravirus (e.g., tobacco rattle virus, tobacco mosaic virus), potexvirus (e.g., potato virus X), or hordeivirus (e.g., barley stripe mosaic virus). The replicating genomes of plant viruses may be non-integrative vectors.

Virus Particle Production from Viral Vectors

### Retroviral Production

In some embodiments, one or more viral vectors and/or system thereof can be delivered to a suitable cell line for production of virus particles containing the polynucleotide or other payload to be delivered to a host cell. Suitable host cells for virus production from viral vectors and systems thereof described herein are known in the art and are commercially available. For example, suitable host cells include HEK 293 cells and its variants (HEK 293T and HEK 293TN cells). In some embodiments, the suitable host cell for virus production from viral vectors and systems thereof described herein can stably express one or more genes involved in packaging (e.g., pol, gag, and/or VSV-G) and/or other supporting genes.

In some embodiments, after delivery of one or more viral vectors to the suitable host cells for or virus production from viral vectors and systems thereof, the cells are incubated for an appropriate length of time to allow for viral gene expression from the vectors, packaging of the polynucleotide to be delivered (e.g., an engineered intein system polynucleotide), and virus particle assembly, and secretion of mature virus particles into the culture media. Various other methods and techniques are generally known to those of ordinary skill in the art.

Mature virus particles can be collected from the culture media by a suitable method. In some embodiments, this can involve centrifugation to concentrate the virus. The titer of the composition containing the collected virus particles can be obtained using a suitable method. Such methods can include transducing a suitable cell line (e.g., NIH 3T3 cells) and determining transduction efficiency, infectivity in that cell line by a suitable method. Suitable methods include PCR-based methods, flow cytometry, and antibiotic selection-based methods. Various other methods and techniques are generally known to those of ordinary skill in the art. The concentration of virus particle can be adjusted as needed. In some embodiments, the resulting composition containing virus particles can contain 1 X101-1 X 1020 particles/mL.

Lentiviruses may be prepared from any lentiviral vector or vector system described herein. In one example embodiment, after cloning pCasES10 (which contains a lentiviral transfer plasmid backbone), HEK293FT at low passage (p=5) can be seeded in a T-75 flask to 50% confluence the day before transfection in DMEM with 10% fetal bovine serum and without antibiotics. After 20 hours, the media can be changed to OptiMEM (serum-free) media and transfection of the lentiviral vectors can done 4 hours later. Cells can be transfected with 10 μg of lentiviral transfer plasmid (pCasES10) and the appropriate packaging plasmids (e.g., 5 μg of pMD2.G (VSV-g pseudotype), and 7.5 ug of psPAX2 (gag/pol/rev/tat)). Transfection can be carried out in 4 mL OptiMEM with a cationic lipid delivery agent (50 uL Lipofectamine 2000 and 100 ul Plus reagent). After 6 hours, the media can be changed to antibiotic-free DMEM with 10% fetal bovine serum. These methods can use serum during cell culture, but serum-free methods are preferred.

Following transfection and allowing the producing cells (also referred to as packaging cells) to package and produce virus particles with packaged cargo, the lentiviral particles can be purified. In an exemplary embodiment, virus-containing supernatants can be harvested after 48 hours. Collected virus-containing supernatants can first be cleared of debris and filtered through a 0.45 um low protein binding (PVDF) filter. They can then be spun in an ultracentrifuge for 2 hours at 24,000 rpm. The resulting virus-containing pellets can be resuspended in 50 ul of DMEM overnight at 4 degrees C. They can be then aliquoted and used immediately or immediately frozen at −80 degrees C. for storage.

### AAV Particle Production

There are two main strategies for producing AAV particles from AAV vectors and systems thereof, such as those described herein, which depend on how the adenovirus helper factors are provided (helper v. helper free). In some embodiments, a method of producing AAV particles from AAV vectors and systems thereof can include adenovirus infection into cell lines that stably harbor AAV replication and capsid encoding polynucleotides along with AAV vector containing the polynucleotide to be packaged and delivered by the resulting AAV particle (e.g., the engineered intein system polynucleotide(s)). In some embodiments, a method of producing AAV particles from AAV vectors and systems thereof can be a “helper free” method, which includes co-transfection of an appropriate producing cell line with three vectors (e.g., plasmid vectors): (1) an AAV vector that contains a polynucleotide of interest (e.g., the engineered intein system polynucleotide(s)) between 2 ITRs; (2) a vector that carries the AAV Rep-Cap encoding polynucleotides; and (helper polynucleotides. One of skill in the art will appreciate various methods and variations thereof that are both helper and -helper free and as well as the different advantages of each system.

### Non-Viral Vectors

In some embodiments, the vector is a non-viral vector or vector system. The term of art “Non-viral vector” and as used herein in this context refers to molecules and/or compositions that are vectors but that are not based on one or more component of a virus or virus genome (excluding any nucleotide to be delivered and/or expressed by the non-viral vector) that can be capable of incorporating engineered intein system polynucleotide(s) and delivering said engineered intein system polynucleotide(s) to a cell and/or expressing the polynucleotide in the cell. It will be appreciated that this does not exclude vectors containing a polynucleotide designed to target a virus-based polynucleotide that is to be delivered. For example, if a gRNA to be delivered is directed against a virus component and it is inserted or otherwise coupled to an otherwise non-viral vector or carrier, this would not make said vector a “viral vector”. Non-viral vectors can include, without limitation, naked polynucleotides and polynucleotide (non-viral) based vector and vector systems.

### Naked Polynucleotides

In some embodiments one or more engineered intein system polynucleotides described elsewhere herein can be included in a naked polynucleotide. The term of art “naked polynucleotide” as used herein refers to polynucleotides that are not associated with another molecule (e.g., proteins, lipids, and/or other molecules) that can often help protect it from environmental factors and/or degradation. As used herein, associated with includes, but is not limited to, linked to, adhered to, adsorbed to, enclosed in, enclosed in or within, mixed with, and the like. Naked polynucleotides that include one or more of the engineered intein system polynucleotides described herein can be delivered directly to a host cell and optionally expressed therein. The naked polynucleotides can have any suitable two- and three-dimensional configurations. By way of non-limiting examples, naked polynucleotides can be single-stranded molecules, double stranded molecules, circular molecules (e.g., plasmids and artificial chromosomes), molecules that contain portions that are single stranded and portions that are double stranded (e.g., ribozymes), and the like. In some embodiments, the naked polynucleotide contains only the engineered intein system polynucleotide(s) of the present invention. In some embodiments, the naked polynucleotide can contain other nucleic acids and/or polynucleotides in addition to the engineered intein system polynucleotide(s) of the present invention. The naked polynucleotides can include one or more elements of a transposon system. Transposons and system thereof are described in greater detail elsewhere herein.

### Non-Viral Polynucleotide Vectors

In some embodiments, one or more of the engineered intein system polynucleotides can be included in a non-viral polynucleotide vector. Suitable non-viral polynucleotide vectors include, but are not limited to, transposon vectors and vector systems, plasmids, bacterial artificial chromosomes, yeast artificial chromosomes, AR (antibiotic resistance)-free plasmids and miniplasmids, circular covalently closed vectors (e.g., minicircles, minivectors, miniknots,), linear covalently closed vectors (“dumbbell shaped”), MIDGE (minimalistic immunologically defined gene expression) vectors, MiLV (micro-linear vector) vectors, Ministrings, mini-intronic plasmids, PSK systems (post-segregationally killing systems), ORT (operator repressor titration) plasmids, and the like. See e.g., Hardee et al. 2017. Genes. 8(2):65.

In some embodiments, the non-viral polynucleotide vector can have a conditional origin of replication. In some embodiments, the non-viral polynucleotide vector can be an ORT plasmid. In some embodiments, the non-viral polynucleotide vector can have a minimalistic immunologically defined gene expression. In some embodiments, the non-viral polynucleotide vector can have one or more post-segregationally killing system genes. In some embodiments, the non-viral polynucleotide vector is AR-free. In some embodiments, the non-viral polynucleotide vector is a minivector. In some embodiments, the non-viral polynucleotide vector includes a nuclear localization signal. In some embodiments, the non-viral polynucleotide vector can include one or more CpG motifs. In some embodiments, the non-viral polynucleotide vectors can include one or more scaffold/matrix attachment regions (S/MARs). See e.g., Mirkovitch et al. 1984. Cell. 39:223-232, Wong et al. 2015. Adv. Genet. 89:113-152, whose techniques and vectors can be adapted for use in the present invention. S/MARs are AT-rich sequences that play a role in the spatial organization of chromosomes through DNA loop base attachment to the nuclear matrix. S/MARs are often found close to regulatory elements such as promoters, enhancers, and origins of DNA replication. Inclusion of one or S/MARs can facilitate a once-per-cell-cycle replication to maintain the non-viral polynucleotide vector as an episome in daughter cells. In certain embodiments, the S/MAR sequence is located downstream of an actively transcribed polynucleotide (e.g., one or more engineered intein system polynucleotides of the present invention) included in the non-viral polynucleotide vector. In some embodiments, the S/MAR can be a S/MAR from the beta-interferon gene cluster. See e.g., Verghese et al. 2014. Nucleic Acid Res. 42:e53; Xu et al. 2016. Sci. China Life Sci. 59:1024-1033; Jin et al. 2016. 8:702-711; Koirala et al. 2014. Adv. Exp. Med. Biol. 801:703-709; and Nehlsen et al. 2006. Gene Ther. Mol. Biol. 10:233-244, whose techniques and vectors can be adapted for use in the present invention.

In some embodiments, the non-viral vector is a transposon vector or system thereof. As used herein, “transposon” (also referred to as transposable element) refers to a polynucleotide sequence that is capable of moving form location in a genome to another. There are several classes of transposons. Transposons include retrotransposons and DNA transposons. Retrotransposons require the transcription of the polynucleotide that is moved (or transposed) in order to transpose the polynucleotide to a new genome or polynucleotide. DNA transposons are those that do not require reverse transcription of the polynucleotide that is moved (or transposed) in order to transpose the polynucleotide to a new genome or polynucleotide. In some embodiments, the non-viral polynucleotide vector can be a retrotransposon vector. In some embodiments, the retrotransposon vector includes long terminal repeats. In some embodiments, the retrotransposon vector does not include long terminal repeats. In some embodiments, the non-viral polynucleotide vector can be a DNA transposon vector. DNA transposon vectors can include a polynucleotide sequence encoding a transposase. In some embodiments, the transposon vector is configured as a non-autonomous transposon vector, meaning that the transposition does not occur spontaneously on its own. In some of these embodiments, the transposon vector lacks one or more polynucleotide sequences encoding proteins required for transposition. In some embodiments, the non-autonomous transposon vectors lack one or more Ac elements.

In some embodiments a non-viral polynucleotide transposon vector system can include a first polynucleotide vector that contains the engineered intein system polynucleotide(s) of the present invention flanked on the 5′ and 3′ ends by transposon terminal inverted repeats (TIRs) and a second polynucleotide vector that includes a polynucleotide capable of encoding a transposase coupled to a promoter to drive expression of the transposase. When both are expressed in the same cell the transposase can be expressed from the second vector and can transpose the material between the TIRs on the first vector (e.g., the engineered intein system polynucleotide(s) of the present invention) and integrate it into one or more positions in the host cell's genome. In some embodiments the transposon vector or system thereof can be configured as a gene trap. In some embodiments, the TIRs can be configured to flank a strong splice acceptor site followed by a reporter and/or other gene (e.g., one or more of the engineered intein system polynucleotide(s) of the present invention) and a strong poly A tail. When transposition occurs while using this vector or system thereof, the transposon can insert into an intron of a gene and the inserted reporter or other gene can provoke a mis-splicing process and as a result it in activates the trapped gene.

Any suitable transposon system can be used. Suitable transposon and systems thereof can include, without limitation, Sleeping Beauty transposon system (Tcl/mariner superfamily) (see e.g., Ivics et al. 1997. Cell. 91(4): 501-510), piggyBac (piggyBac superfamily) (see e.g., Li et al. 2013 110(25): E2279-E2287 and Yusa et al. 2011. PNAS. 108(4): 1531-1536), Tol2 (superfamily hAT), Frog Prince (Tcl/mariner superfamily) (see e.g., Miskey et al. 2003 Nucleic Acid Res. 31(23):6873-6881) and variants thereof.

### Non-Vector Delivery Vehicles

The delivery vehicles may comprise non-viral vehicles. In general, methods and vehicles capable of delivering nucleic acids and/or proteins may be used for delivering the systems compositions herein. Examples of non-viral vehicles include lipid nanoparticles, cell-penetrating peptides (CPPs), DNA nanoclews, metal nanoparticles, streptolysin O, multifunctional envelope-type nanodevices (MENDs), lipid-coated mesoporous silica particles, and other inorganic nanoparticles.

### Lipid Particles

The delivery vehicles may comprise lipid particles, e.g., lipid nanoparticles (LNPs) and liposomes. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, International Patent Publication Nos. WO 91/17424 and WO 91/16024. The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

### Lipid Nanoparticles (LNPs)

LNPs may encapsulate nucleic acids within cationic lipid particles (e.g., liposomes), and may be delivered to cells with relative ease. In some examples, lipid nanoparticles do not contain any viral components, which helps minimize safety and immunogenicity concerns. Lipid particles may be used for in vitro, ex vivo, and in vivo deliveries. Lipid particles may be used for various scales of cell populations.

In some examples. LNPs may be used for delivering DNA molecules (e.g., those comprising coding sequences of engineered intein system proteins). In certain cases, LNPs may be use for delivering RNP complexes of engineered intein system proteins and encoding RNA or co-therapy RNAs.

Components in LNPs may comprise cationic lipids 1,2-dilineoyl-3-dimethylammonium-propane (DLinDAP), 1,2-dilinoleyloxy-3-N,N-dimethylaminopropane (DLinDMA), 1,2-dilinoleyloxyketo-N,N-dimethyl-3-aminopropane (DLinK-DMA), 1,2-dilinoleyl-4-(2-dimethylaminoethyl)-[1,3]-dioxolane (DLinKC2-DMA), (3-o-[2″-(methoxypolyethyleneglycol 2000) succinoyl]-1,2-dimyristoyl-sn-glycol (PEG-S-DMG), R-3-[(ro-methoxy-poly(ethylene glycol)2000) carbamoyl]-1,2-dimyristyloxlpropyl-3-amine (PEG-C-DOMG, and any combination thereof. Preparation of LNPs and encapsulation may be adapted from Rosin et al, Molecular Therapy, vol. 19, no. 12, pages 1286-2200, Dec. 2011).

In some embodiments, an LNP delivery vehicle can be used to deliver a virus particle containing an engineered intein system and/or component(s) thereof. In some embodiments, the virus particle(s) can be adsorbed to the lipid particle, such as through electrostatic interactions, and/or can be attached to the liposomes via a linker.

In some embodiments, the LNP contains a nucleic acid, wherein the charge ratio of nucleic acid backbone phosphates to cationic lipid nitrogen atoms is about 1: 1.5-7 or about 1:4.

In some embodiments, the LNP also includes a shielding compound, which is removable from the lipid composition under in vivo conditions. In some embodiments, the shielding compound is a biologically inert compound. In some embodiments, the shielding compound does not carry any charge on its surface or on the molecule as such. In some embodiments, the shielding compounds are polyethylenglycoles (PEGs), hydroxyethylglucose (HEG) based polymers, polyhydroxyethyl starch (polyHES) and polypropylene. In some embodiments, the PEG, HEG, polyHES, and a polypropylene weight between about 500 to 10,000 Da or between about 2000 to 5000 Da. In some embodiments, the shielding compound is PEG2000 or PEG5000.

In some embodiments, the LNP can include one or more helper lipids. In some embodiments, the helper lipid can be a phosphor lipid or a steroid. In some embodiments, the helper lipid is between about 20 mol % to 80 mol % of the total lipid content of the composition. In some embodiments, the helper lipid component is between about 35 mol % to 65 mol % of the total lipid content of the LNP. In some embodiments, the LNP includes lipids at 50 mol % and the helper lipid at 50 mol % of the total lipid content of the LNP.

Other non-limiting, exemplary LNP delivery vehicles are described in U.S. Patent Publication Nos. US 20160174546, US 20140301951, US 20150105538, US 20150250725, Wang et al., J. Control Release, 2017 Jan. 31. pii: S0168-3659(17)30038-X. doi: 10.1016/j.jconrel.2017.01.037. [Epub ahead of print]; Altmoglu et al., Biomater Sci., 4(12):1773-80, Nov. 15, 2016; Wang et al., PNAS, 113(11):2868-73 Mar. 15, 2016; Wang et al., PloS One, 10(11): e0141860. doi: 10.1371/journal.pone.0141860. eCollection 2015, Nov. 3, 2015; Takeda et al., Neural Regen Res. 10(5):689-90, May 2015; Wang et al., Adv. Healthc Mater., 3(9):1398-403, Sep. 2014; and Wang et al., Agnew Chem Int Ed Engl., 53(11):2893-8, Mar. 10, 2014; James E. Dahlman and Carmen Barnes et al. Nature Nanotechnology (2014) published online 11 May 2014, doi:10.1038/nnano.2014.84; Coelho et al., N Engl J Med 2013; 369:819-29; Aleku et al., Cancer Res., 68(23): 9788-98 (Dec. 1, 2008), Strumberg et al., Int. J. Clin. Pharmacol. Ther., 50(1): 76-8 (January 2012), Schultheis et al., J. Clin. Oncol., 32(36): 4141-48 (Dec. 20, 2014), and Fehring et al., Mol. Ther., 22(4): 811-20 (Apr. 22, 2014); Novobrantseva, Molecular Therapy-Nucleic Acids (2012) 1, e4; doi:10.1038/mtna.2011.3; WO2012135025; US 20140348900; US 20140328759; US 20140308304; WO 2005/105152; WO 2006/069782; WO 2007/121947; US 2015/082080; US 20120251618; 7,982,027; 7,799,565; 8,058,069; 8,283,333; 7,901,708; 7,745,651; 7,803,397; 8,101,741; 8,188,263; 7,915,399; 8,236,943 and 7,838,658 and European Pat. Nos 1766035; 1519714; 1781593 and 1664316;

### Liposomes

In some embodiments, a lipid particle may be liposome. Liposomes are spherical vesicle structures composed of a uni- or multilamellar lipid bilayer surrounding internal aqueous compartments and a relatively impermeable outer lipophilic phospholipid bilayer. In some embodiments, liposomes are biocompatible, nontoxic, can deliver both hydrophilic and lipophilic drug molecules, protect their cargo from degradation by plasma enzymes, and transport their load across biological membranes and the blood brain barrier (BBB).

Liposomes can be made from several different types of lipids, e.g., phospholipids. A liposome may comprise natural phospholipids and lipids such as 1,2-distearoryl-sn-glycero-3-phosphatidyl choline (DSPC), sphingomyelin, egg phosphatidylcholines, monosialoganglioside, or any combination thereof.

Several other additives may be added to liposomes in order to modify their structure and properties. For instance, liposomes may further comprise cholesterol, sphingomyelin, and/or 1,2-dioleoyl-sn-glycero-3-phosphoethanolamine (DOPE), e.g., to increase stability and/or to prevent the leakage of the liposomal inner cargo.

In some embodiments, a liposome delivery vehicle can be used to deliver a virus particle containing a engineered intein system and/or component(s) thereof. In some embodiments, the virus particle(s) can be adsorbed to the liposome, such as through electrostatic interactions, and/or can be attached to the liposomes via a linker.

In some embodiments, the liposome can be a Trojan Horse liposome (also known in the art as Molecular Trojan Horses), see e.g., http://cshprotocols.cshlp.org/content/2010/4/pdb.prot5407.long, the teachings of which can be applied and/or adapted to generated and/or deliver the engineered intein system described herein.

Other non-limiting, exemplary liposomes can be those as set forth in Wang et al., ACS Synthetic Biology, 1, 403-07 (2012); Wang et al., PNAS, 113(11) 2868-2873 (2016); Spuch and Navarro, Journal of Drug Delivery, vol. 2011, Article ID 469679, 12 pages, 2011. doi:10.1155/2011/469679; WO 2008/042973; U.S. Pat. No. 8,071,082; WO 2014/186366; 20160257951; US20160129120; US 20160244761; 20120251618; WO2013/093648; Lipofectin (a combination of DOTMA and DOPE), Lipofectase, LIPOFECTAMINE® (e.g., LIPOFECTAMINE® 2000, LIPOFECTAMINE® 3000, LIPOFECTAMINE® RNAiMAX, LIPOFECTAMINE® LTX), SAINT-RED (Synvolux Therapeutics, Groningen Netherlands), DOPE, Cytofectin (Gilead Sciences, Foster City, Calif.), and Eufectins (JBL, San Luis Obispo, Calif.).

### Stable Nucleic-Acid-Lipid Particles (SNALPs)

In some embodiments, the lipid particles may be stable nucleic acid lipid particles (SNALPs). SNALPs may comprise an ionizable lipid (DLinDMA) (e.g., cationic at low pH), a neutral helper lipid, cholesterol, a diffusible polyethylene glycol (PEG)-lipid, or any combination thereof. In some examples, SNALPs may comprise synthetic cholesterol, dipalmitoylphosphatidylcholine, 3-N-[(w-methoxy polyethylene glycol)2000)carbamoyl]-1,2-dimyrestyloxypropylamine, and cationic 1,2-dilinoleyloxy-3-N,Ndimethylaminopropane. In some examples, SNALPs may comprise synthetic cholesterol, 1,2-distearoyl-sn-glycero-3-phosphocholine, PEG-cDMA, and 1,2-dilinoleyloxy-3-(N;N-dimethyl)aminopropane (DLinDMAo).

Other non-limiting, exemplary SNALPs that can be used to deliver the engineered intein system described herein can be any such SNALPs as described in Morrissey et al., Nature Biotechnology, Vol. 23, No. 8, August 2005, Zimmerman et al., Nature Letters, Vol. 441, 4 May 2006; Geisbert et al., Lancet 2010; 375: 1896-905; Judge, J. Clin. Invest. 119:661-673 (2009); and Semple et al., Nature Niotechnology, Volume 28 Number 2 February 2010, pp. 172-177.

### Other Lipids

The lipid particles may also comprise one or more other types of lipids, e.g., cationic lipids, such as amino lipid 2,2-dilinoleyl-4-dimethylaminoethyl-[1,3]-dioxolane (DLin-KC2-DMA), DLin-KC2-DMA4, C12-200 and colipids disteroylphosphatidyl choline, cholesterol, and PEG-DMG.

In some embodiments, the delivery vehicle can be or include a lipidoid, such as any of those set forth in, for example, US 20110293703.

In some embodiments, the delivery vehicle can be or include an amino lipid, such as any of those set forth in, for example, Jayaraman, Angew. Chem. Int. Ed. 2012, 51, 8529-8533.

In some embodiments, the delivery vehicle can be or include a lipid envelope, such as any of those set forth in, for example, Korman et al., 2011. Nat. Biotech. 29:154-157.

### Lipoplexes/Polyplexes

In some embodiments, the delivery vehicles comprise lipoplexes and/or polyplexes. Lipoplexes may bind to negatively charged cell membrane and induce endocytosis into the cells. Examples of lipoplexes may be complexes comprising lipid(s) and non-lipid components. Examples of lipoplexes and polyplexes include FuGENE-6 reagent, a non-liposomal solution containing lipids and other components, zwitterionic amino lipids (ZALs), Ca2β (e.g., forming DNA/Ca²⁺ microcomplexes), polyethenimine (PEI) (e.g., branched PEI), and poly(L-lysine) (PLL).

### Sugar-Based Particles

In some embodiments, the delivery vehicle can be a sugar-based particle. In some embodiments, the sugar-based particles can be or include GalNAc, such as any of those described in WO2014118272; US 20020150626; Nair, J K et al., 2014, Journal of the American Chemical Society 136 (49), 16958-16961; Østergaard et al., Bioconjugate Chem., 2015, 26(8), pp 1451-1455;

### Cell Penetrating Peptides

In some embodiments, the delivery vehicles comprise cell penetrating peptides (CPPs). CPPs are short peptides that facilitate cellular uptake of various molecular cargo (e.g., from nanosized particles to small chemical molecules and large fragments of DNA).

CPPs may be of different sizes, amino acid sequences, and charges. In some examples, CPPs can translocate the plasma membrane and facilitate the delivery of various molecular cargoes to the cytoplasm or an organelle. CPPs may be introduced into cells via different mechanisms, e.g., direct penetration in the membrane, endocytosis-mediated entry, and translocation through the formation of a transitory structure.

CPPs may have an amino acid composition that either contains a high relative abundance of positively charged amino acids such as lysine or arginine or has sequences that contain an alternating pattern of polar/charged amino acids and non-polar, hydrophobic amino acids. These two types of structures are referred to as polycationic or amphipathic, respectively. A third class of CPPs are the hydrophobic peptides, containing only apolar residues, with low net charge or have hydrophobic amino acid groups that are crucial for cellular uptake. Another type of CPPs is the trans-activating transcriptional activator (Tat) from Human Immunodeficiency Virus 1 (HIV-1). Examples of CPPs include to Penetratin, Tat (48-60), Transportan, and (R-AhX-R4) (Ahx refers to aminohexanoyl), Kaposi fibroblast growth factor (FGF) signal peptide sequence, integrin 33 signal peptide sequence, polyarginine peptide Args sequence, Guanine rich-molecular transporters, and sweet arrow peptide. Examples of CPPs and related applications also include those described in U.S. Pat. No. 8,372,951.

CPPs can be used for in vitro and ex vivo work quite readily, and extensive optimization for each cargo and cell type is usually required. In some examples, CPPs may be covalently attached to the Cas protein directly, which is then complexed with the gRNA and delivered to cells. In some examples, separate delivery of CPP-Cas and CPP-gRNA to multiple cells may be performed. CPP may also be used to delivery RNPs.

CPPs may be used to deliver the compositions and systems to plants. In some examples, CPPs may be used to deliver the components to plant protoplasts, which are then regenerated to plant cells and further to plants.

### DNA Nanoclews

In some embodiments, the delivery vehicles comprise DNA nanoclews. A DNA nanoclew refers to a sphere-like structure of DNA (e.g., with a shape of a ball of yarn). The nanoclew may be synthesized by rolling circle amplification with palindromic sequences that aide in the self-assembly of the structure. The sphere may then be loaded with a payload. An example of DNA nanoclew is described in Sun W et al, J Am Chem Soc. 2014 Oct. 22; 136(42):14722-5; and Sun W et al, Angew Chem Int Ed Engl. 2015 Oct. 5; 54(41):12029-33. A DNA nanoclew may be coated, e.g., coated with PEI to induce endosomal escape.

### Metal Nanoparticles

In some embodiments, the delivery vehicles comprise gold nanoparticles (also referred to AuNPs or colloidal gold). Gold nanoparticles may form complex with cargos, e.g., engineered intein system polypeptides and/or encoding polynucleotides. Gold nanoparticles may be coated, e.g., coated in a silicate and an endosomal disruptive polymer, PAsp(DET). Examples of gold nanoparticles include AuraSense Therapeutics' Spherical Nucleic Acid (SNA™) constructs, and those described in Mout R, et al. (2017). ACS Nano 11:2452-8; Lee K, et al. (2017). Nat Biomed Eng 1:889-901. Other metal nanoparticles can also be complexed with cargo(s). Such metal particles include tungsten, palladium, rhodium, platinum, and iridium particles. Other non-limiting, exemplary metal nanoparticles are described in US 20100129793.

iTOP

In some embodiments, the delivery vehicles comprise iTOP. iTOP refers to a combination of small molecules drives the highly efficient intracellular delivery of native proteins, independent of any transduction peptide. iTOP may be used for induced transduction by osmocytosis and propanebetaine, using NaCl-mediated hyperosmolality together with a transduction compound (propanebetaine) to trigger macropinocytotic uptake into cells of extracellular macromolecules. Examples of iTOP methods and reagents include those described in D'Astolfo D S, Pagliero R J, Pras A, et al. (2015). Cell 161:674-690.

### Polymer-Based Particles

In some embodiments, the delivery vehicles may comprise polymer-based particles (e.g., nanoparticles). In some embodiments, the polymer-based particles may mimic a viral mechanism of membrane fusion. The polymer-based particles may be a synthetic copy of Influenza virus machinery and form transfection complexes with various types of nucleic acids (siRNA, miRNA, plasmid DNA or shRNA, mRNA) that cells take up via the endocytosis pathway, a process that involves the formation of an acidic compartment. The low pH in late endosomes acts as a chemical switch that renders the particle surface hydrophobic and facilitates membrane crossing. Once in the cytosol, the particle releases its payload for cellular action. This Active Endosome Escape technology is safe and maximizes transfection efficiency as it is using a natural uptake pathway. In some embodiments, the polymer-based particles may comprise alkylated and carboxyalkylated branched polyethylenimine. In some examples, the polymer-based particles are VIROMER, e.g., VIROMER RNAi, VIROMER RED, VIROMER mRNA. Example methods of delivering the systems and compositions herein include those described in Bawage S S et al., Synthetic mRNA expressed Cas13a mitigates RNA virus infections, www.biorxiv.org/content/10.1101/370460v1.full doi: doi.org/10.1101/370460, Viromer® RED, a powerful tool for transfection of keratinocytes. doi: 10.13140/RG.2.2.16993.61281, Viromer® Transfection—Factbook 2018: technology, product overview, users' data., doi:10.13140/RG.2.2.23912.16642. Other exemplary and non-limiting polymeric particles are described in US 20170079916, US 20160367686, US 20110212179, US 20130302401, 6,007,845, 5,855,913, 5,985,309, 5,543,158, WO2012135025, US 20130252281, US 20130245107, US 20130244279; US 20050019923, 20080267903.

### Streptolysin O (SLO)

The delivery vehicles may be streptolysin O (SLO). SLO is a toxin produced by Group A streptococci that works by creating pores in mammalian cell membranes. SLO may act in a reversible manner, which allows for the delivery of proteins (e.g., up to 100 kDa) to the cytosol of cells without compromising overall viability. Examples of SLO include those described in Sierig G, et al. (2003). Infect Immun 71:446-55; Walev I, et al. (2001). Proc Natl Acad Sci USA 98:3185-90; Teng K W, et al. (2017). Elife 6:e25460.

### Multifunctional Envelope-Type Nanodevice (MEND)

The delivery vehicles may comprise multifunctional envelope-type nanodevice (MENDs). MENDs may comprise condensed plasmid DNA, a PLL core, and a lipid film shell. A MEND may further comprise cell-penetrating peptide (e.g., stearyl octaarginine). The cell penetrating peptide may be in the lipid shell. The lipid envelope may be modified with one or more functional components, e.g., one or more of: polyethylene glycol (e.g., to increase vascular circulation time), ligands for targeting of specific tissues/cells, additional cell-penetrating peptides (e.g., for greater cellular delivery), lipids to enhance endosomal escape, and nuclear delivery tags. In some examples, the MEND may be a tetra-lamellar MEND (T-MEND), which may target the cellular nucleus and mitochondria. In certain examples, a MEND may be a PEG-peptide-DOPE-conjugated MEND (PPD-MEND), which may target bladder cancer cells. Examples of MENDs include those described in Kogure K, et al. (2004). J Control Release 98:317-23; Nakamura T, et al. (2012). Acc Chem Res 45:1113-21.

### Lipid-Coated Mesoporous Silica Particles

The delivery vehicles may comprise lipid-coated mesoporous silica particles. Lipid-coated mesoporous silica particles may comprise a mesoporous silica nanoparticle core and a lipid membrane shell. The silica core may have a large internal surface area, leading to high cargo loading capacities. In some embodiments, pore sizes, pore chemistry, and overall particle sizes may be modified for loading different types of cargos. The lipid coating of the particle may also be modified to maximize cargo loading, increase circulation times, and provide precise targeting and cargo release. Examples of lipid-coated mesoporous silica particles include those described in Du X, et al. (2014). Biomaterials 35:5580-90; Durfee P N, et al. (2016). ACS Nano 10:8325-45.

### Inorganic Nanoparticles

The delivery vehicles may comprise inorganic nanoparticles. Examples of inorganic nanoparticles include carbon nanotubes (CNTs) (e.g., as described in Bates K and Kostarelos K. (2013). Adv Drug Deliv Rev 65:2023-33.), bare mesoporous silica nanoparticles (MSNPs) (e.g., as described in Luo G F, et al. (2014). Sci Rep 4:6064), and dense silica nanoparticles (SiNPs) (as described in Luo D and Saltzman W M. (2000). Nat Biotechnol 18:893-5).

### Exosomes

The delivery vehicles may comprise exosomes. Exosomes include membrane bound extracellular vesicles, which can be used to contain and delivery various types of biomolecules, such as proteins, carbohydrates, lipids, and nucleic acids, and complexes thereof (e.g., RNPs). Examples of exosomes include those described in Schroeder A, et al., J Intern Med. 2010 January; 267(1):9-21; El-Andaloussi S, et al., Nat Protoc. 2012 December; 7(12):2112-26; Uno Y, et al., Hum Gene Ther. 2011 June; 22(6):711-9; Zou W, et al., Hum Gene Ther. 2011 April; 22(4):465-75.

In some examples, the exosome may form a complex (e.g., by binding directly or indirectly) to one or more components of the cargo. In certain examples, a molecule of an exosome may be fused with first adapter protein and a component of the cargo may be fused with a second adapter protein. The first and the second adapter protein may specifically bind each other, thus associating the cargo with the exosome. Examples of such exosomes include those described in Ye Y, et al., Biomater Sci. 2020 Apr 28. doi: 10.1039/d0bm00427h.

Other non-limiting, exemplary exosomes include any of those set forth in Alvarez-Erviti et al. 2011, Nat Biotechnol 29: 341; [1401] El-Andaloussi et al. (Nature Protocols 7:2112-2126 (2012); and Wahlgren et al. (Nucleic Acids Research, 2012, Vol. 40, No. 17 e130).

### Spherical Nucleic Acids (SNAs)

In some embodiments, the delivery vehicle can be a SNA. SNAs are three dimensional nanostructures that can be composed of densely functionalized and highly oriented nucleic acids that can be covalently attached to the surface of spherical nanoparticle cores. The core of the spherical nucleic acid can impart the conjugate with specific chemical and physical properties, and it can act as a scaffold for assembling and orienting the oligonucleotides into a dense spherical arrangement that gives rise to many of their functional properties, distinguishing them from all other forms of matter. In some embodiments, the core is a crosslinked polymer. Non-limiting, exemplary SNAs can be any of those set forth in Cutler et al., J. Am. Chem. Soc. 2011 133:9254-9257, Hao et al., Small. 2011 7:3158-3162, Zhang et al., ACS Nano. 2011 5:6962-6970, Cutler et al., J. Am. Chem. Soc. 2012 134:1376-1391, Young et al., Nano Lett. 2012 12:3867-71, Zheng et al., Proc. Natl. Acad. Sci. USA. 2012 109:11975-80, Mirkin, Nanomedicine 2012 7:635-638 Zhang et al., J. Am. Chem. Soc. 2012 134:16488-1691, Weintraub, Nature 2013 495:S14-S16, Choi et al., Proc. Natl. Acad. Sci. USA. 2013 110(19):7625-7630, Jensen et al., Sci. Transl. Med. 5, 209ra152 (2013) and Mirkin, et al., and Small, 10:186-192.

### Self-Assembling Nanoparticles

In some embodiments, the delivery vehicle is a self-assembling nanoparticle. The self-assembling nanoparticles can contain one or more polymers. The self-assembling nanoparticles can be PEGylated. Self-assembling nanoparticles are known in the art. Non-limiting, exemplary self-assembling nanoparticles can any as set forth in Schiffelers et al., Nucleic Acids Research, 2004, Vol. 32, No. 19, Bartlett et al. (PNAS, Sep. 25, 2007, vol. 104, no. 39; Davis et al., Nature, Vol 464, 15 Apr. 2010.

### Supercharged Proteins

In some embodiments, the delivery vehicle can be a supercharged protein. As used herein “Supercharged proteins” are a class of engineered or naturally occurring proteins with unusually high positive or negative net theoretical charge. Non-limiting, exemplary supercharged proteins can be any of those set forth in Lawrence et al., 2007, Journal of the American Chemical Society 129, 10110-10112.

### Targeted Delivery

In some embodiments, the delivery vehicle can allow for targeted delivery to a specific cell, tissue, organ, or system. In such embodiments, the delivery vehicle can include one or more targeting moieties that can direct targeted delivery of the cargo(s). In an embodiment, the delivery vehicle comprises a targeting moiety, such as active targeting of a lipid entity of the invention, e.g., lipid particle or nanoparticle or liposome or lipid bilayer of the invention comprising a targeting moiety for active targeting.

With regard to targeting moieties, mention is made of Deshpande et al, “Current trends in the use of liposomes for tumor targeting,” Nanomedicine (Lond). 8(9), doi:10.2217/nnm.13.118 (2013), and the documents it cites, all of which are incorporated herein by reference and the teachings of which can be applied and/or adapted for targeted delivery of one or more engineered intein system components described herein. Mention is also made of International Patent Publication No. WO 2016/027264, and the documents it cites, all of which are incorporated herein by reference, the teachings of which can be applied and/or adapted for targeted delivery of one or more engineered intein system components described herein. And mention is made of Lorenzer et al, “Going beyond the liver: Progress and challenges of targeted delivery of siRNA therapeutics,” Journal of Controlled Release, 203: 1-15 (2015), and the documents it cites, all of which are incorporated herein by reference, the teachings of which can be applied and/or adapted for targeted delivery of one or more engineered intein system components described herein.

An actively targeting lipid particle or nanoparticle or liposome or lipid bilayer delivery system are prepared by conjugating targeting moieties, including small molecule ligands, peptides and monoclonal antibodies, on the lipid or liposomal surface; for example, certain receptors, such as folate and transferrin (Tf) receptors (TfR), are overexpressed on many cancer cells and have been used to make liposomes tumor cell specific. Liposomes that accumulate in the tumor microenvironment can be subsequently endocytosed into the cells by interacting with specific cell surface receptors. To efficiently target liposomes to cells, such as cancer cells, it is useful that the targeting moiety have an affinity for a cell surface receptor and to link the targeting moiety in sufficient quantities to have optimum affinity for the cell surface receptors; and determining these embodiments are within the ambit of the skilled artisan. In the field of active targeting, there are a number of cell-, e.g., tumor-, specific targeting ligands.

Also, as to active targeting, with regard to targeting cell surface receptors such as cancer cell surface receptors, targeting ligands on liposomes can provide attachment of liposomes to cells, e.g., vascular cells, via a noninternalizing epitope; and this can increase the extracellular concentration of that which is being delivered, thereby increasing the amount delivered to the target cells. A strategy to target cell surface receptors, such as cell surface receptors on cancer cells, such as overexpressed cell surface receptors on cancer cells, is to use receptor-specific ligands or antibodies. Many cancer cell types display upregulation of tumor-specific receptors. For example, TfRs and folate receptors (FRs) are greatly overexpressed by many tumor cell types in response to their increased metabolic demand. Folic acid can be used as a targeting ligand for specialized delivery owing to its ease of conjugation to nanocarriers, its high affinity for FRs and the relatively low frequency of FRs, in normal tissues as compared with their overexpression in activated macrophages and cancer cells, e.g., certain ovarian, breast, lung, colon, kidney and brain tumors. Overexpression of FR on macrophages is an indication of inflammatory diseases, such as psoriasis, Crohn's disease, rheumatoid arthritis and atherosclerosis; accordingly, folate-mediated targeting of the invention can also be used for studying, addressing or treating inflammatory disorders, as well as cancers. Folate-linked lipid particles or nanoparticles or liposomes or lipid bilayers can deliver their cargo intracellularly through receptor-mediated endocytosis. Intracellular trafficking can be directed to acidic compartments that facilitate cargo release, and, most importantly, release of the cargo can be altered or delayed until it reaches the cytoplasm or vicinity of target organelles. Delivery of cargo using a lipid entity having a targeting moiety, such as a folate-linked lipid entity of the invention, can be superior to nontargeted lipid entity. The attachment of folate directly to the lipid head groups may not be favorable for intracellular delivery of folate-conjugated lipid, since they may not bind as efficiently to cells as folate attached to the lipid entity surface by a spacer, which may can enter cancer cells more efficiently. A lipid entity coupled to folate can be used for the delivery of complexes of lipid, e.g., liposome, e.g., anionic liposome and virus or capsid or envelope or virus outer protein, such as those herein discussed such as adenovirus or AAV. Tf is a monomeric serum glycoprotein of approximately 80 KDa involved in the transport of iron throughout the body. Tf binds to the TfR and translocates into cells via receptor-mediated endocytosis. The expression of TfR can be higher in certain cells, such as tumor cells (as compared with normal cells and is associated with the increased iron demand in rapidly proliferating cancer cells. Accordingly, the invention comprehends a TfR-targeted lipid, e.g., as to liver cells, liver cancer, breast cells such as breast cancer cells, colon such as colon cancer cells, ovarian cells such as ovarian cancer cells, head, neck and lung cells, such as head, neck and non-small-cell lung cancer cells, cells of the mouth such as oral tumor cells.

Also, as to active targeting, a lipid entity can be multifunctional, i.e., employ more than one targeting moiety such as CPP, along with Tf; a bifunctional system; e.g., a combination of Tf and poly-L-arginine which can provide transport across the endothelium of the blood-brain barrier. EGFR is a tyrosine kinase receptor belonging to the ErbB family of receptors that mediates cell growth, differentiation and repair in cells, especially non-cancerous cells, but EGF is overexpressed in certain cells such as many solid tumors, including colorectal, non-small-cell lung cancer, squamous cell carcinoma of the ovary, kidney, head, pancreas, neck and prostate, and especially breast cancer. The invention comprehends EGFR-targeted monoclonal antibody(ies) linked to a lipid. HER-2 is often overexpressed in patients with breast cancer, and is also associated with lung, bladder, prostate, brain and stomach cancers. HER-2, encoded by the ERBB2 gene. The invention comprehends a HER-2-targeting lipid=, e.g., an anti-HER-2-antibody (or binding fragment thereof)-lipid, a HER-2-targeting-PEGylated lipid entity of the invention (e.g., having an anti-HER-2-antibody or binding fragment thereof), a HER-2-targeting-maleimide-PEG polymer-lipid (e.g., having an anti-HER-2-antibody or binding fragment thereof). Upon cellular association, the receptor-antibody complex can be internalized by formation of an endosome for delivery to the cytoplasm.

With respect to receptor-mediated targeting, the skilled artisan takes into consideration ligand/target affinity and the quantity of receptors on the cell surface, and that PEGylation can act as a barrier against interaction with receptors. The use of antibody-lipid entity of the invention targeting can be advantageous. Multivalent presentation of targeting moieties can also increase the uptake and signaling properties of antibody fragments. In practice of the invention, the skilled person takes into account ligand density (e.g., high ligand densities on a lipid entity of the invention may be advantageous for increased binding to target cells). Preventing early by macrophages can be addressed with a sterically stabilized lipid entity of the invention and linking ligands to the terminus of molecules such as PEG, which is anchored in the lipid entity of the invention (e.g., lipid particle or nanoparticle or liposome or lipid bilayer). The microenvironment of a cell mass such as a tumor microenvironment can be targeted; for instance, it may be advantageous to target cell mass vasculature, such as the tumor vasculature microenvironment. Thus, the invention comprehends targeting VEGF. VEGF and its receptors are well-known proangiogenic molecules and are well-characterized targets for antiangiogenic therapy. Many small-molecule inhibitors of receptor tyrosine kinases, such as VEGFRs or basic FGFRs, have been developed as anticancer agents and the invention comprehends coupling any one or more of these peptides to a lipid entity of the invention, e.g., phage IVO peptide(s) (e.g., via or with a PEG terminus), tumor-homing peptide APRPG such as APRPG-PEG-modified. VCAM, the vascular endothelium plays a key role in the pathogenesis of inflammation, thrombosis and atherosclerosis. CAMs are involved in inflammatory disorders, including cancer, and are a logical target, E- and P-selectins, VCAM-1 and ICAMs. Can be used to target a lipid entity of the invention., e.g., with PEGylation.

Matrix metalloproteases (MMPs) belong to the family of zinc-dependent endopeptidases. They are involved in tissue remodeling, tumor invasiveness, resistance to apoptosis and metastasis. There are four MMP inhibitors called TIMP1-4, which determine the balance between tumor growth inhibition and metastasis; a protein involved in the angiogenesis of tumor vessels is MT1-MMP, expressed on newly formed vessels and tumor tissues. The proteolytic activity of MT1-MMP cleaves proteins, such as fibronectin, elastin, collagen and laminin, at the plasma membrane and activates soluble MMPs, such as MMP-2, which degrades the matrix. An antibody or fragment thereof such as a Fab′ fragment can be used in the practice of the invention such as for an antihuman MT1-MMP monoclonal antibody linked to a lipid entity of the invention, e.g., via a spacer such as a PEG spacer. αβ-integrins or integrins are a group of transmembrane glycoprotein receptors that mediate attachment between a cell and its surrounding tissues or extracellular matrix.

Integrins contain two distinct chains (heterodimers) called α- and β-subunits. The tumor tissue-specific expression of integrin receptors can be utilized for targeted delivery in the invention, e.g., whereby the targeting moiety can be an RGD peptide such as a cyclic RGD.

Aptamers are ssDNA or RNA oligonucleotides that impart high affinity and specific recognition of the target molecules by electrostatic interactions, hydrogen bonding and hydro phobic interactions as opposed to the Watson-Crick base pairing, which is typical for the bonding interactions of oligonucleotides. Aptamers as a targeting moiety can have advantages over antibodies: aptamers can demonstrate higher target antigen recognition as compared with antibodies; aptamers can be more stable and smaller in size as compared with antibodies; aptamers can be easily synthesized and chemically modified for molecular conjugation; and aptamers can be changed in sequence for improved selectivity and can be developed to recognize poorly immunogenic targets. Such moieties as a sgc8 aptamer can be used as a targeting moiety (e.g., via covalent linking to the lipid entity of the invention, e.g., via a spacer, such as a PEG spacer).

Also, as to active targeting, the invention also comprehends intracellular delivery. Since liposomes follow the endocytic pathway, they are entrapped in the endosomes (pH 6.5-6) and subsequently fuse with lysosomes (pH<5), where they undergo degradation that results in a lower therapeutic potential. The low endosomal pH can be taken advantage of to escape degradation. Fusogenic lipids or peptides, which destabilize the endosomal membrane after the conformational transition/activation at a lowered pH. Amines are protonated at an acidic pH and cause endosomal swelling and rupture by a buffer effect Unsaturated dioleoylphosphatidylethanolamine (DOPE) readily adopts an inverted hexagonal shape at a low pH, which causes fusion of liposomes to the endosomal membrane. This process destabilizes a lipid entity containing DOPE and releases the cargo into the cytoplasm; fusogenic lipid GALA, cholesteryl-GALA and PEG-GALA may show a highly efficient endosomal release; a pore-forming protein listeriolysin O may provide an endosomal escape mechanism; and histidine-rich peptides have the ability to fuse with the endosomal membrane, resulting in pore formation, and can buffer the proton pump causing membrane lysis.

The invention comprehends a lipid entity modified with CPP(s), for intracellular delivery that may proceed via energy dependent macropinocytosis followed by endosomal escape. The invention further comprehends organelle-specific targeting. A lipid entity surface-functionalized with the triphenylphosphonium (TPP) moiety or a lipid entity with a lipophilic cation, rhodamine 123 can be effective in delivery of cargo to mitochondria. DOPE/sphingomyelin/stearyl-octa-arginine can delivers cargos to the mitochondrial interior via membrane fusion. A lipid entity surface modified with a lysosomotropic ligand, octadecyl rhodamine B can deliver cargo to lysosomes. Ceramides are useful in inducing lysosomal membrane permeabilization; the invention comprehends intracellular delivery of a lipid entity having a ceramide. The invention further comprehends a lipid entity targeting the nucleus, e.g., via a DNA-intercalating moiety. The invention also comprehends multifunctional liposomes for targeting, i.e., attaching more than one functional group to the surface of the lipid entity, for instance to enhances accumulation in a desired site and/or promotes organelle-specific delivery and/or target a particular type of cell and/or respond to the local stimuli such as temperature (e.g., elevated), pH (e.g., decreased), respond to externally applied stimuli such as a magnetic field, light, energy, heat or ultrasound and/or promote intracellular delivery of the cargo. All of these are considered actively targeting moieties.

It should be understood that as to each possible targeting or active targeting moiety herein discussed, there is an embodiment of the invention wherein the delivery system comprises such a targeting or active targeting moiety. Targeting moieties for specific cell types and/or states are generally known in the art and will be appreciated by those of ordinary skill in the art in view of the description provided herein.

Other exemplary targeting moieties are described elsewhere herein, such as epitope tags and the like.

### Responsive Delivery

In some embodiments, the delivery vehicle can allow for responsive delivery of the cargo(s). Responsive delivery, as used in this context herein, refers to delivery of cargo(s) by the delivery vehicle in response to an external stimulus. Examples of suitable stimuli include, without limitation, an energy (light, heat, cold, and the like), a chemical stimulus (e.g., chemical composition, etc.), and a biologic or physiologic stimulus (e.g., environmental pH, osmolarity, salinity, biologic molecule, etc.). In some embodiments, the targeting moiety can be responsive to an external stimulus and facilitate responsive delivery. In other embodiments, responsiveness is determined by a non-targeting moiety component of the delivery vehicle.

The delivery vehicle can be stimuli-sensitive, e.g., sensitive to an externally applied stimuli, such as magnetic fields, ultrasound or light; and pH-triggering can also be used, e.g., a labile linkage can be used between a hydrophilic moiety such as PEG and a hydrophobic moiety such as a lipid entity of the invention, which is cleaved only upon exposure to the relatively acidic conditions characteristic of the a particular environment or microenvironment such as an endocytic vacuole or the acidotic tumor mass. pH-sensitive copolymers can also be incorporated in embodiments of the invention can provide shielding; diortho esters, vinyl esters, cysteine-cleavable lipopolymers, double esters and hydrazones are a few examples of pH-sensitive bonds that are quite stable at pH 7.5, but are hydrolyzed relatively rapidly at pH 6 and below, e.g., a terminally alkylated copolymer ofN-isopropylacrylamide and methacrylic acid that copolymer facilitates destabilization of a lipid entity of the invention and release in compartments with decreased pH value; or, the invention comprehends ionic polymers for generation of a pH-responsive lipid entity of the invention (e.g., poly(methacrylic acid), poly(diethylaminoethyl methacrylate), poly(acrylamide) and poly(acrylic acid)).

Temperature-triggered delivery is also within the ambit of the invention. Many pathological areas, such as inflamed tissues and tumors, show a distinctive hyperthermia compared with normal tissues. Utilizing this hyperthermia is an attractive strategy in cancer therapy since hyperthermia is associated with increased tumor permeability and enhanced uptake. This technique involves local heating of the site to increase microvascular pore size and blood flow, which, in turn, can result in an increased extravasation of embodiments of the invention. Temperature-sensitive lipid entity of the invention can be prepared from thermosensitive lipids or polymers with a low critical solution temperature. Above the low critical solution temperature (e.g., at site such as tumor site or inflamed tissue site), the polymer precipitates, disrupting the liposomes to release. Lipids with a specific gel-to-liquid phase transition temperature are used to prepare these lipid entities of the invention; and a lipid for a thermosensitive embodiment can be dipalmitoylphosphatidylcholine. Thermosensitive polymers can also facilitate destabilization followed by release, and a useful thermosensitive polymer is poly (N-isopropylacrylamide). Another temperature triggered system can employ lysolipid temperature-sensitive liposomes.

The invention also comprehends redox-triggered delivery. The difference in redox potential between normal and inflamed or tumor tissues, and between the intra- and extra-cellular environments has been exploited for delivery, e.g., GSH is a reducing agent abundant in cells, especially in the cytosol, mitochondria and nucleus. The GSH concentrations in blood and extracellular matrix are just one out of 100 to one out of 1000 of the intracellular concentration, respectively. This high redox potential difference caused by GSH, cysteine and other reducing agents can break the reducible bonds, destabilize a lipid entity of the invention and result in release of payload. The disulfide bond can be used as the cleavable/reversible linker in a lipid entity of the invention, because it causes sensitivity to redox owing to the disulfideto-thiol reduction reaction; a lipid entity of the invention can be made reduction sensitive by using two (e.g., two forms of a disulfide-conjugated multifunctional lipid as cleavage of the disulfide bond (e.g., via tris(2-carboxyethyl)phosphine, dithiothreitol, L-cysteine or GSH), can cause removal of the hydrophilic head group of the conjugate and alter the membrane organization leading to release of payload. Calcein release from reduction-sensitive lipid entity of the invention containing a disulfide conjugate can be more useful than a reduction-insensitive embodiment.

Enzymes can also be used as a trigger to release payload. Enzymes, including MMPs (e.g., MMP2), phospholipase A2, alkaline phosphatase, transglutaminase or phosphatidylinositol-specific phospholipase C, have been found to be overexpressed in certain tissues, e.g., tumor tissues. In the presence of these enzymes, specially engineered enzyme-sensitive lipid entity of the invention can be disrupted and release the payload. An MMP2-cleavable octapeptide (Gly-Pro-Leu-Gly-Ile-Ala-Gly-Gln (SEQ ID NO: 113)) can be incorporated into a linker, and can have antibody targeting, e.g., antibody 2C5.

The invention also comprehends light- or energy-triggered delivery, e.g., the lipid entity of the invention can be light-sensitive, such that light or energy can facilitate structural and conformational changes, which lead to direct interaction of the lipid entity of the invention with the target cells via membrane fusion, photo-isomerism, photofragmentation or photopolymerization; such a moiety therefor can be benzoporphyrin photosensitizer. Ultrasound can be a form of energy to trigger delivery; a lipid entity of the invention with a small quantity of particular gas, including air or perfluorated hydrocarbon can be triggered to release with ultrasound, e.g., low-frequency ultrasound (LFUS). Magnetic delivery: A lipid entity of the invention can be magnetized by incorporation of magnetites, such as Fe3O4 or γ-Fe2O3, e.g., those that are less than 10 nm in size. Targeted delivery can be then by exposure to a magnetic field.

### Cells and Organisms

Also provided herein are cells, cell populations, and organisms comprising an engineered intein system, polypeptide component thereof, encoding polynucleotide, vector, and/or vector or vector system described herein. In some embodiments, the cell is a prokaryotic cell. In some embodiments, the cell is a eukaryotic cell. In some embodiments, the eukaryotic cell is a mammalian cell. In some embodiments, the eukaryotic cell is a non-human mammalian cell. In some embodiments, the cell is a human cell. In some embodiments, the cell is a plant cell. In some embodiments, the cell is an algal cell. In some embodiments, the cell is a fungal cell. In some embodiments, the cell is a bacterium. In some embodiments, the cell is an insect cell. The cells can be modified in vitro, ex vivo, or in vivo. The engineered intein system, polypeptide component thereof, encoding polynucleotide, vector, and/or vector or vector system described herein can be delivered by any suitable technique, composition, system, or method. Suitable delivery methods and techniques include but are not limited to, transfection via a vector, transduction with viral particles, electroporation, endocytic methods, and others, which are described elsewhere herein and that will be appreciated by those of ordinary skill in the art in view of this disclosure.

The cells can be further optionally cultured and/or expanded in vitro or ex vivo using any suitable cell culture techniques or conditions, which unless specified otherwise herein, will be appreciated by one of ordinary skill in the art in view of this disclosure. In some embodiments, the cells can be modified, optionally cultured and/or expanded, and administered to a subject in need thereof, such as in a cell-based therapy. In some embodiments, cells can be isolated from a subject, subsequently modified (such as via introducing a system of the present invention herein or other modifying agent) and optionally cultured and/or expanded and administered back to the subject. Such administration can be referred to as autologous administration. In some embodiments, cells can be isolated from a first subject, subsequently modified, optionally cultured and/or expanded, and administered to a second subject, where the first subject and the second subject are different. Such administration can be referred to as non-autologous administration.

In some embodiments, one or more cells of a microbiome comprise an engineered intein system, polypeptide component thereof, encoding polynucleotide, vector, and/or vector or vector system described herein. In some embodiments, the cells containing the engineered intein system, polypeptide component thereof, encoding polynucleotide, vector, and/or vector or vector system described herein are delivered to a subject such that the cells become part of a microbiome (e.g., gut microbiome, skin microbiome, vaginal microbiome or other microbiome) of the subject.

The cells can also be used in e.g., a screen, such as in a screen to evaluate candidate agents. In some embodiments, the engineered intein system of the present invention can be used as a sensor within the cell in the screen. The cells can also be used in e.g., a disease model.

The cells can also be used as bioreactors to produce the engineered intein system of the present invention or components thereof. The cells can also be used as bioreactors to produce another bioproduct besides the engineered intein system of the present invention or components thereof. For example, the engineered intein system within the cell can facilitate production and/or harvesting of the bioproduct.

In some embodiments, the engineered intein system can be used to facilitate gene or polynucleotide delivery or modification in the cell, thus producing a modified cell.

Described in several exemplary embodiments herein are engineered (also referred to in this context as modified) organisms that comprise one or more cells described herein that contain an engineered intein system, polypeptide component thereof, encoding polynucleotide, vector, and/or vector or vector system of the present invention described herein or are otherwise modified by the an engineered intein system, polypeptide component thereof, encoding polynucleotide, vector, and/or vector or vector system of the present invention described herein.

In some embodiments, the modified organism is a non-human animal. In some embodiments, the non-human animal is an avian or a reptile. In some embodiments, the modified organism is a non-human mammal. In some embodiments, the modified organism is a modified plant. In some embodiments, the modified organism is an insect. In some embodiments, the modified organism is a fungus. Methods of making modified organisms are described in greater detail elsewhere herein and will be appreciated by those of ordinary skill in the art in view of this disclosure.

It will be appreciated that the engineered intein system, polypeptide component thereof, encoding polynucleotide, vector, and/or vector or vector system described herein can be provided to a non-human animal or non-animal organism (e.g., plant) as a treatment, sensor or for another purpose or provided to the non-human animal or non-animal organism (e.g., plant) so as to generate a bioreactor to produce the engineered intein system, polypeptide components thereof, encoding polynucleotide, vector, and/or vector or vector system described herein.

In general, the term “plant” relates to any various photosynthetic, eukaryotic, unicellular or multicellular organism of the kingdom Plantae characteristically growing by cell division, containing chloroplasts, and having cell walls comprised of cellulose. The term plant encompasses monocotyledonous and dicotyledonous plants. The term also encompasses progeny of the plants. The term plant also encompasses Algae, which are mainly photoautotrophs unified primarily by their lack of roots, leaves and other organs that characterize higher plants. A part of a plant, e.g., a “plant tissue” may be treated according to the methods of the present invention to produce an improved plant. Plant tissue also encompasses plant cells. The term “plant cell” as used herein refers to individual units of a living plant, either in an intact whole plant or in an isolated form grown in in vitro tissue cultures, on media or agar, in suspension in a growth media or buffer or as a part of higher organized unites, such as, for example, plant tissue, a plant organ, or a whole plant. Modified plants also encompass gametes, seeds, germplasm, embryos, either zygotic or somatic, progeny or hybrids of plants comprising the engineered intein system, polypeptide component thereof, encoding polynucleotide, vector, and/or vector or vector system described herein.

Engineered plants can be made for example via transformation methods. The term “transformation” broadly refers to the process by which a plant host is genetically modified by the introduction of DNA by means of Agrobacteria or one of a variety of chemical or physical methods. As used herein, the term “plant host” refers to plants, including any cells, tissues, organs, or progeny of the plants. Many suitable plant tissues or plant cells can be transformed and include, but are not limited to, protoplasts, somatic embryos, pollen, leaves, seedlings, stems, calli, stolons, microtubers, and shoots. A plant tissue also refers to any clone of such a plant, seed, progeny, propagule whether generated sexually or asexually, and descendants of any of these, such as cuttings or seed. A “protoplast” refers to a plant cell that has had its protective cell wall completely or partially removed using, for example, mechanical or enzymatic means resulting in an intact biochemical competent unit of living plant that can reform their cell wall, proliferate and regenerate grow into a whole plant under proper growing conditions.

Algae modification using polynucleotide modifying agents has been described in, for example U.S. Pat. No. 8,945,839 and WO 2015086795, which can be adapted to modifying algae and similar organisms with the polynucleotide modifying agents and systems described herein.

As used herein, a “fungal cell” refers to any type of eukaryotic cell within the kingdom of fungi. Phyla within the kingdom of fungi include Ascomycota, Basidiomycota, Blastocladiomycota, Chytridiomycota, Glomeromycota, Microsporidia, and Neocallimastigomycota. Fungal cells may include yeasts, molds, and filamentous fungi. In some embodiments, the fungal cell is a yeast cell.

As used herein, the term “yeast cell” refers to any fungal cell within the phyla Ascomycota and Basidiomycota. Yeast cells may include budding yeast cells, fission yeast cells, and mold cells. Without being limited to these organisms, many types of yeast used in laboratory and industrial settings are part of the phylum Ascomycota. In some embodiments, the yeast cell is an S. cerervisiae, Kluyveromyces marxianus, or Issatchenkia orientalis cell. Other yeast cells may include without limitation Candida spp. (e.g., Candida albicans), Yarrowia spp. (e.g., Yarrowia lipolytica), Pichia spp. (e.g., Pichia pastoris), Kluyveromyces spp. (e.g., Kluyveromyces lactis and Kluyveromyces marxianus), Neurospora spp. (e.g., Neurospora crassa), Fusarium spp. (e.g., Fusarium oxysporum), and Issatchenkia spp. (e.g., Issatchenkia orientalis, a.k.a. Pichia kudriavzevii and Candida acidothermophilum). In some embodiments, the fungal cell is a filamentous fungal cell. As used herein, the term “filamentous fungal cell” refers to any type of fungal cell that grows in filaments, i.e., hyphae or mycelia. Examples of filamentous fungal cells may include without limitation Aspergillus spp. (e.g., Aspergillus niger), Trichoderma spp. (e.g., Trichoderma reesei), Rhizopus spp. (e.g., Rhizopus oryzae), and Mortierella spp. (e.g., Mortierella isabellina). Methods for transforming yeast cells which can be used to introduce polynucleotides encoding the systems components are well known to the artisan and are reviewed by Kawai et al., 2010, Bioeng Bugs. 2010 November-December; 1(6): 395-403).

In some embodiments, the fungal cell is an industrial strain. As used herein, “industrial strain” refers to any strain of fungal cell used in or isolated from an industrial process, e.g., production of a product on a commercial or industrial scale. Industrial strain may refer to a fungal species that is typically used in an industrial process, or it may refer to an isolate of a fungal species that may be also used for non-industrial purposes (e.g., laboratory research). Examples of industrial processes may include fermentation (e.g., in production of food or beverage products), distillation, biofuel production, production of a compound, and production of a polypeptide. Examples of industrial strains may include, without limitation, JAY270 and ATCC4124.

In some embodiments, the fungal cell is a polyploid cell. As used herein, a “polyploid” cell may refer to any cell whose genome is present in more than one copy. A polyploid cell may refer to a type of cell that is naturally found in a polyploid state, or it may refer to a cell that has been induced to exist in a polyploid state (e.g., through specific regulation, alteration, inactivation, activation, or modification of meiosis, cytokinesis, or DNA replication).

In some embodiments, the fungal cell is a diploid cell. As used herein, a “diploid” cell may refer to any cell whose genome is present in two copies. A diploid cell may refer to a type of cell that is naturally found in a diploid state, or it may refer to a cell that has been induced to exist in a diploid state (e.g., through specific regulation, alteration, inactivation, activation, or modification of meiosis, cytokinesis, or DNA replication). For example, the S. cerevisiae strain S228C may be maintained in a haploid or diploid state. A diploid cell may refer to a cell whose entire genome is diploid, or it may refer to a cell that is diploid in a particular genomic locus of interest. In some embodiments, the fungal cell is a haploid cell. As used herein, a “haploid” cell may refer to any cell whose genome is present in one copy. A haploid cell may refer to a type of cell that is naturally found in a haploid state, or it may refer to a cell that has been induced to exist in a haploid state (e.g., through specific regulation, alteration, inactivation, activation, or modification of meiosis, cytokinesis, or DNA replication). For example, the S. cerevisiae strain S228C may be maintained in a haploid or diploid state. A haploid cell may refer to a cell whose entire genome is haploid, or it may refer to a cell that is haploid in a particular genomic locus of interest.

The engineered non-human organisms can be used for a variety of applications. For example, the engineered non-human organisms can be used as a disease or condition model or as a bioreactor to produce an engineered intein system, polypeptide component thereof, encoding polynucleotide, vector, and/or vector or vector system of the present invention described herein. In some embodiments, the engineered non-human organisms can be used as environmental or contaminant sensors. For example, the engineered intein system can be expressed in a plant and configured to catalyze a bioconjugation reaction under certain environmental conditions or when contaminants are present. The product of the bioconjugation reaction can therefore indicate the presence of the specific environmental condition or contaminant.

In some embodiments, the engineered intein system, polypeptide components thereof, encoding polynucleotide, vector, and/or vector or vector system of the present invention described herein are provided to a non-human organism so as to treat or prevent a condition or disease.

### Formulations

Also described herein are formulations, including pharmaceutical formulations, that can contain an amount, effective amount, and/or least effective amount, and/or therapeutically effective amount of one or more compounds, molecules, compositions, vectors, vector systems, cells, or a combination thereof (which are also referred to as the primary active agent or ingredient elsewhere herein) described in greater detail elsewhere herein and optionally a pharmaceutically acceptable carrier or excipient. As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo. As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient. When present, the compound can optionally be present in the pharmaceutical formulation as a pharmaceutically acceptable salt.

In some embodiments, an active ingredient (e.g., primary, secondary, etc. active agent) is present as a pharmaceutically acceptable salt of the active ingredient. As used herein, “pharmaceutically acceptable salt” refers to any acid or base addition salt whose counter-ions are non-toxic to the subject to which they are administered in pharmaceutical doses of the salts. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

The pharmaceutical formulations described herein can be administered to a subject in need thereof via any suitable method or route to a subject in need thereof. Suitable administration routes can include, but are not limited to auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratympanic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated and/or the active ingredient(s).

Where appropriate, compounds, molecules, compositions, vectors, vector systems, cells, or a combination thereof described in greater detail elsewhere herein can be provided to a subject in need thereof as an ingredient, such as an active ingredient or agent, in a formulation or pharmaceutical formulation. As such, also described are pharmaceutical formulations containing one or more of the compositions or systems, and/or where appropriate, salts thereof, or pharmaceutically acceptable salts thereof described herein. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

As used herein, “agent” refers to any substance, compound, molecule, and the like, which can be biologically active or otherwise can induce a biological and/or physiological effect on a subject to which it is administered to. As used herein, “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

### Pharmaceutically Acceptable Carriers and Secondary Ingredients and Agents

The pharmaceutical formulation can include a pharmaceutically acceptable carrier. Suitable pharmaceutically acceptable carriers include, but are not limited to water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxy methylcellulose, and polyvinyl pyrrolidone, which do not deleteriously react with the active composition.

The pharmaceutical formulations can be sterilized, and if desired, mixed with agents, such as lubricants, preservatives, stabilizers, wetting agents, emulsifiers, salts for influencing osmotic pressure, buffers, coloring, flavoring and/or aromatic substances, and the like which do not deleteriously react with the active compound.

In some embodiments, the pharmaceutical formulation can also include an effective amount of secondary active agents, including but not limited to, biologic agents or molecules including, but not limited to, e.g., polynucleotides, amino acids, peptides, polypeptides, antibodies, aptamers, ribozymes, hormones, immunomodulators, antipyretics, anxiolytics, antipsychotics, analgesics, antispasmodics, anti-inflammatories, anti-histamines, anti-infectives, chemotherapeutics, and any combination thereof.

### Effective Amounts

In some embodiments, the amount of the primary active agent and/or optional secondary agent can be an effective amount, least effective amount, and/or therapeutically effective amount. As used herein, “effective amount”, “effective concentration”, and/or the like refers to the amount, concentration, etc. of the primary and/or optional secondary agent included in the pharmaceutical formulation that achieve one or more therapeutic effects or desired effect. As used herein, “least effective”, “least effective concentration”, and/or the like amount refers to the lowest amount, concentration, etc. of the primary and/or optional secondary agent that achieves the one or more therapeutic or other desired effects. As used herein, “therapeutically effective amount”, “therapeutically effective concentration” and/or the like refers to the amount, concentration, etc. of the primary and/or optional secondary agent included in the pharmaceutical formulation that achieves one or more therapeutic effects. In some embodiments, the one or more therapeutic effects are to catalyze a bioconjugation reaction, such as a protein trans splicing reaction.

The effective amount, least effective amount, and/or therapeutically effective amount of the primary and optional secondary active agent described elsewhere herein contained in the pharmaceutical formulation can be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, μg, mg, or g or be any numerical value or subrange within any of these ranges.

In some embodiments, the effective amount, least effective amount, and/or therapeutically effective amount can be an effective concentration, least effective concentration, and/or therapeutically effective concentration, which can each be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pM, nM, μM, mM, or M or be any numerical value or subrange within any of these ranges.

In other embodiments, the effective amount, least effective amount, and/or therapeutically effective amount of the primary and optional secondary active agent be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 IU or be any numerical value or subrange within any of these ranges.

In some embodiments, the primary and/or the optional secondary active agent present in the pharmaceutical formulation can be any non-zero amount ranging from about 0 to 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.9, to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the pharmaceutical formulation or be any numerical value or subrange within any of these ranges.

In some embodiments where a cell or cell population is present in the pharmaceutical formulation (e.g., as a primary and/or or secondary active agent), the effective amount of cells can be any amount ranging from about 1 or 2 cells to 1×10¹ cells/mL, 1×10²⁰ cells/mL or more, such as about 1×10¹ cells/mL, 1×10² cells/mL, 1×10³ cells/mL, 1×10⁴ cells/mL, 1×10⁵ cells/mL, 1×10⁶ cells/mL, 1×10⁷ cells/mL, 1×10⁸ cells/mL, 1×10⁹ cells/mL, 1×10¹⁰ cells/mL, 1×10¹¹ cells/mL, 1×10¹² cells/mL, 1×10¹³ cells/mL, 1×10¹⁴ cells/mL, 1×10¹⁵ cells/mL, 1×10¹⁶ cells/mL, 1×10¹⁷ cells/mL, 1×10¹⁸ cells/mL, 1×10¹⁹ cells/mL, to/or about 1×0²⁰/cells mL or any numerical value or subrange within any of these ranges or any numerical value or subrange within any of these ranges.

In some embodiments, the amount or effective amount, particularly where an infective particle is being delivered (e.g., a virus particle having the primary or secondary agent as a cargo), the effective amount of virus particles can be expressed as a titer (plaque forming units per unit of volume) or as a MOI (multiplicity of infection). In some embodiments, the effective amount can be about 1×10¹ particles per pL, nL, μL, mL, or L to 1×10²⁰/particles per pL, nL, L, mL, or L or more, such as about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, to/or about 1×10²⁰ particles per pL, nL, μL, mL, or L. In some embodiments, the effective titer can be about 1×10¹ transforming units per pL, nL, μL, mL, or L to 1×10²⁰/ transforming units per pL, nL, L, mL, or L or more, such as about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×0¹³, 1×10¹⁴, 1×10¹⁵, 1×10¹⁶, 1×10¹⁷, 1×10¹⁸, 1×10¹⁹, to/or about 1×10²⁰ transforming units per pL, nL, μL, mL, or L or any numerical value or subrange within these ranges. In some embodiments, the MOI of the pharmaceutical formulation can range from about 0.1 to 10 or more, such as 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10 or more or any numerical value or subrange within these ranges.

In some embodiments, the amount or effective amount of the one or more of the active agent(s) described herein contained in the pharmaceutical formulation can range from about 1 μg/kg to about 10 mg/kg based upon the bodyweight of the subject in need thereof or average bodyweight of the specific patient population to which the pharmaceutical formulation can be administered.

In embodiments where there is a secondary agent contained in the pharmaceutical formulation, the effective amount of the secondary active agent will vary depending on the secondary agent, the primary agent, the administration route, subject age, disease, stage of disease, among other things, which will be one of ordinary skill in the art.

When optionally present in the pharmaceutical formulation, the secondary active agent can be included in the pharmaceutical formulation or can exist as a stand-alone compound or pharmaceutical formulation that can be administered contemporaneously or sequentially with the compound, derivative thereof, or pharmaceutical formulation thereof.

In some embodiments, the effective amount of the secondary active agent, when optionally present, is any non-zero amount ranging from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total active agents present in the pharmaceutical formulation, or any numerical value or subrange within these ranges. In additional embodiments, the effective amount of the secondary active agent is any non-zero amount ranging from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total pharmaceutical formulation or any numerical value or subrange within these ranges.

### Dosage Forms

In some embodiments, the pharmaceutical formulations described herein can be provided in a dosage form. The dosage form can be administered to a subject in need thereof. The dosage form can be effective generate specific concentration, such as an effective concentration, at a given site in the subject in need thereof. As used herein, “dose,” “unit dose,” or “dosage” can refer to physically discrete units suitable for use in a subject, each unit containing a predetermined quantity of the primary active agent, and optionally present secondary active ingredient, and/or a pharmaceutical formulation thereof calculated to produce the desired response or responses in association with its administration. In some embodiments, the given site is proximal to the administration site. In some embodiments, the given site is distal to the administration site. In some cases, the dosage form contains a greater amount of one or more of the active ingredients present in the pharmaceutical formulation than the final intended amount needed to reach a specific region or location within the subject to account for loss of the active components such as via first and second pass metabolism.

The dosage forms can be adapted for administration by any appropriate route. Appropriate routes include, but are not limited to, oral (including buccal or sublingual), rectal, intraocular, inhaled, intranasal, topical (including buccal, sublingual, or transdermal), vaginal, parenteral, subcutaneous, intramuscular, intravenous, internasal, and intradermal. Other appropriate routes are described elsewhere herein. Such formulations can be prepared by any method known in the art.

Dosage forms adapted for oral administration can discrete dosage units such as capsules, pellets or tablets, powders or granules, solutions, or suspensions in aqueous or non-aqueous liquids; edible foams or whips, or in oil-in-water liquid emulsions or water-in-oil liquid emulsions. In some embodiments, the pharmaceutical formulations adapted for oral administration also include one or more agents which flavor, preserve, color, or help disperse the pharmaceutical formulation. Dosage forms prepared for oral administration can also be in the form of a liquid solution that can be delivered as a foam, spray, or liquid solution. The oral dosage form can be administered to a subject in need thereof. Where appropriate, the dosage forms described herein can be microencapsulated.

The dosage form can also be prepared to prolong or sustain the release of any ingredient. In some embodiments, compounds, molecules, compositions, vectors, vector systems, cells, or a combination thereof described herein can be the ingredient whose release is delayed. In some embodiments the primary active agent is the ingredient whose release is delayed. In some embodiments, an optional secondary agent can be the ingredient whose release is delayed. Suitable methods for delaying the release of an ingredient include, but are not limited to, coating or embedding the ingredients in material in polymers, wax, gels, and the like. Delayed release dosage formulations can be prepared as described in standard references such as “Pharmaceutical dosage form tablets,” eds. Liberman et. al. (New York, Marcel Dekker, Inc., 1989), “Remington—The science and practice of pharmacy”, 20th ed., Lippincott Williams & Wilkins, Baltimore, MD, 2000, and “Pharmaceutical dosage forms and drug delivery systems”, 6th Edition, Ansel et al., (Media, PA: Williams and Wilkins, 1995). These references provide information on excipients, materials, equipment, and processes for preparing tablets and capsules and delayed release dosage forms of tablets and pellets, capsules, and granules. The delayed release can be anywhere from about an hour to about 3 months or more.

Examples of suitable coating materials include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings may be formed with a different ratio of water-soluble polymer, water insoluble polymers, and/or pH dependent polymers, with or without water insoluble/water soluble non-polymeric excipient, to produce the desired release profile. The coating is either performed on the dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Where appropriate, the dosage forms described herein can be a liposome. In these embodiments, primary active ingredient(s), and/or optional secondary active ingredient(s), and/or pharmaceutically acceptable salt thereof where appropriate are incorporated into a liposome. In embodiments where the dosage form is a liposome, the pharmaceutical formulation is thus a liposomal formulation. The liposomal formulation can be administered to a subject in need thereof.

Dosage forms adapted for topical administration can be formulated as ointments, creams, suspensions, lotions, powders, solutions, pastes, gels, sprays, aerosols, or oils. In some embodiments for treatments of the eye or other external tissues, for example the mouth or the skin, the pharmaceutical formulations are applied as a topical ointment or cream. When formulated in an ointment, a primary active ingredient, optional secondary active ingredient, and/or pharmaceutically acceptable salt thereof where appropriate can be formulated with a paraffinic or water-miscible ointment base. In other embodiments, the primary and/or secondary active ingredient can be formulated in a cream with an oil-in-water cream base or a water-in-oil base. Dosage forms adapted for topical administration in the mouth include lozenges, pastilles, and mouth washes.

Dosage forms adapted for nasal or inhalation administration include aerosols, solutions, suspension drops, gels, or dry powders. In some embodiments, a primary active ingredient, optional secondary active ingredient, and/or pharmaceutically acceptable salt thereof where appropriate can be in a dosage form adapted for inhalation is in a particle-size-reduced form that is obtained or obtainable by micronization. In some embodiments, the particle size of the size reduced (e.g., micronized) compound or salt or solvate thereof, is defined by a D₅₀ value of about 0.5 to about 10 microns as measured by an appropriate method known in the art. Dosage forms adapted for administration by inhalation also include particle dusts or mists. Suitable dosage forms wherein the carrier or excipient is a liquid for administration as a nasal spray or drops include aqueous or oil solutions/suspensions of an active (primary and/or secondary) ingredient, which may be generated by various types of metered dose pressurized aerosols, nebulizers, or insufflators. The nasal/inhalation formulations can be administered to a subject in need thereof.

In some embodiments, the dosage forms are aerosol formulations suitable for administration by inhalation. In some of these embodiments, the aerosol formulation contains a solution or fine suspension of a primary active ingredient, secondary active ingredient, and/or pharmaceutically acceptable salt thereof where appropriate and a pharmaceutically acceptable aqueous or non-aqueous solvent. Aerosol formulations can be presented in single or multi-dose quantities in sterile form in a sealed container. For some of these embodiments, the sealed container is a single dose or multi-dose nasal or an aerosol dispenser fitted with a metering valve (e.g., metered dose inhaler), which is intended for disposal once the contents of the container have been exhausted.

Where the aerosol dosage form is contained in an aerosol dispenser, the dispenser contains a suitable propellant under pressure, such as compressed air, carbon dioxide, or an organic propellant, including but not limited to a hydrofluorocarbon. The aerosol formulation dosage forms in other embodiments are contained in a pump-atomizer. The pressurized aerosol formulation can also contain a solution or a suspension of a primary active ingredient, optional secondary active ingredient, and/or pharmaceutically acceptable salt thereof. In further embodiments, the aerosol formulation also contains co-solvents and/or modifiers incorporated to improve, for example, the stability and/or taste and/or fine particle mass characteristics (amount and/or profile) of the formulation. Administration of the aerosol formulation can be once daily or several times daily, for example 2, 3, 4, or 8 times daily, in which 1, 2, 3 or more doses are delivered each time. The aerosol formulations can be administered to a subject in need thereof.

For some dosage forms suitable and/or adapted for inhaled administration, the pharmaceutical formulation is a dry powder inhalable-formulations. In addition to a primary active agent, optional secondary active ingredient, and/or pharmaceutically acceptable salt thereof where appropriate, such a dosage form can contain a powder base such as lactose, glucose, trehalose, mannitol, and/or starch. In some of these embodiments, a primary active agent, secondary active ingredient, and/or pharmaceutically acceptable salt thereof where appropriate is in a particle-size reduced form. In further embodiments, a performance modifier, such as L-leucine or another amino acid, cellobiose octaacetate, and/or metals salts of stearic acid, such as magnesium or calcium stearate. In some embodiments, the aerosol formulations are arranged so that each metered dose of aerosol contains a predetermined amount of an active ingredient, such as the one or more of the compositions, compounds, vector(s), molecules, cells, and combinations thereof described herein.

Dosage forms adapted for vaginal administration can be presented as pessaries, tampons, creams, gels, pastes, foams, or spray formulations. Dosage forms adapted for rectal administration include suppositories or enemas. The vaginal formulations can be administered to a subject in need thereof.

Dosage forms adapted for parenteral administration and/or adapted for injection can include aqueous and/or non-aqueous sterile injection solutions, which can contain antioxidants, buffers, bacteriostats, solutes that render the composition isotonic with the blood of the subject, and aqueous and non-aqueous sterile suspensions, which can include suspending agents and thickening agents. The dosage forms adapted for parenteral administration can be presented in a single-unit dose or multi-unit dose containers, including but not limited to sealed ampoules or vials. The doses can be lyophilized and re-suspended in a sterile carrier to reconstitute the dose prior to administration. Extemporaneous injection solutions and suspensions can be prepared in some embodiments, from sterile powders, granules, and tablets. The parenteral formulations can be administered to a subject in need thereof.

For some embodiments, the dosage form contains a predetermined amount of a primary active agent, secondary active ingredient, and/or pharmaceutically acceptable salt thereof where appropriate per unit dose. In an embodiment, the predetermined amount of primary active agent, secondary active ingredient, and/or pharmaceutically acceptable salt thereof where appropriate can be an effective amount, a least effect amount, and/or a therapeutically effective amount. In other embodiments, the predetermined amount of a primary active agent, secondary active agent, and/or pharmaceutically acceptable salt thereof where appropriate, can be an appropriate fraction of the effective amount of the active ingredient.

### Co-Therapies and Combination Therapies

In some embodiments, the pharmaceutical formulation(s) described herein are part of a combination treatment or combination therapy. The combination treatment can include the pharmaceutical formulation described herein and an additional treatment modality. The additional treatment modality can be a chemotherapeutic, a biological therapeutic, surgery, radiation, diet modulation, environmental modulation, a physical activity modulation, and combinations thereof.

In some embodiments, the co-therapy or combination therapy can additionally include but not limited to, polynucleotides, amino acids, peptides, polypeptides, antibodies, aptamers, ribozymes, hormones, immunomodulators, antipyretics, anxiolytics, antipsychotics, analgesics, antispasmodics, anti-inflammatories, anti-histamines, anti-infectives, chemotherapeutics, and combinations thereof.

### Administration of the Pharmaceutical Formulations

The pharmaceutical formulations or dosage forms thereof described herein can be administered one or more times hourly, daily, monthly, or yearly (e.g., 1, 2, 3, 4, 5, 6, 7, 8, 9, 10, 11, 12, 13, 14, 15, 16, 17, 18, 19, 20, or more times hourly, daily, monthly, or yearly). In some embodiments, the pharmaceutical formulations or dosage forms thereof described herein can be administered continuously over a period of time ranging from minutes to hours to days. Devices and dosages forms are known in the art and described herein that are effective to provide continuous administration of the pharmaceutical formulations described herein. In some embodiments, the first one or a few initial amount(s) administered can be a higher dose than subsequent doses. This is typically referred to in the art as a loading dose or doses and a maintenance dose, respectively. In some embodiments, the pharmaceutical formulations can be administered such that the doses over time are tapered (increased or decreased) overtime so as to wean a subject gradually off of a pharmaceutical formulation or gradually introduce a subject to the pharmaceutical formulation.

As previously discussed, the pharmaceutical formulation can contain a predetermined amount of a primary active agent, secondary active agent, and/or pharmaceutically acceptable salt thereof where appropriate. In some of these embodiments, the predetermined amount can be an appropriate fraction of the effective amount of the active ingredient. Such unit doses may therefore be administered once or more than once a day, month, or year (e.g., 1, 2, 3, 4, 5, 6, or more times per day, month, or year). Such pharmaceutical formulations may be prepared by any of the methods well known in the art.

Where co-therapies or multiple pharmaceutical formulations are to be delivered to a subject, the different therapies or formulations can be administered sequentially or simultaneously. Sequential administration is administration where an appreciable amount of time occurs between administrations, such as more than about 15, 20, 30, 45, 60 minutes or more. The time between administrations in sequential administration can be on the order of hours, days, months, or even years, depending on the active agent present in each administration. Simultaneous administration refers to administration of two or more formulations at the same time or substantially at the same time (e.g., within seconds or just a few minutes apart), where the intent is that the formulations be administered together at the same time.

### Devices

In some embodiments, one or more of the engineered intein systems or components thereof are contained in a device. In some embodiments, the engineered intein system or component within the device is configured to capture a protein of interest, tag a protein of interest, sense a protein of interest, perform a bioconjugation of a protein of interest that may be present in a sample that is passed through the device. In some embodiments, the device is configured as a biosensor. In some embodiments, the devices are configured as a BioMEMs. In some embodiments, the device is a microfluidic device. In some embodiments, the device is a flow, such as a lateral flow device.

In some embodiments, the engineered intein system or component(s) thereof are contained in individual discrete volumes within the device. In some embodiments, the engineered intein system or component(s) thereof are attached to surface, such as surface on a support, within the device. In some embodiments, the engineered intein system or component(s) thereof of the present invention are contained at discrete location within the device. In some embodiments, the engineered intein system or component(s) thereof of the present invention are contained in discrete locations within an array in the device.

### Kits

Any of the compounds, compositions, systems, formulations (e.g., pharmaceutical formulations), particles, cells, devices, or any combination thereof described herein, or a combination thereof, can be presented as a combination kit. As used herein, the terms “combination kit” or “kit of parts” refers to the compounds, compositions, systems, formulations (e.g., pharmaceutical formulations), particles, cells, devices and any additional components that are used to package, sell, market, deliver, and/or administer the combination of elements or a single element, such as the active ingredient, contained therein. Such additional components include, but are not limited to, packaging, syringes, blister packages, bottles, and the like. When one or more of the compounds, compositions, systems, formulations (e.g., pharmaceutical formulations), particles, cells, and devices described herein or a combination thereof (e.g., agents) contained in the kit are administered simultaneously, the combination kit can contain the active agents in a single formulation, such as a pharmaceutical formulation, (e.g., a tablet, suspension, liquid, or other dosage form) or in separate formulations. When the compounds, compositions, systems, formulations (e.g., pharmaceutical formulations), particles, cells, and devices described herein or a combination thereof and/or kit components are not administered simultaneously, the combination kit can contain each agent or other component in separate pharmaceutical formulations. The separate kit components can be contained in a single package or in separate packages within the kit.

In some embodiments, the combination kit also includes instructions printed on or otherwise contained in a tangible medium of expression. The instructions can provide information regarding the content of the compounds, compositions, systems, formulations (e.g., pharmaceutical formulations), particles, cells, and devices described herein or a combination thereof contained therein, safety information regarding the content of the compounds, compositions, systems, formulations (e.g., pharmaceutical formulations), particles, cells, and devices described herein or a combination thereof contained therein, information regarding the dosages, indications for use, and/or recommended treatment regimen(s) for the c compounds, compositions, systems, formulations (e.g., pharmaceutical formulations), particles, cells, and devices contained therein. In some embodiments, the instructions can provide directions for administering the compounds, compositions, formulations (e.g., pharmaceutical formulations), particles, and cells described herein or a combination thereof to a subject in need thereof.

### Methods

As previously discussed, the engineered intein systems of the present invention can be used to perform a bioconjugation reaction (e.g., a protein trans-splicing reaction). As one of ordinary skill in the art will appreciate, the applications of the engineered intein systems of the present invention are broad. Split intein systems have been used for a variety of applications ranging from screening and detection (such as within a device) to biosensors, labeling, synthetic protein synthesis, protein purification, gene replacement and the like, all of which are suitable applications for the engineered intein systems of the present invention. See e.g., Wood et al., J Biol Chem. 2014 May 23; 289(21):14512-9; Li, Y., Biotechnol Lett. 2015 November; 37(11):2121-37. doi: 10.1007/s10529-015-1905-2; Yao et al., Nat Commun. 2020 May 15; 11(1):2440; Romero-Casanas et al., Methods Mol Biol. 2020; 2133:15-29; Cheriyan and Perler. dv Drug Deliv Rev. 2009 Sep. 30; 61(11):899-907; Wood and Camaero. J Biol Chem. 2014 May 23; 289(21):14512-9; Schmelas and Grimm. Biotechnol J. 2018 September; 13(9):e1700432; Mootz, H. D., Chembiochem. 2009 Nov. 2; 10(16):2579-89; Sarmiento and Camarero. Curr Protein Pept Sci. 2019; 20(5):408-424; Volkmann and Iwai. Mol Biosyst. 2010 November; 6(11):2110-21. doi: 10.1039/c0mb00034e; Li et al., Int J Biol Macromol. 2021 Sep. 1; 186:40-46; Zettler et al., PLoS One. 2013 Sep. 2; 8(9):e72925; Wang et al., Proc Natl Acad Sci USA. 2018 Apr. 10; 115(15):3900-3905; Bryson et al., Nucleic Acids Res. 2022 Jan. 11; 50(1):549-560; Bachman and Mootz. J Pept Sci. 2017 July; 23(7-8):624-630. doi: 10.1002/psc.2996; Bachman et al., Methods Mol Biol. 2015; 1266:145-59. doi: 10.1007/978-1-4939-2272-7_10; Li et al., Int J Biol Macromol. 2018 Apr. 1; 109:921-931. doi: 10.1016/j.ijbiomac.2017.11.077; Truong et al., Nucleic Acids Res. 2015 Jul. 27; 43(13):6450-8. doi: 10.1093/nar/gkv601; Lim et al., Mol Ther. 2020 Apr. 8; 28(4):1177-1189. doi: 10.1016/j.ymthe.2020.01.005; Tornabene et al., Sci Transl Med. 2019 May 15; 11(492):eaav4523. doi: 10.1126/scitranslmed.aav4523; Liu et al., Nat Biotechnol. 2022 September; 40(9):1388-1393. doi: 10.1038/s41587-022-01255-9; Liu et al., Nat Commun. 2021 Apr. 9; 12(1):2121; Yuan et al., ACS Synth Biol. 2022 Jul. 15; 11(7):2513-2517; Zhu et al., Sci China Life Sci. 2010 June; 53(6):683-9; Kang et al., Biosensors (Basel). 2022 Apr. 28; 12(5):283. doi: 10.3390/bios12050283; Renteln, M. Gene Ther. 2018 January; 25(1):1-3. doi: 10.1038/gt.2017.99; Jeon et al., Anal Chem. 2018 Aug. 21; 90(16):9779-9786; Ryu et al., Int J Mol Sci. 2021 Apr. 29; 22(9):4747; Selgrade et al., J Am Chem Soc. 2013 May 22; 135(20):7713-9; Guerreiro et al., Sensors (Basel). 2020 Dec. 23; 21(1):24. doi: 10.3390/s21010024; Shah and Muir et al., Isr J Chem. 2011 Nov. 1; 51(8-9): 854-861; Ciragan et al., Front Chem. 2020 Mar. 19; 8:136. doi: 10.3389/fchem.2020.00136. eCollection 2020; Volkmann, G. PLoS One. 2009 Dec. 21; 4(12):e8381. doi: 10.1371/journal.pone.0008381; Schutz and Mootz. Angew Chem Int Ed Engl. 2014 Apr. 14; 53(16):4113-7. doi: 10.1002/anie.201309396; Lu et al., J Chromatogr A. 2011 May 6; 1218(18):2553-60; Lee et al., Protein Sci. 2018 September; 27(9):1568-1574; Sakar et al., Methods Enzymol. 2021; 654:19-48; Yang and Yang. J Am Chem Soc. 2009 Aug. 26; 131(33):11644-5; Matern et al., Methods Mol Biol. 2015; 1266:129-43; Prescott and David. Methods Mol Biol. 2020; 2133:201-219; Charalambous et al., J Nanobiotechnology. 2011 Sep. 15; 9:37; Borra and Camarero. Methods Mol Biol. 2017; 1495:111-130; Muona et al., Nat Protoc. 2010 March; 5(3):574-87; which are incorporated by reference herein and can be adapted for use with the engineered intein system of the present invention.

Described in certain example embodiments herein are method of bioconjugation comprising: mixing a recombinant first amino acid sequence comprising an N-terminal intein sequence with a recombinant second amino acid sequence comprising a C-terminal intein sequence under conditions sufficient to allow bioconjugation of the first recombinant amino acid sequence and the second recombinant amino acid sequence, wherein the N-terminal intein sequence, the C-terminal intein sequence, or both are derived from a split intein of Richelia sp., Pseudomonas aeruginosa 18, Candidatus Brocadiales, or any combination thereof.

In certain example embodiments, the split intein is a cysteine-less split intein.

In certain example embodiments, the N-terminal intein sequence comprises an amino acid sequence having at least 80%, 85%, 90%, 95%, 98% sequence identity to any one of SEQ ID NO: 1, 3, 5, or 7.

In certain example embodiments, the C-terminal intein sequence comprises an amino acid sequence having at least 80%, 85%, 90%, 95%, 98% sequence identity to any one of SEQ ID NO: 2, 4, 6, or 8.

In certain example embodiments, the N-terminal intein sequence is attached to a C-terminus of the first amino acid sequence with a peptide bond.

In certain example embodiments, the C-terminal intein sequence is attached to a N-terminus of the first amino acid sequence with a peptide bond.

In certain example embodiments, a linker is operatively coupled between the first amino acid sequence and the N-terminal intein sequence, optionally wherein the linker is a peptide linker.

In certain example embodiments, a linker is operatively coupled between the first amino acid sequence and the C-terminal intein sequence, optionally wherein the linker is a peptide linker.

In certain example embodiments, the linker is not more than 1, 2, 3, 4, 5, 6, 7, 8, 9, 10, 11, 12, 13, 14, 15, or 20 amino acids in length.

In certain example embodiments, the linker is a Gly-Ser linker, optionally wherein the linker comprises an amino acid sequence of at least 80%, 85%, 90%, 95%, 98% sequence identity to GSGSGSGSGSGSGSGSGSGSG (SEQ ID NO: 11).

In certain example embodiments, the linker is an Asparagine-Serine linker, optionally wherein the linker comprises an amino acid sequence of at least 80%, 85%, 90%, 95%, 98% sequence identity to ASASASASASASASASAS (SEQ ID NO: 12).

In certain example embodiments, a localization tag, affinity tag, reporter tag, or any combination thereof, is operatively coupled to the first amino acid sequence, the second amino acid sequence, or both.

In certain example embodiments, the C-terminal intein sequence comprises X₁PYFFX₂NNILVHNS (SEQ ID NO: 10), wherein X₁ and X₂ are each independently selected from any amino acid.

In certain example embodiments, (a) wherein X₁ is selected from N or T, (b) wherein X₂ is selected from A or G, or (c) both (a) and (b).

In certain example embodiments, the C-terminal sequence comprises SEQ ID NO: 9.

In certain example embodiments, the conditions sufficient to allow bioconjugation comprise a pH ranging from about 6 to about 8, comprise a temperature ranging from about 20° C. to about 50° C., comprise a reducing agent, optionally wherein the reducing agent is dithiothreitol (DTT), beta mercaptoethanol (BME), tris(2-carboxyethyl)phosphine (TCEP), or cysteine, comprise NaCl at a concentration ranging from about 0.05 M NaCl to about 2 M NaCl, or any combination thereof.

In certain example embodiments, the bioconjugation reaction occurs to completion in about 1 minute to about 90 minutes. In certain example embodiments, the bioconjugation reaction occurs to completion in about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50, 50.5, 51, 51.5, 52, 52.5, 53, 53.5, 54, 54.5, 55, 55.5, 56, 56.5, 57, 57.5, 58, 58.5, 59, 59.5, 60, 60.5, 61, 61.5, 62, 62.5, 63, 63.5, 64, 64.5, 65, 65.5, 66, 66.5, 67, 67.5, 68, 68.5, 69, 69.5, 70, 70.5, 71, 71.5, 72, 72.5, 73, 73.5, 74, 74.5, 75, 75.5, 76, 76.5, 77, 77.5, 78, 78.5, 79, 79.5, 80, 80.5, 81, 81.5, 82, 82.5, 83, 83.5, 84, 84.5, 85, 85.5, 86, 86.5, 87, 87.5, 88, 88.5, 89, 89.5, to/or about 90 minutes.

Further embodiments are illustrated in the following Examples which are given for illustrative purposes only and are not intended to limit the scope of the invention.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure. The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

### Example 1

### Introduction

Inteins are protein splicing sequences that are post-translationally excised out in an auto-catalytic manner to produce mature host proteins whose genetic information is split into two parts at the DNA level^{1, 2, 3, 4, 5}. In many cases, the resulting mature host proteins called mature exteins are the enzymes involved in DNA processing, such as DNA polymerases, helicases, and endonucleases³. Inteins are considered to be of very ancient origin and often have been described as selfish genetic materials due to the lack of apparent cellular functions^{6, 7}. Consistently, although inteins are found in all three domains of life, higher organisms, including humans and other animals, do not encode the intein systems in their genomes^{8, 9, 10, 11}. While inteins can be found in many different forms, split-inteins are encoded in two separate genetic locations translating to two respective polypeptides^{4, 12, 13, 14, 15}. The N-terminal and C-terminal split extein-intein halves recognize each other and catalyze protein trans-splicing reactions leading to mature proteins consisting of the N-terminal and C-terminal extein halves without intein polypeptides⁴.

Nearly all native split-intein polypeptides contain multiple cysteine residues. This feature limits the flexibility of extein choices in taking advantage of these otherwise highly applicable bioconjugation systems. This limitation is associated with the vital role of cysteine residues in protein structure and function. The addition of reducing agents is often required for the cysteine-containing split-intein-mediated trans-splicing reactions, which can, unfortunately, render mature proteins non-functional. Consistently, there have been searches for the cysteine-less (CL) split-intein systems primarily by two approaches: (1) point-mutations of cysteine residues found in the native cysteine-containing split-inteins and (2) hunts for new CL split-inteins. As a result, four CL split-inteins—two native and two engineered—have been studied thus far. The native Neq Pol split-intein found in the thermophile Nanoarcheum equitans does not contain cysteine, but the required reaction conditions limit the application of this system^{16, 17}. This thermophilic split-intein does not function in a mesophilic reaction condition because it requires a high temperature (50 to 70° C.) for its trans-splicing activity to happen¹⁶. Another native CL split-intein system is encoded in a jumbo phage infecting Pseudomonas aeruginosa, but this system has not been characterized thus far¹⁸.

An engineered Psp-GDB Pol split-intein was generated by removing the lone internal cysteine found in its native form. However, this engineered system still requires denaturation and refolding for their trans-splicing functionality despite the mutation¹⁹. An engineered Aes PolB1 CL split-intein derivative of its native form found in a bacteriophage infecting Aeromonas sp. appears to be the best CL system among the four. This derivative was generated to improve the seemingly disadvantageous feature that the native form possesses. The native form was unable to drive the trans-splicing reaction to completion, leading to a low reaction yield²⁰. The engineered derivative that contains a new artificial split-site resulting in a shorter N-terminal intein half and a longer C-terminal intein half catalyzes the protein trans-splicing reaction in 0° C. to 37° C. and with reducing agents²⁰. However, it is worth noting that difference in pI values of the engineered Aes PolB1 CL split-intein pair is smaller than other naturally-evolved split-intein pairs (e.g., pI differences of ˜2.5 vs. 4-6 between the spilt-intein pair, respectively; Table C and Discussion).

In this Example, through a series of in silico, molecular, and biochemical approaches, Applicant discovered and characterized the Rsp CL split-intein. These results indicate that the Rsp CL split-intein catalyzes the protein trans-splicing reaction to completion within minutes and is compatible with various pH, temperatures, reducing agents, salts, and denaturing agents, covering many relevant conditions used for protein engineering and therapeutic applications.

### Results

Cyanobacteria Richelia sp. RM2_1_2 Encodes an Unusual CL Split-Intein Pair, the Rsp CL System

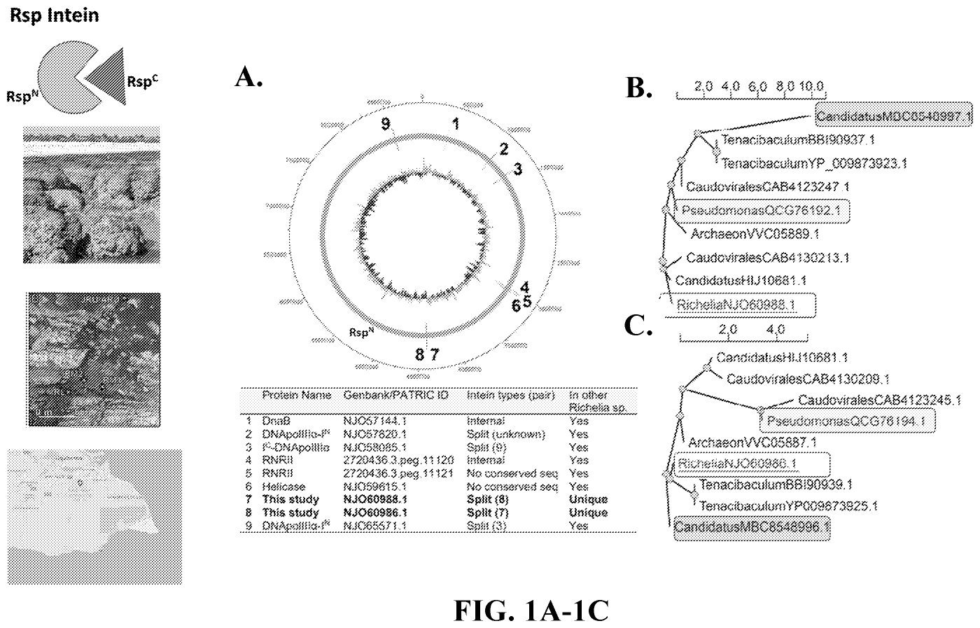

To identify new CL split-intein systems, Applicant conducted in silico analysis with an emphasis on samples that likely preserved the ancient microbial community information. Resultantly, Applicant found a new CL split-intein system encoded by the recently sequenced genome of Richelia sp. RM2_1_2 isolated from peritidal tufa stromatolites in Cape Recife, South Africa²¹. Through the bacterial bioinformatics tool PATRIC²², Applicant found that Richelia sp. RM2_1_2 encodes nine intein systems; Among them, two pairs are split-inteins, while only one system consisting of NJO60988.1 and NJO60986.1 is cysteine-less (FIG. 1A). NJO60988.1 and NJO60986.1 are unique, as this system is not conserved in other Richelia sp. strains (FIG. 1A). Consequently, these two gene products have been annotated as hypothetical protein(s) (FIG. 1A). HHpred analyses²³ that use the pairwise comparison of profile hidden Markov models suggest that the extein parts of NJO60988.1 and NJO60986.1 produce DNA polymerase subunits with over 99% probability (Tables 1 and 2). This prediction is not surprising, which is in line with the features associated with other mature extein products involved in DNA processing.

Applicant then analyzed the N-terminal and C-terminal split-intein sequences, referred to as ‘Rsp CL I^{N}’ and ‘Rsp CL I^{C}’ respectively, using protein BLAST. The Rsp CL I^{C} sequence resulted in only eight hits, which was further connected with the protein BLAST search result of Rsp CL I^{N} to examine their phylogenetic relationships to Rsp CL spilt-intein (FIG. 1B-1C). Rsp CL split-intein halves are distantly located in the phylogenetic tree, which is in line with the uniqueness of this split-intein system (FIG. 1B-1C). Moreover, among the eight systems homologous to the Rsp CL split-intein, only two other systems, Candidatus MBC and Pseudomonas QCG, are free from cysteine residues, which is consistent with the rarity of the CL split-intein systems.

the Rsp CL Intein System is Equipped with Extein-Tolerance and Catalyzes the Reaction to Completion

To investigate whether the Rsp CL split-intein system is equipped with extein-tolerance/flexibility and whether it catalyzes protein trans-splicing reaction to completion—two critical features towards wide applications of this biosystem—Applicant fused Rsp CL I^{N} and Rsp CL I^{C} to fluorescent proteins, resulting in mTurquoise2::I^{N} and I^{C}::mCherry2 (FIG. 2A). If the Rsp CL split-intein catalyzes protein trans-splicing reaction for the fused fluorescent proteins located in place of the original predicted DNA polymerase exteins, the reaction outcome would be mTurquoise2 linked to mCherry2 (FIG. 2A). The recombinant fusion proteins, mTurquoise2::I^{N} and I^{C}::mCherry2, were expressed as soluble proteins and purified through Ni-NTA and size exclusion chromatography (SEC). These protein preparations retained their folding and fluorescence, as indicated in SEC and SDS-PAGE of the samples with and without heat-treatments (FIG. 2B and FIGS. 6A-6B and 7A-7B). The folded proteins with fluorescence ran faster in SDS-PAGE gels and appeared as smaller-sized proteins. In contrast, the protein bands of the heated intein halves and PTS products migrated slower and corresponded to the right theoretical molecular weight ladders in SDS-PAGE gels (FIG. 2B and FIGS. 6A-6B and 7A-7B). These results indicate that the Rsp CL biosystem is equipped with extein-tolerance and flexibility, supporting the potential of its wide applications compatible with many other fusion partners.

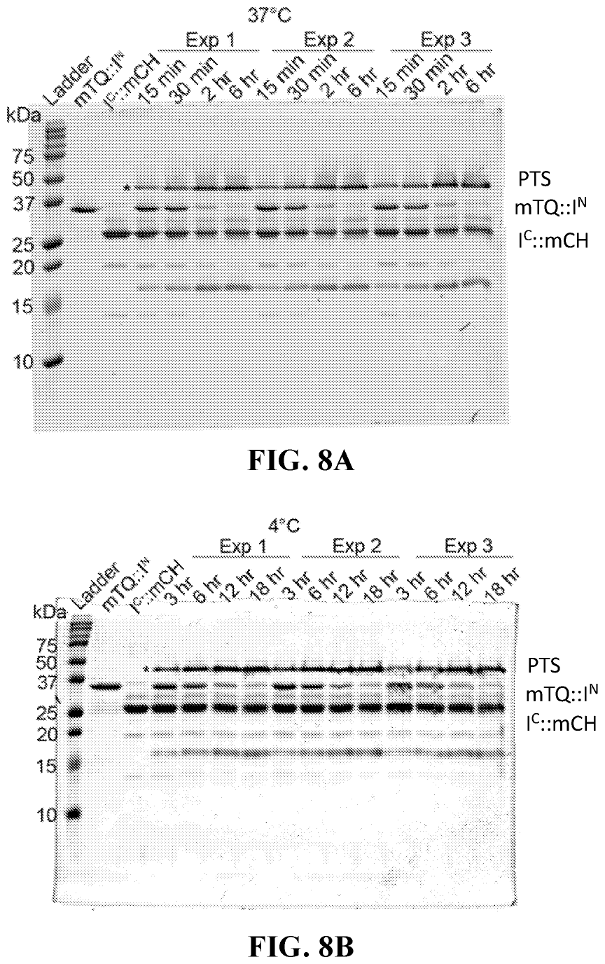

Applicant found that fluorescent signals reflecting protein trans-splicing reaction outcomes were faithfully matched with the intensities of Coomassie-stained protein bands (FIG. 2B and FIGS. 6A-6B and 7A-7B). This quantitative feature of the dual-reporter system fused to the Rsp CL split-intein halves enabled the ratiometric calculation of the apparent protein trans-splicing rate constant Kapp. The protein trans-splicing reaction was started immediately after mixing the two intein halves, which was completed within 6 hours at 37° C. in the pH 7.5 condition (FIG. 2C-2D and FIG. 8A-8B). The Kapp at 37° C. was measured to be 2.35±0.08×10⁻⁴ s⁻¹. When the temperature was lowered to 4° C., Kapp was reduced by approximately 10 times to 2.35±0.14×10⁻⁵ s⁻¹, and the reaction took 18 hr to near-completion (FIG. 2C-2D and FIG. 8A-8B). These results indicate that the Rsp CL system catalyzes the reaction to completion within hours in this pH 7.5 condition.

The Rsp CL Intein System Completes its Protein Trans-Splicing Reaction within Minutes to Hours

The surface charges of the intein halves are known to play a potential role in each other's recognition and association^{24, 25}, which is closely associated with their pI values and pH conditions used in experiments for the PTS reaction. Therefore, Applicant next examined various pH conditions to determine the optional pH condition for the PTS reaction of the Rsp CL split-intein. Since theoretical pI values of Rsp CL I^{N} and Rsp CL I^{C} are 4.32 and 9.70, respectively (Table C), we tested a range of pH from 4 to 10 (FIG. 9A-9B). Unlike the observations that the completion of the PTS reaction at pH 7.5 and pH 8.0 took 6 hrs, the reaction went to completion at pH 6.0 and 7.0 within 1 hr (FIG. 2D and FIG. 9A-9B). This result indicates that the optimal pH for the PTS reaction catalyzed by the Rsp CL split-intein is between 6 and 7, which is approximately the halfway point of the pI difference between the N- and C-terminal split-intein halves (Table C).

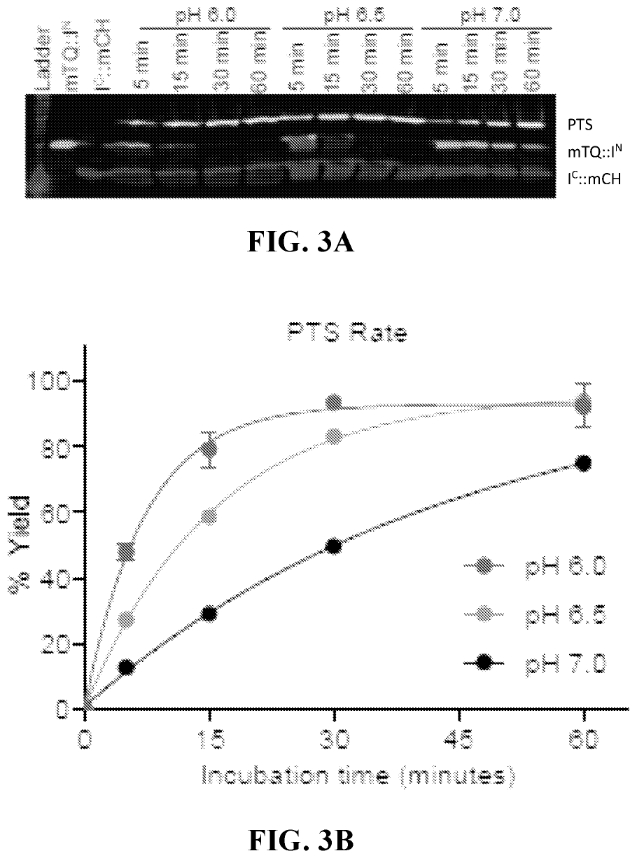

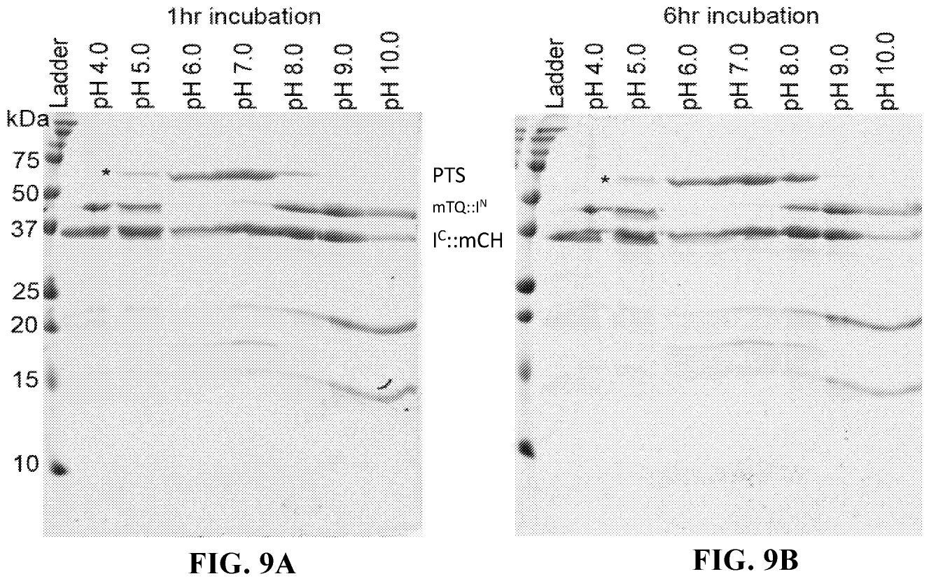

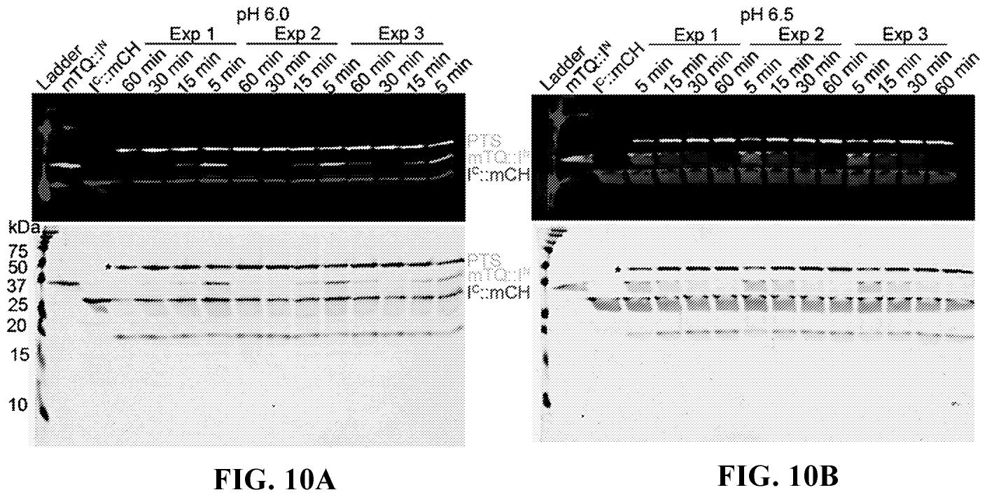

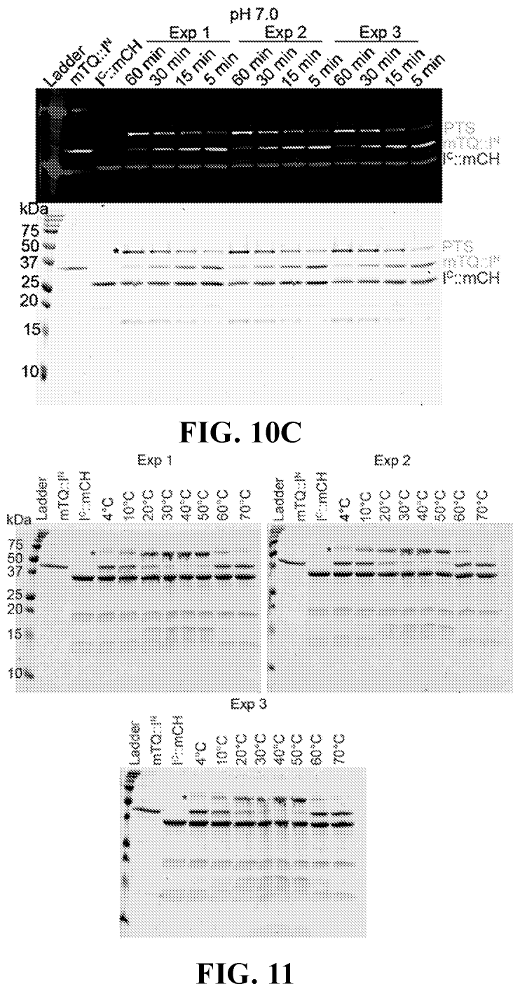

To determine the PTS reaction rates, Applicant carried out kinetic analyses of the PTS reaction catalyzed by the Rsp CL split-intein at pH 6, 6.5, and 7, revealing that the Rsp CL split-intein can catalyze the reaction to near-completion within 15 minutes at pH 6.0 (FIG. 3A-3B and FIG. 10A-10C). The Kapp at pH 6 is calculated to be 2.31±0.18×10⁻³ s⁻¹, Kapp at pH 6.5 is 1.08±0.02×10⁻³ s⁻¹, Kapp at pH 7 is 3.73±0.19×10⁻⁴ s⁻¹ (FIG. 3A-3B and FIG. 10A-10C). These values indicate that the protein trans-splicing rate of the Rsp CL split-intein at pH 6 is over ten times faster than the rate at pH 7.5, highlighting the speedy PTS reaction to completion catalyzed by the Rsp CL system.

### The Rsp CL Intein System Completes its Protein Trans-Splicing Reaction to Completion in a Wide Range of Temperatures, Reducing Agents, Salts, and Denaturing Agents

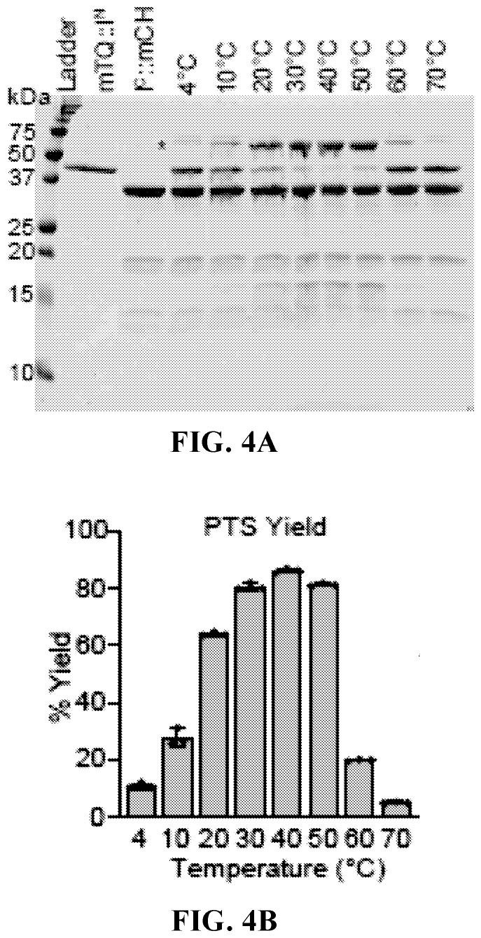

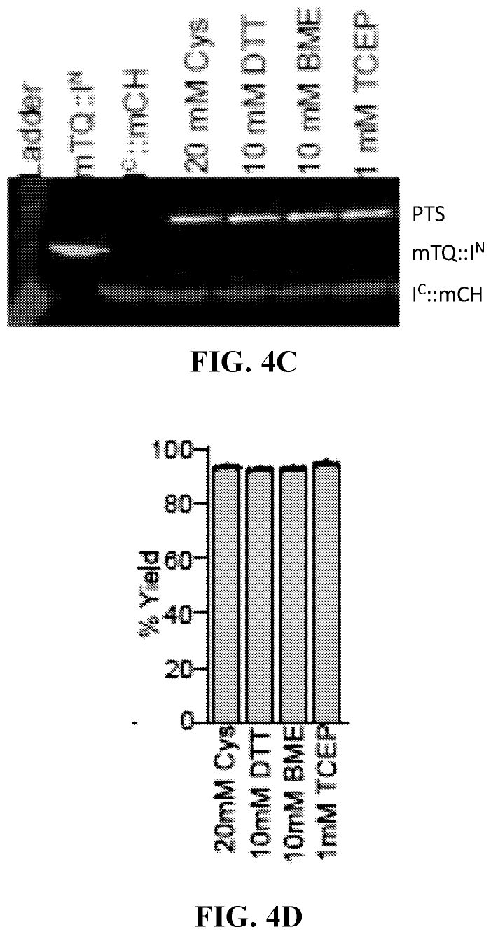

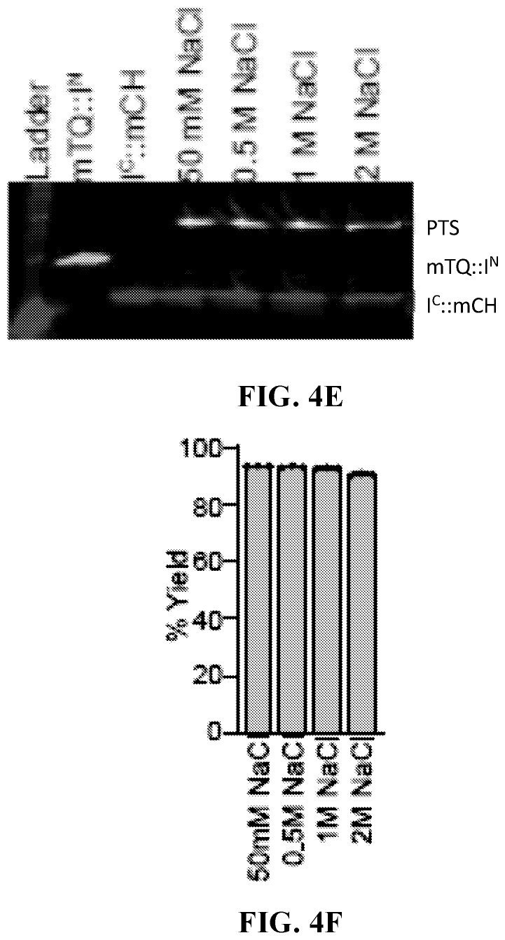

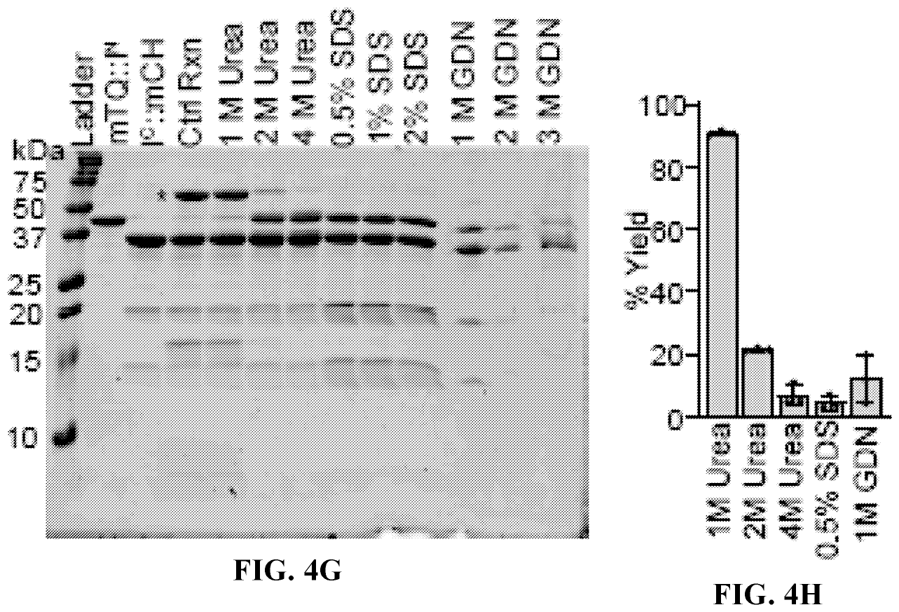

To learn more about the Rsp CL split-intein system, Applicant examined its trans-splicing activity in various conditions relevant to natural environments where this Richelia sp was isolated²¹ and in vitro and in vivo environments where this biosystem would be applied. Remarkably, the Rsp CL split-intein system catalyzed the PTS reaction in a wide temperature range from 4° C. to 70° C. An hour incubation of the split-intein mixtures resulted in over 60% completion of the PTS reaction at 20° C., and over 80% completion at 30, 40, and 50° C. (FIG. 4A-4B and FIG. 11). The Rsp CL split-intein exhibited a remarkable tolerance to common reducing agents, including cysteine, dithiothreitol (DTT), β-mercaptoethanol (BME), and tris-2-carboxyethyl-phosphine (TCEP), as the Rsp CL split-intein catalyzed the reactions to completion in the presence of these reducing agents (FIG. 4C-4D and FIG. 12A-12B). This feature supports its wide applications in laboratory and in vivo settings, including the oxidizing cellular organelle environment.

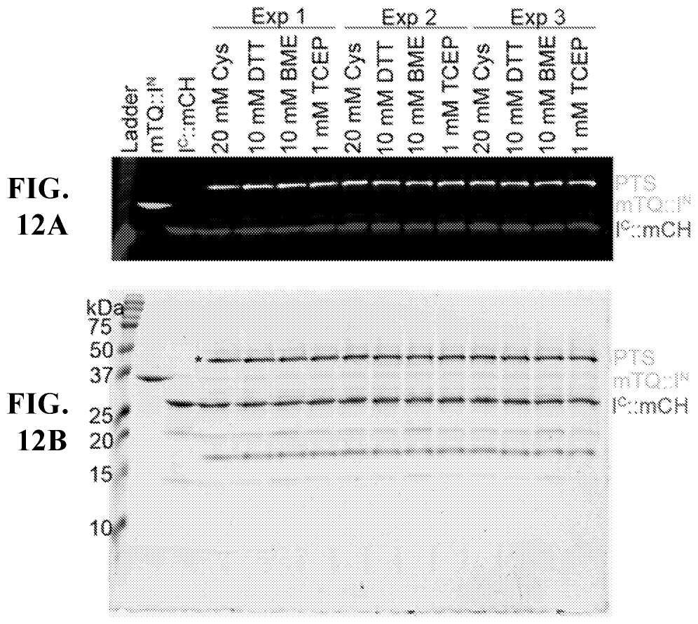

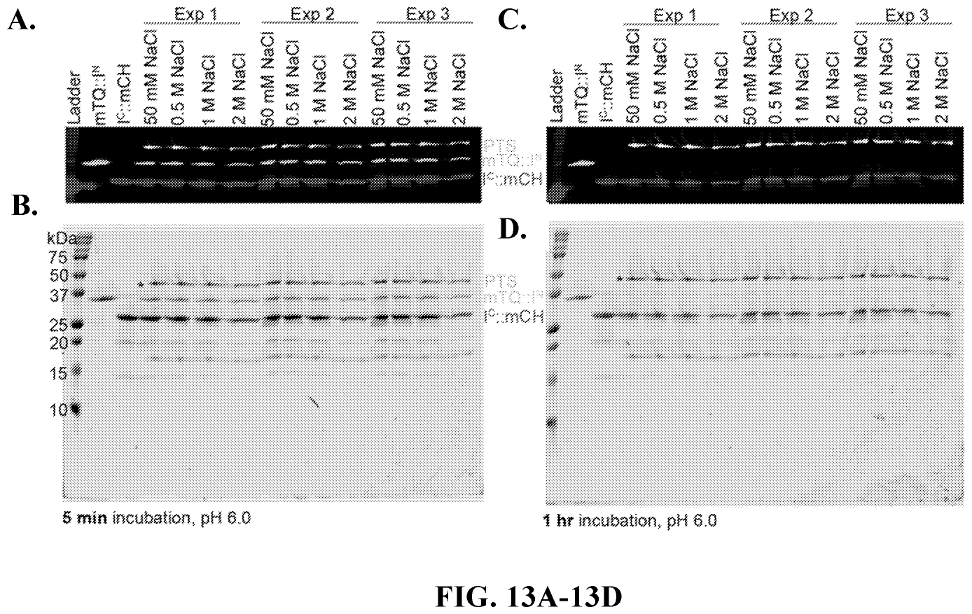

Similarly, the PTS reactions reached completion within an hour in the presence of various salt concentrations ranging from 0.05 to 2 M NaCl (FIG. 4E-4F and FIG. 13A-13D). Of note, the significant amounts of the PTS reaction products were apparent even only after incubation for 5 minutes (FIG. 13A-13B). This speedy reaction and compatibility with low and high salt concentrations of the Rsp CL system are remarkable, but these features align with the natural environment in Cape Recife, South Africa, where the tufa stromatolite containing this cyanobacterium was isolated²¹.

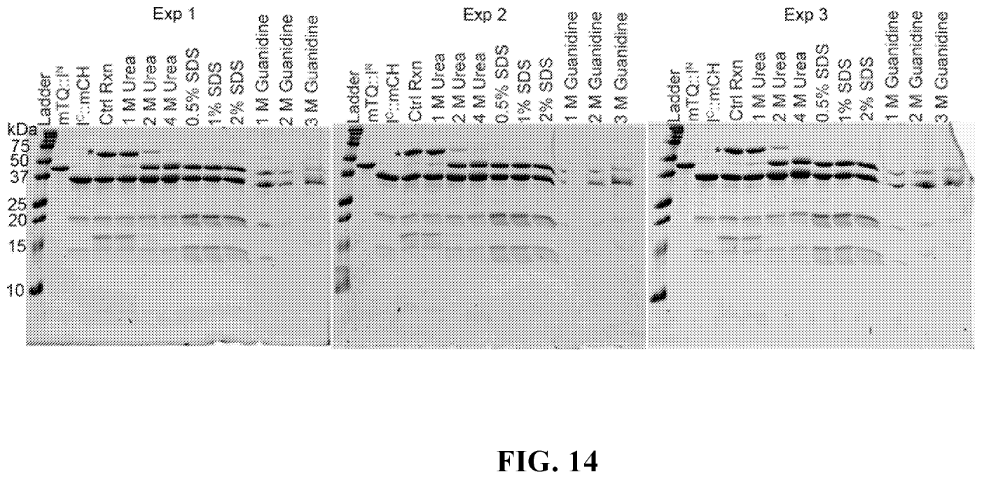

Since some extein partners for the Rsp CL split-intein might require the use of denaturing agents in protein preparation and/or reaction steps, Applicant also examined the tolerance of the Rsp CL split-intein to common denaturing agents frequently used in a laboratory setting, revealing the moderate level of tolerance to denaturing agents. The Rsp CL split-intein catalyzed the PTS reaction to near completion in the presence of 1 M Urea, while the reaction was partially completed in the presence of 2 M Urea (FIG. 4G-4H and FIG. 14). Small but detectable PTS reaction products were also observed in the reaction mixtures containing 4 M Urea, 0.5% sodium dodecyl sulfate (SDS), or 1 M guanidine (GDN) (FIG. 4G-4H and FIG. 14).

### The Rsp CL Intein System Completes its Protein Trans-Splicing Reaction in a Precise Manner

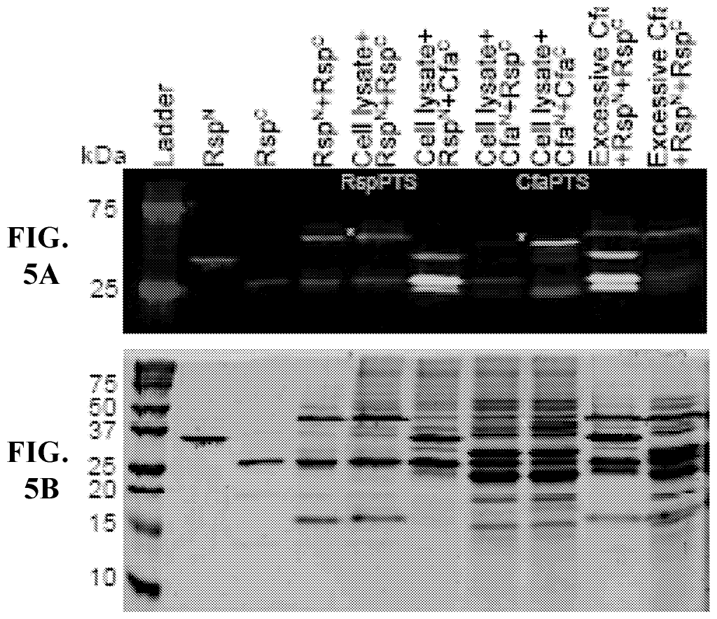

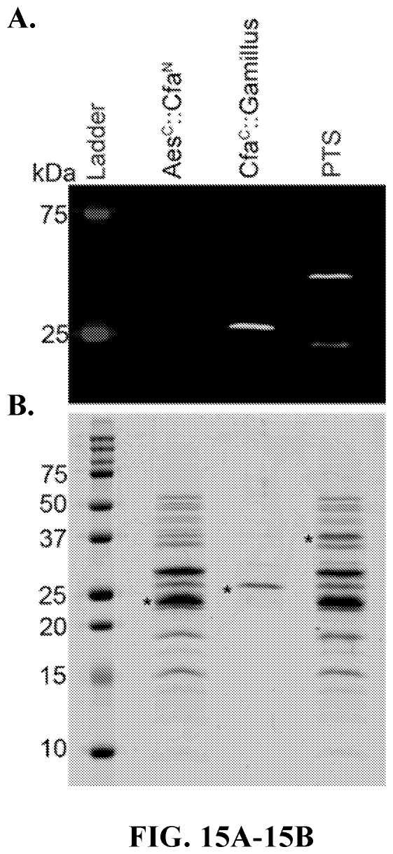

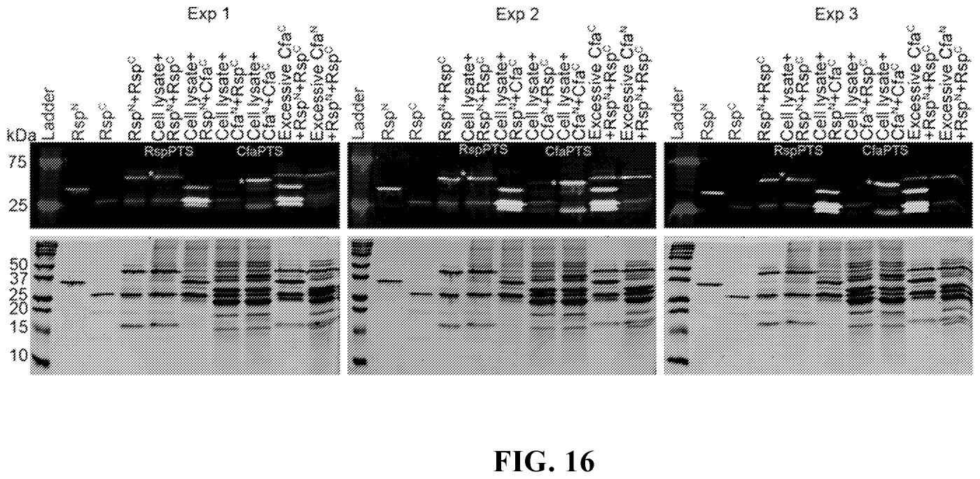

Lastly, to learn more about the specificity of Rsp CL split-intein-mediated PTS reactions, Applicant set up trans-splicing reactions in the presence of an excessive amount of host cell proteins with or without another non-pair split-intein half Host cell cytoplasmic proteins were prepared by mechanically disrupting human intestinal epithelial Henle-407 cells and added to the PTS reaction mixtures when indicated. Furthermore, the N-terminal and C-terminal intein halves of the Cfa split-intein and the C-terminal intein half of the Aes split-intein were expressed and prepared via partial purification for this experiment (FIG. 15A-15B). Although these protein preparations contained contaminant proteins originated from E. co/i cells, the fusion of Cfa^{C} to another fluorescent protein Gamillus (Cfa^{C}::Gamillus) enabled the tracing of the PTS reaction product catalyzed by the Cfa split-intein system (FIG. 15A-15B). This green Cfa-mediated PTS product also allowed us to distinguish it from the yellow Rsp-mediated PTS product (FIG. 5A and FIG. 16).

Using this experimental set-up, Applicant demonstrated that the Rsp CL split-intein catalyzed protein trans-splicing reactions in a precise manner regardless of the presence of host cell proteins, bacterial cell proteins, and/or another non-pair split-intein half. Consistently, the yellow Rsp PTS products were formed exclusively when both N- and C-terminal intein halves of the Rsp CL system were present (FIG. 5A-5B and FIG. 16).

### Discussion

Applicant demonstrated that the Rsp CL split-intein catalyzes protein trans-splicing reactions to completion or near-completion in an exact manner even under certain extreme conditions known to be unfavorable for the PTS reaction to occur. The Rsp CL system is better than most CL split-intein systems characterized thus far, based on its speedier reaction to completion, leading to an efficient and high protein conjugation yield. This newly characterized bioconjugation system is also equipped with impressive compatibilities with various conditions, distinguishing the Rsp CL intein from other counterparts. These results highlight the promise of this newly discovered bioconjugation system in various protein engineering and therapeutic applications.

The use of fluorescent extein proteins fused to the N- and C-terminal split-intein halves of the Rsp CL system supports the extein tolerance and flexibility of this bioconjugation system. In support of the extein tolerance/flexibility, the Rsp CL system has been successfully conjugated to different sets of proteins and peptides in the laboratory (unpublished). Furthermore, as proof of principle studies demonstrating the Rsp CL split-intein specificity in complex situations, Applicant mixed the fused Rsp CL intein halves with host proteins, bacterial proteins, and/or another non-pair split-intein half. Using this system, Applicant demonstrated the bioconjugation catalyzed by the Rsp CL system occurs in a highly specific manner.

The CL split-intein systems are rare to find, despite the broad application promise. Consistently, besides the Rsp CL split-intein, only a few such systems have been characterized. The Rsp CL split-intein system stands out in the group in that it can tolerate and catalyze the PTS reaction in a wide range of conditions, including several extreme environments. The optimal condition associated with the speedy “minutes-scale” PTS reaction by the Rsp CL system is a pH range of 6 to 8 and a temperature range of 20 to 50° C., which is generally not influenced by the presence of common reducing agents and a wide range of salt concentrations. Furthermore, Rsp CL split-intein can tolerate common denaturing agents and concentrations. These features will make the Rsp CL bioconjugation system compatible with extein partners whose preparations require lower or higher temperatures, reducing agents, high salt concentrations, and/or denaturing agents.

It is worth highlighting that the Rsp CL system is compatible with a broad pH range and tolerates to reducing agents in therapeutic application perspective, since it supports the promise of this bioconjugation system's functionality in cellular organelles maintaining lower pH and oxidizing states, such as vesicles, lysosomes, and endoplasmic reticulum, cellular organelles associated with various devastating diseases without effective therapeutic strategies, including various infectious diseases and neurological disorders^{26, 27, 28, 29, 30, 31, 32, 33, 34, 35}.

Using ProtParam, Applicant calculated theoretical pI values and protein stabilities of the CL split-inteins studied thus far. The pI difference between N- and C-terminal split-intein halves is suggested to play a critical role in each other's recognition and association. The pI values of split-intein halves seem to favor an acidic value for the N-term half and a basic value for the C-terminal half, and the pI value differences are 4˜6 between the split-intein pair (Table C). It is worth noting that the engineered Aes split-intein pair exhibits a smaller pI difference, which is ˜2.5 (Table C). Further careful studies need to be conducted, but this feature of the engineered Aes CL split-intein may be associated with a narrower application potential than the Rsp CL system. The Rsp CL split-intein system is also highlighted by the best protein stability prediction results among the CL split-inteins (Table C.

Like other split-inteins, the native extein proteins transcribed with the Rsp CL intein polypeptides are putative DNA polymerase subunits as predicted with over 99% probability through the pairwise comparison of profile hidden Markov models (Tables 1 and 2). This prediction is in line with the features associated with other mature extein products involved in DNA processing. Further experimentation is required to demonstrate whether the mature extein product catalyzed by the Rsp CL split-intein is DNA polymerase subunit(s). It is also intriguing to conduct future studies designed to address a relationship between split-intein protein stabilities and the possible transient requirement of mature extein products, as a significant number of CL split-intein halves are predicted to be unstable (Table 3).

Having the visual means to trace and measure the enzymatic reactions during the processes is powerful in several ways. For such reasons, Applicant established the dual fluorescent protein trans-splicing assay in characterizing the Rsp CL split-intein. First, this system allowed us to monitor the tight protein folding of investigated proteins, calculate the apparent protein trans-splicing rate constant Kapp, and distinguish and trace the PTS reaction product associated exclusively with the Rsp CL split-intein system. For instance, Applicant compared unboiled and boiled samples separated in SDS-PAGE gels, allowing for monitoring protein foldings and their related mobility behaviors in protein gels. Second, a series of time-course experiments let us conduct the kinetic analyses of this bioconjugation system. Third, the use of a different fluorescent protein for another spilt-intein half was made to trace the Rsp CL split-intein-mediated PTS reaction. Therefore, the methodology used in this study can serve as an example for other similar studies aimed at characterizing new split-intein systems.

In summary, the Rsp CL split-intein is equipped with many remarkable features critical for wide applications, highlighting the promise of this newly discovered bioconjugation system in various protein engineering and therapeutic applications.

### Materials and Methods

Richelia sp. RM2_1_2 Sequence Analysis

PATRIC (www.patricbrc.org)²² was used to analyze the genome of Richelia sp. RM_1_2, resulting in identifying the nine intein systems listed in the table shown in FIG. 1A. The N-terminal and C-terminal split-intein sequences of NJO60988.1 and NJO60986.1 were used as queries in Protein BLAST (National Institutes of Health). The C-terminal Rsp CL intein half resulted in only eight hits that are listed in FIG. 1C. The evolutionary relationships of the N-terminal and C-terminal Rsp CL intein halves with these eight hits were further analyzed via the FastME 2.0 phylogeny inference program with the protein alignment option (atgc-montpellier.fr/fastme)³⁶. These results are shown in FIG. 1B-1C.

### Computational Calculations of the Isoelectric Point (pI) and Stability of the CL Split-Intein Systems

The ProtParam program available through Expasy (web.expasy.org/protparam) was used to compute various physical and chemical parameters of indicated CL split-intein halves¹⁷. Table 3 shows some of the significant calculated values.

### Cloning Expression, and Purification of Proteins

Codon optimized version of intein-fused proteins was obtained as gBlocks through IDT DNA. The gene was amplified with Herculase DNA Polymerase II and cloned into pET28a plasmid using Gibson assembly. The final plasmid construct sequence was confirmed via Sanger sequencing by the Cornell Biotechnology Resource Center Genomic Core.

Acella E. coli strain (Edgebio), a derivative of BL21(DE3) with endA and recA mutant, was used for cloning and expression of the intein fusion protein. E. coli Acella strains containing the intein fusion proteins expression plasmids (Table D) were grown in LB broth to approximately OD₆₀₀=0.7 at 37° C. before switching to 28° C. to equilibrate for 15 min. Isopropyl β-D-1-thiogalactopyranoside (IPTG) was added to a final concentration at 0.5 mM to induce recombinant protein expression. The culture was incubated for 16 hr at 28° C. and 200 rpm. The bacteria were harvested with centrifugation at 5000×g for 15 min at 4° C. The bacteria pellets were resuspended in 15 mM Tris-HCl, pH 8.0 and 150 mM NaCl, 1×EDTA-free protease inhibitor cocktail, 0.2 mg/ml lysozyme, and 80 μg/ml DNAse I. The sample was then lysed by sonication, and the lysate was clarified with ultracentrifugation at 18,000×g for 30 min at 4° C. The clarified lysate was passed through 10 mL of Ni-NTA resin (Cytiva) equilibrated in the wash buffer containing 15 mM Tris-HCl, pH 8.0, and 150 mM NaCl. The machine was applied to the AKTA Pure FPLC machine (Cytiva). The column was washed with 100 mL of the wash buffer, and the bound protein was eluted with an increasing gradient of an elution buffer containing 15 mM Tris-HCl, pH 8.0, 150 mM NaCl, and 300 mM Imidazole. The fractions containing visually indicative fluorescent proteins were pooled and concentrated with a 10 KDa Amicon protein concentrator (EMD-Millipore). The concentrated protein was then injected onto a Superdex 75 Increase 10/300 column equilibrated with 1×PBS, pH 7.5. For experiments required to alter salt concentrations, denaturants, and reducing agents, the concentrated protein after the Ni-NTA chromatography was injected onto the same size exclusion chromatography column equilibrated with 100 mM Sodium Citrate, pH 6.0, and 150 mM NaCl. The fraction containing the desired recombinant fusion protein product was collected. The protein concentration was determined using the BCA assay (Thermo Fisher), aliquoted, flash-frozen in liquid nitrogen, and stored in a −80° C. freezer until use. For purification of fusion proteins containing the Cfa intein systems, 1 mM TCEP was included in all purification buffers to retain the Cfa intein activity.

### Protein Trans-Splicing Assay

Protein trans-splicing assays were conducted in PCR tubes with temperature control provided by the C1000 Thermal Cyclers (Bio-Rad Laboratories). The concentrated intein-containing fusion protein was adjusted to the desired protein concentration before adding to a reaction mixture. For pseudo-unimolecular trans-splicing reaction, the reaction mixture was made on ice to contains 50 μg/mL of mTurquoise2-Rsp^{N} and 150 to 200 μg/mL of Rsp^{C}-mCherry2 together with the desired pH and/or indicated reducing agents, salts, and denaturants. The reaction was then incubated at the desired temperature. The reactions were quenched by adding 6×SDS sample loading buffer (375 mM Tris-HCl, 9% SDS, 50% Glycerol, 0.03% bromophenol blue, and 9% v/v β-mercaptoethanol) before analysis using 15% SDS-PAGE gels. For the experiment testing the pH range of the intein system, the pH adjustment was achieved by 1:1 addition of 1 M crystallographic grade buffer solution by Hamilton Research. To the tolerant test against reducing agents, the TCEP-HCl solution was pH-confirmed before the reaction since TCEP-HCl generates extreme acidic pH if it is dissolved directly in water. For the high-temperature range- and denaturant-tolerance experiments, the quenched reaction product was heated to 95° C. for 5 min before SDS-PAGE analysis. It is worth noting that mCherry2 undergoes a small degree auto-proteolysis that generates non-fluorescing fragments with different molecular weights than that of the intein products (obtained from a singular peak in the size exclusion chromatography). The specificity and cross-reactivity of the Rsp CL Intein system were tested in the C-terminal half of the Aes CL intein system and both halves of the Cfa intein system with and without 0.5 mg/mL of human epithelial cell lysates.

### Gel Image Quantification

SDS-PAGE gels were imaged directly in the glass pane with the Krypton and Pro Emerald 488 preset-settings of a Bio-Rad ChemiDoc MP machine. The gel was then extracted from the glass pane and stained overnight with AquaStain (BullDog Bio). The stained gel was then recorded with the Odyssey CLx machine. Images obtained via the ChemiDoc were opened with ImageJ, adjusted for the brightness following the protein ladder, and merged with the Krypton filter channel pseudocolored red and the Pro Emerald 488 filter channel pseudocolored green. With this setting, the PTS product, where the two-channels were overlapped, appears as yellowish-orange.

### Fluorescent Image Quantification:

The Rsp CL split-intein reactivity was quantified based on the observations indicating that Rsp^{N} is a limiting reagent between the two intein halves. Raw tiff files exported from a Chemidoc MP machine (Bio-Rad) were analyzed in ImageJ as follows. A rectangular box was drawn using a cursor around the area where mTurquoise2::Rsp^{N} was located, and the signal intensity was measured using the Measurement tool in ImageJ to give value A. The same box was moved without altering its size onto the region where the expected splicing product would be within the same lane. The signal intensity was measured again to give value B. The same box was moved without altering its size onto the region below the 25 kDa molecular weight marker within the same lane where no fluorescent signal was expected. A background measurement was measured within the same lane to give value C. These three measurements per lane served as the basis of the percentage intein trans-splicing yield calculation.

The signal ratio was calculated using the following formula:

$$r = \frac{A - C}{B - C} .$$

The percentage yield was then calculated using the formula:

$$\% Yield = \frac{100 * ( 1 - r )}{1 + r} .$$

The % yield for each intein reaction was finalized by adding the % Yield value of the negative control lane, which contains only mTurquoise2::Rsp^{N}.

### Coomassie-Stained Gel Image Quantification:

Raw tiff files exported from the Odyssey CLx ImageS software were analyzed in ImageJ as follows. A rectangular box was drawn using a cursor around the area where mTurquoise2::Rsp^{N} was located, and the signal intensity was measured using the Measurement tool in ImageJ to give value A. The same box was moved without altering its size onto the region where the expected splicing product would be within the same lane. The signal intensity was measured again to give value B. Lastly, the same box was moved without altering its size onto the region above the 100 kDa molecular weight marker within the same lane where there is no visible protein band, and a background measurement was measured within the same lane to give value C. These three measurements per lane served as the basis of the percentage intein trans-splicing yield calculation.

The signal ratio was calculated using the following formula:

$$r = \frac{( A - C ) / 41961.9}{( B - C ) / 55970.91}$$

where the theoretical molecular weight of mTurquoise2::Rsp^{N} fusion protein is 41961.90 Da, and the theoretical molecular weight of mTurquoise2::mCherry2 trans-spliced product is 55970.91 Da.

The percentage yield was then calculated using the formula:

$$\% Yield = \frac{100 * ( 1 - r )}{1 + r} .$$

The % yield for each intein reaction was finalized by adding the % Yield value of the negative control lane, which contains only mTurquoise2::Rsp^{N}.

For kinetic calculation and statistical analysis, the calculated percentage yields as a function of time were input into the Graphpad Prism under the Exponential Decay Data Input. The program analyzed using the Nonlinear Fit of Exponential Decay, which yields the splicing rates K per minute and its standard deviation. The resulting graph was examined to ensure the correct fitting of curves along with data points. The output K value is then converted into splicing rates per second by dividing the value by 60.

### Example 2

### Results

Cyanobacteria Richelia sp. RM2 1 2 Encodes an Unusual CL Split-Intein Pair, the Rsp CL System

To identify new CL split-intein systems, Applicant conducted in silico analysis with an emphasis on samples that likely preserved the ancient microbial community information. Resultantly, Applicant found a new CL split-intein system encoded by the recently sequenced genome of Richelia sp. RM2_1_2 isolated from peritidal tufa stromatolites in Cape Recife, South Africa.21 Through the bacterial informatics tool PATRIC22, Applicant found that Richelia sp. RM2_1_2 encodes nine intein systems; Among them, two pairs are split-inteins, while only one system consisting of NJO60988.1 and NJO60986.1 is cysteine-less (FIG. 1A). NJO60988.1 and NJO60986.1 are unique, as this system is not conserved in other Richelia sp. strains (FIG. 1A). Consequently, these two gene products have been annotated as hypothetical protein(s). (FIG. 1A). HHpred analyses23 that use the pairwise comparison of profile hidden Markov models suggest that the extein parts of NJO60988.1 and NJO 60986.1 produce DNA polymerase subunits with over 99% probability (Tables 1 and 2). This prediction is not surprising which is in line with the features associated with other mature extein products involved in DNA processing.

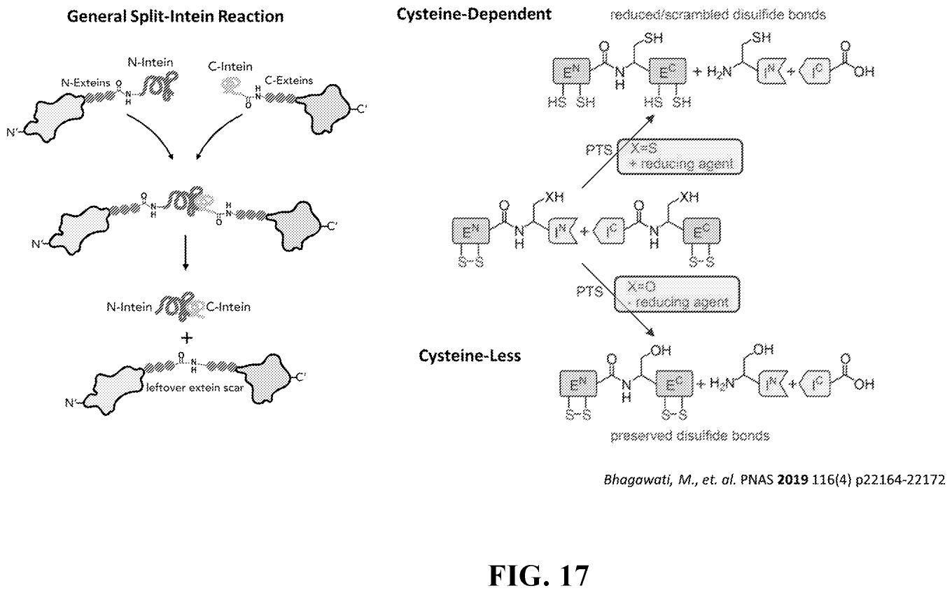

TABLE 1
HHpred analysis of NJO60988.1. Hits with over 99% probability are shown. Related to FIGS. 1A-1C and 17-18.

| | | | | | Query | | |
|---|---|---|---|---|---|---|---|
| Hits | Probability | E-value | P-value | Score | HMM | Template HMM | |
| 6P1H_A DNA polymerase delta catalytic | 99.6 | 8.5E−16 | 1.6E−20 | 145.3 | 35-168 | 679-823 | (1119) |
| subunit | | | | | | | |
| 2GV9_B DNA polymerase (E.C.2.7.7.7) | 99.6 | 3.1E−15 | 5.7E−20 | 142.0 | 37-132 | 728-853 | (1193) |
| 5EXR_C DNA primase small subunit | 99.6 | 3.5E−15 | 6.6E−20 | 141.5 | 34-155 | 570-694 | (1128) |
| (E.C.2.7.7.—) | | | | | | | |
| 6TNY_A DNA polymerase delta | 99.6 | 2.4E−15 | 4.5E−20 | 142.3 | 37-168 | 652-794 | (1107) |
| catalytic subunit | | | | | | | |
| 4QCL_A DNA polymerase alpha | 99.6 | 5.6E−15 | 1.1E−19 | 137.6 | 34-160 | 569-698 | (922) |
| catalytic subunit | | | | | | | |
| 6Q4T_A DNA polymerase | 99.6 | 3.2E−14 | 6.1E−19 | 130.2 | 32-210 | 440-607 | (774) |
| 3IAY_A DNA polymerase delta catalytic | 99.6 | 3.1E−14 | 5.8E−19 | 132.7 | 34-168 | 590-735 | (919) |
| subunit(2.7.7.7) | | | | | | | |
| 6V93_A DNA polymerase zeta catalytic | 99.5 | 3.8E−14 | 7.1E−19 | 137.5 | 34-168 | 1073-1215 | (1538) |
| subunit | | | | | | | |
| 1S5J_A DNA polymerase I (E.C.2.7.7.7) | 99.5 | 2.7E−13 | 5E−18 | 125.4 | 34-210 | 521-680 | (847) |
| 3K59_A DNA polymerase II | 99.5 | 1.8E−13 | 3.5E−18 | 125.5 | 37-152 | 465-572 | (786) |
| (E.C.2.7.7.7) | | | | | | | |
| 4FVM_A DNA polymerase alpha | 99.5 | 1.6E−13 | 3E−18 | 127.8 | 37-132 | 554-657 | (910) |
| catalytic subunit | | | | | | | |
| 5MDN_B DNA polymerase (E.C.2.7.7.7) | 99.5 | 1.6E−13 | 3E−18 | 125.8 | 32-132 | 460-569 | (783) |
| 3QEX_A DNA polymerase (E.C.2.7.7.7) | 99.5 | 3.4E−13 | 6.3E−18 | 125.6 | 33-132 | 463-630 | (903) |
| 4M8O_A DNA polymerase epsilon | 99.5 | 3.5E−13 | 6.6E−18 | 128.2 | 17-132 | 780-884 | (1228) |
| catalytic subunit | | | | | | | |
| 5N2E_A DNA polymerase | 99.5 | 3.6E−13 | 6.7E−18 | 126.7 | 33-168 | 619-795 | (1010) |
| (E.C.2.7.7.7,3.1.11.—) | | | | | | | |
| 6WJV_A DNA polymerase epsilon | 99.4 | 4.2E−12 | 7.8E−17 | 126.6 | 17-132 | 780-884 | (2222) |
| catalytic subunit | | | | | | | |

TABLE 2
HHpred analysis of NJO60986.1. Hits with over 99% probability are shown. Related to FIGS. 1A-1C and 17-18.

| | | | | | Query | | |
|---|---|---|---|---|---|---|---|
| Hits | Probability | E-value | P-value | Score | HMM | Template HMM | |
| 3K59_A DNA polymerase II | 100.0 | 7.5E−27 | 1.4E−31 | 243.7 | 1-329 | 517-780 | (786) |
| (E.C.2.7.7.7) | | | | | | | |
| 5EXR_C DNA primase small subunit | 100.0 | 2.9E−27 | 5.4E−32 | 255.5 | 1-326 | 637-900 | (1128) |
| (E.C.2.7.7.—) | | | | | | | |
| 6Q4T_A DNA polymerase | 100.0 | 2.9E−27 | 5.4E−32 | 246.3 | 1-343 | 508-772 | (774) |
| 4QCL_A DNA polymerase alpha | 100.0 | 1.8E−27 | 3.4E−32 | 252.3 | 1-335 | 636-908 | (922) |
| catalytic subunit | | | | | | | |
| 3QEX_A DNA polymerase (E.C.2.7.7.7) | 99.9 | 7E−26 | 1.3E−30 | 239.8 | 1-339 | 581-903 | (903) |
| 6TNY_A DNA polymerase delta | 99.9 | 1.1E−26 | 2E−31 | 250.5 | 1-323 | 715-976 | (1107) |
| catalytic subunit | | | | | | | |
| 6P1H_A DNA polymerase delta catalytic | 99.9 | 4.7E−27 | 8.8E−32 | 253.2 | 1-323 | 744-998 | (1119) |
| subunit | | | | | | | |
| 3IAY_A DNA polymerase delta catalytic | 99.9 | 2.8E−26 | 5.2E−31 | 243.3 | 1-325 | 656-912 | (919) |
| subunit(2.7.7.7) | | | | | | | |
| 4FVM_A DNA polymerase alpha | 99.9 | 7.7E−26 | 1.4E−30 | 239.7 | 2-327 | 618-889 | (910) |
| catalytic subunit | | | | | | | |
| 6V93_A DNA polymerase zeta catalytic | 99.9 | 4.5E−26 | 8.4E−31 | 251.6 | 1-327 | 1142-1403 | (1538) |
| subunit | | | | | | | |
| 5MDN_B DNA polymerase (E.C.2.7.7.7) | 99.9 | 9.9E−26 | 1.9E−30 | 235.0 | 1-341 | 529-783 | (783) |
| 2GV9_B DNA polymerase (E.C.2.7.7.7) | 99.9 | 1.1E−24 | 2E−29 | 235.8 | 1-343 | 790-1153 | (1193) |
| 1S5J_A DNA polymerase I (E.C.2.7.7.7) | 99.9 | 1.2E−24 | 2.3E−29 | 228.8 | 1-342 | 589-844 | (847) |
| 5N2E_A DNA polymerase | 99.9 | 3.2E−23 | 6.1E−28 | 221.9 | 1-337 | 686-1007 | (1010) |
| (E.C.2.7.7.7,3.1.11.—) | | | | | | | |
| 4M8O_A DNA polymerase epsilon | 99.9 | 6.3E−22 | 1.2E−26 | 214.4 | 1-344 | 845-1204 | (1228) |
| catalytic subunit | | | | | | | |
| 6WJV_A DNA polymerase epsilon | 99.8 | 2.7E−19 | 5.1E−24 | 202.8 | 1-344 | 845-1204 | (2222) |
| catalytic subunit | | | | | | | |

Applicant then analyzed the N-terminal and C-terminal split-intein sequences, referred to as ‘RspN’ and ‘RspC’ respectively, using protein BLAST. The RspC sequence resulted in only eight hits, which was further connected with the protein BLAST search result of RspN to examine their phylogenetic relationships to Rsp CL split-intein (FIG. 1B-1C). Rsp CL split-intein halves are distantly located in the phylogenetic tree, which is in line with the uniqueness of this split-intein system (FIG. 1B-1C). Moreover, among the eight systems homologous to the Rsp CL split-intein, only two other systems, Candidatus MBC (refer to as Cand CL intein) and Pseudomonas QCG (refer to as Pae CL intein), are free from cysteine residues, which is consistent with the rarity of the CL split-intein systems. (FIG. 1A).

The Rsp CL Intein System is Equipped with Extein-Tolerance and Catalyzes the Reaction to Completion

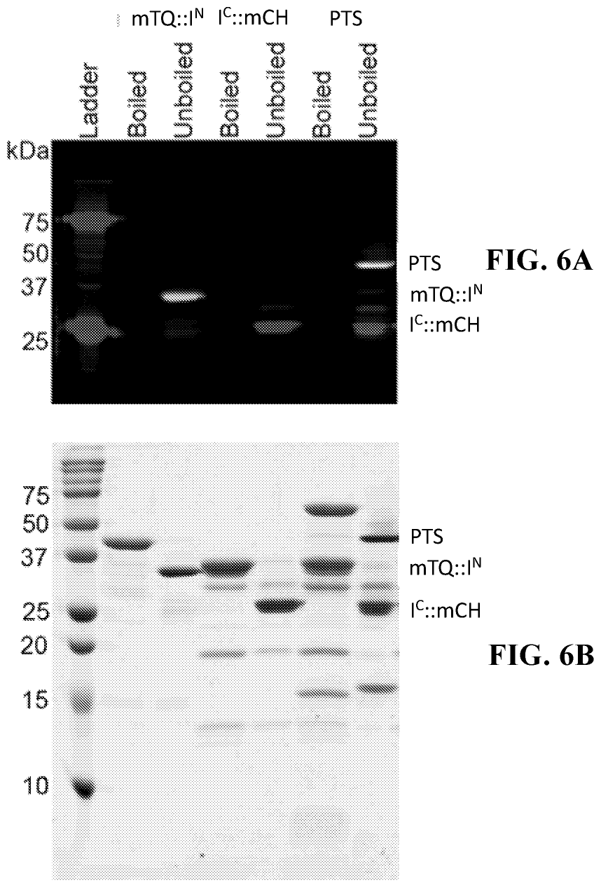

To investigate whether these CL split-intein systems in equipped with extein-tolerance/flexibility and whether it catalyzes protein trans-splicing reaction to completion—two critical features towards wide applications of this biosystem—Applicant fused each CL Intein N- and C-terminal halves to fluorescent proteins, resulting in mTurquoise2::IN and IC::mCherry2 (FIG. 2A). If the split-intein catalyzes protein trans-splicing reaction for the fused fluorescent protein located in place of the original predicted DNA polymerase exteins, the reaction outcome would be mTurquoise2 linked to mCherry2 (FIG. 2A). The recombinant fusion proteins, mTurquoise2::IN and IC::mCherry2, were expressed as soluble protein and purified through Ni-NTA and size exclusion chromatography (SEC). These protein preparations retained their folding and fluorescence, as indicated in SDS-PAGE of the samples with and without heat treatments FIGS. 2A-2D and 6A-6B.

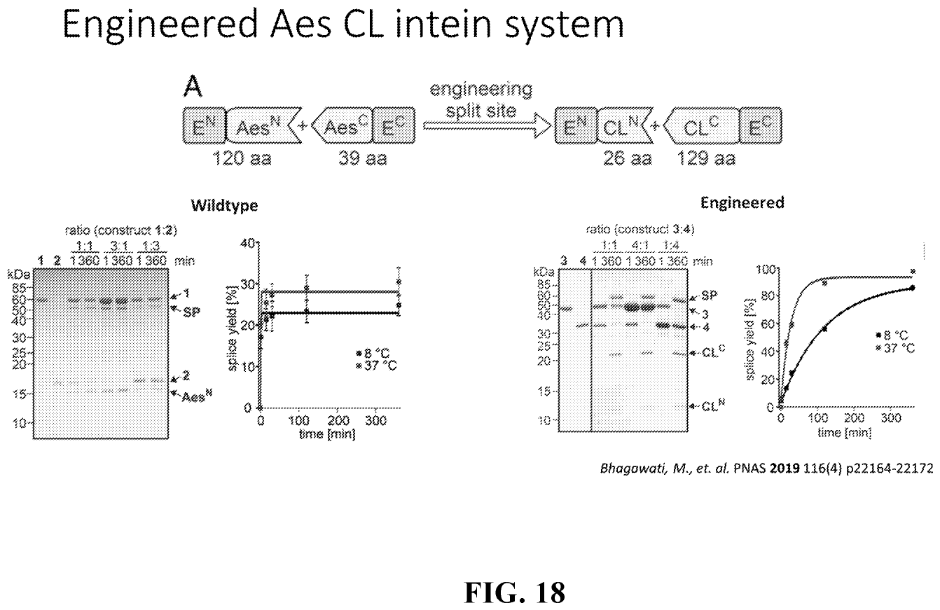

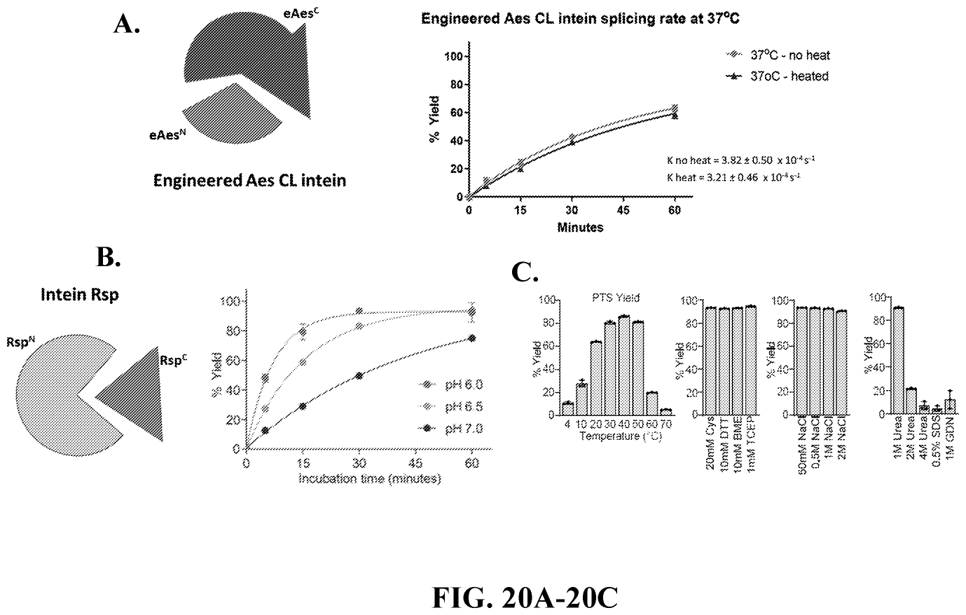

The folded proteins with fluorescence ran faster in SDS-PAGE gels and appeared as smaller-sized proteins. In contrast, the protein bands of the heated intein halves and PTS products migrated slower and corresponded to the right theoretical molecular weight ladders in SDS-PAGE gels (FIGS. 2A-2D and 6A-6B). Applicant proceeded to benchmark the previously reported engineered Aes CL split-intein systems²⁰ using the dual-fluorescent reporter system and its native N and C-terminal extein linkers DTD and VYLN, respectively. The initial pH screening results indicate that the Aes CL intein have optimal pH of splicing at 6.0. (FIG. 20A-20B). The splicing kinetic at pH 6.0 for engineered Aes CL intein was quantified by Coomassie stained SDS-PAGE of boiled intein reaction products and fluorescent imaged SDS-PAGE of non-boiled intein reaction products (FIG. 20B). For the fluorescent imaged SDS-PAGE of non-boiled intein reaction products, Applicant found that fluorescent signals reflecting protein trans-splicing reaction outcomes were faithfully matched with the intensities of Coomassie-stained protein bands of the same gel (FIG. 18). The resulting splicing kinetics Kapp value are within marginal error of each other with the fluorescent-based approach have slightly higher value potentially due to cleaner background and less heat-induced proteolysis effect from boiling. This suggest that the quantitative feature of the dual-reporter system fused to CL split-intein halves enable ratio-metric calculation of the apparent protein trans-splicing rate constant Kapp. With these results, Applicant proceeded to use fluorescent-based kinetic characterization for our other intein system of interest in mesophilic nondenaturing condition.

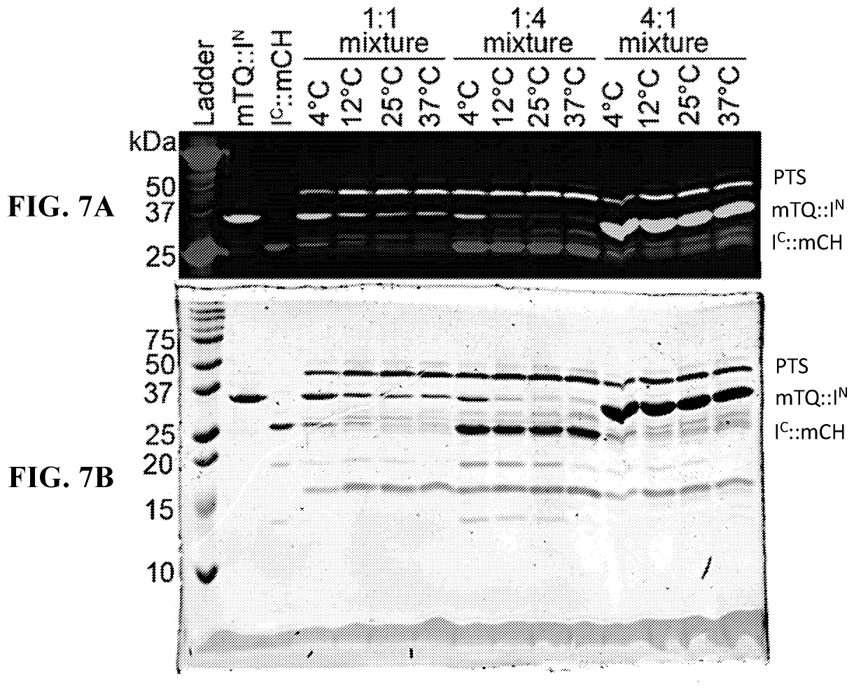

Applicant then characterize the PTS reactivity of the Rsp CL bioconjugation system and observed that this system is equipped with extein-tolerance and flexibility, supporting the potential of its wide applications compatible with many other fusion partners. The protein trans-splicing reaction was started immediately after mixing the two intein halves, which was completed within 6 hours at 37° C. in the pH 7.5 condition FIGS. 2C-2D, 6A-6B, and FIG. 7A-7B. The Kapp at 37° C. was measured to be 2.35±0.08×10^{-4 s-1}. When the temperature was lowered to 4° C., Kapp was reduced by approximately 10 times to 2.35±0.14×10^{-5, s-1}, and the reaction took 18 hrs to near-completion (FIG. 2C-2D). These results indicate that the Rsp CL system catalyzes the reaction to completion within hours in this pH 7.5 condition. In order to determine whether the Rsp CL intein system suffer from the same splicing defect found in the native Aes CL intein, Applicant tested PTS reaction using mixture containing different ratio of Rsp CL intein halves (FIG. 7A-7B). When there is molar excess of one intein halves, complete consumption of the molar limiting intein half are observed.

The surface charges of the intein halves are known to play a potential role in each other's recognition and association²⁴⁻²⁵, which is closely associated with their pI values and pH conditions used in experiments for the PTS reaction. Therefore, Applicant next examined various pH conditions to determine the optimal pH condition for the PTS reaction of the Rsp CL split-intein. Since theoretical p values of Rsp^{N} and Rsp^{C} are 4.32 and 9.70, respectively (Table C), Applicant tested a range of pH from 4 to 10 (FIG. 9A-9B). Unlike the observation that the completion of the PTS reaction at pH 7.5 took 6 hrs, the reaction went to completion at pH 6.0 and 7.0 within 1 hr (FIGS. 3A-3B and 10A-10C). This result indicates that the optimal pH for PTS reaction catalyzed by the Rsp CL split-intein is between 6 and 7, which is approximately the halfway point of the pI difference between the N- and C-terminal split-intein halves (Table 3).

TABLE 3
Physical and chemical properties of the CL split-intein systems.

| | | | | | | Instability |
|---|---|---|---|---|---|---|
| | Molecular | Theoretical | | Extinction | Estimated | index and |
| Names | Weight | pI | Atomic formula | coefficient * | half-life** | classification |
| Rsp^{N} | 13448.85 | 4.32 | C₅₉₂H₉₂₂N₁₆₀O₁₉₆S₁ | 15470 | 1.9 hr, >10 hr | 37.07, stable |
| Rsp^{C} | 5100.97 | 9.70 | C₂₃₅H₃₆₅N₆₁O₆₂S₂ | 4470 | 30 hr, >10 hr | 20.44, stable |
| Npu^{N} | 11476.96 | 5.58 | C₅₂₅H₈₀₀N₁₃₀O₁₅₇S₁ | 22920 | 1.9 hr, >10 hr | 34.03, stable |
| Npu^{C} | 3640.22 | 9.70 | C₁₆₅H₂₆₀N₄₆O₄₅S₁ | 4470 | >20 hr, >10 hr | 46.70, unstable |
| PsP-GDB | 50660.56 | 9.47 | C₂₂₉₈H₃₆₅₈N₆₂₈O₆₄₇S₇ | 64290 | 1.4 hr, >10 hr | 28.10, stable |
| PolB^{N}* | | | | | | |
| PsP-GDB | 11831.50 | 8.75 | C₅₄₃H₈₃₈N₁₄₄O₁₅₃ | 15930 | 1 hr, 2 min | 32.92, stable |
| PolB^{C} | | | | | | |
| Aes^{N} | 137010.98 | 9.76 | C₆₀₃H₉₉₃N₁₇₁O₁₇₈S₇ | 15470 | 1.9 hr, >10 hr | 26.53, stable |
| Aes^{C} | 4673.16 | 3.69 | C₂₁₀H₃₀₈N₄₈O₆₉S₂ | 1490 | 30 hr, >10 hr | 77.06, unstable |
| Aes^{N} | 2789.10 | 4.04 | C₁₂₂H₁₉₄N₂₈O₄₄S₁ | 1490 | 1.9 hr, >10 hr | 34.89, stable |
| (engineered) | | | | | | |
| Aes^{C} | 14957.23 | 6.60 | C₆₆₃H₁₀₆₀N₁₈₂O₁₉₇S₇ | 15470 | 1 hr, >10 hr | 35.49, stable |
| (engineered) | | | | | | |
| Pae^{N} | 13312.91 | 4.49 | C₅₈₇H₉₂₂N₁₅₀O₁₉₄S₄ | 11460 | 1.9 hr, >10 hr | 40.96, unstable |
| Pae^{C} | 9884.22 | 9.36 | C₄₄₀H₆₉₅N₁₂₃O₁₃₂S₂ | 5960 | 30 hr, >10 hr | 49.42, unstable |

ProtParam in the ExPASy server was used
* units in M⁻¹ cm⁻¹, at 280 nm measured in water
**1^{st} value, in mammalian cells and 2^{nd} value, in E. coli

Based on this observation, Applicant carried out kinetic analyses of the PTS reaction catalyzed by the Rsp CL split intein at pH 6, 6.5 and 7, revealing that the Rsp CL split-intein can catalyze the reaction to near-completion within 15 minutes at pH 6.0 (FIG. 13A-3B). The Kapp at pH 6 is calculated to be 2.31±0.18×10^{-3 s-1}, Kapp at pH 6.5 is 1.08±0.02×10^{-3 s-1} Kapp at pH 7 is 3.73±0.19×10^{-4 s-1} (FIGS. 3A-3B and 10A-10C). These values indicate that the protein trans-splicing rate of the Rsp CL split-intein at pH 6 is over ten times faster than the rate at pH 7.5, highlighting the speedy PTS reaction to completion catalyzed by Rsp CL system.

To confirm that the Rsp CL split-intein is insensitive to reducing agent, Applicant examined its trans-splicing activity in the presence of common reducing agents including cysteine, dithiothreitol (DTT), β-mercaptoethanol (BME), and tris-2-carboxyethyl-phosphine (TCEP) (FIGS. 4C-4D and 12A-12B). The Rsp CL split-intein reactivity was not impacted by the presence of these reducing agents. This feature supports its wide applications in laboratory and in vivo settings, including the oxidizing cellular organelle environment. Applicant also examined its trans-splicing activity in conditions relevant to the natural environment where this Richelia sp. was isolated 21 and in vitro and in vivo environments where this biosystem would be applied. The PTS reactions reached completion within an hour in the presence of various salt concentration ranging from 0.05 and 2M NaCl (FIGS. 4E-4F and 13A-13D). This speedy reaction and compatibility with low and high salt concentration of the Rsp CL system are remarkable, but these features align with the seasonal salinity changes observed in the natural environment in Cape Recife, South Africa, where the tufa stromatolite containing this cyanobacterium was isolated 21.

### The Pae and Cand CL Intein System Demonstrate Rapid Protein Trans-Splicing Reactivity and can Tolerate a Wide Range of Temperatures and Denaturing Agents

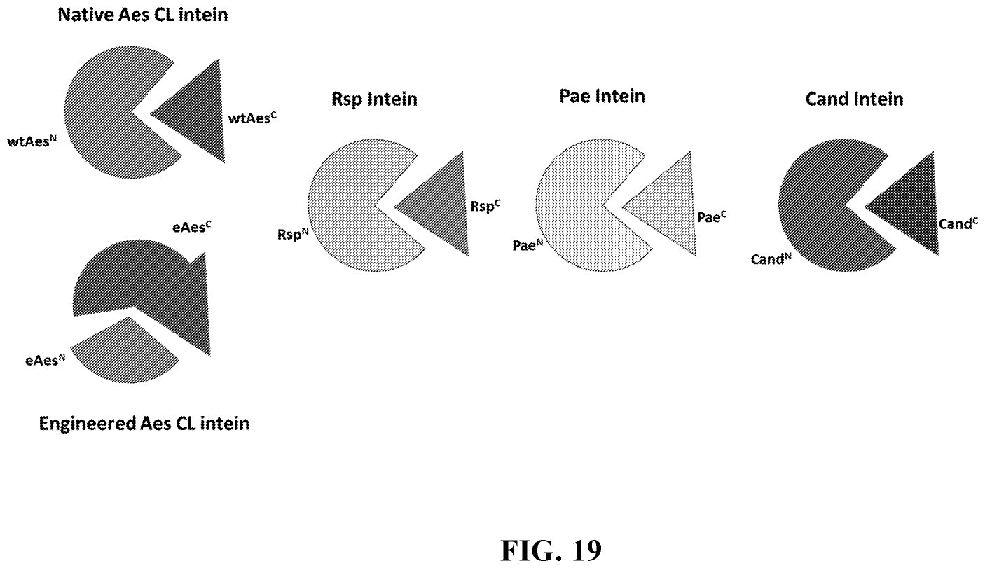

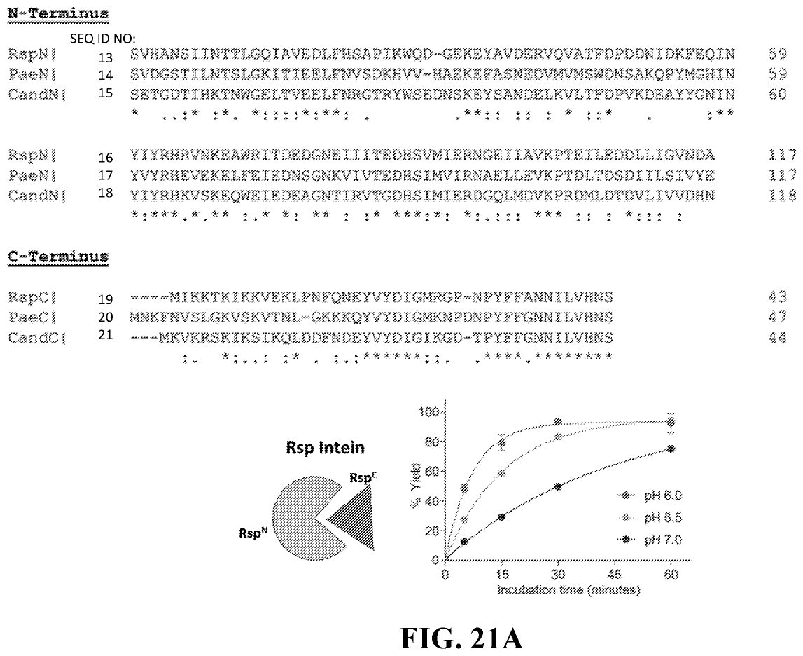

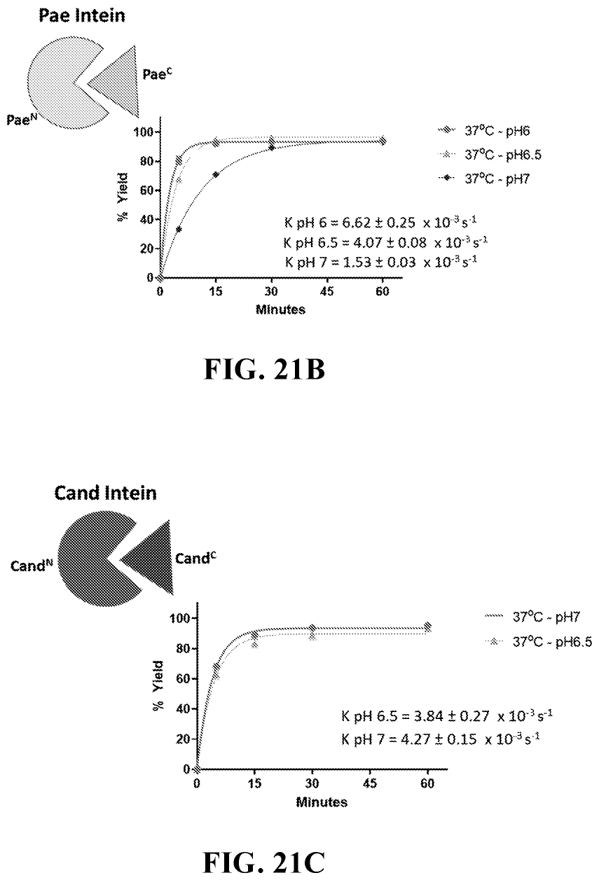

Applicant also characterized two other CL split-intein systems identified based on sequence similarity to Rsp CL system: the Pae CL intein system found in jumbo phages infecting Pseudomonas aeruginosa 18, and the C and CL intein system found in an unassigned Candidatus Brocadiales bacterium (FIGS. 1A-1C and 19). Both these intein systems also display optimal pH for splicing reactivity between pH 6.0 and pH 7.0. The Pae CL intein system reach near completion within 5 minutes at pH 6.0 with Kapp measured to be 6.62±0.25×10^{-3 s-1}. This intein retain rapid splicing kinetic at pH 6.5 and pH 7.0 with Kapp measured to be 4.07±0.08×10^{-3 s-1} and 1.53±0.03×10^{-3 s-1} respectively (FIG. 21A-21C). The Cand CL intein system display incomplete splicing activity at pH 6.0 thus the splicing kinetic for this intein was only characterized at pH 6.5 and pH 7.0 and measured to be 3.84±0.27×10^{-3 s-1} and 4.27±0.15×10^{-3 s-1}, respectively (FIG. 21A-21C). Both the Pae CL and Cand CL intein systems demonstrate rapid splicing reactivity out-stretch that of Rsp CL intein.

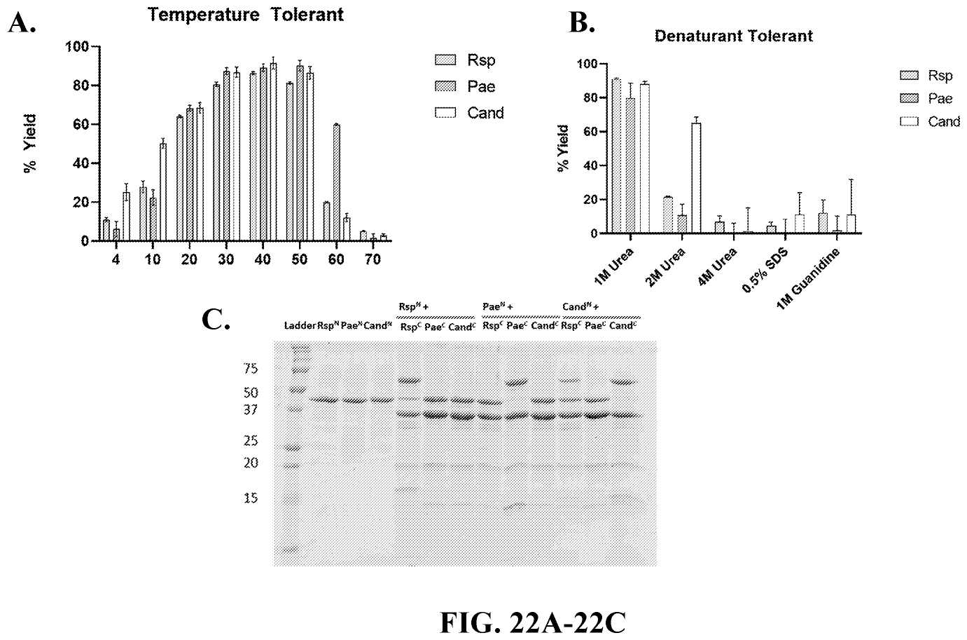

To learn more about the three Rsp, Cand, and Pae CL split intein systems, Applicant examined their trans-splicing activity in various conditions relevant to bioconjugation applications. These CL split-intein system catalyzed the PTS reaction in a wide temperature range from 4° C. to 70° C. An hour incubation of the split-intein mixtures resulted in over 60% completion of the PTS reaction at 20° C., and over 80% completion at 30° C., 40° C. and 50° C. (FIG. 22A). Since some extein partners for CL split-intein might require the use of denaturing agents in protein preparation and/or reaction steps, Applicant also examined the tolerance of these CL split-intein systems to common denaturing agents frequently used in a laboratory setting, revealing the moderate level of tolerance to denaturing agents. (FIG. 22B). All three CL split-intein system catalyzed the PTS reaction to near completion in the presence of 1M Urea, while the Cand CL intein display most activity in the presence of 2M Urea among the inteins tested. Small but detectable PTS reaction products were also observed in the reaction mixture containing 4M Urea, 0.5% sodium dodecyl sulfate (SDS) or 1M guanidine (GDN).

Since these three CL intein systems were identified through sequence similarity, Applicant want to determine the specificity of each CL split-intein halves for each other within the same system. N-terminal intein halves was mixed with C-terminal intein halves for 1 hour at 37° C. and pH 7.0 and the PTS reaction was then determined through SDS-PAGE analysis (FIG. 22C). The results indicate that Rsp and Pae CL split-intein does not cross-react with each other. The results also indicate that only Cand C-terminal intein half can recognize and react with Rsp N-terminal intein half in addition to the native Cand N-terminal intein half.

### CL Intein System Retain Traditional Intein Hedgehog Fold and Other Conserved Active Site Motif.

Applicant first determined through X-ray crystallography of the structure of the excised Rsp CL intein complex resultant of PTS splicing reaction. The complex between excised RspN and RspC was observed to be highly stable as they appear as a single band on SDS-PAGE gel when left unboiled. Scaled up reaction of mTurquoise2::RspN and RspC::mCherry2 was subjected to size exclusion chromatography and the intein complex byproduct was isolated and crystallized. The octahedron crystals formed within 4 days. X-ray diffraction data was then obtained through synchrotron beamline with diffraction resolution of 1.75 A.

In order to obtain additional structural understanding, the three intein system was recombinantly expressed as S1A mutant and fused together by a glycine residue. The recombinant S1A whole inteins was expressed and purified with the N-terminal extein linker DTD and C-terminal extein linker AYISA followed by a C-terminal 6×Histidine tag. The proteins were purified through Ni-NTA and Size Exclusion chromatography prior to crystallography study. The fused Pae whole intein formed octahedron crystal within one week. X-ray diffraction data was then obtained through synchrotron beamline with diffraction resolution of 1.6 A.

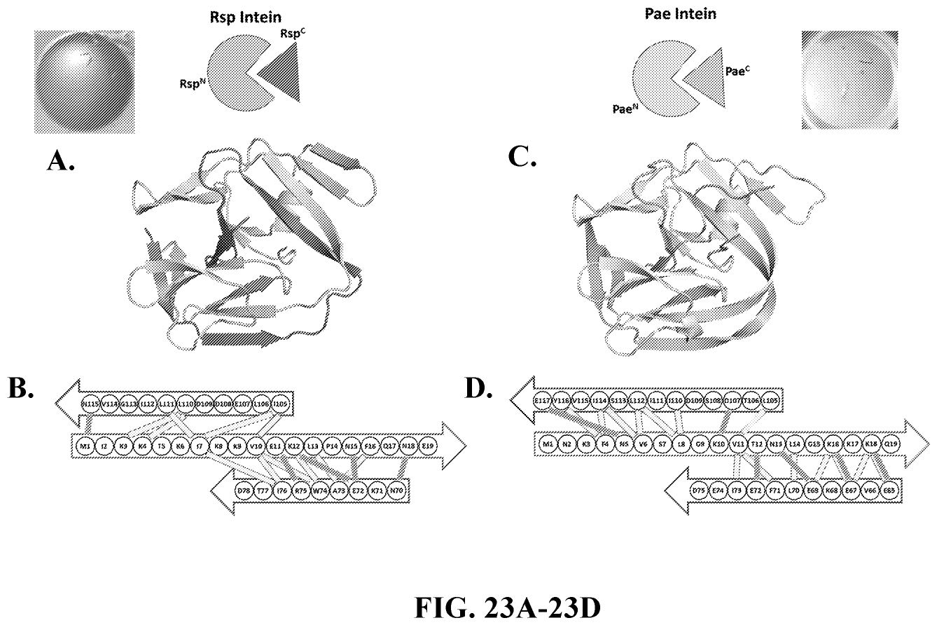

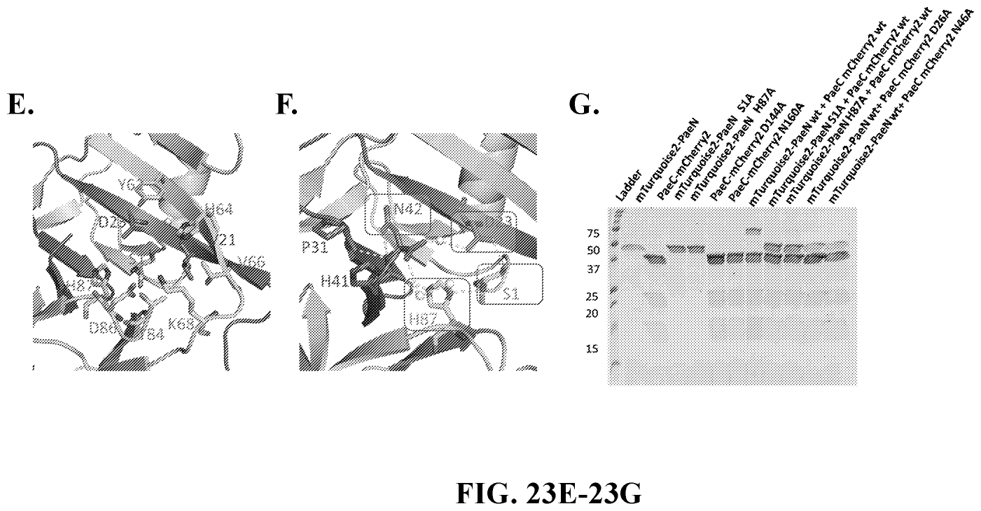

Both Rsp and Pae intein adopt the horse-shoe shaped traditional intein/hedgehog fold (FIGS. 23A and 23C). Both structures contain the extremophile hairpin (EXH) motif.26 Detail analysis of residues interaction suggest a combination of charge interaction and hydrophobic interaction is responsible for split-intein complex formation in both intein (FIGS. 23B and 23D). The S1 residue of Rsp Intein is highly coordinated in the intein active site. (FIG. 23E). The O3 atom of this serine is coordinated by RspN Y62, H64, K68, T84, and RspC D23. The C3 atom of this serine is also coordinated by hydrophobic interaction with RspN V66 and RspC V21. The N1 atom of this serine is coordinated by the RspN T84, D86, and H87 residues that is part of the active site. The active sites of Rsp intein retain highly conserved interactions (FIG. 23F). RspN H87 and RspC D23 help coordinate the interaction between S1 and S+1 residues. The RspC H41 and N42 help coordinate the S+1 residue.

The S1A Pae CL whole intein structure indicate that the mutation causes deformation of active sites in Pae. (FIG. 23G) The key PaeN H87 residue is pushed away from the active site and the PaeN D-1 residue from the N-term extein linker occupied the active site in place of the S1A residue. However, to confirm that the active sites residues involved in splicing reactivity of Pae CL intein is conserved, Applicant conducted mutagenesis study accompany by PTS reactivity determination. The following four mutants PaeN S1A and H87A and PaeC D26A and N46A show no PTS reactivity (FIG. 23G).

These New CL Intein System Contain Unique Proline Residues in its F-Block Loop that Contribute to their Increase Reactivity

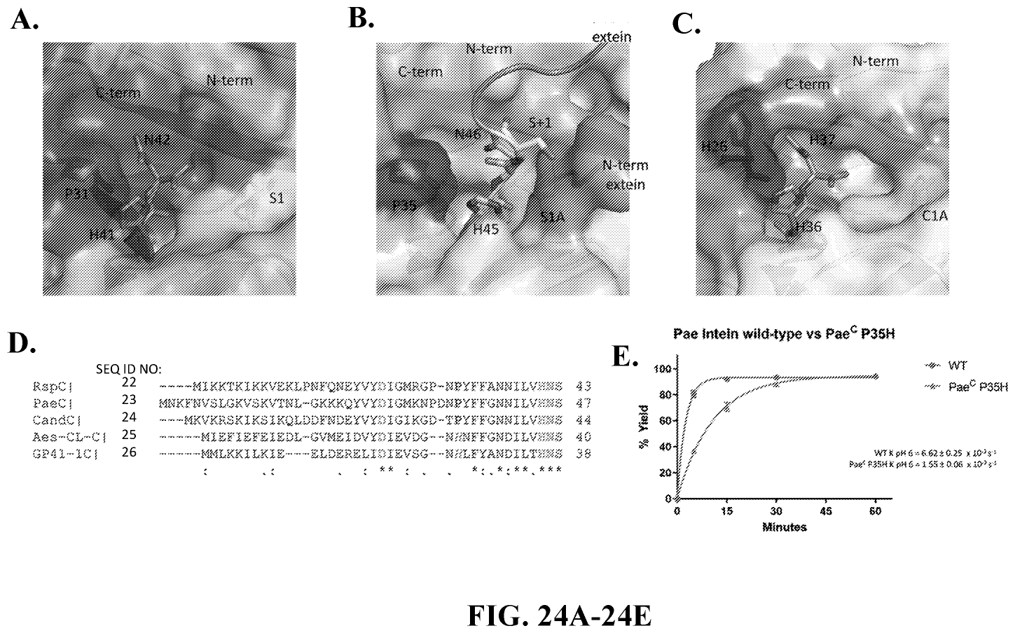

Structural analysis of the active sites of Rsp and Pae intein reveal a unique structural feature where a proline is taking the place traditionally occupied by a catalytic Histidine found in other intein structure like in the GP41-1 intein system²⁷. (FIG. 24A-24C) The location of this proline residue assists in the coordination of catalytic asparagine and penultimate histidine that are involved in the final intein rearrangement of and release of the PTS product from the C-terminal intein half. Sequence alignment of C-terminal intein half between our three CL intein system against the Aes CL intein system 20 and the ultrafast GP41-1 intein system 27 indicate a conserved Proline mutation. (FIG. 24D). When Applicant introduced PaeC P35H mutation to the Pae intein, the PTS reactivity is markedly reduced (FIG. 24E). This suggest that the proline mutation contributes to the increase in splicing reactivity within these new CL intein systems.

Chimeric CL Intein System Derived from the C-Terminal Intein of Pae and Cand CL Intein System Demonstrated Increase Both Reactivity and Tolerant to Temperature and Denaturant

When conducting pH range characterization for Cand CL intein, Applicant noted a flaw where the reaction does not go to completion and the molecular weight indicate that the C-terminal intein half is not efficiently released from the PTS extein product at pH 6.0. However, the reactivity of Cand CL intein at pH 7.0 as well as reactivity in broad temperature and in the presence of 2M Urea make it a desired system to be engineered further. Applicant also seek to eliminate the observed cross reactivity of the Cand^{C} with Rsp^{N}.

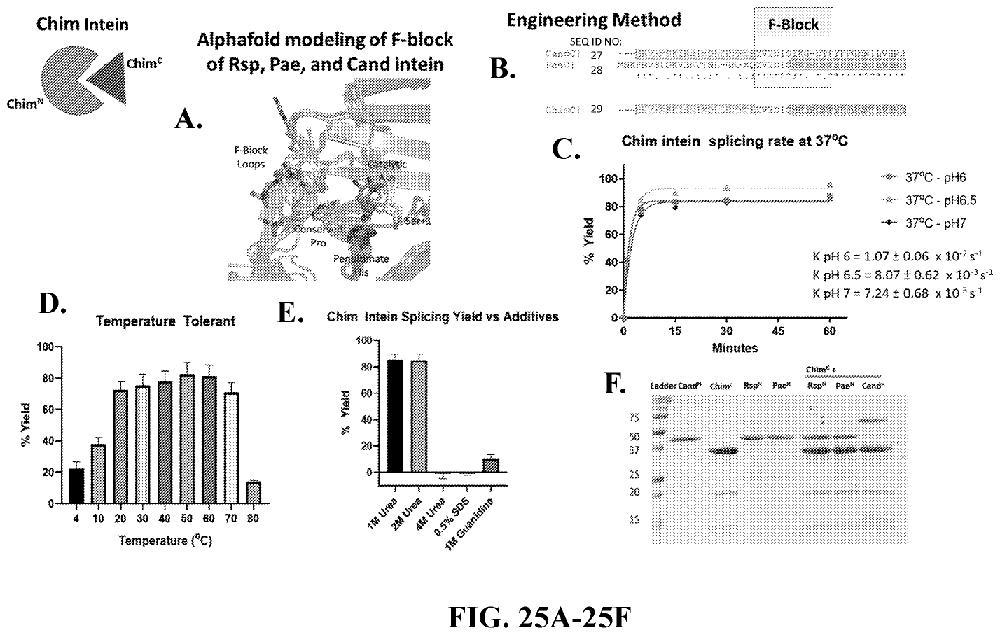

Using AlphaFold 28 for structural prediction, Applicant observed that the F-block loop of Cand intein display great flexibility around the C-terminal intein catalytic asparagine and penultimate histidine and Ser+1 (FIG. 25A). Applicant generated a chimeric C-terminal intein (Chim^{C}) by introducing the F-block loop from Pae^{C} onto Cand^{C} intein. (FIG. 25B). This chimeric intein system consist of Cand^{N} and Chim^{C}, which will be prefer to as Chim CL intein system, was able carry out PTS reaction and have highest Kapp reported thus far: 1.07±0.06×10^{-2 s-1} at pH 6.0, 8.07±0.62×10^{-3 s-1} at pH 6.5 and 7.24±0.68×10^{-3 s-1} at pH 7.0 (FIG. 25C). At all pH tested, the reactions approached near completion within 5 minutes.

The Chim CL intein system also has increased temperature tolerance (FIG. 25D) with observable reactivity up to 80° C. within 1 hour. It also retains reactivity at lower temperature similar to the parent Cand CL intein system. Similar to the Cand CL intein system, the Chim CL intein system show increase reactivity in the presence of 2M Urea (FIG. 25E). Lastly, the Chim^{C} intein half was tested against Rsp^{N} and Pae^{N} intein half and show no observable cross-reactivity after one hour of incubation at 37° C. and pH 7.0 (FIG. 25F).

### Discussion

Applicant demonstrated the three native CL split-inteins Rsp, Pae and Cand as well as the engineered Chim CL split-intein are capable of catalyzing protein trans-splicing reactions to completion in an exact manner even under certain extreme conditions known to be unfavorable for the PTS reaction to occur. These CL systems are a starch improvement to most CL split-intein systems characterized thus far, based on their speedier reaction to completion, leading to an efficient and high protein conjugation yield. These newly characterized bioconjugation systems are also equipped with impressive compatibility with various conditions, distinguishing them from other counter parts. These results highlight the promise of these newly discovered bioconjugation system in various protein engineering and therapeutic applications.

While many previous intein characterization study utilizing solubility enhancing fusion protein as exteins, the use of fluorescent extein protein fused to the N- and C-terminal split intein halves of these CL system supports the extein tolerance and flexibility of this bioconjugation system. In support of the extein tolerance/flexibility, the Rsp CL system has been successfully conjugated to different sets of proteins and peptides in the laboratory (unpublished). Furthermore, Applicant demonstrated that the three CL split-intein systems Rsp, Pae and Chim have no cross-reactivity favoring their potential use in multiplex bioconjugation applications.

The structural determination of the Rsp CL post-reaction complex and the S1A Pae fused-intein give us a better structural understanding of these CL intein complexes. They retain the traditional intein/hedgehog fold while carry some amino acid changes that contribute to their increase PTS reactivity in the absence of the more reactive thiol group found in their cysteine-based intein counterpart. Applicant discovered the presence of a proline on F-block loop that help in the coordination of the active site catalytic asparagine and penultimate histidine, is important for improve reactivity. Previous studies show that modification to this conserved F-block residue in other intein slow down the splicing kinetic significantly.²⁹⁻³⁰ While structurally, the S1A mutant of Pae CL whole intein display a disturbed active site, Applicant demonstrated through mutagenesis study that the Pae CL intein retain the traditional catalytic active sites residues.

In addition, through the design of Chim^{C} intein, Applicant further validate the importance in the interaction between the F-block loop with the C-terminal extein and the reaction center surrounding S+1 residue. In our system, the proline on the F-block loop is found to replace the traditional catalytic position held by a histidine residue. It was shown that when a histidine is presence at this position, the F-loop required further modification in order to tolerate less bulky amino residues at the +2 position of the C-terminal extein.³¹ For all of our intein systems, an AYISA polypeptide linker were used to link the C-terminal intein half with its extein. This linker thus introduced an alanine at the +2 C-terminal extein position which reportedly causes unwanted flexibility and lower Kapp in Npu Intein system. Hence, the rapid reactivity observed in these new CL intein in the presence of Ala+2 residue 31 suggests a potential for greater flexibility for exteins amino acid composition. In addition, this newly available structural information for CL intein systems will serve as invaluable template for structural-based engineering of new CL intein with artificial split sites 2 that can retain the desired selectivity, PTS reaction speed and folding stability.

Applicant also successfully designed a chimeric CL split-intein system based on the Cand CL system, resulting in increases in both PTS reaction speed as well as overall reactivity over a broader range of pH and denaturant concentrations. This Chim CL intein show no cross reactivity with the Rsp and Pae CL inteins. This engineering approach highlight the significant of the F-block loops in catalyzing the C-terminal intein rearrangement and release.

These new CL split-intein system stands out in the group in that they can tolerate and catalyze the PTS reaction in a wide range of conditions, including several extreme environments. The optimal condition associated with the speedy PTS reaction by these CL systems is a pH range of 6 to 8 and a temperature range of 20° C. to 50° C., which is generally not influence by the presence of common reducing agents and a wide range of salt concentrations. Furthermore, the CL split-intein can tolerate common denaturing agents and concentrations. These features will make these new CL bioconjugation system compatible with extein partners whose preparations required lower or higher temperatures, reducing agents, high salt concentrations, and/or denaturing agents.

It is worth highlighting that these CL system is compatible with a broad pH range and does not required reducing agents for activity, in therapeutic application perspective, since it supports the promise of these bioconjugation system's functionality in cellular organelles maintaining lower pH and oxidizing states, such as vesicles, lysosomes, and endoplasmic reticulum, cellular organelles associated with various devastating diseases without effective therapeutic strategies, including various infectious disease and neurological disorders.³²⁻⁴¹

Using ProtParam, Applicant calculated theoretical pI values and protein stabilities of the CL split-inteins studied thus far. The pI difference between N- and C-terminal split intein halves is suggested to play a critical role in each other's recognition and association. The pI values of split-intein halves seem to favor an acidic value for the N-terminal half and a basic value for the C-terminal half, and the pI value differences are 4-6 between the split-intein pair (Table 3). It is worth noting that the engineered Aes CL split-intein may be associated with a narrower application potential than these new CL systems. The CL split-intein systems reported in this paper are also highlighted by the best protein stability prediction results among the CL split-inteins (Table 3).

Like other split-inteins, the native extein proteins transcribed by these new CL intein poplypeptides are putative DNA polymerase subunits as predicted with over 99% probability through the pairwise comparison of profile hidden Markov models (Tables 1 and 2) This prediction is in line with the features associated with other mature extein products involved in DNA processing. Further experimentation is required to demonstrate whether the mature extein products catalyzed by these CL split-intein system are DNA polymerase subunit(s). It is also intriguing to conduct future studies designed to address a relationship between split-intein protein stabilities and the possible transient requirement of mature extein products, as a significant number of CL split-intein halves are predicted to be unstable (Table 3).

Having the visual means to trace and measure the enzymatic reactions during the processes is powerful in several ways. For such reasons, Applicant established the dual fluorescent protein trans-splicing assay in characterizing these CL split-intein. First, this system allowed us to monitor the tight protein folding of investigated proteins, calculate the apparent protein trans-splicing rate constant Kapp, and distinguish and trace the PTS reaction product associated exclusively with the Rsp CL split intein system. For instance, Applicant compared unboiled and boiled samples as part of benchmarking against the reported engineered Aes CL intein system and demonstrate the Kapp value to be within marginal error of each other. In addition, a series of time-course experiments let us conduct the kinetic analyses of this bioconjugation system. Lastly, the use of different fluorescent proteins for each split-intein half was made to trace the split-intein-mediated PTS reaction in mesophilic nondenaturing conditions. Therefore, the methodology used in this study can serve as an example for other similar studies aimed at characterizing new split-intein systems.

In summary, the new CL split-inteins reported here are equipped with many remarkable features critical for wide applications, highlighting the promise of these newly discovered bioconjugation systems in various protein engineering and therapeutic applications.

### Example 3

This Example provides recombinant proteins used in Examples 1 and 2. See e.g., Table D. This Example also provides sequences of split-intein system proteins, such as those demonstrated in Examples 1 and 2.

TABLE D
Recombinant proteins used Examples 1 and 2.

| Plasmid | | Vector | E. coli | |
|---|---|---|---|---|
| names | Protein names | backbone | strain | Primer sequences used |
| pJS0095 | mTurquoise::Rsp^{N} | pET28a | DH5a | Insert_F: CTTTAAGAAGGAGATATACCATGGTTTCCAAAGG |
| | | | | GGAGG (SEQ ID NO: 114) |
| | | | | Insert_R: TCCCGCGTCGTTCACACC (SEQ ID NO: 115) |
| | | | | Vector_F: GTGAACGACGCGGGACACCACCACCACCACCAC |
| | | | | TGAG (SEQ ID NO: 116) |
| | | | | Vector_R: CATGTATATCTCCTTC (SEQ ID NO: 117) |
| pJS0096 | Rsp^{C}::mCherry | pET28a | DH5a | Insert_F: GAAGGAGATATACCATGATTAAAAAGACG (SEQ |
| | | | | ID NO: 118) |
| | | | | Insert_R: GTTAGCAGCCGGATCTCAGTGATGGTGGTGATG |
| | | | | (SEQ ID NO: 119) |
| | | | | Vector_F: TGAGATCCGGCTGCTAACAAAGCC (SEQ ID NO: |
| | | | | 120) |
| | | | | Vector_R:CGTCTTTTTAATCATGGTATATCTCCTTC (SEQ ID |
| | | | | NO: 121) |
| pJS0097 | Aes^{C}::Cfa^{N} | pET28a | DH5a | Insert_F: GAAGGAGATATACCATGATGGGCGCGAGTG (SEQ |
| | | | | ID NO: 122) |
| | | | | Insert_R: GGCTTTGTTAGCAGCCGGATCTCAATGGTGATGA |
| | | | | TGATGGTG (SEQ ID NO: 123) |
| | | | | Vector_F: ACACCTGACGGTTCCCACCACCACCACCACCAC |
| | | | | (SEQ ID NO: 124) |
| | | | | Vector_R: CATGTATATCTCCTTC (SEQ ID NO: 125) |
| pJS0098 | Cfa^{C}::Gamillus | pET28a | DH5a | Insert_F: GAAGGAGATATACCATGGGGCACCATCATCATCA |
| | | | | CCATTCTGG (SEQ ID NO: 126) |
| | | | | Insert_R:GGCTTTGTTAGCAGCCGGATCTCACTTGTATAATT |
| | | | | CGTCCATGC (SEQ ID NO: 127) |
| | | | | Vector_F: CACGGTAAACGAAGTCTACCCCATGGTGATGAT |
| | | | | GATGGTGC (SEQ ID NO: 128) |
| | | | | Vector_R: TGAGATCCGGCTGCTAACAAAGCC (SEQ ID NO: |
| | | | | 129) |

SEQ ID NO: 1, NJO60988.1 hypothetical protein
[Richelia sp. RM2_1_2]-RSPN
SVHANSIINTTLGQIAVEDLFHSAPIKWQDGEKEYAVDERVQVATFDP
DDNIDKFEQINYIYRHRVNKEAWRITDEDGNEIIITEDHSVMIERNGE
IIAVKPTEILEDDLLIGVNDA
SEQ ID NO: 2, NJO60986.1 hypothetical protein
[Richelia sp. RM2_1_2]-RSPC
MIKKTKIKKVEKLPNFQNEYVYDIGMRGPNPYFFANNILVHNS
SEQ ID NO: 3, Candidatus MBC cysteine-less
split-intein system containing IN
GDTIHKTNWGELTVEELFNRGTRYWSEDNSKEYSANDELKVLTFDPVK
DEAYYGNINYIYRHKVSKEQWEIEDEAGNTIRVTGDHSIMIERDGQLM
DVKPRDMLDTDVLIVVD
SEQ ID NO: 4, Candidatus MBC cysteine-less
split-intein system containing IC
VKRSKIKSIKQLDDFNDEYVYDIGIKGDTPYFFGNNILVHNS
SEQ ID NO: 5, Pseudomonas QCG cysteine-less
split-intein system containing IN
SVDGSTILNTSLGKITIEELFNVSDKHVVHAEKEFASNEDVMVMSWDN
SAKQPYMGHINYVYRHEVEKELFEIEDNSGNKVIVTEDHSIMVIRNAE
LLEVKPTDLTDSDIILSI
SEQ ID NO: 6, Pseudomonas QCG cysteine-less
split-intein system containing IC
LGKVSKVTNLGKKKQYVYDIGMKNPDNPYFFGNNILVHNS
SEQ ID NO: 7, Synthetic Chimera N-terminal:
SETGDTIHKTNWGELTVEELFNRGTRYWSEDNSKEYSANDELKVLTFD
PVKDEAYYGNINYIYRHKVSKEQWEIEDEAGNTIRVTGDHSIMIERDG
QLMDVKPRDMLDTDVLIVVDHN
SEQ ID NO: 8, Synthetic Chimera C-terminal:
MKVKRSKIKSIKQLDDFNDEYVYDIGMKNPDNPYFFGNNILVHNS
SEQ ID NO: 9, Proline that increases activity
NPYFFANNILVHNS

### REFERENCES RELATED TO EXAMPLES 1 AND 2

1. Shah N H, Muir T W. Inteins: Nature's Gift to Protein Chemists. Chem Sci 5, 446-461 (2014).
2. Aranko A S, Wlodawer A, Iwai H. Nature's recipe for splitting inteins. Protein Eng Des Sel 27, 263-271 (2014).
3. Novikova O, Topilina N, Belfort M. Enigmatic distribution, evolution, and function of inteins. J Biol Chem 289, 14490-14497 (2014).
4. Mills K V, Johnson M A, Perler F B. Protein splicing: how inteins escape from precursor proteins. J Biol Chem 289, 14498-14505 (2014).
5. Perler F B. InBase: the Intein Database. Nucleic Acids Res 30, 383-384 (2002).
6. Belfort M. Mobile self-splicing introns and inteins as environmental sensors. Curr Opin Microbiol 38, 51-58 (2017).
7. Naor A, et al. Impact of a homing intein on recombination frequency and organismal fitness. Proc Natl Acad Sci USA 113, E4654-4661 (2016).
8. Pietrokovski S. Conserved sequence features of inteins (protein introns) and their use in identifying new inteins and related proteins. Protein Sci 3, 2340-2350 (1994).
9. Perler F B, Olsen G J, Adam E. Compilation and analysis of intein sequences. Nucleic Acids Res 25, 1087-1093 (1997).
10. Derbyshire V, Wood D W, Wu W, Dansereau J T, Dalgaard J Z, Belfort M. Genetic definition of a protein-splicing domain: functional mini-inteins support structure predictions and a model for intein evolution. Proc Natl Acad Sci USA 94, 11466-11471 (1997).
11. Gogarten J P, Senejani A G, Zhaxybayeva O, Olendzenski L, Hilario E. Inteins: structure, function, and evolution. Annu Rev Microbiol 56, 263-287 (2002).
12. Galburt E A, Stoddard B L. Catalytic mechanisms of restriction and homing endonucleases. Biochemistry 41, 13851-13860 (2002).
13. Duan X, Gimble F S, Quiocho F A. Crystal structure of PI-SceI, a homing endonuclease with protein splicing activity. Cell 89, 555-564 (1997).
14. Wu H, Hu Z, Liu X Q. Protein trans-splicing by a split intein encoded in a split DnaE gene of Synechocystis sp. PCC6803. Proc Natl Acad Sci USA 95, 9226-9231 (1998).
15. Southworth M W, Benner J, Perler F B. An alternative protein splicing mechanism for inteins lacking an N-terminal nucleophile. Embo j 19, 5019-5026 (2000).
16. Choi J J, Nam K H, Min B, Kim S J, Söll D, Kwon S T. Protein trans-splicing and characterization of a split family B-type DNA polymerase from the hyperthermophilic archaeal parasite Nanoarchaeum equitans. J Mol Biol 356, 1093-1106 (2006).
17. Gordo V, et al. Structural Insights into Subunits Assembly and the Oxyester Splicing Mechanism of Neq pol Split Intein. Cell Chem Biol 25, 871-879.e872 (2018).
18. Imam M, et al. vB_PaeM_MIJ3, a Novel Jumbo Phage Infecting Pseudomonas aeruginosa, Possesses Unusual Genomic Features. Front Microbiol 10, 2772 (2019).
19. Southworth M W, Adam E, Panne D, Byer R, Kautz R, Perler F B. Control of protein splicing by intein fragment reassembly. Embo j 17, 918-926 (1998).
20. Bhagawati M, et al. A mesophilic cysteine-less split intein for protein trans-splicing applications under oxidizing conditions. Proc Natl Acad Sci USA 116, 22164-22172 (2019).
21. Waterworth S C, Isemonger E W, Rees E R, Dorrington R A, Kwan J C. Conserved bacterial genomes from two geographically isolated peritidal stromatolite formations shed light on potential functional guilds. Environ Microbiol Rep, (2020).
22. Davis J J, et al. The PATRIC Bioinformatics Resource Center: expanding data and analysis capabilities. Nucleic Acids Res 48, D606-d612 (2020).
23. Soding J, Biegert A, Lupas A N. The HHpred interactive server for protein homology detection and structure prediction. Nucleic Acids Res 33, W244-248 (2005).
24. Shi J, Muir T W. Development of a tandem protein trans-splicing system based on native and engineered split inteins. J Am Chem Soc 127, 6198-6206 (2005).
25. Dassa B, Amitai G, Caspi J, Schueler-Furman O, Pietrokovski S. Trans protein splicing of cyanobacterial split inteins in endogenous and exogenous combinations. Biochemistry 46, 322-330 (2007).
26. Toyofuku M, Nomura N, Eberl L. Types and origins of bacterial membrane vesicles. Nat Rev Microbiol 17, 13-24 (2019).
27. Roy C R. Exploitation of the endoplasmic reticulum by bacterial pathogens. Trends Microbiol 10, 418-424 (2002).
28. Ozcan L, Tabas I. Role of endoplasmic reticulum stress in metabolic disease and other disorders. Annu Rev Med 63, 317-328 (2012).
29. Roussel B D, Kruppa A J, Miranda E, Crowther D C, Lomas D A, Marciniak S J. Endoplasmic reticulum dysfunction in neurological disease. Lancet Neurol 12, 105-118 (2013).
30. Kim I, Xu W, Reed J C. Cell death and endoplasmic reticulum stress: disease relevance and therapeutic opportunities. Nat Rev Drug Discov 7, 1013-1030 (2008).
31. Platt F M, d'Azzo A, Davidson B L, Neufeld E F, Tifft C J. Lysosomal storage diseases.
32. Ballabio A, Gieselmann V. Lysosomal disorders: from storage to cellular damage. Biochim Biophys Acta 1793, 684-696 (2009).
33. Yarwood R, Hellicar J, Woodman P G, Lowe M. Membrane trafficking in health and disease. Dis Model Mech 13, (2020).
34. Gissen P, Maher E R. Cargos and genes: insights into vesicular transport from inherited human disease. J Med Genet 44, 545-555 (2007).
35. Sharp T M, Estes M K. An inside job: subversion of the host secretory pathway by intestinal pathogens. Curr Opin Infect Dis 23, 464-469 (2010).
36. Lefort V, Desper R, Gascuel O. FastME 2.0: A Comprehensive, Accurate, and Fast Distance-Based Phylogeny Inference Program. Mol Biol Evol 32, 2798-2800 (2015).
37. Gasteiger E, et al. Protein Identification and Analysis Tools on the ExPASy Server.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

Further attributes, features, and embodiments of the present invention can be understood by reference to the following numbered aspects of the disclosed invention. Reference to disclosure in any of the preceding aspects is applicable to any preceding numbered aspect and to any combination of any number of preceding aspects, as recognized by appropriate antecedent disclosure in any combination of preceding aspects that can be made. The following numbered aspects are provided:
1. An engineered intein system comprising: a recombinant first amino acid sequence comprising an N-terminal intein sequence; and a recombinant second amino acid sequence comprising a C-terminal intein sequence, wherein the N-terminal intein sequence, the C-terminal intein sequence, or both are derived from a split intein of Richelia sp., Pseudomonas aeruginosa 18, Candidatus Brocadiales, or any combination thereof.
2. The engineered intein system of aspect 1, wherein the split intein is a cysteine-less split intein.
3. The engineered intein system of any one of aspects 1-2, wherein the N-terminal intein sequence comprises an amino acid sequence having at least 80%, 85%, 90%, 95%, 98% or 99% sequence identity to any one of SEQ ID NO: 1, 3, 5, or 7.
4. The engineered intein system of any one of aspects 1-3, wherein the C-terminal intein sequence comprises an amino acid sequence having at least 80%, 85%, 90%, 95%, 98% or 99% sequence identity to any one of SEQ ID NO: 2, 4, 6, or 8.
5. The engineered intein system of any one of aspects 1-4, wherein the wherein the N-terminal intein sequence is attached to a C-terminus of the first amino acid sequence with a peptide bond.
6. The engineered intein system of any one of aspects 1-5, wherein the C-terminal intein sequence is attached to a N-terminus of the first amino acid sequence with a peptide bond.
7. The engineered intein system of any one of aspects 1-6, further comprising a linker between the first amino acid sequence and the N-terminal intein sequence, optionally wherein the linker is a peptide linker.
8. The engineered intein system of any one of aspects 1-7, further comprising a linker between the first amino acid sequence and the C-terminal intein sequence, optionally wherein the linker is a peptide linker.
9. The engineered intein system of any one of aspects 7-8, wherein the linker is not more than 1, 2, 3, 4, 5, 6, 7, 8, 9, 10, 11, 12, 13, 14, 15, or 20 amino acids in length.
10. The engineered intein system of any one of aspects 7-9, wherein linker is a Gly-Ser linker, optionally wherein the linker comprises an amino acid sequence of at least 80%, 85%, 90%, 95%, 98% or 99% sequence identity to GSGSGSGSGSGSGSGSGSGSG (SEQ ID NO: 11).
11. The engineered intein system of any one of aspects 7-10, wherein the linker is an Asparagine-Serine linker, optionally wherein the linker comprises an amino acid sequence of at least 80%, 85%, 90%, 95%, 98% or 99% sequence identity to ASASASASASASASASAS (SEQ ID NO: 12).
12. The engineered intein system of any one of aspects 1-11, further comprising a localization tag, affinity tag, reporter tag, or any combination thereof, wherein the localization tag, affinity tag, reporter tag, or any combination thereof is operatively coupled to the first amino acid sequence, the second amino acid sequence, or both.
13. The engineered intein system of any one of aspects 1-12, wherein the C-terminal intein sequence comprises X₁PYFFX₂NNILVHNS (SEQ ID NO: 10), wherein X₁ and X₂ are each independently selected from any amino acid.
14. The engineered intein system of aspect 13, (a) wherein X₁ is selected from N or T, (b) wherein X₂ is selected from A or G, or (c) both (a) and (b).
15. The engineered intein system of aspect 13, wherein the C-terminal sequence comprises SEQ ID NO: 9.
16. The engineered intein system of any one of aspects 1-15, wherein the system is capable of catalyzing a bioconjugation reaction at a pH ranging from about 6 to about 8.
17. The engineered intein system of any one of aspects 1-16, wherein the system is capable of catalyzing a bioconjugation reaction at a temperature ranging from about 20° C. to about 50° C.
18. The engineered intein system of any one of aspects 1-17, wherein the system is capable of catalyzing a bioconjugation reaction, wherein the system is capable of catalyzing a bioconjugation reaction in the presence of a reducing agent, optionally wherein the reducing agent is dithiothreitol (DTT), beta mercaptoethanol (BME), tris(2-carboxyethyl)phosphine (TCEP), or cysteine.
19. The engineered intein system of any one of aspects 1-18, wherein the system is capable of catalyzing a bioconjugation reaction in the presence of about 0.05 M NaCl to about 2 M NaCl.
20. An engineered polynucleotide encoding the engineered intein system of any one of aspects 1-19 or a component thereof.
21. A vector or vector system comprising: one or more engineered polynucleotides of aspect 20, optionally wherein at least one of the one or more engineered polynucleotides is operatively coupled to a regulatory element.
22. A cell or population thereof comprising: a. engineered intein system of any one of aspects 1-19; b. one or more engineered polynucleotides of aspect 20; c. one or more vector or vector systems of aspect 21; or d. any combination of (a)-(c).
23. A non-human organism comprising: a. engineered intein system of any one of aspects 1-19; b. one or more engineered polynucleotides of aspect 20; c. one or more vector or vector systems of aspect 21; or d. cell or population thereof of aspect 22; or e. any combination of (a)-(d).
24. A formulation comprising: a. engineered intein system of any one of aspects 1-19; b. one or more engineered polynucleotides of aspect 20; c. one or more vector or vector systems of aspect 21; d. cell or population thereof of aspect 22; or e. any combination of (a)-(d); and a carrier.
25. The formulation of aspect 24, wherein the carrier is a pharmaceutically acceptable carrier.
26. A kit comprising: a. engineered intein system of any one of aspects 1-19; b. one or more engineered polynucleotides of aspect 20; c. one or more vector or vector systems of aspect 21; d. cell or population thereof of aspect 22; e. a formulation of any one of aspects 24-25; or f. any combination of (a)-(e).
27. A method of bioconjugation comprising: mixing a recombinant first amino acid sequence comprising an N-terminal intein sequence with a recombinant second amino acid sequence comprising a C-terminal intein sequence under conditions sufficient to allow bioconjugation of the first recombinant amino acid sequence and the second recombinant amino acid sequence, wherein the N-terminal intein sequence, the C-terminal intein sequence, or both are derived from a split intein of Richelia sp., Pseudomonas aeruginosa 18, Candidatus Brocadiales, or any combination thereof.
28. The method of aspect 27, wherein the split intein is a cysteine-less split intein.
29. The method of any one of aspects 27-28, wherein the N-terminal intein sequence comprises an amino acid sequence having at least 80%, 85%, 90%, 95%, 98% or 99% sequence identity to any one of SEQ ID NO: 1, 3, 5, or 7.
30. The method of any one of aspects 27-29, wherein the C-terminal intein sequence comprises an amino acid sequence having at least 80%, 85%, 90%, 95%, 98% or 99% sequence identity to any one of SEQ ID NO: 2, 4, 6, or 8.
31. The method of any one of aspects 27-30, wherein the N-terminal intein sequence is attached to a C-terminus of the first amino acid sequence with a peptide bond.
32. The method of any one of aspects 27-31, wherein the C-terminal intein sequence is attached to a N-terminus of the first amino acid sequence with a peptide bond.
33. The method of any one of aspects 27-32, further comprising a linker between the first amino acid sequence and the N-terminal intein sequence, optionally wherein the linker is a peptide linker.
34. The method of any one of aspects 27-33, further comprising a linker between the first amino acid sequence and the C-terminal intein sequence, optionally wherein the linker is a peptide linker.
35. The method of any one of claims 33-34, wherein the linker is not more than 1, 2, 3, 4, 5, 6, 7, 8, 9, 10, 11, 12, 13, 14, 15, or 20 amino acids in length.
36. The method of any one of claims 33-35, wherein linker is a Gly-Ser linker, optionally wherein the linker comprises an amino acid sequence of at least 80%, 85%, 90%, 95%, 98% or 99% sequence identity to GSGSGSGSGSGSGSGSGSGSG (SEQ ID NO: 11).
37. The method of any one of claims 33-35, wherein the linker is an Asparagine-Serine linker, optionally wherein the linker comprises an amino acid sequence of at least 80%, 85%, 90%, 95%, 98% or 99% sequence identity to ASASASASASASASASAS (SEQ ID NO: 12).
38. The method of any one of aspects 27-37, further comprising a localization tag, affinity tag, reporter tag, or any combination thereof, wherein the localization tag, affinity tag, reporter tag, or any combination thereof is operatively coupled to the first amino acid sequence, the second amino acid sequence, or both.
39. The method of any one of aspects 27-38, wherein the C-terminal intein sequence comprises X₁PYFFX₂NNILVHNS (SEQ ID NO: 10), wherein X₁ and X₂ are each independently selected from any amino acid.
40. The method of claim 39, (a) wherein X₁ is selected from N or T, (b) wherein X₂ is selected from A or G, or (c) both (a) and (b).
41. The engineered intein system of claim 39, wherein the C-terminal sequence comprises SEQ ID NO: 9.
42. The method of any one of aspects 27-41, wherein the conditions sufficient to allow bioconjugation comprise a pH ranging from about 6 to about 8.
43. The method of any one of aspects 27-42, wherein the conditions sufficient to allow bioconjugation comprise a temperature ranging from about 20° C. to about 50° C.
44. The method of any one of aspects 27-43, wherein the conditions sufficient to allow bioconjugation comprise a reducing agent, optionally wherein the reducing agent is dithiothreitol (DTT), beta mercaptoethanol (BME), tris(2-carboxyethyl)phosphine (TCEP), or cysteine.
45. The method of any one of aspects 27-44, wherein the conditions sufficient to allow bioconjugation comprise NaCl at a concentration ranging from about 0.05 M NaCl to about 2 M NaCl.

1. An engineered intein system comprising:
a recombinant first amino acid sequence comprising an N-terminal intein sequence; and
a recombinant second amino acid sequence comprising a C-terminal intein sequence,
wherein the N-terminal intein sequence, the C-terminal intein sequence, or both are derived from a split intein of Richelia sp., Pseudomonas aeruginosa 18, Candidatus Brocadiales, or any combination thereof.

2. The engineered intein system of claim 1, wherein the split intein is a cysteine-less split intein.

3. The engineered intein system of claim 1, wherein (a) the N-terminal intein sequence comprises an amino acid sequence having at least 80%, 85%, 90%, 95%, 98%, or 99% sequence identity to any one of SEQ ID NO: 1, 3, 5, or 7; (b) the C-terminal intein sequence comprises an amino acid sequence having at least 80%, 85%, 90%, 95%, 98%, or 99% sequence identity to any one of SEQ ID NO: 2, 4, 6, or 8; or (c) both (a) and (b).

4. (canceled)

5. The engineered intein system of claim 1, wherein (a) the N-terminal intein sequence is attached to a C-terminus of the first amino acid sequence with a peptide bond; (b) the C-terminal intein sequence is attached to a N-terminus of the first amino acid sequence with a peptide bond; or (c) both (a) and (b).

6. (canceled)

7. The engineered intein system of claim 1, further comprising (a) a first linker between the first amino acid sequence and the N-terminal intein sequence, optionally wherein the first linker is a peptide linker; (b) a second linker between the first amino acid sequence and the C-terminal intein sequence, optionally wherein the second linker is a peptide linker; or (c) both (a) and (b).

8. (canceled)

9. The engineered intein system of claim 7, wherein the first linker, the second linker, or both is not more than 1, 2, 3, 4, 5, 6, 7, 8, 9, 10, 11, 12, 13, 14, 15, or 20 amino acids in length; (b) the first linker, the second linker, or both is a Gly-Ser linker, optionally wherein the first linker, the second linker, or both comprises an amino acid sequence of at least 80%, 85%, 90%, 95%, 98%, or 99% sequence identity to GSGSGSGSGSGSGSGSGSGSG (SEQ ID NO: 11); (c) the first linker, the second linker, or both is an Asparagine-Serine linker, optionally wherein the first linker, the second linker, or both comprises an amino acid sequence of at least 80%, 85%, 90%, 95%, 98% or 99% sequence identity to ASASASASASASASASAS (SEQ ID NO: 12); or (d) any combination of (a)-(c).

10. (canceled)

11. (canceled)

12. The engineered intein system of claim 1, further comprising a localization tag, affinity tag, reporter tag, or any combination thereof, wherein the localization tag, affinity tag, reporter tag, or any combination thereof is operatively coupled to the first amino acid sequence, the second amino acid sequence, or both.

13. The engineered intein system of claim 1, wherein the C-terminal intein sequence comprises X₁PYFFX₂NNILVHNS (SEQ ID NO: 10), wherein X₁ and X₂ are each independently selected from any amino acid.

14. The engineered intein system of claim 13, (a) wherein X₁ is selected from N or T, (b) wherein X₂ is selected from A or G, or (c) both (a) and (b).

15. The engineered intein system of claim 13, wherein the C-terminal sequence comprises SEQ ID NO: 9.

16. The engineered intein system of claim 1, wherein (a) the system is capable of catalyzing a bioconjugation reaction at a pH ranging from about 6 to about 8; (b) wherein the system is capable of catalyzing a bioconjugation reaction at a temperature ranging from about 20° C. to about 50° C.; (c) the system is capable of catalyzing a bioconjugation reaction, wherein the system is capable of catalyzing a bioconjugation reaction in the presence of a reducing agent, optionally wherein the reducing agent is dithiothreitol (DTT), beta mercaptoethanol (BME), tris(2-carboxyethyl)phosphine (TCEP), or cysteine; (d) wherein the system is capable of catalyzing a bioconjugation reaction in the presence of about 0.05 M NaCl to about 2 M NaCl; or (e) any combination of (a)-(d).

17. (canceled)

18. (canceled)

19. (canceled)

20. An engineered polynucleotide encoding the engineered intein system of claim 1 or a component thereof.

21. A vector or vector system comprising:
one or more engineered polynucleotides of claim 20, optionally wherein at least one of the one or more engineered polynucleotides is operatively coupled to a regulatory element.

22. A cell or population thereof comprising:
a. an engineered intein system of claim 1;
b. one or more engineered polynucleotides encoding the engineered intein system;
c. one or more vector or vector systems comprising the one or more engineered polynucleotides; or
d. any combination of (a)-(c).

23. A non-human organism comprising:
a. an engineered intein system of claim 1;
b. one or more engineered polynucleotides encoding the engineered intein system;
c. one or more vector or vector systems comprising the one or more engineered polynucleotides; or
d. a cell or population thereof of comprising (a), (b), (c), or any combination thereof; or
e. any combination of (a)-(d).

24. A formulation comprising:
a. an engineered intein system of claim 1;
b. one or more engineered polynucleotides encoding the engineered intein system;
c. one or more vector or vector systems comprising the one or more engineered polynucleotides;
d. a cell or population thereof comprising (a), (b), (c), or any combination thereof; or
e. any combination of (a)-(d); and
a carrier, optionally wherein the carrier is a pharmaceutically acceptable carrier.

25. (canceled)

26. A kit comprising:
a. an engineered intein system of claim 1;
b. one or more engineered polynucleotides encoding the engineered intein system;
c. one or more vector or vector systems comprising the one or more engineered polynucleotides;
d. a cell or population thereof comprising (a), (b), (c) or any combination thereof;
e. a formulation of comprising (a), (b), (c), (d) or any combination thereof; and a carrier, optionally wherein the carrier is a pharmaceutically acceptable carrier; or
f. any combination of (a)-(e).

27. A method of bioconjugation, comprising:
mixing a recombinant first amino acid sequence comprising an N-terminal intein sequence with a recombinant second amino acid sequence comprising a C-terminal intein sequence under conditions sufficient to allow bioconjugation of the first recombinant amino acid sequence and the second recombinant amino acid sequence,
wherein the N-terminal intein sequence, the C-terminal intein sequence, or both are derived from a split intein of Richelia sp., Pseudomonas aeruginosa 18, Candidatus Brocadiales, or any combination thereof.

28. The method of claim 27, wherein the split intein is a cysteine-less split intein.

29. The method of claim 27, wherein (a) the N-terminal intein sequence comprises an amino acid sequence having at least 80%, 85%, 90%, 95%, 98% or 99% sequence identity to any one of SEQ ID NO: 1, 3, 5, or 7; (b) the C-terminal intein sequence comprises an amino acid sequence having at least 80%, 85%, 90%, 95%, 98% sequence identity to any one of SEO ID NO: 2, 4, 6, or 8; or (c) both (a) and (b).

30. (canceled)

31. The method of claim 27, wherein (a) the N-terminal intein sequence is attached to a C-terminus of the first amino acid sequence with a peptide bond; (b) the C-terminal intein sequence is attached to a N-terminus of the first amino acid sequence with a peptide bond; or (c) both (a) and (b).

32. (canceled)

33. The method of claim 27, further comprising (a) a first linker between the first amino acid sequence and the N-terminal intein sequence, optionally wherein the first linker is a peptide linker; (b) a second linker between the first amino acid sequence and the C-terminal intein sequence, optionally wherein the second linker is a peptide linker; or (c) both (a) and (b).

34. (canceled)

35. The method of claim 33 wherein the wherein the first linker, the second linker, or both is not more than 1, 2, 3, 4, 5, 6, 7, 8, 9, 10, 11, 12, 13, 14, 15, or 20 amino acids in length; (b) the first linker, the second linker, or both is a Gly-Ser linker, optionally wherein the first linker, the second linker, or both comprises an amino acid sequence of at least 80%, 85%, 90%, 95%, 98%, or 99% sequence identity to GSGSGSGSGSGSGSGSGSGSG (SEQ ID NO: 11); (c) the first linker, the second linker, or both is an Asparagine-Serine linker, optionally wherein the first linker, the second linker, or both comprises an amino acid sequence of at least 80%, 85%, 90%, 95%, 98% or 99% sequence identity to ASASASASASASASASAS (SEQ ID NO: 12); or (d) any combination of (a)-(c).

36. (canceled)

37. (canceled)

38. The method of claim 27, further comprising a localization tag, affinity tag, reporter tag, or any combination thereof, wherein the localization tag, affinity tag, reporter tag, or any combination thereof is operatively coupled to the first amino acid sequence, the second amino acid sequence, or both.

39. The method of claim 27, wherein the C-terminal intein sequence comprises X₁PYFFX₂NNILVHNS (SEQ ID NO: 10), wherein X₁ and X₂ are each independently selected from any amino acid.

40. The method of claim 39, (a) wherein X₁ is selected from N or T, (b) wherein X₂ is selected from A or G, or (c) both (a) and (b).

41. The engineered intein system of claim 34, wherein the C-terminal sequence comprises SEQ ID NO: 9.

42. The method of claim 27, wherein (a) the conditions sufficient to allow bioconjugation comprise a pH ranging from about 6 to about 8; (b) the conditions sufficient to allow bioconjugation comprise a temperature ranging from about 20° C. to about 50° C.; (c) the conditions sufficient to allow bioconjugation comprise a reducing agent, optionally wherein the reducing agent is dithiothreitol (DTT), beta mercaptoethanol (BME), tris(2-carboxyethyl)phosphine (TCEP), or cysteine; (d) the conditions sufficient to allow bioconjugation comprise NaCl at a concentration ranging from about 0.05 M NaCl to about 2 M NaCl; or (e) any combination of (a)-(d).

43. (canceled)

44. (canceled)

45. (canceled)
